(19) United States
(12) Patent Application Publication
(10) Pub. No.: US 2004/0001801 A1
(43) Pub. Date: Jan. 1, 2004

(54) Conjugates activated by cell surface proteases and therapeutic uses thereof

(75) Inventors: Edwin L. Madison, San Diego, CA (US); Joseph Edward Semple, San Diego, CA (US); George P. Vlasuk, Carlsbad, CA (US); Scott Jeffrey Kemp, San Diego, CA (US); Mallareddy Komandla, San Diego, CA (US); Daniel Vanna Siev, San Diego, CA (US)

Correspondence Address: HELLER EHRMAN WHITE & MCAULIFFE LLP, 4350 LA JOLLA VILLAGE DRIVE, 7TH FLOOR, SAN DIEGO, CA, 92122-1246, US

(73) Assignee: Corvas International, Inc.

(21) Appl. No.: 10/156,214

(22) Filed: May 23, 2002

(51) Int. Cl. A61K 38/20; A61K 38/19; A61K 38/18; C07K 14/52; C07K 14/475; C07K 14/415; A61K 39/02

(52) U.S. Cl. 424/85.1; 530/351; 530/370; 530/395; 530/399; 424/85.2; 514/8; 514/12; 424/236.1

(57) ABSTRACT

Conjugates, compositions and methods for treatment, prevention, or amelioration of one or more symptoms of cell surface protease-related diseases, including MTSP-related, urokinase-type plasminogen activator (uPA) or endotheliase-related diseases, are provided. The conjugates for use in the compositions and methods are peptidic conjugates that contain therapeutic, including cytotoxic, agents.

### RELATED APPLICATIONS

Benefit of priority under 35 U.S.C. §119(e) to U.S. provisional application Serial No. 60/293,267, filed May 23, 2001, to Edwin L. Madison, Joseph Edward Semple and George P. Vlasuk, entitled “CONJUGATES ACTIVATED BY CELL SURFACE PROTEASES AND THERAPEUTIC USES THEREOF” is claimed. The subject matter of the above-referenced application is incorporated by reference in its entirety.

### FIELD OF THE INVENTION

Conjugates, compositions and methods for localized delivery of therapeutic agents for treating a variety of disorders, such as, proliferative diseases, autoimmune diseases, infectious diseases and inflammatory diseases, are provided. The conjugates, which act as prodrugs, contain therapeutic agents and peptidic substrates that are cleaved by cell surface proteases to release therapeutic agents in the vicinity of the targeted cells.

### BACKGROUND

Effective treatment of cancer and other proliferative diseases involves administration of chemotherapeutic agents, typically systemic administration. Typically chemotherapeutic agents are cytotoxic agents that act by inhibiting proliferation or other metabolic processes, so that actively proliferating and growing cells will be targeted by the agent. Such targeting, however, is not highly specific, and the side-effects are often devastating.

Thus, a goal in pharmacology is the design of specific agents that act with high specific activity on targeted cells or tissues. This aim is of particular importance, for example, in the design of agents for treatments of diseases, such as proliferative diseases, including neoplastic disease, and diseases of viral origin, in which the ratio of toxic dose to therapeutic dose is generally close to one and the dosage must be restricted. Numerous approaches to achieving this goal have been developed. Among these are the use of conjugates that contain a targeting agent, such as an antibody and/or growth factor, and a therapeutic agent, that act on specific cells; the use of antisense technology that is targeted to specific genes and/or proteins; the use of genetic therapy to provide, for example, correct copies of defective genes or pharmaceutically active compounds, and the use of toxins that are relatively non-toxic unless delivered intracellularly. Thus far success has been limited. There are only a limited number and type of potential targeting agents, and the specificity of such agents is optimal.

Hence there is a need to develop means for delivery of therapeutic agents to targeted cells and tissues. Therefore, it is an object herein, among others, to provide methods and compounds for targeted delivery of therapeutic agents.

### SUMMARY OF THE INVENTION

Provided herein are compounds and methods for targeted delivery of therapeutic agents. The compounds are conjugates that contain a peptidic substrate for a cell surface protease, or a soluble, shed or released form thereof, and an agent that upon cleavage by the protease is a therapeutic agent or in a form that can be activated by the targeted cell or tissue or in the localter thereof. The agents include therapeutic agents, such as a cytotoxic agents, drugs, therapeutic nucleic acid moleulces, and diagnostic agents, such as labelled moieties and imaging agents. The cell surface proteases are proteases located at a cell surface and, include, but are not limited to, membrane-bound proteases such as membrane-bound serine proteases (SPs), including, for example, proteases designated MTSPs and endotheliases. Also contemplated are proteases that are located at the cell surface by virtue of a specific binding interaction with a receptor therefor. Included among such proteases is urokinase plasminogen activator (u-PA; see, e.g., Hung (1984) Adv. Exp. Med. Biol. 172:281-293; Cheng et al. (1989) Gene 69:357-363) bound to urokinase plasminogen activator receptor (u-PAR).

The conjugates contain one or more substrates for one or a plurality of cell surface proteases linked either directly or via a linker to a targeted agent, including a therapeutic agent, such as a cytotoxic agent. The conjugates provided herein contain the following components: (peptidic substrate)ₛ, (linker)_{q}, and (targeted agent)ₜ in which: at least one peptidic substrate moiety is linked with or without a linker (L) to at least one therapeutic agent, s is 1 or more and each substrate is the same or different, and is typically is between 1 and 6, generally 1, 2 or 3; q is 0 or more as long as cell surface protease(s) cleaves the peptidic substrate(s) and releases active therapeutic agent or, releases the agent in a form that is converted by the cell, tissue or surrounding environment to an active form, q is 0 to t, generally 1 to 4; t is 1 or more, generally 1 or 2 and each targeted agent are the same or different; linker refers to any linker; and the targeted agent is any agent, typically a therapeutic agent, such as a cytotoxic agent, a nucleic acid, a diagnostic agent, such as an imaging agent or labeled moiety, or a drug, including, but not limited to, anti-tumor, anti-cancer, anti-angiogenic, pro-apoptotic and anti-mitotic agents or treatments.

The therapeutic agents include any biologically active molecule. These agents include toxins, cytokines and lymphokines, growth factors, nucleic acid molecules, such as antisense nucleic acid, dsRNA, and DNA molecules. The therapeutic agents include those that are active intracellularly, such as cytotoxins, or extracellularly, such as modulators of the activity of extracellular receptors. When in the conjugates the therapeutic agents are substantially inactive, and when cleaved are released in active form or in a form that can be activated by the targeted cell or tissue or environment thereof.

In an exemplary embodiment, the conjugates for use in the methods and compositions provided herein can be represented by the formula:

(peptide^{l})ₛ-(linker)_{q}-(therapeutic agent)ₜ

or a derivative thereof, where peptide^{l} is a peptidic substrate for a cell surface protease; s is greater than or equal to 1, or is 1 to 6, or is 1 or 2, or is 1; linker is any linker; q is greater than or equal to 0, or is 0 to 4, or is 0 or 1; the therapeutic agent is, for example, a cytotoxic agent, including, but not limited to, an anti-tumor, anti-angiogenic, anti-cancer, pro-apoptotic and anti-mitotic agents; and t is 1 or more, or is 1 or 2. In these conjugates, the therapeutic agent is covalently attached, optionally via a linker L, to either the C-terminus or the N-terminus of the peptidic substrate.

In certain embodiments, peptide^{l} is a substrate for a cell surface protease whereby, upon action of the protease, the conjugate, which is substantially inactive, is cleaved at a point on the peptidic substrate chain to release a compound of the formula:

(peptide^{a})ₛ-(linker)_{q}-(therapeutic agent)ₜ

or a derivative thereof, that exhibits therapeutic activity in vitro and/or in vivo. In these conjugates, the therapeutic agent is, for example, a cytotoxic agent, and peptide^{a} is a truncated version of peptide^{l} resulting from cleavage at the P1-P1′ bond.

The conjugates can be used to target and deliver the targeted agents to specific cells, and hence can be used for the treatment any diseases that are associated with cells or tissues that express a cell surface protease, including cell-associated and cell-localized proteases. The cells on which or near which such proteases are expressed are not necessarily involved in the disease or disease process, but are present and can serve to present the protease, which cleaves the targeted conjugate.

Methods of treatment of diseases associated with cells or tissues that express a cell surface protease, including cell-associated and cell-localized proteases. The diseases include, but not limited to, proliferative diseases, autoimmune diseases, infectious diseases and inflammatory diseases. For example, diseases include e, but are not limited to, rheumatoid arthritis, lupus, multiple sclerosis, psoriasis, diabetic retinopathies, other ocular disorders, including recurrence of pterygii, scarring excimer laser surgery and glaucoma filtering surgery, various disorders of the anterior eye, cardiovascular disorders, restenosis, chronic inflammatory diseases, wounds, circulatory disorders, crest syndromes, bacterial infections, viral diseases, including AIDS, dermatological disorders, and cancer, including solid neoplasms and vascular tumors, including, but are not limited to, lung, colon, esophageal, breast, ovarian and prostate cancers.

Also provided are methods for identifying proteases to target conjugates for treatment of diseases. The methods involve identifying cell-surface protease-associated disease by identifying a cell involved in the disease process or a cell in the vicinity of the cell involved in the disease process; and identifying a cell surface protease on the cell. Conjugates that target such proteases as provided herein can then be prepared.

### DESCRIPTION OF THE FIGURES

FIGS. 1-5 provide in vitro CT₅₀ (time for 50% cleavage) (min) for exemplary conjugates provided herein: A=0.1-25 min; B=25-100 min; C=100-250 min; D=>250 min.

FIG. 1 shows exemplary doxorubicin conjugates provided herein and in vitro CT₅₀ (min) data for cleavage of the conjugates by MTSP1.

FIG. 2 shows exemplary doxorubicin conjugates provided herein and in vitro CT₅₀ (min) data for cleavage of the conjugates by u-PA.

FIG. 3 shows exemplary taxol conjugates provided herein and in vitro CT₅₀ (min) data for cleavage of the conjugates by MTSP1.

FIG. 4 shows exemplary taxol conjugates provided herein and in vitro CT₅₀ (min) data for cleavage of the conjugates by u-PA.

FIG. 5 shows exemplary doxorubicin and taxol conjugates provided herein and in vitro CT₅₀ (min) data for cleavage of the conjugates by ET1 (endotheliase 1).

### DETAILED DESCRIPTION OF EMBODIMENTS

### A. Definitions

Unless defined otherwise, all technical and scientific terms used herein have the same meaning as is commonly understood by one of skill in the art to which the invention(s) belong. All patents, patent applications, published applications and publications, Genbank sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such indentifier or address, it understood that such identifiers can change and particular information on the internet can come and go, but equivalent information can be found by searching the internet. Reference thereto evidences the availability and public dissemination of such information.

As used herein, a targeted agent is any agent intended for targeted delivery and includes therapeutic agents and diagnostic agents and any other agent intended for targeted delivery.

As used herein, targeted delivery means delivery to a selected cell or tissue that expresses a protease that releases the targeted agent. Such delivery does not have to be exclusively to such selected cell or tissue, but must include it, and generally deliveries higher amounts to such selected cells or tissues. Delivery includes introduction into a cell or tissue or binding to the cell or tissue or release in the vicinity of the cell or tissue. For example, in some instances, a tumor induces production of proteases, receptors, co-factors or substrates asssociated with the stroma; delivery, thus, includes targeting such induced stromal activities, such as proteases, receptors and/or enzyme co-factors, in invading cells or cells in the tumor that is targeted.

As used herein, therapeutic index is the ratio of LD₅₀/ED₅₀.

As used herein, a therapeutic agent is any drug or other agent that is intended for delivery to a targeted cell or tissue, such as proliferating cells, including tumor cells and cells involved in a proliferative, typically an undesirable, response. Therapeutic agents, include, but are not limited to, anti-cancer agents, anti-angiogenic agents, pro-apoptotic agents, anti-mitotic growth factors, cytokines, such as tumor necrosis factors and interleukins, and cytotoxic agents and other such agents as described herein and known to those of skill in the art. Therapeutic agents include those that are active upon internalization and also those that act extracellularly, such modulators of the activities of certain cell surface receptors, such as G proteins that transduce extracellular signals.

As used herein, an inactive therapeutic agent is a therapeutic agent that is conjugated to a peptide and thereby, either by virtue of conformational changes or size or other factors such as steric hinderance does not exhibit any or exhibits substantially reduced activity compared to the released active therapeutic agent. For example, conjugated doxorubicin is not toxic to cells until it is released from the conjugate in a form that can enter the cell. Upon cleavage of the agent from the conjugate it is in active form or in a form that is further processed by one or a plurality of steps, including enzymatically or chemically, in or on the cell, into an active form.

As used herein, an active therapeutic agent is a therapeutic agent that has been released from the conjugate by cleavage of the peptidic substrate portion of the conjugate. The active therapeutic agent is by virtue of cleavage able to exhibit its intended activity, typically by entering the cell. When conjugated the therapeutic agents have reduced or no activity as therapeutic agents, and upon cleavage are released in the vicinity of a cell.

As used herein, an anti-cancer agent (used interchangeably with “anti-tumor or anti-neoplasm agent”) refers to any agents used in the treatment of cancer. These include any agents, when used alone or in combination with other compounds, that can alleviate, reduce, ameliorate, prevent, or place or maintain in a state of remission of clinical symptoms or diagnostic markers associated with neoplasm, tumor or cancer, and can be used in methods, combinations and compositions provided herein. Non-limiting examples of anti-neoplasm agents include anti-angiogenic agents, alkylating agents, antimetabolite, certain natural products, platinum coordination complexes, anthracenediones, substituted ureas, methylhydrazine derivatives, adrenocortical suppressants, certain hormones, antagonists and anti-cancer polysaccharides.

As used herein, substantially inactive with reference to the conjugated thereapeutic agent means at least 1%, generally 10, 20, 30, 50, 60, 70, 80 or 90 or 100% inactive compared to the unconjugated therapeutic agent in a standard or art-recognized assays, such as in vitro or in vivo assays, that assess the therapeutic activity of the agent.

As used herein, a targeted cell or tissue refers to the cells or tissues that include cell surface proteases that cleave the conjugates. The cells or tissues can be involved in the disease or can be present at the disease loci or locus by virtue of participation in the disease process or merely serendipitously.

As used herein, angiogenesis is intended to broadly encompass the totality of processes directly or indirectly involved in the establishment and maintenance of new vasculature (neovascularization), including, but not limited to, neovascularization associated with tumors.

As used herein, anti-angiogenic treatment or agent refers to any therapeutic regimen and compound, that, when used alone or in combination with other treatment or compounds, can alleviate, reduce, ameliorate, prevent, or place or maintain in a state of remission, one or more clinical symptoms or diagnostic markers associated with undesired and/or uncontrolled angiogenesis. Thus, for purposes herein an anti-angiogenic agent refers to an agent that inhibits the establishment or maintenance of vasculature. Such agents include, but are not limited to, anti-tumor agents, and agents for treatments of other disorders associated with undesirable angiogenesis, such as diabetic retinopathies, hyperproliferative disorders and others.

As used herein, non-anti-angiogenic anti-tumor agents refer to anti-tumor agents that do not act primarily by inhibiting angiogenesis. Whether anti-tumor agents act primarily by inhibiting angiogenesis can be determined using the assays provided herein, or using other assays well known to those of skill in the art.

As used herein, undesired and/or uncontrolled angiogenesis refers to pathological angiogenesis wherein the influence of angiogenesis stimulators outweighs the influence of angiogenesis inhibitors. As used herein, deficient angiogenesis refers to pathological angiogenesis associated with disorders where there is a defect in normal angiogenesis resulting in aberrant angiogenesis or an absence or substantial reduction in angiogenesis.

As used herein, a cell surface protease is any protease that is located on or at a cell surface and/or proteases that are located at the cell surface by virtue of a specific binding interaction with a receptor therefor, or that is localized at or near or associated with the cell surface. An exemplary protease located at the cell surface by virtue of a specific binding interaction with a receptor therefor is urokinase plasminogen activator (u-PA) bound to urokinase plasminogen activator receptor (u-PAR). Hence cell surface proteases contemplated herein include cell surface-associated proteases. It also includes all forms thereof that can be circulating or inside a cell. To be categorized as a cell surface protease, there must be at least one form thereof that is located (i.e. on the surfaces such as transmembrane protease or bound to receptor therefor) on the surface of a cell at some point in its cycle. Cell surface protease include serine proteases, such as, but are not limited to, the transmembrane serine protease (MTSPs) and endotheliases and urokinases.

As used herein, a serine protease (SP) refers to a diverse family of proteases in which a serine residue is involved in the hydrolysis of proteins or peptides. The serine residue can be part of the catalytic triad mechanism, which includes a serine, a histidine and an aspartic acid in the catalysis, or be part of the hydroxyl/ε-amine or hydroxyl/α-amine catalytic dyad mechanism, which involves a serine and a lysine in the catalysis. Of particular interest are SPs of mammalian, including human, origin. Those of skill in this art recognize that, in general, single amino acid substitutions in non-essential regions of a polypeptide do not substantially alter biological activity (see, e.g., Watson et al. (1987) Molecular Biology of the Gene, 4th Edition, The Bejacmin/Cummings Pub. co., p.224).

As used herein shed, soluble and released forms of cell surface proteases are contemplated. Such forms include, for example, forms found in serum upon proteolytic degradation or other removal of the extracellular portion of membrane bound protease, and splice variants that do not include a transmembrane domain.

As shown herein, the protease activity of cell surface proteases and proteases associated with cells can be exploited to provide a means to concentrate therapeutic agents, such as cytotoxic agents, near such cells by providing conjugates that are activated upon cleavage by such enzymes. Such conjugates, upon the action of a cell surface protease or cell-associate protease, release the therapeutic agent, such as a cytotoxic agent, or a derivative thereof that can be converted to a therapeutic agent, locally at the site of action. As noted above, the substrates are designed to be substrates of targeted proteases that are expressed or are active on the surfaces of cells, such as tumor cells or endothelial cells, involved in or present at the site(s) or locus or loci of the disease. By virtue of specific expression, localization or activation of such proteases or the presence of receptors, substrates or enzyme co-factors therefor, administration of the conjugates provided herein permits targeting of therapeutic agents to such cells. Upon contacting with the proteases, active therapeutic agents are released in the immediate vicinity of the targeted cells. For example, specific profiles of some of the MTSPs are as follows.

As used herein, “transmembrane serine protease (MTSP)” refers to a family of transmembrane serine proteases that share common structural features as described herein (see, also Hooper et al. (2001) J. Biol. Chem.276:857-860). Thus, reference, for example, to “MTSP” encompasses all proteins encoded by the MTSP genes, including but are not limited to: MTSP1, MTSP3, MTSP4, MTSP6, MTSP7, MTSP9, MTSP10, MTSP12, MTSP20, MTSP22 and MTSP25 or an equivalent molecule obtained from any other source or that has been prepared synthetically or that exhibits the same activity. Other MTSPs include, but are not limited to, corin, enteropeptidase, human airway trypsin-like protease (HAT), TMPRSS2 and TMPRSS4. The MTSPs described herein can be used to identify other MTSPs. Methods for isolating nucleic acid encoding other MTSPs, including nucleic acid molecules encoding full-length molecules and splice variants and MTSPs from species, such as cows, sheep, goats, pigs, horses, primates, including chimpanzees and gorillas, rodents, dogs, cats and other species of interest, such as domesticated animals, farm and zoo animals are known to those of skill in the art and are outlined herein. The nucleic acid molecules described herein including those set forth in SEQ IDs can be used to obtain nucleic acid molecules encoding full-length MTSP polypeptides from human sources or from other species, such as by screening appropriate libraries using the nucleic acid molecules or selected primers or probes based thereon.

Sequences of encoding nucleic acid molecules and the encoded amino acid sequences of exemplary MTSPs and/or domains thereof are set forth in SEQ ID Nos. 1-45, 269-270 and 272-276. The term also encompasses MTSPs with amino acid substitutions that do not substantially alter activity of each member and also encompasses polyeptides encoded by splice variants thereof. Hence, encompassed are MTSPs with amino acid substitutions such that the resulting polypeptide retains at least 1%, 5%, 10%, 20%, 30%, 40%, 50%, 60%, 70%, 80%, 90% of the proteolytic activity of the unaltered polypeptide, and also encompasses MTSPs encoded by splice variants thereof and MTSPs encoded by allelic variants, such as single nucleotide polymorphisms (SNPs). Suitable substitutions, including, although not necessarily, conservative substitutions of amino acids, are known to those of skill in this art and can be made without eliminating the biological activity, such as the catalytic activity, of the resulting molecule. MTSPs include those of animal, such as mammalian, including human, origin.

As used herein, a “protease domain of an MTSP” refers to an extracellular protease domain of an MTSP that exhibits proteolytic activity and shares homology and structural features with the chymotryp-sin/trypsin family protease domains. Hence it is at least the minimal portion of the domain that exhibits proteolytic activity as assessed by standard in vitro assays. Contemplated herein are such protease domains and catalytically active portions thereof.

Exemplary MTSP polypeptides, with the protease domains indicated, are set forth in SEQ ID Nos. 1-45, 269-270 and 272-276, and including smaller portions thereof that retain or exhibit protease activity. The protease domains vary in size and constitution, including insertions and deletions in surface loops. They retain conserved structure, including at least one of the active site triad, primary specificity pocket, oxyanion hole and/or other features of serine protease domains of proteases. Thus, for purposes herein, the protease domain is a portion of a MTSP, as defined herein, and is homologous to a domain of other MTSPs. MTSPs include, MTSP1, MTSP3, MTSP4, MTSP6, MTSP7, MTSP9, MTSP10, MTSP12, MTSP20, MTSP22 and MTSP25 (see SEQ ID Nos. 1-19, 42-45, 269-270 and 272-276; see, also International PCT application No. WO 02/00860 (see SEQ ID Nos. 38 and 97 therein, which provide an MTSP12 variant); corin (SEQ ID Nos. 28 and 29), enteropeptidase (SEQ ID Nos. 30 and 31) human airway trypsin-like protease (HAT) (SEQ ID Nos. 32 and 33), hepsin (SEQ ID Nos. 34 and 35), TMPRSS2 (SEQ ID Nos. 36 and 37) and TMPRSS4 (SEQ ID Nos. 38 and 39). As with the larger class of enzymes of the chymotrypsin (S1) fold (see, e.g., Internet accessible MEROPS data base), the MTSPs protease domains share a high degree of amino acid sequence identity. The His, Asp and Ser residues necessary for activity are present in conserved motifs. In those that are activated by cleavage, the activation site, which results in the N-terminus of second chain in the two chain forms has a conserved motif and readily can be identified (see, e.g., amino acids 801-806, SEQ ID No. 29, amino acids 406-410, SEQ ID No. 31; amino acids 186-190, SEQ ID No. 33; amino acids 161-166, SEQ ID No. 35; amino acids 255-259, SEQ ID No. 37; amino acids 190-194, SEQ ID No. 39 and other as known to those of skill and the art and/or as described herein).

For example, with reference to MTSP10 (see SEQ ID Nos. 44 and 45), there disulfide bonds as follows: C₄₈₈-C₅₀₄, C₅₈₇-C₆₅₃; C₆₁₉-C₆₃₂; C₆₄₃-C₆₇₃ (see SEQ ID Nos. 44 and 45) (chymotrypsin numbering 42 to 58; 136-201; 168-182 and 191-220). Disulfide bonds form between the Cys residues C₅₇₃-C₂₉₆ to link the protease domain to another domain so that upon activation cleavage (between residues R₄₆₂ and I₄₆₃ of SEQ ID No. 45) the resulting polypeptide is a two chain molecule. The C₅₇₃ (SEQ ID NO. 45 is a free Cys in a single chain form of the protease domain. As noted the protease also can be provided as a two chain molecule. Single chain and two chain forms are proteolytically active. A two chain form is produced by bonding, typically between the C₅₇₃ and a Cys outside the protease domain, such as Cys₂₉₆. Upon activation cleavage the disulfide bond remains resulting in a two chain polypeptide. The size of chain “A” is a function the starting length of the polypeptide prior to activation cleavage between the R₄₆₂ and I₄₆₃. Any length polypeptide that includes the protease domain (residues 463-692 of SEQ ID No. 45) or catalytically active fragments thereof, is contemplated herein. Two chain forms include at least the protease domain a polypeptide from C₂₉₆ up to and including C₅₇₃.

As used herein, a two-chain form of the protease domain refers to a two-chain form that is formed from a single chain form of the protease in which the Cys pairing between, e.g., a Cys outside the protease domain such as, for example Cys₅₇₃ (SEQ ID No. 45 for MTSP), which links the protease domain to the remainder of the polypeptide, the “A” chain. A two chain protease domain form refers to any form in which the “remainder of the polypeptide”, i.e., “A” chain, is shortened and includes a Cys from outside the protease domain.

As used herein, the catalytically active domain of an MTSP refers to the protease domain. Reference to the protease domain of an MTSP generally refers to a single chain form of the protein. If the two-chain form or both forms is intended, it is so-specified. The zymogen form of each protein is a single chain, which is converted to the active two or multi chain form by activation cleavage. By active form is meant a form active in vivo or in vitro.

As used herein, activation cleavage refers to the cleavage of the protease at the N-terminus of the protease domain (generally between an R and I or V in the full-length protein. By virtue of the Cys-Cys pairing between a Cys outside the protease domain and a Cys in the protease domain (see, e.g., Cys₅₇₃ SEQ ID No. 45, upon cleavage the resulting polypeptide has two chains (“A” chain and the “B” chain, which is the protease domain of an MTSP). Cleavage can be effected by another protease or autocatalytically. The conjugates provided herein advantageously contain sites that are recognized by the active cell surface protease (or cell-associated protease) and are cleaved thereby to release active or an inactive prodrug form of a therapeutic agent.

As used herein an MTSP1, whenever referenced herein, includes at least one or all of or any combination of:

a polypeptide encoded by the sequence of nucleotides set forth in SEQ ID No. 1 or 40;

a polypeptide encoded by a sequence of nucleotides that hybridizes under conditions of low, moderate or high stringency to the sequence of nucleotides set forth in SEQ ID No. 1 or 40;

a polypeptide that comprises the sequence of amino acids set forth in SEQ ID No. 2 or 41;

a polypeptide that comprises a sequence of amino acids having at least about 40%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence of amino acids set forth in SEQ ID No. 2 or 41; and/or

a polypeptide encoded by a splice variant of the MTSP1 set forth in SEQ ID No. 1 or 40.

The MTSP1 can be from any animal, particularly a mammal, and includes but is not limited to, humans, rodents, fowl, ruminants and other animals. The full length zymogen or two chain activated form is contemplated or any domain thereof, including the protease domain, which can be a two chain activated form, or a single chain form. MTSP1 also is referred to TADG-15 and matriptase. As described below, the protein originally designated matriptase appears to be an MTSP1 splice variant or processed product.

As used herein an MTSP3, whenever referenced herein, includes at least one or all of or any combination of:

a polypeptide encoded by the sequence of nucleotides set forth in SEQ ID No. 3;

a polypeptide encoded by a sequence of nucleotides that hybridizes under conditions of low, moderate or high stringency to the sequence of nucleotides set forth in SEQ ID No. 3;

a polypeptide that comprises the sequence of amino acids set forth as amino acids 205-437 of SEQ ID No. 4;

a polypeptide that comprises a sequence of amino acids having at least about 40%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence of amino acids set forth in SEQ ID No. 4; and/or

a polypeptide encoded by a splice variant of the MTSP3 set forth in SEQ ID Nos. 3 and 4.

The MTSP3 can be from any animal, particularly a mammal, and includes but are not limited to, humans, rodents, fowl, ruminants and other animals. The full length zymogen or two chain activated form is contemplated or any domain thereof, including the protease domain, which can be a two chain activated form, or a single chain form.

As used herein an MTSP4, whenever referenced herein, includes at least one or all of or any combination of:

a polypeptide encoded by the sequence of nucleotides set forth in any of SEQ ID No. 5, 7 or 9;

a polypeptide encoded by a sequence of nucleotides that hybridizes under conditions of low, moderate or high stringency to the sequence of nucleotides set forth in any of SEQ ID Nos. 5, 7 or 9;

a polypeptide that comprises the sequence of amino acids set forth in any of SEQ ID Nos. 6, 8 or 10;

a polypeptide that comprises a sequence of amino acids having at least about 40%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence of amino acids set forth in SEQ ID No. 6, 8 or 10; and/or

a polypeptide encoded by a splice variant of the MTSP4s set forth in SEQ ID Nos. 7-10.

The MTSP4 can be from any animal, particularly a mammal, and includes but are not limited to, humans, rodents, fowl, ruminants and other animals. The full length zymogen or two chain activated form is contemplated or any domain thereof, including the protease domain, which can be a two chain activated form, or a single chain form.

As used herein an MTSP6, whenever referenced herein, includes at least one or all of or any combination of:

a polypeptide encoded by the sequence of nucleotides set forth in any of SEQ ID No. 11;

a polypeptide encoded by a sequence of nucleotides that hybridizes under conditions of low, moderate or high stringency to the sequence of nucleotides set forth in any of SEQ ID Nos. 11;

a polypeptide that comprises the sequence of amino acids set forth in any of SEQ ID No. 12;

a polypeptide that comprises a sequence of amino acids having at least about 40%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence of amino acids set forth in SEQ ID No. 12; and/or

a polypeptide encoded by a splice variant of the MTSP6 set forth in SEQ ID No. 12.

The MTSP6 can be from any animal, particularly a mammal, and includes but are not limited to, humans, rodents, fowl, ruminants and other animals. The full length zymogen or two chain activated form is contemplated or any domain thereof, including the protease domain, which can be a two chain activated form, or a single chain form. Of particular interest herein is the MTSP6 of SEQ ID No. 12.

As used herein an MTSP7, whenever referenced herein, includes at least one or all of or any combination of:

a polypeptide encoded by the sequence of nucleotides set forth in SEQ ID No. 13;

a polypeptide encoded by a sequence of nucleotides that hybridizes under conditions of low, moderate or high stringency to the sequence of nucleotides set forth in SEQ ID No. 13;

a polypeptide that comprises the sequence of amino acids set forth in SEQ ID No. 13;

a polypeptide that comprises a sequence of amino acids having at least about 40%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence of amino acids set forth in SEQ ID No. 14; and/or

a polypeptide encoded by a splice variant of the MTSP7 set forth in SEQ ID No. 13.

The MTSP7 can be from any animal, particularly a mammal, and includes but are not limited to, humans, rodents, fowl, ruminants and other animals. The full length zymogen or two chain activated form is contemplated or any domain thereof, including the protease domain, which can be a two chain activated form, or a single chain form.

As used herein an MTSP9, whenever referenced herein, includes at least one or all of or any combination of:

a polypeptide encoded by the sequence of nucleotides set forth in SEQ ID No. 17 or SEQ ID No. 42;

a polypeptide encoded by a sequence of nucleotides that hybridizes under conditions of low, moderate or high stringency to the sequence of nucleotides set forth in SEQ ID No. 17 or 42;

a polypeptide that comprises the sequence of amino acids set forth in SEQ ID No. 18 or 43;

a polypeptide that comprises a sequence of amino acids having at least about 40%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence of amino acids set forth in SEQ ID No. 18 or 270; and/or

a polypeptide encoded by a splice variant of the MTSP9 set forth in SEQ ID No. 17.

The MTSP9 can be from any animal, particularly a mammal, and includes but are not limited to, humans, rodents, fowl, ruminants and other animals. The full length zymogen or two chain activated form is contemplated or any domain thereof, including the protease domain, which can be a two chain activated form, or a single chain form.

As used herein an MTSP10, whenever referenced herein, includes at least one or all of or any combination of:

a polypeptide encoded by the sequence of nucleotides set forth in SEQ ID No. 44;

a polypeptide encoded by a sequence of nucleotides that hybridizes under conditions of low, moderate or high stringency to the sequence of nucleotides set forth in SEQ ID No. 44;

a polypeptide that comprises the sequence of amino acids set forth in SEQ ID No. 45;

a polypeptide that comprises a sequence of amino acids having at least about 40%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence of amino acids set forth in SEQ ID No. 45; and/or

a polypeptide encoded by a splice variant of the MTSP10 set forth in SEQ ID No. 44.

The MTSP10 can be from any animal, particularly a mammal, and includes but are not limited to, humans, rodents, fowl, ruminants and other animals. The full length zymogen or two chain activated form is contemplated or any domain thereof, including the protease domain, which can be a two chain activated form, or a single chain form.

MTSP10 polypeptides, including, but not limited to splice variants thereof, and nucleic acids encoding MTSPs, and domains, derivatives and analogs thereof are provided herein. Single chain protease domains that have an N-terminus functionally equivalent to that generated by activation of the zymogen form of MTSP10 are also provided. The cleavage site for the protease domain of MTSP10 is between amino acid R and amino acids I (R↓IIGGT) (residues 462-467 SEQ ID No. 45).

As used herein an MTSP12, whenever referenced herein, includes at least one or all of or any combination of: SEQ ID No. 19 and 20

a polypeptide encoded by the sequence of nucleotides set forth in SEQ ID No. 19 or by a sequence of nucleotides that includes nucleotides that encode the sequence of amino acids set forth in SEQ ID No. 20;

a polypeptide encoded by a sequence of nucleotides that hybridizes under conditions of low, moderate or high stringency to the sequence of nucleotides set forth in is set forth as SEQ ID No. 19;

a polypeptide that includes the sequence of amino acids set forth in SEQ ID No. 20 or a catalytically active portion thereof;

a polypeptide that includes a sequence of amino acids having at least about 40%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence of amino acids set forth in SEQ ID No. 20; and/or

a polypeptide encoded by a splice variant of the MTSP12 that includes the sequence of amino acids set forth in SEQ ID No. 20.

In particular, the MTSP12 polypeptide, with the protease domains as indicated in SEQ ID Nos. 19 and 20, is provided. The polypeptide is a single or multi-chain polypeptide. A protease domain of an MTSP12, whenever referenced herein, includes at least one or all of or any combination of or a catalytically active portion of:

a polypeptide that includes the sequence of amino acids set forth in SEQ ID No. 20 or a catalytically active portion thereof but that does not include the sequence of amino acids set forth in SEQ ID No. 271;

a polypeptide that includes the sequence of amino acids set forth in SEQ ID No. 272 or a catalytically active fragment thereof;

a polyeptide containing amino acids 237 to 456 of SEQ ID No. 6, a polypeptide containing amino aicds 538 to 765 of SEQ ID No. 20, and a polypeptide containing amino acids 861 to 1087 of SEQ ID No. 20, but that does not include the sequence of amino acids set forth in SEQ ID No. 271;

a polypeptide encoded by a sequence of nucleotides that hybridizes under conditions of low, moderate or high stringency to a sequence of nucleotides that encodes any of the polypeptides of a)-c);

a polypeptide encoded by a sequence of nucleotides that hybridizes under conditions of low, moderate or high stringency to the sequence of nucleotides set forth in SEQ ID No. 20 but that does not encode the sequence of amino acids set forth in SEQ ID No. 271;

a polypeptide that includes a sequence of amino acids having at least about 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence of amino acids set forth in SEQ ID No. 20;

a polypeptide that includes a sequence of amino acids having at least about 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence of amino acids of the polypeptides of a)-e);

a polypeptide encoded by a splice variant of a sequence of nucleotides that encodes an MTSP12 of any of the above.

Smaller portions thereof that retain protease activity are also provided. The MTSP12 can be from any animal, particularly a mammal, and includes but are not limited to, humans, rodents, fowl, ruminants and other animals. The full-length zymogen or two-chain activated form is contemplated or any domain thereof, including the protease domain, which can be a two-chain activated form, or a single chain form. MTSP12 also includes the variant described International PCT application No. WO 02/00860 (see SEQ ID Nos. 38 and 97 therein).

As used herein an MTSP20, whenever referenced herein, includes at least one or all of or any combination of:

a polypeptide encoded by the sequence of nucleotides set forth in SEQ ID No. 273;

a polypeptide encoded by a sequence of nucleotides that hybridizes under conditions of low, moderate or high stringency to the sequence of nucleotides set forth in SEQ ID No. 273;

a polypeptide that comprises the sequence of amino acids set forth in SEQ ID No. 273;

a polypetide that comprises a sequence of amino acids having at least about 40%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence of amino acids set forth in SEQ ID No. 274; and/or a polypeptide encoded by a splice variant of the MTSP20 encoded by the sequence of nucleotides that includes the sequence set forth in SEQ ID No. 273.

The MTSP20 may be from any animal, particularly a mammal, and includes but are not limited to, humans, rodents, fowl, ruminants and other animals. The full length zymogen or two-chain activated form is contemplated or any domain thereof, including the protease domain, which can be a two-chain activated form, or a single chain form.

As used herein an MTSP22, whenever referenced herein, includes at least one or all of or any combination of:

a polypeptide encoded by the sequence of nucleotides set forth in SEQ ID No. 275;

a polypeptide encoded by a sequence of nucleotides that hybridizes under conditions of low, moderate or high stringency to the sequence of nucleotides set forth in SEQ ID No. 275;

a polypeptide that comprises the sequence of amino acids set forth in SEQ ID No. 276;

a polypetide that comprises a sequence of amino acids having at least about 40%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence of amino acids set forth in SEQ ID No. 276; and/or

a polypeptide encoded by a splice variant of the MTSP22 set forth in SEQ ID No. 275.

The MTSP22 may be from any animal, particularly a mammal, and includes but are not limited to, humans, rodents, fowl, ruminants and other animals. The full length zymogen or two-chain activated form is contemplated or any domain thereof, including the protease domain, which can be a two-chain activated form, or a single chain form.

As used herein an MTSP25, whenever referenced herein, includes at least one or all of or any combination of:

a polypeptide encoded by the sequence of nucleotides set forth in SEQ ID No. 269;

a polypeptide encoded by a sequence of nucleotides that hybridizes under conditions of low, moderate or high stringency to the sequence of nucleotides set forth in SEQ ID No. 269;

a polypeptide that comprises the sequence of amino acids set forth in SEQ ID No. 270;

a polypetide that comprises a sequence of amino acids having at least about 40%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence of amino acids set forth in SEQ ID No. 270; and/or

a polypeptide encoded by a splice variant of the MTSP25 set forth in SEQ ID No. 269.

The MTSP25 may be from any animal, particularly a mammal, and includes but are not limited to, humans, rodents, fowl, ruminants and other animals. The full length zymogen or two-chain activated form is contemplated or any domain thereof, including the protease domain, which can be a two-chain activated form, or a single chain form.

As used herein, a human protein is one encoded by nucleic acid present in the genome of a human, including all allelic variants and conservative variations as long as they are not variants found in other mammals.

As used herein, not substantially cleaved by plasmin or prostate specific antigen (PSA) (or other non-cell surface-associated protease), means in comparable in vitro assays (under optimal conditions for each enzyme) in which the activity of a targeted cell surface membrane protease or catalytically active portion of the activity of the protease domain (or a catalytically active form thereof) of prostate specific antigen (PSA) or plasmin for cleavage of the conjugate is compared, the relative activity is greater than at least 2:1, 3:1, 4:1, 5:1, 10:1, 50:1 or 100:1.

As used herein, activity refers to the ratio k_{cat}/Kₘ, where k_{cat} is the rate of catalytic turnover for a particular enzyme, and Kₘ is the Michaelis constant for the binding of the substrate.

As used herein, a “nucleic acid encoding a protease domain or catalytically active portion of a MTSP” shall be construed as referring to a nucleic acid encoding only the recited single chain protease domain or active portion thereof, and not the other contiguous portions of the MTSP as a continuous sequence.

As used herein, a CUB domain is a motif that mediates protein-protein interactions in complement components C1r/C1s and has also been identified in various proteins involved in developmental processes.

As used herein, a zymogen is an enzymatically inactive protein (i.e, typically, but not necessarily, less than 1% of active form) that is converted to a proteolytic enzyme by the action of an activator, including by autoactivation. Inactive means less active than the form those of skill in the art consider to be the active form of the enzyme. The ratio of activity of a zymogen to the activated form varies from enzyme-to-enzyme.

As used herein, “disease or disorder” refers to a pathological condition in an organism resulting from, e.g., infection or genetic defect, and characterized by identifiable symptoms. The diseases contemplated for treatment herein are any for which a cell surface protease, including a cell-localized or cell-associated protease is asssociated with a targeted cell or tissue involved in the disease or disease process. Such association can be because the protease is involved in the disease or is serendipitously associated with cells involved with the disease. These diseases herein are called cell surface protease-associated diseases. Hence, to treat th disease a cellsurface protease is identified that is expressed on cells associated with the disorder, such as, for example, immune cells for treating inflammatory diseases, and virally infected cells for treating viral diseases. The conjugate is designed as described herein for cleavage by the selected protease.

As used herein, neoplasm (neoplasia) refers to abnormal new growth, and thus means the same as tumor, which can be benign or malignant. Unlike hyperplasia, neoplastic proliferation persists even in the absence of the original stimulus.

As used herein, neoplastic disease refers to any disorder involving cancer, including tumor development, growth, metastasis and progression.

As used herein, cancer refers to a general term for diseases caused by any type of malignant tumor.

As used herein, malignant, as applied to tumors, refers to primary tumors that have the capacity of metastasis with loss of growth control and positional control.

As used herein, endotheliase refers to a mammalian protein, including human protein, that has a transmembrane domain and is expressed or active on the surface of endothelial cells and includes a protease domain, particularly an extracellular protease domain, and is generally a serine protease (see, also U.S. application Ser. No. 09/717,473 and International PCT application No. WO 01/36604). Thus, reference, for example, to endotheliase encompasses all proteins encoded by the endotheliase gene family, or an equivalent molecule obtained from any other source or that has been prepared synthetically or that exhibits the same activity. The endotheliase gene family are transmembrane proteases expressed or active in endothelial cells. These proteases include serine proteases. These include proteins that have these features and also include a protease domain that exhibits sequence homology to the endotheliases 1 and 2. Endotheliase 1 and 2, for example exhibit about 40% or 45% identity. Sequence homology means sequence identity along its length when aligned to maximize identity of at least about 25%, 40%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% or greater number of residues. Sequence homology also is assessed by determining whether the encoding sequences of nucleic acids hybridize under conditions of at least moderate, or for more closely related proteins, high stringency to the nucleic acid molecules provided herein or to those that encode the same proteins but differ in sequence by virtue of the degeneracy of the genetic code. In addition, “endotheliases” encompasses endotheliases with amino acid substitutions, including those set forth in Table 1, such that the resulting polypeptide retains at least 1%, 5%, 10%, 20%, 30%, 40%, 50%, 60%, 70%, 80%, 90% of the proteolytic activity of the unaltered polypeptide. Suitable substitutions of amino acids are known to those of skill in this art and can be made generally without altering the biological activity of the resulting molecule. As noted, those of skill in this art recognize that, in general, single amino acid substitutions in non-essential regions of a polypeptide do not substantially alter biological activity (see, e.g., Watson et al. Molecular Biology of the Gene, 4th Edition, 1987, The Bejacmin/Cummings Pub. Co., p.224). Also included within the definition of “endotheliases”, is the catalytically active fragment or shed forms of an endotheliase.

As used herein an endotheliase 1, whenever referenced herein, includes at least one or all of or any combination of:

a polypeptide encoded by the sequence of nucleotides set forth in SEQ ID No. 21;

a polypeptide encoded by a sequence of nucleotides that hybridizes under conditions of low, moderate or high stringency to the sequence of nucleotides set forth in SEQ ID No. 21;

a polypeptide that comprises the sequence of amino acids set forth in SEQ ID No. 22;

a polypeptide that comprises a sequence of amino acids having at least about 40%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence of amino acids set forth in SEQ ID No. 22; and/or

a polypeptide encoded by a splice variant of a nucleic acid molecule that encodes a protein containing the polypeptide set forth in SEQ ID No. 22.

The endotheliase 1 can be from any animal, particularly a mammal, and includes but are not limited to, humans, rodents, fowl, ruminants and other animals. The full length zymogen or two chain activated form is contemplated or any domain thereof, including the protease domain, which can be a two chain activated form, or a single chain form.

As used herein an endotheliase 2, whenever referenced herein, includes at least one or all of or any combination of:

a polypeptide encoded by the sequence of nucleotides set forth in SEQ ID No. 23 or 25;

a polypeptide encoded by a sequence of nucleotides that hybridizes under conditions of low, moderate or high stringency to the sequence of nucleotides set forth in SEQ ID No. 23 or 25;

a polypeptide that comprises the sequence of amino acids set forth in SEQ ID No. 24 or 26;

a polypeptide that comprises a sequence of amino acids having at least about 40%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence of amino acids set forth in SEQ ID No. 24 or 26; and/or

a polypeptide encoded by a splice variant of a nucleic acid set forth in SEQ ID No. 23 or 25.

The endotheliase 2 can be from any animal, particularly a mammal, and includes but are not limited to, humans, rodents, fowl, ruminants and other animals. The full length zymogen or two chain activated form is contemplated or any domain thereof, including the protease domain, which can be a two chain activated form, or a single chain form.

As used herein, the protease domain of an endotheliase refers to the polypeptide portion of the endotheliase that is the extracellular portion that exhibits protease activity. The protease domain is a polypeptide that includes at least the minimum number of amino acids, generally more than 50 or 100, required for protease activity. Protease activity can be assessed empirically, such as by testing the polypeptide for its ability to act as a protease. Assays, such as those described in the EXAMPLES, with the exception that a known endotheliase substrate is employed in place of the test compounds, can be used to assess protease activity. Furthermore, since proteases, particularly serine proteases, have characteristic structures and sequences or motifs, the protease domain can be readily identified by such structure and sequence or motif.

As used herein, a portion of protease domain of endotheliase refers to a portion of protease domain of endotheliase that is located within or is the extracellular domain of an endotheliase and exhibits serine proteolytic activity. Hence, it is at least the minimal portion of the extracellular domain that exhibits proteolytic activity as assessed by standard assays. An exemplary protease domain of an endotheliase is set forth in SEQ ID No. 22 and as amino acids 321-688 and 321-562 of SEQ ID Nos. 24 and 26, respectively. Smaller portions thereof that retain protease activity are contemplated. The protease domains vary in size and constitution, including insertions and deletions in surface loops. Such domains exhibit conserved structure, including at least one structural feature, such as the active site triad, primary specificity pocket, oxyanion hole and/or other features of serine protease domains of proteases. Thus, for purposes herein, the protease domain is a portion of an endotheliase, as defined herein, but is homologous in terms of structural features and retention of sequence of similarity or homology the protease domain of chymotrypsin or trypsin.

As used herein, homologous means about greater than about 25%, 40%, 60%, 80%, 90%, 95%, 98% or greater sequence identity. By sequence identity, the number of conserved amino acids as determined by standard alignment algorithms programs, and used with default gap penalties established by each supplier. Also homology can be assessed by conserved nucleic acid sequence, which includes anything that hybridizes under at least low stringency conditions and encodes the domain. Similarly, nucleic acid sequence alignment programs are commercially available (DNAStar “MegAlign” program (Madison, Wis.) and the University of Wisconsin Genetics Computer Group (UWG) “Gap” program (Madison, Wis.)). Substantially homologous nucleic acid molecules would hybridize typically at moderate stringency or at high stringency all along the length of the nucleic acid of interest. Also contemplated are nucleic acid molecules that contain degenerate codons in place of codons in the hybridizing nucleic acid molecule.

As used herein, recitation that a polypeptide consists essentially of the protease domain means that the only endotheliase portion of the polypeptide is a protease domain or a catalytically active portion thereof. The polypeptide can optionally include additional non-endotheliase-derived sequences of amino acids.

As used herein, domain refers to a portion of a molecule, e.g., proteins or nucleic acids, that is structurally and/or functionally distinct from other portions of the molecule.

As used herein, an active form of a protease refers to an enzyme that catalyzes hydrolysis of proteins or peptides. Reference to a protease includes the active and zymogen or other less active form.

As used herein, nucleic acids include DNA, RNA and analogs thereof, including peptide nucleic acids (PNA) and mixtures thereof. Nucleic acids can be single or two stranded. When referring to probes or primers, optionally labeled, with a detectable label, such as a fluorescent or radiolabel, single-stranded molecules are contemplated. Such molecules are typically of a length such that their targets are statistically unique or of low copy number (typically less than 5, generally less than 3) for probing or priming a library. Generally a probe or primer contains at least 14, 16 or 30 contiguous of sequence complementary to or identical to a gene of interest. Probes and primers can be 10, 20, 30, 50, 100 or more nucleic acids long.

As used herein, nucleic acid encoding a fragment or portion of an endotheliase refers to a nucleic acid encoding only the recited fragment or portion of endotheliase protein, and not the other contiguous portions of the endotheliase as a continuous sequence.

As used herein, heterologous nucleic acid is nucleic acid that, if it is DNA encodes RNA, or, if RNA, encodes proteins that generally are not normally produced in vivo by the cell in which it is expressed or that mediates or encodes mediators that alter expression of endogenous nucleic acid, such as DNA, by affecting transcription, translation, or other regulatable biochemical processes or that is located in a different locus from its normal locus. Heterologous nucleic acid is generally not endogenous to the cell into which it is introduced, but has been obtained from another cell or prepared synthetically. Generally, although not necessarily, such nucleic acid encodes RNA and proteins that are not normally produced by the cell in which it is now expressed.

Heterologous nucleic acid, such as DNA, also be referred to as foreign nucleic acid, such as DNA. Any nucleic acid, such as DNA, that one of skill in the art would recognize or consider as heterologous or foreign to the cell in which is expressed is herein encompassed by heterologous nucleic acid; heterologous nucleic acid includes exogenously added nucleic acid that is also expressed endogenously. Examples of heterologous nucleic acid include, but are not limited to, nucleic acid that encodes traceable marker proteins, such as a protein that confers drug resistance, nucleic acid that encodes therapeutically effective substances, such as anti-cancer agents, enzymes and hormones, and nucleic acid, such as DNA, that encodes other types of proteins, such as antibodies, and RNA, such as RNA interference (RNAi) or other double-stranded RNA, and antisense RNA. Antibodies that are encoded by heterologous nucleic acid can be secreted or expressed on the surface of the cell in which the heterologous nucleic acid has been introduced.

For example, nucleic acid can be the the targeted agent, such as the therapeutic or diagnostic agent, in the conjugate. Nucleic acids, include ds RNA use for RNA interference (RNAi) (see, e.g. Chuang et al. (2000) Proc. Natl. Acad. Sci. U.S.A. 97:4985) which is employed to inhibit the expression of a targeted gene by generating loss-of-function. Methods relating to the use of RNAi to silence genes in organisms including, mammals, C. elegans, Drosophila and plants, and humans are known (see, e.g., Fire et al. (1998) Nature 391:806-811 Fire (1999) Trends Genet. 15:358-363; Sharp (2001) Genes Dev. 15:485-490; Hammond, et al. (2001) Nature Rev. Genet.2:110-1119; Tuschl (2001) Chem. Biochem. 2:239-245; Hamilton et al. (1999) Science 286:950-952; Hammond et al. (2000) Nature 404:293-296; Zamore et al. (2000) Cell 101:25-33; Bernstein et al. (2001) Nature 409: 363-366; Elbashir et al. (2001) Genes Dev. 15:188-200; Elbashir et al. (2001) Nature 411:494-498; International PCT application No. WO 01/29058; International PCT application No. WO 99/32619). By selecting appropriate sequences, expression of dsRNA can interfere with accumulation of endogenous mRNA encoding a targeted gene product. Regions that include at least about 21 nucleotides and that are selective (i.e. whose target is unique) for the nucleic acid encoding a targeted gene product are used to prepare the RNAi.

As used herein, genetic therapy involves the transfer of heterologous nucleic acid, such as DNA, into certain cells, target cells, of a mammal, particularly a human, with a disorder or conditions for which such therapy is sought. The nucleic acid molecules are included in a conjugate linked via a cell surface protein cleavage site. The nucleic acid, such as DNA, is introduced into the selected target cells in a manner such that the heterologous nucleic acid, such as DNA, is expressed and a therapeutic product encoded thereby is produced. Alternatively the heterologous nucleic acid, such as DNA, can in some manner mediate expression of DNA that encodes the therapeutic product, or it can encode a product, such as a peptide or RNA that in some manner mediates, directly or indirectly, expression of a therapeutic product. Genetic therapy can also be used to deliver nucleic acid encoding a gene product that replaces a defective gene or supplements a gene product produced by the mammal or the cell in which it is introduced. The introduced nucleic acid can encode a therapeutic compound, such as a growth factor inhibitor thereof, or a tumor necrosis factor or inhibitor thereof, such as a receptor therefor, that is not normally produced in the mammalian host or that is not produced in therapeutically effective amounts or at a therapeutically useful time. The heterologous nucleic acid, such as DNA, encoding the therapeutic product can be modified prior to introduction into the cells of the afflicted host in order to enhance or otherwise alter the product or expression thereof. Genetic therapy can also involve delivery of an inhibitor or repressor or other modulator of gene expression, such dsRNA or antisense or other nucleic acid molecule. The conjugates herein can be used to deliver a product, such as a nucleic acid for gene therapy.

As used herein, a therapeutically effective product for gene therapy is a product that is encoded by heterologous nucleic acid, typically DNA, that, upon introduction of the nucleic acid into a host, a product is expressed that ameliorates or eliminates the symptoms, manifestations of an inherited or acquired disease or that cures the disease. Also included are biologically active nucleic acid molecules, such as RNAi and antisense.

As used herein, a sequence complementary to at least a portion of an RNA, with reference to antisense oligonucleotides, means a sequence having sufficient complementarily to be able to hybridize with the RNA, generally under moderate or high stringency conditions, forming a stable duplex; in the case of double-stranded SP antisense nucleic acids, a single strand of the duplex DNA (or dsRNA) can thus be tested, or triplex formation can be assayed. The ability to hybridize depends on the degree of complementarily and the length of the antisense nucleic acid. Generally, the longer the hybridizing nucleic acid, the more base mismatches with a SP encoding RNA it can contain and still form a stable duplex (or triplex, as the case can be). One skilled in the art can ascertain a tolerable degree of mismatch by use of standard procedures to determine the melting point of the hybridized complex.

Amino acid substitutions can be made or occur in any SPs and protease domains thereof. Amino acid substitutions include conservative substitutions, such as those set forth in Table 1, which do not eliminate proteolytic activity. As described herein, substitutions that alter properties of the proteins, such as removal of cleavage sites and other such sites are also contemplated; such substitutions are generally non-conservative, but can be readily effected by those of skill in the art.

Suitable conservative substitutions of amino acids are known to those of skill in this art and can be made generally without altering the biological activity, for example enzymatic activity, of the resulting molecule. Also included within the definition, is the catalytically active fragment of an SP, particularly a single chain protease portion. Conservative amino acid substitutions are made, for example, in accordance with those set forth in TABLE 1 as follows:

TABLE 1

| | |
|---|---|
| Ala (A) | Gly; Ser |
| Arg (R) | Lys, Orn |
| Asn (N) | Gln; His |
| Asp (D) | Glu |
| Cys (C) | Ser |
| Gln (Q) | Asn |
| Glu (E) | Asp |
| Gly (G) | Ala; Pro |
| His (H) | Asn; Gln |
| Ile (I) | Leu; Val; Nle; Met |
| Leu (L) | Ile; Val; Nle; Met |
| Lys (K) | Arg; Gln; Glu |
| Met (M) | Leu; Tyr; Ile; Nle |
| Phe (F) | Met; Leu; Tyr, Trp |
| Ser (S) | Thr |
| Thr (T) | Ser |
| Trp (W) | Tyr; Phe |
| Tyr (Y) | Trp; Phe |
| Val (V) | Ile; Leu; Nle; Met |

Other substitutions are also permissible and can be determined empirically or in accord with known conservative substitutions. For example, one or more amino acid residues within the sequence can be substituted by another amino acid of a similar polarity which acts as a functional equivalent, resulting in a silent alteration. Substitutes for an amino acid within the sequence can be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

As used herein, the amino acids, which occur in the various amino acid sequences appearing herein, are identified according to their well-known, three-letter or one-letter abbreviations. The nucleotides, which occur in the various DNA fragments, are designated with the standard single-letter designations used routinely in the art. Other abbreviations, include: hR or hArg for homoarginine; hY or hTyr for homotyrosine; Cha for cyclohexylalanine; Amf for 4-aminomethylphenylalanine; DPL for 2-(4,6-dimethylpyrimidinyl)lysine; (imidazolyl)K for N′-(2-imidazolyl)lysine; Me2PO3-Y for 0-dimethylphosphotyrosine; O—Me—Y for O-methyltyrosine; TIC for tetrahydro-3-isoquinoline carboxylic acid; MeL for 2-keto-3-amino-5-methylhexane; DAP for 1,3-diaminopropane; TFA for trifluoroacetic acid; AA for acetic acid.

As used herein, a splice variant refers to a variant produced by differential processing of a primary transcript of genomic DNA that results in more than one type of mRNA.

As used herein, a probe or primer based on a nucleotide sequence disclosed herein, includes at least 10, 14, generally at least 16 or 30 or 100 contiguous sequence of nucleotides.

As used herein, antisense polynucleotides refer to synthetic sequences of nucleotide bases complementary to mRNA or the sense strand of double-stranded DNA. Admixture of sense and antisense polynucleotides under appropriate conditions leads to the binding of the two molecules, or hybridization. When these polynucleotides bind to (hybridize with) mRNA, inhibition of protein synthesis (translation) occurs. When these polynucleotides bind to double-stranded DNA, inhibition of RNA synthesis (transcription) occurs. The resulting inhibition of translation and/or transcription leads to an inhibition of the synthesis of the protein encoded by the sense strand. Antisense nucleic acid molecules typically contain a sufficient number of nucleotides to specifically bind to a target nucleic acid, generally at least 5 contiguous nucleotides, often at least 14 or 16 or 30 contiguous nucleotides or modified nucleotides complementary to the coding portion of a nucleic acid molecule that encodes a gene of interest, for example, nucleic acid encoding a single chain protease domain of an SP.

As used herein, an array refers to a collection of elements, such as antibodies, containing three or more members. An addressable array is one in which the members of the array are identifiable, typically by position on a solid phase support. Hence, in general the members of the array are immobilized on discrete identifiable loci on the surface of a solid phase.

As used herein, antibody refers to an immunoglobulin, whether natural or partially or wholly synthetically produced, including any derivative thereof that retains the specific binding ability of the antibody. Hence antibody includes any protein having a binding domain that is homologous or substantially homologous to an immunoglobulin binding domain. Antibodies include members of any immunoglobulin claims, including IgG, IgM, IgA, IgD and IgE.

As used herein, antibody fragment refers to any derivative of an antibody that is less than full-length, retaining at least a portion of the full-length antibody's specific binding ability. Examples of antibody fragments include,but are not limited to, Fab, Fab′, F(ab)₂, single-chain Fvs (scFV), FV, dsFV diabody and Fd fragments. The fragment can include multiple chains linked together, such as by disulfide bridges. An antibody fragment generally contains at least about 50 amino acids and typically at least 200 amino acids.

As used herein, an Fv antibody fragment is composed of one variable heavy domain (V_{H}) and one variable light domain linked by noncovalent interactions.

As used herein, a dsFV refers to an Fv with an engineered intermolecular disulfide bond, which stabilizes the V_{H}-V_{L} pair.

As used herein, an F(ab)₂ fragment is an antibody fragment that results from digestion of an immunoglobulin with pepsin at pH 4.0-4.5; it can be recombinantly expressed to produce the equivalent fragment.

As used herein, Fab fragments are antibody fragments that result from digestion of an immunoglobulin with papain; they can be recombinantly expressed to produce the equivalent fragment.

As used herein, scFVs refer to antibody fragments that contain a variable light chain (V_{L}) and variable heavy chain (V_{H}) covalently connected by a polypeptide linker in any order. The linker is of a length such that the two variable domains are bridged without substantial interference. Exemplarly linkers include, but are not limited to, (Gly-Ser)ₙ residues, which can include ome Glu or Lys residues dispersed throughout, for example, to increase solubility.

As used herein, humanized antibodies refer to antibodies that are modified to include human sequences of amino acids so that administration to a human does not provoke an immune response. Methods for preparation of such antibodies are known. For example, to produce such antibodies, the encoding nucleic acid in the hybridoma or other prokaryotic or eukaryotic cell, such as an E. coli or a CHO cell, that expresses the monoclonal antibody is altered by recombinant nucleic acid techniques to express an antibody in which the amino acid composition of the non-variable region is based on human antibodies. Computer programs have been designed to identify such non-variable regions.

As used herein, diabodies are dimeric scFV; diabodies typically have shorter peptide linkers than scFvs, and they generally dimerize.

As used herein, production by recombinant means by using recombinant DNA methods means the use of the well known methods of molecular biology for expressing proteins encoded by cloned DNA.

As used herein, the term assessing is intended to include quantitative and qualitative determination in the sense of obtaining an absolute value for the activity of an SP, or a domain thereof, present in the sample, and also of obtaining an index, ratio, percentage, visual or other value indicative of the level of the activity. Assessment can be direct or indirect and the chemical species actually detected need not of course be the proteolysis product itself but can for example be a derivative thereof or some further substance.

As used herein, biological activity refers to the in vivo activities of a compound or physiological responses that result upon in vivo administration of a compound, composition or other mixture. Biological activity, thus, encompasses therapeutic effects and pharmaceutical activity of such compounds, compositions and mixtures. Biological activities can be observed in in vitro systems designed to test or use such activities.

As used herein, a combination refers to any association between two or among more items.

As used herein, fluid refers to any composition that can flow. Fluids thus encompass compositions that are in the form of semi-solids, pastes, solutions, aqueous mixtures, gels, lotions, creams and other such compositions.

As used herein, an effective amount of a compound for treating a particular disease is an amount that is sufficient to ameliorate, or in some manner reduce the symptoms associated with the disease. Such amount can be administered as a single dosage or can be administered according to a regimen, whereby it is effective. The amount can cure the disease but, typically, is administered in order to ameliorate the symptoms of the disease. Repeated administration can be required to achieve the desired amelioration of symptoms.

As used herein, equivalent, when referring to two sequences of nucleic acids, means that the two sequences in question encode the same sequence of amino acids or equivalent proteins. When equivalent is used in referring to two proteins or peptides, it means that the two proteins or peptides have substantially the same amino acid sequence with amino acid substitutions (see, e.g., Table 1, above) that do not substantially alter the activity or function of the protein or peptide (i.e, retain at least about 1% of the activity). When equivalent refers to a property, the property does not need to be present to the same extent (e.g., two peptides can exhibit different rates of the same type of enzymatic activity), but the activities are generally substantially the same. Complementary, when referring to two nucleotide sequences, means that the two sequences of nucleotides are capable of hybridizing, typically with less than 25%, often with less than 15%, or even less than 5% or with no mismatches between opposed nucleotides. Generally the two molecules hybridize under conditions of high stringency.

As used herein, a method for treating or preventing disease or disorder associated with undesired and/or uncontrolled angiogenesis means that the diseases or the symptoms associated with the undesired and/or uncontrolled angiogenesis are alleviated, reduced, ameliorated, prevented, placed in a state of remission, or maintained in a state of remission. It also means that the hallmarks of pathological angiogenesis are eliminated, reduced or prevented by the treatment. Non-limiting examples of the hallmarks of the pathological angiogenesis include uncontrolled degradation of the basement membrane and proximal extracellular matrix of the endothelial cells, migration, division, and organization of the endothelial cells into new functioning capillaries, and the persistence of such functioning capillaries.

As used herein, operatively linked or operationally associated refers to the functional relationship of DNA with regulatory and effector sequences of nucleotides, such as promoters, enhancers, transcriptional and translational stop sites, and other signal sequences. For example, operative linkage of DNA to a promoter refers to the physical and functional relationship between the DNA and the promoter such that the transcription of such DNA is initiated from the promoter by an RNA polymerase that specifically recognizes, binds to and transcribes the DNA. In order to optimize expression and/or in vitro transcription, it can be necessary to remove, add or alter 5′ untranslated portions of the clones to eliminate extra, potential inappropriate alternative translation initiation (i.e., start) codons or other sequences that can interfere with or reduce expression, either at the level of transcription or translation. Alternatively, consensus ribosome binding sites (see, e.g., Kozak (1991) J. Biol. Chem. 266:19867-19870) can be inserted immediately 5′ of the start codon and can enhance expression. The desirability of (or need for) such modification can be empirically determined.

As used herein, a promoter region or promoter element refers to a segment of DNA or RNA that controls transcription of the DNA or RNA to which it is operatively linked. The promoter region includes specific sequences that are sufficient for RNA polymerase recognition, binding and transcription initiation. This portion of the promoter region is referred to as the promoter. In addition, the promoter region includes sequences that modulate this recognition, binding and transcription initiation activity of RNA polymerase. These sequences can be cis acting or can be responsive to trans acting factors. Promoters, depending upon the nature of the regulation, can be constitutive or regulated. Exemplary promoters contemplated for use in prokaryotes include the bacteriophage T7 and T3 promoters.

As used herein, sample refers to anything which can contain an analyte for which an analyte assay is desired. The sample can be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, to hybridize under conditions of a specified stringency is used to describe the stability of hybrids formed between two single-stranded DNA fragments and refers to the conditions of ionic strength and temperature at which such hybrids are washed, following annealing under conditions of stringency less than or equal to that of the washing step. Typically high, medium and low stringency encompass the following conditions or equivalent conditions thereto:

1) high stringency: 0.1×SSPE or SSC, 0.1% SDS, 65° C.

2) medium stringency: 0.2×SSPE or SSC, 0.1% SDS, 50° C.

3) low stringency: 1.0×SSPE or SSC, 0.1% SDS, 50° C. Equivalent conditions refer to conditions that select for substantially the same percentage of mismatch in the resulting hybrids. Additions of ingredients, such as formamide, Ficoll, and Denhardt's solution affect parameters such as the temperature under which the hybridization should be conducted and the rate of the reaction. Thus, hybridization in 5×SSC, in 20% formamide at 42° C. is substantially the same as the conditions recited above hybridization under conditions of low stringency. The recipes for SSPE, SSC and Denhardt's and the preparation of deionized formamide are described, for example, in Sambrook et al. (1989) Molecular Cloning, A Laboratory Manual, Cold Spring Harbor Laboratory Press, Chapter 8; see, Sambrook et al, vol. 3, p. B.13, see, also, numerous catalogs that describe commonly used laboratory solutions). It is understood that equivalent stringencies can be achieved using alternative buffers, salts and temperatures.

The terms substantially identical or similar varies with the context as understood by those skilled in the relevant art and generally means at least 40, 60, 80, 90, 95 or 98%.

As used herein, substantially identical to a product means sufficiently similar so that the property of interest is sufficiently unchanged so that the substantially identical product can be used in place of the product.

As used herein, target cell refers to a cell that expresses a cell surface protease.

As used herein, test substance, including compounds provided herein, refers to a chemically defined compound (e.g., organic molecules, inorganic molecules, organic/inorganic molecules, proteins, peptides, nucleic acids, oligonucleotides, lipids, polysaccharides, saccharides, or hybrids among these molecules such as glycoproteins, etc.) or mixtures of compounds (e.g., a library of test compounds, natural extracts or culture supernatants, etc.) whose effect on or interaction with a cell surface protein or cell surface-associated protein, or a domain thereof, is determined by the methods herein.

As used herein, the terms a therapeutic agent, therapeutic regimen, radioprotectant, chemotherapeutic mean conventional drugs and drug therapies, including vaccines, which are known to those skilled in the art. Radiotherapeutic agents are well known in the art.

As used herein, vector (or plasmid) refers to discrete elements that are used to introduce heterologous DNA into cells for expression and/or replication thereof. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art. An expression vector includes vectors capable of expressing DNA that is operatively linked with regulatory sequences, such as promoter regions, that are capable of effecting expression of such DNA fragments. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, chemically stable means that the compound is stable enough to be formulated for pharmaceutical use. Such chemical stability is well known to those of skill in the art and can be determined by well known routine methods. Whether a given compound is chemically stable enough to be formulated for pharmaceutical use depends on a number of factors including, but not limited to, the type of formulation and route of administration desired, the disease to be treated, and the method of preparing the pharmaceutical formulation.

As used herein, a “functional equivalent” of a side chain of an amino acid is a group or moiety that functions in substantially the same way as the naturally occurring side chain to achieve substantially the same result (e.g., a substrate for a cell surface protease). For example, functional equivalents of the side chain of arginine include, but are not limited to, homoarginine, guanidinoaminopropyl, guanidinoaminoethyl, (Me)₂arginine side chain, (Et)₂arginine side chain, (4-aminomethyl)phenylmethyl, 4-amidinophenylmethyl, 4-guanidinophenyl-methyl, or a conformationally constrained arginine side chain analog such as:

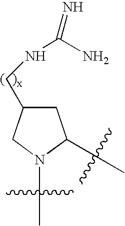

where x is 0 or 1 (see, e.g., Webb et al. (1991) J. Org. Chem. 56:3009), or a conformationally constrained arginine side chain analog such as:

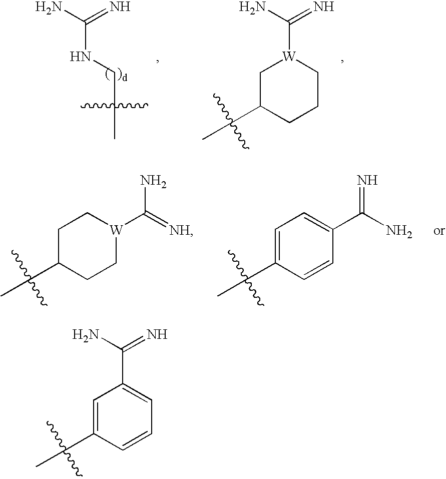

where d is an integer from 0 to 5, or 1 to 3; and W is N or CH; or a mono- or di-substituted N-alkyl derivative of the above groups, where alkyl is, in certain embodiments, lower alkyl, such as methyl.

As used herein, pharmaceutically acceptable derivatives of a compound include salts, esters, enol ethers, enol esters, acids, bases, solvates, hydrates or prodrugs thereof. Such derivatives can be readily prepared by those of skill in this art using known methods for such derivatization. The compounds produced can be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl ar heterocyclyl. Pharmaceutically acceptable solvates and hydrates are complexes of a compound with one or more solvent or water molecule, generally 1 to about 100, typically 1 to about 10, such as 1 to about 2, 3 or 4, solvent or water molecules.

As used herein, treatment means any manner in which one or more of the symptoms of a condition, disorder or disease are ameliorated or otherwise beneficially altered. Treatment also encompasses any pharmaceutical use of the compositions herein, such as use for treating cancer.

As used herein, amelioration of the symptoms of a particular disorder by administration of a particular pharmaceutical composition refers to any lessening, whether permanent or temporary, lasting or transient that can be attributed to or associated with administration of the composition.

As used herein, a prodrug is a compound that, upon in vivo administration, is metabolized or otherwise converted to the biologically, pharmaceutically or therapeutically active form of the compound. To produce a prodrug, the pharmaceutically active compound is modified such that the active compound is regenerated by metabolic processes. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

It is to be understood that the conjugates provided herein can contain chiral centers. Such chiral centers can be of either the (R) or (S) configuration, or can be a mixture thereof. Thus, the compounds provided herein can be enantiomerically pure, or be stereoisomeric or diastereomeric mixtures. In the case of amino acid residues, such residues can be of either the L- or D-form. The configuration for naturally occurring amino acid residues is generally L. When not specified the residue is the L form. It is to be understood that the chiral centers of the compounds provided herein can undergo epimerization in vivo. As such, one of skill in the art will recognize that administration of a compound in its (R) form is equivalent, for compounds that undergo epimerization in vivo, to administration of the compound in its (S) form.

The conjugates provided herein are prodrugs because they include a therapeutic agent in an inactive form that is ultimately converted to an active form at the targeted cell or tissue or in the environment thereof. Upon exposure to targeted protease either a biologically, pharmaceutically or therapeutically active form of a compound is released, or, a derivative that can be further metabolized into a biologically, pharmaceutically or therapeutically active form of a compound.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not alter the physical and chemical properties, such as enzymatic and biological activities, of the substance for its intended purpose. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, a peptidic substrate includes peptides and molecules, such as peptide mimetics and peptides that include peptide bond surrogates.

As used herein, conventional terminology (Schecter et al. (1967) Biochem. Biophys. Res. Commun. 27:157-162) is used to refer to specific subsites of a protease substrate: Pn . . . P3-P2-P1↓P1′-P2′-P3′ . . . Pn′. The scissile bond (i.e., the cleavage site) of a substrate is indicated by the arrow. Positions N-terminal of that bond are referred to as unprimed positions. Subsites are then assigned a number based on their distance from the scissile bond. Amino acids (or amino acid surrogates) that form the scissile bond are assigned the number 1, adjacent residues the number 2, and so on, counting away from the scissile bond. Each specific subsite of the substrate, therefore, is uniquely identified by a number and the designation as primed or unprimed.

As used herein, a surrogate of a peptide bond is a divalent group that possesses similar steric and/or electronic characteristics to —C(O)NH—. Peptide bond surrogates include, but are not limited to, alkene isosteres (—CR═CR—), particularly (E)-alkene isosteres of formula —CH═CH—, hydroxyethylene isosteres (—CH(OH)CH₂—), enamine isosteres (—C(═CRR)NH—), aminoalcohol isosteres (—CH(OH)CH₂NH—), difluoroketone isosteres (—C(O)CF₂—), retroinverso compounds (—NHC(O)—), divalent heterocyclyl or heteroaryl groups, and cyclopropyl isosteres such as:

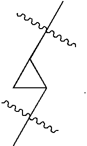

As used herein, alkyl, alkenyl and alkynyl carbon chains, if not specified, contain from 1 to 20 carbons, generally 1 to 16 carbons, and are straight or branched. Alkenyl carbon chains of from 2 to 20 carbons typically contain 1 to 8 double bonds, and the alkenyl carbon chains of 2 to 16 carbons and typically contain 1 to 5 double bonds. Alkynyl carbon chains of from 2 to 20 carbons typically contain 1 to 8 triple bonds, and the alkynyl carbon chains of 2 to 16 carbons and generally contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl and isohexyl. The alkyl, alkenyl and alkynyl groups, unless otherwise specified, optionally can be substituted, with one or more groups, generally alkyl group substituents that are the same or different. As used herein, lower alkyl, lower alkenyl, and lower alkynyl refer to carbon chains having less than about 6 carbons. As used herein, “alk(en)(yn)yl” refers to an alkyl group containing at least one double bond and at least one triple bond.

As used herein, “cycloalkyl” refers to a saturated mono- or multi-cyclic ring system, typically 3 to 10 carbon atoms, such as, for example, 3 to 6 carbon atoms; cycloalkenyl and cycloalkynyl refer to mono- or multicyclic ring systems that respectively include at least one double bond and at least one triple bond. Cycloalkenyl and cycloalkynyl groups contain, for example, 3 to 10 carbon atoms, with cycloalkenyl groups generally containing 4 to 7 carbon atoms and cycloalkynyl groups that contain, for example 8 to 10 carbon atoms. The ring systems of the cycloalkyl, cycloalkenyl and cycloalkynyl groups can be composed of one ring or two or more rings which can be joined together in a fused, bridged or spiro-connected fashion, and optionally can be substituted with one or more alkyl group substituents. “Cycloalk(en)(yn)yl” refers to a cycloalkyl group containing at least one double bond and at least one triple bond.

As used herein, “substituted alkyl,” “substituted alkenyl,” “substituted alkynyl,” “substituted cycloalkyl,” “substituted cycloalkenyl,” and “substituted cycloalkynyl” refer to alkyl, alkenyl, alkynyl, cycloalkyl, cycloalkenyl and cycloalkynyl groups, respectively, that are substituted with one or more substituents, in certain embodiments one to three substituents, independently selected from alkyl, halo, haloalkyl, such as halo lower alkyl, pseudohalo, aryl, amino, dialkylamino, nitro, cyano, azido, alkylsulfinyl, alkylsulfonyl, alkylcarbonylamino, alkoxycarbonylamino, aminoimino, hydroxy, alkoxy, aryloxy, alkyloxy, alkylthio, arylthio, aralkyloxy, aralkylthio, carboxy, alkylcarbonyl, alkoxycarbonyl, oxo and cycloalkyl.

As used herein, “aryl” refers to cyclic groups containing from 6 to 19 carbon atoms. Aryl groups include, but are not limited to groups, such as fluorenyl, substituted fluorenyl, phenyl, substituted phenyl, naphthyl and substituted naphthyl. As used herein, “aryl” also refers to aryl-containing groups, including, but not limited to, aryloxy, arylthio, arylcarbonyl and arylamino groups.

As used herein, “heteroaryl” refers to a monocyclic or multicyclic aromatic ring system, generally about 5 to about 15 members where one or more, such as 1 to 3 of the atoms in the ring system is a heteroatom, that is, an element other than carbon, for example, nitrogen, oxygen and sulfur atoms. The heteroaryl group optionally can be fused to a benzene ring. Exemplary heteroaryl groups include, for example, furyl, imidazolyl, pyrrolidinyl, pyrimidinyl, tetrazolyl, thienyl, pyridyl, pyrrolyl, N-methylpyrrolyl, quinolinyl and isoquinolinyl, with pyridyl, thienyl and quinolinyl as examples thereof.

As used herein, “heteroaryl” also refers to heteroaryl-containing groups, including, but not limited to, heteroaryloxy, heteroarylthio, heteroarylcarbonyl and heteroarylamino.

As used herein, “heterocyclyl” refers to a monocyclic or multicyclic non-aromatic ring system, such as systems of 3 to 10 members, for exmaple 4 to 7 members or 5 to 6 members, where one or more, such as 1 to 3 of the atoms in the ring system is a heteroatom, that is, an element other than carbon, for example, nitrogen, oxygen and/or sulfur atoms.

As used herein, “substituted aryl,” “substituted heteroaryl” and “substituted heterocyclyl” refer to aryl, heteroaryl and heterocyclyl groups, respectively, that are substituted with one or more substituents, in certain embodiments one to three substituents, independently selected from alkyl, cycloalkyl, cycloalkylalkyl, aryl, heteroaryl optionally substituted with 1 or more, such as 1 to 3, substituents selected from halo, halo alkyl and alkyl, aralkyl, heteroaralkyl, alkenyl containing 1 to 2 double bonds, alkynyl containing 1 to 2 triple bonds, alk(en)(yn)yl groups, halo, pseudohalo, cyano, hydroxy, haloalkyl and polyhaloalkyl, such as halo lower alkyl, especially trifluoromethyl, formyl, alkylcarbonyl, arylcarbonyl that optionally is substituted with 1 or more, generally 1 to 3, substituents selected from halo, halo alkyl and alkyl, heteroarylcarbonyl, carboxy, alkoxycarbonyl, aryloxycarbonyl, aminoimino, alkoxycarbonylamino, aryloxycarbonylamino, aminocarbonyl, alkylaminocarbonyl, dialkylaminocarbonyl, arylaminocarbonyl, diarylaminocarbonyl, aralkylaminocarbonyl, alkoxy, aryloxy, perfluoroalkoxy, alkenyloxy, alkynyloxy, arylalkoxy, aminoalkyl, alkyl-aminoalkyl, dialkylaminoalkyl, arylaminoalkyl, amino, alkylamino, dialkyl-amino, arylamino, alkylarylamino, alkylcarbonylamino, arylcarbonylamino, azido, nitro, mercapto, alkylthio, arylthio, perfluoroalkylthio, thiocyano, isothiocyano, alkylsulfinyl, alkylsulfonyl, arylsulfinyl, arylsulfonyl, aminosulfonyl, alkylaminosulfonyl, dialkylaminosulfonyl and arylamino-sulfonyl.

As used herein, “aralkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, the nomenclature alkyl, alkoxy, carbonyl, etc. is used as is generally understood by those of skill in this art. For example, as used herein alkyl refers to saturated carbon chains that contain one or more carbons; the chains can be straight or branched or include cyclic portions or be cyclic.

Where the number of any given substituent is not specified (e.g., “haloalkyl”), there can be one or more substituents present. For example, “haloalkyl” can include one or more of the same or different halogens. As another example, “C₁₋₃alkoxyphenyl” can include one or more of the same or different alkoxy groups containing one, two or three carbons.

As used herein, “halo”, “halogen” or “halide” refers to F, Cl, Br or I.

As used herein, pseudohalides are compounds that behave substantially similar to halides. Such compounds can be used in the same manner and treated in the same manner as halides (X⁻, in which X is a halogen, such as Cl or Br). Pseudohalides include, but are not limited to, cyanide, cyanate, thiocyanate, selenocyanate, trifluoromethoxy, difluoromethoxy, dichloromethoxy and azide.

As used herein, “haloalkyl” refers to a lower alkyl radical in which one or more of the hydrogen atoms are replaced by halogen. Such groups include, but not limited to, chloromethyl, trifluoromethyl, 1-chloro-2-fluoroethyl and the like.

As used herein, “haloalkoxy” refers to RO— in which R is a haloalkyl group.

As used herein, “sulfinyl” or “thionyl” refers to —S(O)—. As used herein, “sulfonyl” or “sulfuryl” refers to —S(O)₂—. As used herein, “sulfo” refers to —S(O)₂O—.

As used herein, “carboxy” refers to a divalent radical, —C(O)O—.

As used herein, “aminocarbonyl” refers to —C(O)NH₂.

As used herein, “alkylaminocarbonyl” refers to —C(O)NHR in which R is hydrogen or alkyl, such as, for example, lower alkyl.

As used herein “dialkylaminocarbonyl” as used herein refers to —C(O)NR′R in which R′ and R are independently selected from hydrogen or alkyl, such as, for example, lower alkyl; “carboxamide” refers to groups of formula —NR′COR.

As used herein, “diarylaminocarbonyl” refers to —C(O)NRR′ in which R and R′ are independently selected from aryl, such as lower aryl, for example, phenyl.

As used herein, “aralkylaminocarbonyl” refers to —C(O)NRR′ in which one of R and R′ is aryl, such as, lower aryl, for example, phenyl, and the other of R and R′ is alkyl, such as, for example, lower alkyl.

As used herein, “arylaminocarbonyl” refers to —C(O)NHR in which R is aryl, such as lower aryl, for example, phenyl.

As used herein, “hydroxycarbonyl” refers to —COOH.

As used herein, “alkoxycarbonyl” refers to —C(O)OR in which R is alkyl, such as lower alkyl.

As used herein, “aryloxycarbonyl” refers to —C(O)OR in which R is aryl, such lower aryl, for example phenyl.

As used herein, “alkoxy” and “alkylthio” refer to RO— and RS—, in which R is alkyl, such as, for example, lower alkyl.

As used herein, “aryloxy” and “arylthio” refer to RO— and RS—, in which R is aryl, such lower aryl, for example, phenyl.

As used herein, “alkylene” refers to a straight, branched or cyclic, such as, for example, straight or branched, divalent aliphatic hydrocarbon group, for example, having from 1 to about 20 carbon atoms such as 1 to 12 carbons, and for exmaple, is lower alkylene. There optionally can be inserted along the alkylene group one or more oxygen, sulphur or substituted or unsubstituted nitrogen atoms, where the nitrogen substituent is alkyl as previously described. Exemplary alkylene groups include methylene (—CH₂—), ethylene (—CH₂CH₂—), propylene (—(CH₂)₃—), cyclohexylene (—C₆H₁₀—), methylenedioxy (—O—CH₂—O—) and ethylenedioxy (—O—(CH₂)₂—O—). The term “lower alkylene” refers to alkylene groups having 1 to 6 carbons. Exemplary alkylene groups are lower alkylene, such as, for example, alkylene of 1 to 3 carbon atoms.

As used herein, “alkenylene” refers to a straight, branched or cyclic, typically straight or branched, divalent aliphatic hydrocarbon group, such as, for example, having from 2 to about 20 carbon atoms and at least one double bond, generally 1 to 12 carbons, and is for example, lower alkenylene. There optionally can be inserted along the alkenylene group one or more oxygen, sulphur or substituted or unsubstituted nitrogen atoms, where the nitrogen substituent is alkyl as previously described. Exemplary alkenylene groups include —CH═CH—CH═CH— and —CH═CH═CH₂—. The term “lower alkenylene” refers to alkenylene groups having 2 to 6 carbons. Examplary alkenylene groups are lower alkenylene, such as, for example, alkenylene of 3 to 4 carbon atoms.

As used herein, “alkynylene” refers to a straight, branched or cyclic, generally straight or branched, divalent aliphatic hydrocarbon group, such those having from 2 to about 20 carbon atoms and at least one triple bond, generally 1 to 12 carbons, such as, for example, lower alkynylene. There optionally can be inserted along the alkynylene group one or more oxygen, sulphur or substituted or unsubstituted nitrogen atoms, where the nitrogen substituent is alkyl as previously described. Exemplary alkynylene groups include —C≡C—C≡C—, —C≡C— and —C≡C—CH₂—. The term “lower alkynylene” refers to alkynylene groups having 2 to 6 carbons. Exemplary alkynylene groups are lower alkynylene, such as, for example, alkynylene of 3 to 4 carbon atoms.

As used herein, “alk(en)(yn)ylene” refers to a straight, branched or cyclic, generally straight or branched, divalent aliphatic hydrocarbon group, having, for example, from 2 to about 20 carbon atoms and at least one triple bond, and at least one double bond; typically 1 to 12 carbons, such as, for example, lower alk(en)(yn)ylene. There optionally can be inserted along the alkynylene group one or more oxygen, sulphur or substituted or unsubstituted nitrogen atoms, where the nitrogen substituent is alkyl as previously described. Exemplary alk(en)(yn)ylene groups include —C═C—(CH₂)ₙ—C≡C—, where n is 1 or 2. The term “lower alk(en)(yn)ylene” refers to alk(en)(yn)ylene groups having up to 6 carbons. Exemplary alk(en)(yn)ylene groups are lower alk(en)(yn)ylene, such as, for example, alk(en)(yn)ylene of 4 carbon atoms.

As used herein, “cycloalkylene” refers to a divalent saturated mono- or multicyclic ring system, generally 3 to 10 carbon atoms, such as 3 to 6 carbon atoms; cycloalkenylene and cycloalkynylene refer to divalent mono- or multicyclic ring systems that respectively include at least one double bond and at least one triple bond. Cycloalkenylene and cycloalkynylene groups can contain 3 to 10 carbon atoms, with, for example, cycloalkenylene groups containing 4 to 7 carbon atoms and cycloalkynylene groups containing 8 to 10 carbon atoms. The ring systems of the cycloalkylene, cycloalkenylene and cycloalkynylene groups can be composed of one ring or two or more rings that can be joined together in a fused, bridged or spiro-connected fashion. “Cycloalk(en)(yn)ylene” refers to a cycloalkylene group containing at least one double bond and at least one triple bond.

As used herein, “substituted alkylene,” “substituted alkenylene,” “substituted alkynylene,” “substituted cycloalkylene,” “substituted cycloalkenylene,” and “substitued cycloalkynylene” refer to alkylene, alkenylene, alkynylene, cycloalkylene, cycloalkenylene and cycloalkynylene groups, respectively, that are substituted with one or more substituents, in certain embodiments one to three substituents, independently selected from halo, haloalkyl, such as, for example, halo lower alkyl, aryl, hydroxy, alkoxy, aryloxy, alkyloxy, alkylthio, arylthio, aralkyloxy, aralkylthio, carboxy alkoxycarbonyl, oxo and cycloalkyl.

As used herein, “arylene” refers to a monocyclic or polycyclic, such as monocyclic, divalent aromatic group, for example, having from 5 to about 20 carbon atoms and at least one aromatic ring, such as 5 to 12 carbons, and, is, for example, lower arylene. There optionally can be inserted around the arylene group one or more oxygen, sulphur or substituted or unsubstituted nitrogen atoms, where the nitrogen substituent is alkyl as previously described. Exemplary arylene groups include 1,2-, 1,3- and 1,4-phenylene. The term “lower arylene” refers to arylene groups having 5 or 6 carbons. Exemplary arylene groups are lower arylene.

As used herein, “heteroarylene” refers to a divalent monocyclic or multicyclic aromatic ring system, such as of about 5 to about 15 members where one or more, typically, for example, 1 to 3 of the atoms in the ring system is a heteroatom, that is, an element other than carbon, for example, nitrogen, oxygen and/or sulfur atom(s).

As used herein, “heterocyclylene” refers to a divalent monocyclic or multicyclic non-aromatic ring system, generally of 3 to 10 members, such as, for example, 4 to 7 members or 5 to 6 members, where one or more, such as, for example, 1 to 3 of the atoms in the ring system is a heteroatom, that is, an element other than carbon, for example, nitrogen, oxygen and/or sulfur atom(s).

As used herein, “substituted arylene,” “substituted heteroarylene” and “substituted heterocyclylene” refer to arylene, heteroarylene and heterocyclylene groups, respectively, that are substituted with one or more substituents, in certain embodiments one to three substituents, independently selected from alkyl, cycloalkyl, cycloalkylalkyl, aryl, heteroaryl optionally substituted with 1 or more, such as 1 to 3, substituents selected from halo, halo alkyl and alkyl, aralkyl, heteroaralkyl, alkenyl containing 1 to 2 double bonds, alkynyl containing 1 to 2 triple bonds, alk(en)(yn)yl groups, halo, pseudohalo, cyano, hydroxy, haloalkyl and polyhaloalkyl, such as, halo lower alkyl, for example trifluoromethyl, formyl, alkylcarbonyl, arylcarbonyl that optionally is substituted with 1 or more, such as 1 to 3, substituents selected from, for example, halo, halo alkyl and alkyl, heteroarylcarbonyl, carboxy, alkoxycarbonyl, aryloxycarbonyl, aminocarbonyl, alkylaminocarbonyl, dialkylaminocarbonyl, arylaminocarbonyl, diarylaminocarbonyl, aralkylaminocarbonyl, alkoxy, aryloxy, perfluoroalkoxy, alkenyloxy, alkynyloxy, arylalkoxy, aminoalkyl, alkylaminoalkyl, dialkylaminoalkyl, arylaminoalkyl, amino, alkylamino, dialkylamino, arylamino, alkylarylamino, alkylcarbonylamino, arylcarbonylamino, azido, nitro, mercapto, alkylthio, arylthio, perfluoroalkylthio, thiocyano, isothiocyano, alkylsulfinyl, alkylsulfonyl, arylsulfinyl, arylsulfonyl, aminosulfonyl, alkylaminosulfonyl, dialkylaminosulfonyl and arylaminosulfonyl.

As used herein, “alkylidene” refers to a divalent group, such as ═CR′R″, which is attached to one atom of another group, forming a double bond. Exemplary alkylidene groups are methylidene (═CH₂) and ethylidene (═CHCH₃). As used herein, “aralkylidene” refers to an alkylidene group in which either R′ or R″ is an aryl group. “Cycloalkylidene” groups are those where R′ and R″ are linked to form a carbocyclic ring. “Heterocyclylidene” groups are those where at least one of R′ and R″ contain a heteroatom in the chain, and R′ and R″ are linked to form a heterocyclic ring.

As used herein, “amido” refers to the divalent group —C(O)NH—. “Thioamido” refers to the divalent group —C(S)NH—. “Oxyamido” refers to the divalent group —OC(O)NH—. “Thiaamido” refers to the divalent group —SC(O)NH—. “Dithiaamido” refers to the divalent group —SC(S)NH—. “Ureido” refers to the divalent group —HNC(O)NH—. “Thioureido” refers to the divalent group —HNC(S)NH—.

As used herein, “semicarbazide” refers to —NHC(O)NHNH—. “Carbazate” refers to the divalent group —OC(O)NHNH—. “Isothiocarbazate” refers to the divalent group —SC(O)NHNH—. “Thiocarbazate” refers to the divalent group —OC(S)NHNH—. “Sulfonylhydrazide” refers to the group —SO₂NHNH—. “Hydrazide” refers to the divalent group —C(O)NHNH—. “Azo” refers to the divalent group —N═N—. “Hydrazinyl” refers to the divalent group —NH—NH—.

As used herein, the term “amino acid” refers to α-amino acids which are racemic, or of either the D- or L-configuration. The designation “d” preceding an amino acid designation (e.g., dAla, dSer, dVal, etc.) refers to the D-isomer of the amino acid. The designation “dI” preceding an amino acid designation (e.g., dIPip) refers to a mixture of the L- and D-isomers of the amino acid.

As used herein, when any particular group, such as phenyl or pyridyl, is specified, this means that the group is unsubstituted or is substituted. Exemplary substituents where not specified are halo, halo lower alkyl, and lower alkyl.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

As used herein, HHT and CHT refer to hexahydrotyrosyl (also known as cyclohexyltyrosyl or p-hydroxycyclohexylalanyl), CHA is cyclohexylalanyl, Pyr and pyroGlu refer to pyroglutamic acid, Pip is pipecolinic acid, Sar is sarcosine, nLeu and Nle are norleucine, nVal is norvaline, Aib is 2-aminoisobutyric acid, Quat is (R)-Glu(α-(3-amidinobenzyl)), and Abu and But are 2-aminobutyric acid.

As used herein, PEG represents a polyethylene glycol containing substituent having the designated number of ethyleneoxy subunits. Thus, the term PEG(2) represents:

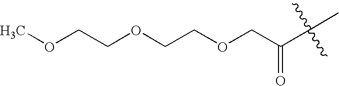

and the term PEG(6) represents:

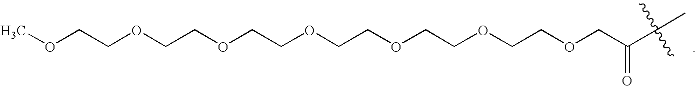

When R¹ and R² are combined to form —(CH₂)ₕ—, the cyclic moieties and heteroatom-containing cyclic moieties so defined include, but are not limited to:

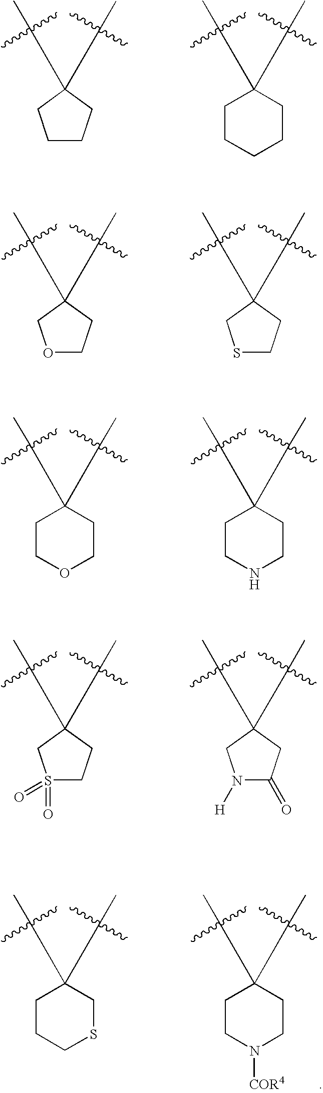

As used herein, the term “hydroxylated” represents substitution on a substitutable carbon of the ring system being so described by a hydroxyl moiety. As used herein, the term “polyhydroxylated” represents substitution on two or more substitutable carbons of the ring system being so described by 2, 3 or 4 hydroxyl moieties.

As used herein, the term “(d)(2,3-dihydroxypropionyl)” represents the following structure:

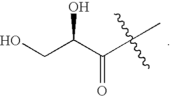

As used herein, the term “(2R,3S)-2,3,4-trihydroxybutanoyl” represents the following structure:

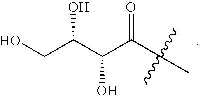

As used herein, the term “quinyl” represents the following structure:

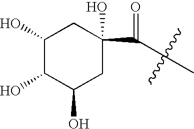

or a diastereomer thereof.

As used herein, the term “gulonyl” represents the following structure:

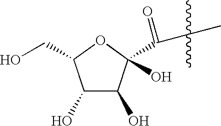

or a diastereomer thereof.

As used herein, the term “cotininyl” represents the following structure:

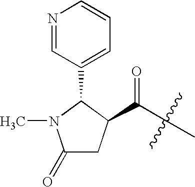

or a diastereomer thereof.

As used herein, the term “gallyl” represents the following structure:

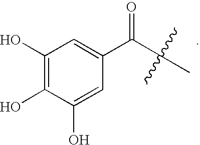

As used herein, the term “4-ethoxysquaryl” represents the following structure:

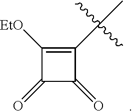

As used herein, 1-methylHis or (1Me)H refers to the structure:

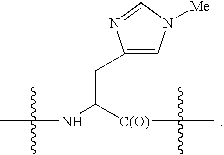

As used herein, 3-methylHis or (3Me)H refers to the structure:

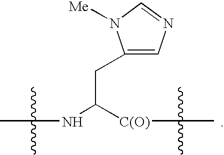

As used herein, Quat² refers to:

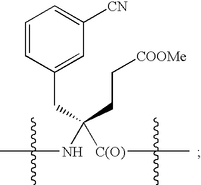

Quat³ refers to:

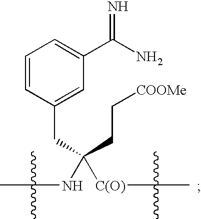

Quat⁴ refers to:

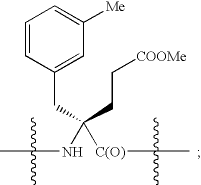

and

Quat⁵ refers to:

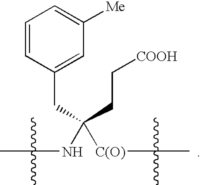

Other abbreviations as used herein are as follows:

| Abbreviation | Refers to |
|---|---|
| Aib | 2-aminoisobutyryl |
| 4,4-dimethylThr | 2-amino-3-hydroxy-4-methylpentanoyl |
| Met(O₂) | methioninyl-S,S-dioxide |
| Ser(OMe) | the O-methyl ether of serinyl, also known as |
| | 2-amino-3-methoxypropanoyl |
| hSer | homoserinyl, also known as 2-amino-4- |
| | hydroxybutanoyl |
| (hS)Gly | N-(2-hydroxyethyl)glycyl |
| N,N-dimethylGly | N,N-dimethylglycyl |
| β-Ala | 3-aminopropanoyl |
| Cys(Me) | S-methylcysteinyl |
| t-butylGly | 2-amino-3,3-dimethylbutanoyl |
| F(Gn) | 4-guanidinyiphenylalanyl |
| hCHA | homocyclohexylalanyl, or 2-amino-4- |
| | cyclohexylbutanoyl |
| hexylGly | 2-aminooctanoyl |
| allylGly | 2-amino-4-pentenoyl |
| Inact. | inactive |
| NT | not tested |
| MeOEtCO | 3-methoxypropanoyl |
| 3,4-MethyldioxyPhAc | 3,4-methylenedioxyphenylacetyl |
| L-3-PhLactyl | L-2-hydroxy-3-phenylpropanoyl |
| MeOEtOCO | 2-methoxyethoxycarbonyl |
| MeOCO | methoxycarbonyl |
| MeO(EtO)2Ac | 2-(2-methoxyethoxy)ethoxyacetyl |
| 2-PyridylAc | 2-pyridylacetyl |
| PhOAc | phenoxyacetyl |
| MeOAc | methoxyacetyl |
| PhAc | phenylacetyl |
| MeOEtOAc | 2-methoxyethoxyacetyl |
| HOOCButa | glutaryl |
| Z | benzyloxycarbonyl |
| EtOCO | ethoxycarbonyl |
| βA | beta-alanyl or 3-aminopropanoyl |
| NapAc | 1-naphthylacetyl |
| iBoc | isobutoxycarbonyl |
| HOAc | hydroxyacetyl |
| MeSucc | 3-methoxycarbonylpropanoyl |
| Succ | succinyl |
| HCO | formyl |
| 4-(guan)Phg | 4-guanidinylphenylglycyl |
| Dox | doxorubicin |
| Tax | taxol |
| dA(Chx) or dCha | d-cyclohexylalanyl |
| dhF | d-homophenylalanyl |
| P(OH) | 4-hydroxyprolyl |

### B. Protease Targets

The conjugates herein are designed to target proteases that are located on cell surfaces, particularly tumor cells and cells involved in tumorigenic processes and angiogenesis and other proliferative processes. The conjugates, described in detail below, contain a peptidic substrate for a selected targeted cell surface protease linked, either directly or via a linker, to a therapeutic agent, typically a cytotoxic agent, which is substantially inactive when in the conjugate. The therapeutic agent is released in a form that is active or that can be activated in the vicinity of the targeted cell or tissue to which it is delivered. As a result, active therapeutic agent accumulates at the targeted cells or tissue or in the targeted cells.

The targeted protease is selected by identifying a protease that is located on a cell or tissue (or associated therewith) that is involved in the disease process or serendipitously present in the locale of cells or tissues involved in the disease or disease process, and, generally, is not located at all or present or active at lower levels, generally substantially lower levels, or exhibits altered activity or specificity, on many, if not all, other cells or tissues. The variety and numbers of non-targeted cells or tissues that expresss the active protease varies for particular proteases and diseases intended for treatment. Those of skill in the art will select a target based upon the disease, targeted agents and tolerable or acceptable levels of side-effects. The goal is to achieve enhanced therapeutic index compared with administration of the targeted agent by itself.

The targeted protease may or may not be involved in the disease process and its expression can be serendiptous; for purposes herein its particular role or lack thereof is not important; it is the fact that it is active in the locale of targeted tissues or cells that is important. For example, many of the cell surface proteases of interest herein are expressed or active on tumor cells or cells involved in the tumorigenic processes. Any method known to one of skill in the art for determining or detecting a tissue or cell expression profile can be used. For example, RNA blots composed of RNA from numerous tissues (e.g., a multiple tissue expression (MTE) array available from CLONTECH, Palo Alto, Calif.), can be screened with probes based upon the nucleic acid sequence of the protease of interest to identify cells that express the protease. Northern analysis of the blots to test for expression also can be used.

Included among the targeted proteases are those designated type II membrane-bound serine proteases (MTSPs; see, e.g., U.S. application Ser. No. 09/776,191, filed Feb. 2, 2001 and International PCT application No. PCT/US01/03471 published as International PCT application No. WO 01/57194; see International PCT application No. PCT/US02/07903; see, also U.S. provisional application Serial Nos. 60/275,592, 60/278,166, 60/279,228, 60/291,001, 60/291,501 60/316,818, 60/302,939, 60/316,818, 60/328,529, 60/328,530, 60/332,015, 60/328,939, and provisional application, filed on May 20, 2002 under attorney docket no. 24745-P1624; U.S. application Ser. Nos. 10/099,700, 10/104,271, 10/112,221, application filed on May 14, 2002 under attorney docket no. 24745-1616) and those found on endothelial cells designated endotheliases (see, U.S. application Ser. No. 09/717,473, filed Nov. 20, 2000, and International PCT application No. PCT/US00/31803 published as International PCT application No. WO 01/36604); see, also SEQ ID Nos. 3-26, 269-270 and 272-276.

Also contemplated are proteases that are located at the cell surface by virtue of a specific interaction with a cell surface protein. Urokinase plasminogen activator (u-PA) bound to urokinase plasminogen activator receptor (u-PAR) is exemplary of such proteases. Nucleic acid sequence information and expression profiles of exemplary MTSPs and endotheliases are as follows (see, also EXAMPLE 6).

1. MTSPs

Cell surface proteolysis is a mechanism for the generation of biologically active proteins that mediate a variety of cellular functions. These membrane-anchored proteins, include a disintegrin-like and metalloproteinase (ADAM) and membrane-type matrix metalloproteinase (MT-MMP). In addition to the MMPs, serine proteases have been implicated in neoplastic disease progression. Most serine proteases, which are either secreted enzymes or are sequestered in cytoplasmic storage organelles, have roles in blood coagulation, wound healing, digestion, immune responses and tumor invasion and metastasis.

Transmembrane serine proteases (MTSPs) appear to be involved in the etiology and pathogenesis of tumors. These enzymes are expressed in certain cancerous and tumor cells and in other cells associated with other proliferative disorders and other disease states, such as in inflammatory cells and and can be tissue or organ-specific. In mammals, more than 20 members of the family are known (see, Hooper et al. (2001) J. Biol. Chem. 276:857-860, see, also U.S. application Ser. No. 09/776,191, filed Feb. 2, 2001 and International PCT application No. PCT/US01/03471; see, also U.S. provisional application Serial Nos. 60/275,592 and 60/278,166; and see SEQ ID Nos. 1-37). These include corin (accession nos. AF133845 and AB013874; see, Yan et al. (1999) J. Biol. Chem. 274:14926-14938; Tomia et al. (1998) J. Biochem. 124:784-789; Uan et al. (2000) Proc. Natl. Acad. Sci. U.S.A. 97:8525-8529); enterpeptidase (also designated enterokinase; accession no. U09860 for the human protein; see, Kitamoto et al. (1995) Biochem. 27: 4562-4568; Yahagi et al. (1996) Biochem. Biophys. Res. Commun. 219:806-812; Kitamoto et al. (1994) Proc. Natl. Acad. Sci. U.S.A. 91:7588-7592; Matsushima et al. (1994) J. Biol. Chem. 269:19976-19982;); human airway trypsin-like protease (HAT; accession no. AB002134; see Yamaoka et al. J. Biol. Chem. 273:11894-11901); MTSP1 (also called TADG-15 and matriptase, see SEQ ID Nos. 1 and 2; accession nos. AF133086/AF118224, AF04280022; Takeuchi et al. (1999) Proc. Natl. Acad. Sci. U.S.A. 96:11054-1161; Lin et al. (1999) J. Biol. Chem. 274:18231-18236; Takeuchi et al. (2000) J. Biol. Chem. 275:26333-26342; and Kim et al. (1999) Immunogenetics 49:420-429); hepsin (see, accession nos. M18930, AF030065, X70900; Leytus et al. (1988) Biochem. 27: 11895-11901; Vu et al. (1997) J. Biol. Chem. 272:31315-31320; and Farley et al. (1993) Biochem. Biophys. Acta 1173:350-352; and see, U.S. Pat. No. 5,972,616); TMPRS2 (see, Accession Nos. U75329 and AF113596; Paoloni-Giacobino et al. (1997) Genomics 44:309-320; and Jacquinet et al. (2000) FEBS Lett. 468: 93-100); and TMPRSS4 (see, Accession No. NM 016425; Wallrapp et al. (2000) Cancer 60:2602-2606). Also known MTSP3, MTSP4, MTSP6, MTSP7, MTSP9, MTSP10, MTSP12, MTSP20, MTSP22 and MTSP25 (see, SEQ ID NOs. 3-26, 269-270 and 272-276; see, also U.S. application Ser. No. 09/776,191, filed Feb. 2, 2001 and International PCT application No. PCT/US01/03471 published as International PCT application No. WO 01/57194; see International PCT application No. PCT/US02/07903; see, also U.S. provisional application Serial Nos. 60/275,592, 60/278,166, 60/279,228, 60/291,001, 60/291,50160/316,818, 60/302,939, 60/316,818, 60/328,529, 60/328,530, 60/332,015, 60/328,939, and provisional application, filed on May 20 2002, under attorney docket no. 24745-P1624; U.S. application Ser. Nos. 10/099,700, 10/104,271, 10/112,221, application filed on May 14, 2002 under attorney docket no. 24745-1616)).

Serine proteases, including transmembrane serine proteases, have been implicated in processes involved in neoplastic development and progression. While the precise role of these proteases has not been elaborated, serine proteases and inhibitors thereof are involved in the control of many intra- and extracellular physiological processes, including degradative actions in cancer cell invasion, metastatic spread, and neovascularization of tumors, that are involved in tumor progression. It is believed that proteases are involved in the degradation of extracellular matrix (ECM) and contribute to tissue remodeling, and are necessary for cancer invasion and metastasis. The activity and/or expression of some proteases have been shown to correlate with tumor progression and development, and also are shown to be active in specific cell types.

For example, a membrane-type serine protease MTSP1 (also called matriptase; see SEQ ID Nos. 1 and 2 from U.S. Pat. No. 5,972,616; and GenBank Accession No. AF118224; (1999) J. Biol. Chem. 274:18231-18236; U.S. Pat. No. 5,792,616; see, also Takeuchi (1999) Proc. Natl. Acad. Sci. U.S.A. 96:11054-1161) that is expressed in epithelial cancer and normal tissue (Takeucuhi et al. (1999) Proc. Natl. Acad. Sci. USA 96:11054-61) has been identified. It has been proposed that it plays a role in the metastasis of breast cancer. Its primary cleavage specificity is Arg-Lys residues. Matriptase also is expressed in a variety of epithelial tissues with high levels of activity and/or expression in the human gastrointestinal tract and the prostate.

Hepsin, a cell surface serine protease identified in hepatoma cells, is overexpressed in ovarian cancer (Tanimoto et al. (1997) Cancer Res., 57:2884-7). The hepsin transcript appears to be abundant in carcinoma tissue and is almost never expressed in normal adult tissue, including normal ovary. It has been suggested that hepsin is frequently overexpressed in ovarian tumors and therefore can be a candidate protease in the invasive process and growth capacity of ovarian tumor cells.

A serine protease-like gene, designated normal epithelial cell-specific 1 (NES1) (Liu et al. (1996) Cancer Res. 56:3371-9) has been identified. Although expression of the NES1 mRNA is observed in all normal and immortalized nontumorigenic epithelial cell lines, the majority of human breast cancer cell lines show a drastic reduction or a complete lack of its expression. The structural similarity of NES1 to polypeptides known to regulate growth factor activity and a negative correlation of NES1 expression with breast oncogenesis suggest a direct or indirect role for this protease-like gene product in the suppression of tumorigenesis.

Exemplary MTSPs

Each MTSP has a characteristic tissue expression profile; the MTSPs in particular, although not exclusively expressed or activated in tumors, exhibit characteristic tumor tissue expression or activation profiles. In some instances, MTSPs can have different activity in a tumor cell from a non-tumor cell by virtue of a change in a substrate or cofactor therefor or other factor that would alter functional activity of the MTSP. Hence each can serve as a diagnostic marker for particular tumors, by virtue of a level of activity and/or expression or function in a subject (i.e. a mammal, particularly a human) with neoplastic disease, compared to a subject or subjects that do not have the neoplastic disease. In addition, detection of activity (and/or expression) in a particular tissue can be indicative of neoplastic disease. Also, by virtue of the activity and/or expression profiles of each, they can serve as therapeutic targets, such as by administration of modulators of the activity thereof, or, as by administration of a prodrug specifically activated by one of the MTSPs. Each or any of the MTSPs can exhibit activity or expression levels or substrate specificities that differ in tumor cells from the levels in normal cells. Such tumor cells include, but are not limited to, colon, lung, prostate, breast, esophagous, pancreas, cervic, uterus, endometrium, and other solid tumors and in blood and lymphatic tumors. Hence, conjugates provided herein can be designed by selection of substrate specificity for treatment of any of such tumors and neoplastic conditions.

Tissue Expression Profiles

The following are exemplary tissue and gene (see also, EXAMPLE 8) profiles of some exemplary MTSPs. These profiles are not intended to define the full scope of expression or activation of these MTPSs, but demonstrate that MTSPs are expressed in tumors, and, hence there expression or activation or substrate specificity on the surface of tumor cells can be exploited in the methods herein and conjugates, designed in accord with the methods herein and as exemplified herein, that are cleaved by one or more of these MTSPs can be prepared and employed for treatment of neoplastic or other diseases or conditions or to target to cells that express these proteins on there surfaces.

MTSP1 (Matriptase)

MTSP1 (also called matriptase) is a trypsin-like serine protease with broad spectrum cleavage activity and two potential regulatory modules. It was named “matriptase” based on its ability to degrade the extra-cellular matrix and its trypsin-like activity. When isolated from breast cancer cells (or T-47D cell conditioned medium), MTSP1 has been reported to be primarily in an uncomplexed form. MTSP1 has been isolated from human milk; when isolated from human milk, it was reported to be in one of two complexed forms, 95 kDa (the predominant form) and 110 kDa; uncomplexed MTSP1 was not detected (Liu, et al. (1999) J. Biol. Chem. 274:18237-18242). It has been proposed that MTSP1 exists as an uncomplexed protease when in its active state. In breast milk, it has been reported to exist in complex with a fragment of hepatocyte growth factor inhibitor-1 (HAI-1), a Kunitz-type serine protease inhibitor having activity against trypsin-like serine proteases.

Nucleic acids encoding the protein designed matriptase were cloned from T-47D human breast cancer cell-conditioned medium (Lin et al. (1999) J. Biol. Chem. 274:18231-18236). Upon analysis of the cDNA, it was determined that the full length protease has 683 amino acids and contains three main structural regions: a serine protease domain near the carboxyl-terminal region, four tandem low-density lipoprotein receptor domains, and two tandem complement subcomponents C1r and C1s (see SEQ ID No. 1). Studies to identify additional serine proteases made by cancer cells were done using PC-3 cells. A serine protease termed “MT-SP1” (MTSP1) by the authors, reported to be a transmembrane protease was cloned (Takeuchi et al. (1999) Proc. Natl. Acad. Sci. U.S.A. 96:11054-11061). It was subsequently found that originally identified matriptase sequence is included in the translated sequence of the cDNA that encodes MTSP1. The nucleic acid encoding the protein originally designated matriptase is a partial MTSP1 clone that lacks 516 of the coding nucleotides (Takeuchi, et al., J. Biol. Chem 275:26333-26342 (2000).) Since the reported matriptase encoding cDNA sequence encoded a possible initiating methionine, it was proposed that alternative splicing could yield a protein lacking the N-terminal region of MTSP1. Hence, matriptase herein is a variant form of MTSP1.

MTSP1 demonstrates trypsin-like protease activity and is a Type II transmembrane protein with an extracellular protease domain. Studies of substrate specificity of MTSP1 reveal that protease-activated receptor 2 (PAR2), pro-hepatacyte growth factor (pro-HGF) and single-chain urokinase-type plasminogen activator (sc-uPA) are macromolecular substrates of MTSP1. PAR2 functions in inflammation, cytoprotection and/or cell adhesion, while sc-uPa functions in tumor cell invasion and metastasis. HGF serves a growth and pro-angiogenic factor.

An exemplary nucleotide sequence encoding a human MTSP1 is set forth in SEQ ID Nos 1 and 2. As previously noted SEQ ID No. 1 sets for an MTSP1-encoding nucleic acid sequence. This sequence is the longer version and includes the protease domain, which is common to both variants.

MTSP1 is expressed in breast, prostate and colorectal tumors. Hence conjugates with substrates therefor can be used for treatment of such tumors.

MTSP3

The MTSP3 transcript was detected in lung carcinoma (LX-1), colon adenocarcinoma (CX-1), colon adenocarcinoma (GI-112) and ovarian carcinoma (GI-102). No apparent signal was detected in another form of lung carcinoma (GI-117), breast carcinoma (GI-101), pancreatic adenocarcinoma (GI-103) and prostatic adenocarcinoma (PC3).

MTSP4

The MTSP4 transcript, a DNA fragment encoding part of the LDL receptor domain and the protease domain was used to probe an RNA blot composed of 76 different human tissues (catalog number 7775-1; human multiple tissue expression (MTE) array; CLONTECH). As in the northern analysis of gel blot, a very strong signal was observed in the liver. Signals in other tissues were observed in (decreasing signal level): fetal liver>heart=kidney=adrenal gland=testis=fetal heart and kidney=skeletal muscle=bladder=placenta>brain=spinal cord=colon=stomach=spleen=lymph node=bone marrow=trachea=uterus=pancreas=salivary gland=mammary gland=lung. MTSP4 also is expressed less abundantly in several tumor cell lines including HeLa S3=leukemia K-562=Burkitt's lymphomas (Raji and Daudi)=colorectal adenocarcinoma (SW480)>lung carcinoma (A549)=leukemia MOLT-4=leukemia HL-60. PCR of the MTSP4 transcript from cDNA libraries made from several human primary tumors xenografted in nude mice (human tumor multiple tissue cDNA panel, catalog number K1522-1, CLONTECH) was performed using MTSP4-specific primers. The MTSP4 transcript was detected in breast carcinoma (GI-101), lung carcinoma (LX-1), colon adenocarcinoma (GI-112) and pancreatic adenocarcinoma (GI-103). No apparent signal was detected in another form of lung carcinoma (GI-117), colon adenocarcinoma (CX-1), ovarian carcinoma (GI-102). and prostatic adenocarcinoma (PC3). The MTSP4 transcript was also detected in LNCaP and PC-3 prostate cancer cell lines as well as in HT-1080 human fibrosarcoma cell line.

MTSP6

MTSP6 is expressed at high levels in the colon. It also is expressd in the, stomach, trachea, mammary gland, thyroid gland, salivary gland, pituitary gland and pancreas. It is expressed at lower levels in other tissues (see EXAMPLE 6).

MTSP6 also is expressed in several tumor cell lines including HeLa S3>colorectal adenocarcinoma (SW480)>leukemia MOLT-4>leukemia K-562. In mouse xenograft models, the MTSP6 transcript was strongly detected in lung carcinoma (LX-1), moderately detected in pancreatic adenocarcinoma (GI-103), weakly detected in ovarian carcinoma (GI-102); and weakly detected in colon adenocarcinoma (GI-112 and CX-1), breast carcinoma (GI-101), lung carcinoma (GI-117) and prostatic adenocarcinoma (PC3). The MTSP6 transcript was also detected in breast cancer cell line MDA-MB-231, prostate cancer cell line PC-3, but not in HT-1080 human fibrosarcoma cell line. MTSP6 also is expressed in mammary gland carcinoma cDNA (Clontech). MTSP6 also is over expressed in ovarian tumor cells.

MTSP7

The MTSP7 transcript was detected in lung carcinoma (A549 cell line), leukemia (K-562 cell line) and cervical carcinoma (HeLaS3 cell line). MTSP7 is believed to be expressed in lung, colon, prostate, breast, cervical and other tumors.

MTSP9

MTSP9 is, for example, expressed in esophageal tumor tissues, in lung carcinoma, in colorectal carcinoma, lymphoma, a cervical carcinoma (HeLaS3) and leukemia cell lines as well as in certain normal cells and tissues. MTSP9 also can be a marker for breast, prostate, cervical and colon cancer.

MTSP9 is highly expressed in the esophagus and expressed at a low level in many other tissues. The MTSP9 transcript is found in kidney (adult and fetal), spleen (adult and fetal), placenta, liver (adult and fetal), thymus, peripheral blood leukocyte, lung (adult and fetal), pancreas, lymph node, bone marrow, trachea, uterus, prostate, testes, ovary and the gland organs (mammary, adrenal, thyroid, pituitary and salivary). MTSP9 also is expressed in esophagus tumor tissues, in a lung carcinoma and, at a lower level, in a colorectal carcinoma, lymphoma, a cervical carcinoma (HeLaS3) and leukemia cell lines.

MTSP10

MTSP10, for example, is expressed in esophageal tumor tissues, in lung carcinoma, prostate cancers, pancreatic and breast cancers and in cell lines as well as in certain normal cells and tissues (see e.g., EXAMPLES for tissue-specific expression profile). The level of activated MTSP10 can be diagnostic of prostate, uterine, lung esophagus, or colon cancer or leukemia or other cancer. The expression and/or activation of MTSP10 on or in the vicinity of a cell or in a bodily fluid in a subject can be a marker for breast, prostate, lung, colon, esophageal and other cancers.

MTSP10 transcript was detected in pancreas, lung and kidney. MTSP10 transcript was also detected in small intestine Marathon-Ready cDNA (Clontech). The MTSP10 transcript was detected in breast carcinoma (GI-101), lung carcinoma (LX-1 and GI-117), ovarian carcinoma (GI-102), and pancreatic adenocarcinoma (GI-103). The MTSP10 transcript was weakly detected in prostatic adenocarcinoma (PC3). The MTSP10 transcript was also detected in CWR22R prostate tumor grown in nude mice. No apparent signal was detected in two forms of colon adenocarcinomas (GI-112 and CX-1).

MTSP12

MTSP12 transcript was detected in pancreas, lung and kidney. MTSP12 transcript was also detected in small intestine Marathon-Ready cDNA (Clontech). The MTSP12 transcript was detected in breast carcinoma (GI-101), lung carcinoma (LX-1 and GI-117), ovarian carcinoma (GI-102), and pancreatic adenocarcinoma (GI-103). The MTSP12 transcript was weakly detected in prostatic adenocarcinoma (PC3). The MTSP12 transcript was also detected in CWR22R prostate tumor grown on nude mice. No apparent signal was detected in two forms of colon adenocarcinomas (GI-112 and CX-1).

MTSP20

MTSP20 is expressed in the lung, colon, cervical tumors and in leukemic cells. It may also be expressed in breast, ovarian, pancreatic, prostate and in other tumors. MTSP20 transcript was detected in liver, lymph node, cerebellum, pancreas, prostate, uterus, testis, glands (adrenal, thyroid and salivary), thymus, kidney and spleen. Lower transcript level was found in lung, placenta, bladder, ovary, digestive system, circulatory system and other parts of the the brain. MTSP20 is also expressed in certain tumor cell lines including lung carcinoma (A519), colorectal carcinoma (SW480), lymphoma (Raji and Daudi), cervical carcinoma (HeLaS3) and leukemia (HL-60, K-562 and MOLT-4) cell lines.

MTSP22

MTSP22 is expressed in the uterine tissue, thymus, adipose tissue, and lymph node. It may also be expressed in lung, stomach, uterine, breast, ovarian, prostate and in other tumors. MTSP22 transcript was detected in some uterus tissue samples, but not in their matched tumor samples. In one of 42 uterus samples, MTSP22 is expressed in tumor and its metastatic tissues, but not in the normal tissue counterpart. MTSP22 is also expressed in some stomach tumors and lung tumors, but not in their normal tissue counterparts. MTSP22 is also expressed in the normal tissue of a pancreas matched cDNA pair. MTSP22-encoding cDNA was detected in thymus, adipose tissue, and lymph node

MTSP25

MTSP25 is expressed in breast, colon, uterine, ovarian, kidney, prostate, testicular cancer tissue. It may also be expressed in lung, stomach, prostate and in other tumors. MTSP25 transcript was expressed weakly in the lymph node. In the cancer profiling array analysis, MTSP25 is highly expressed in prostate samples (in normal and cancer samples). MTSP25 was highly expressed in a kidney tumor sample, but not in its normal tissue counterpart. MTSP25 was also expressed a breast cancer samples, but not in its normal tissue counterpart. MTSP25 was expressed in normal uterus samples, but not in their tumor counterparts. MTSP25 expression was also ovarian cancer samples. Among these three samples, the expression of MTSP25 was also detected in one of the matched normal tissue counterparts. MTSP25 expression was also detected in tumor samples in colon cDNA pairs.

PCR analysis revealed that MTSP25 cDNA was strongly detected in testis and mammary gland adenocarcinoma, weakly detected in brain, placenta, lung, spleen, prostate, small intestine, colon, and leukocyte, and very weakly detected in heart, liver and pancreas.

2. Endotheliases

Endotheliases are a class of cell surface proteases that are expressed on cells, particularly endothelial cells, particularly those proliferating endothelial cells, which are involved in a variety of proliferative processes, including undesirable angiogenesis associated with tumor growth and metastasis, and with other hyperproliferative disorders, such as restenosis, scarring, diabetic retinopathies, diseases and disorders of the anterior eye (see, U.S. application Ser. No. 09/717,473, filed Nov. 20, 2000, and International PCT application No. PCT/US00/31803).

Proliferative Diseases

Endotheliases are particularly useful targets for delivery of therapeutic agents for treatment of any disorder involving aberrant angiogenesis. Endothelial cells play a key role in angiogenesis, which is is the generation of new blood vessels from parent microvessels. Angiogenesis plays a major role in the metastasis of cancer and in the pathology of a variety of other disorders.

Controlled and uncontrolled angiogenesis proceed in a similar manner. Endothelial cells and pericytes, surrounded by a basement membrane, form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating the new blood vessel.

Angiogenesis, Modulators and Associated Diseases

Angiogenesis is highly regulated by a system of angiogenic stimulators and inhibitors. Known examples of angiogenesis stimulators include certain growth factors, cytokines, proteins, peptides, carbohydrates and lipids (Norrby (1997) APMIS 105:417-437); Polverini (1995) Crit. Rev. Oral. Biol. Med. 6:230-247). A variety of endogenous and exogenous angiogenesis inhibitors are known in the art (Jackson et al. (1997) FASEB 11:457-465; Norrby (1997) APMIS 105:417-437); and O'Reilly (1997) Investigational New Drugs, 15:5-13).

Angiogenesis is essential for normal placental, embryonic, fetal and post-natal development and growth, but almost never occurs physiologically in adulthood except in very specific restricted situations. For example, angiogenesis is normally observed in wound healing, fetal and embryonal development and formation of the corpus luteum, endometrium and placenta. Angiogenesis in the adult is often associated with disease states.

Persistent, unregulated angiogenesis occurs in a multiplicity of disease states, tumor metastasis and abnormal growth by endothelial cells and supports the pathological damage seen in these conditions. The diverse pathological disease states in which unregulated angiogenesis is present have been grouped together as angiogenic dependent or angiogenic associated diseases.

The control of angiogenesis is altered in certain disease states and, in many cases, the pathological damage associated with the disease is related to uncontrolled angiogenesis (see generally, Norrby (1997) APMIS 105:417-437); and O'Reilly (1997) Investigational New Drugs 15:5-13). Thus, angiogenesis is involved in the manifestation or progress of various diseases, for example, various inflammatory diseases, such as rheumatoid arthritis, psoriasis, diabetic retinopathies, certain ocular disorders, including recurrence of pterygii, scarring excimer laser surgery and glaucoma filtering surgery, various disorders of the anterior eye, cardiovascular disorders, chronic inflammatory diseases, wound repair, circulatory disorders, crest syndromes, dermatological disorders (see, e.g., U.S. Pat. Nos. 5,593,990, 5,629,327 and 5,712,291) and notably cancer, including solid neoplasms and vascular tumors. Angiogenesis is essential for the growth and persistence of solid tumors and their metastases. Repressing, eliminating or modulating this activity, should impact the etiology of these diseases and serve as a point of therapeutic intervention. In the disease state, prevention of angiogenesis could avert the damage caused by the invasion of the new microvascular system. Therapies directed at control of the angiogenic processes could lead to the abrogation or mitigation of these diseases. Hence there is a need to develop therapeutics that target angiogenesis and modulate, particularly, inhibit aberrant or uncontrolled angiogenesis.

Hence conjugates that contain endotheliase substrates can be used to deliver therapeutic agents for the treatment of diseases including, but are not limited to, rheumatoid arthritis, psoriasis, diabetic retinopathies, other ocular disorders, including recurrence of pterygii, scarring from excimer laser surgery and glaucoma filtering surgery, various disorders of the anterior eye, cardiovascular disorders, autoimmune diseases, chronic inflammatory diseases, wounds, circulatory disorders, crest syndromes, restenosis, psoriasis and other dermatological disorders (see, e.g., U.S. Pat. Nos. 5,593,990, 5,629,327 and 5,712,291) and notably cancer, including solid neoplasms and vascular tumors.

Endotheliases 1 and 2

Exemplary of endotheliases are two different endotheliases and variant forms thereof designated endotheliase 1 and endotheliase 2 (see SEQ ID Nos. 21-27. Other members of the family can be identified by probing for genes or searching libraries for genes that have sequence identity, particularly at least 40%, 60%, 80%, 90%, 95%, 98% or greater sequence identity to the protease domain of an endotheliase identified herein, or that hybridize under conditions of high stringency to the full-length of the nucleic acid encoding a protease domain of an endotheliase provided herein, and that are expressed on endothelial cells.

Alternatively, and as a way of identifying endotheliases that can have lower sequence identity, an endotheliase can be identified by the methods, such by identifying ESTs or other nucleic acid fragments that have sequences similar to a protease and then using such fragments as probes to identify and select cDNA clones encoding full-length proteases or protease domains thereof, identifying those that have the characteristics of transmembrane proteins, and then determining the gene expression profile to identify those that are expressed on the surface of endothelial cells. Encoded proteins that have protease activity, that include a transmembrane domain and an extracellular domain, and that are expressed in endothelial cells are endotheliases. Any method for identification of genes encoding proteins (or proteins) that encode a transmembrane protease expressed on an endothelial cell is contemplated herein.

Endotheliase 1

Exemplary of the endotheliase are endotheliase 1 and endotheliase 2. These are expressed on endothelial cells. Exemplary of a full-length endotheliase 1 is one that includes the sequence of amino acids set forth in SEQ ID No. 42 (see, International PCT application No. WO 00/5006, which describes a gene it designates DESC1 that is expressed in squamous cell carcinomas and prostate tumors). As noted endotheliases are expressed on endothelial cells. A protease domain thereof is set forth in SEQ ID NO: 22.

Expression Profile of Endotheliase 1

To obtain information regarding the tissue distribution of endotheliase 1, the DNA insert of clone H117 was used to probe an RNA blot composed of 76 different human tissues (catalog number 7775-1; human multiple tissue expression (MTE) array; CLONTECH, Palo Alto, Calif.). Significant expression was observed in the esophagus, with minor expression levels in the stomach, salivary gland, pancreas, prostate, bladder, trachea and uterus. Northern analysis using RNA blots (catalog numbers 7765-1 & 7782-1; human muscle and digestive system multiple tissue northern (MTN) blots; CLONTECH) confirmed that the expression was restricted to the esophagus. Two transcripts (approximately 1.7 and 2 kb) were detected in the esophagus. Endotheliase 1 also is expressed in umbilical vein endothelial cells, PC3 and LnCAP cells.

Endotheliase 2 and Nucleic Acids Encoding Endotheliase 2

Two splice variant forms of endotheliase 2 designated endotheliase 2-S and endotheliase 2-L are exemplified herein (see SEQ ID Nos. 23-26). The open reading frame of the nucleic acid encoding endotheliase 2-S (SEQ ID No. 23) is composed of 1,689 bp, which translates to a 562-amino acid protein (SEQ ID No. 24), while the ORF of endotheliase 2-L is composed of 2,067 bp (SEQ ID No. 25), which translates to a 688-amino acid protein (SEQ ID No. 26).

The nucleic acid encoding the protease domain of endotheliase 2-S is composed of 729 bp which translates to a 242-amino acid protein (amino acids 321-562 of SEQ ID Nos. 23 and 24), while that of endotheliase 2-L is composed of 1,107 bp, which translates to a 368-amino acid protein (amino acids 321-688 of SEQ ID Nos. 25 and 26).

Endotheliase-2 Proteins

Any and all of the above-noted endotheliases and/or protease domains thereof, such as those that include the sequences of amino acids in SEQ ID Nos. 22, 24, 26 and 27 or are encoded by nucleic acid that hybridize thereto under the conditions as described above are contemplated for use in the methods herein. Also contemplated herein are proteins that include amino acid sequence changes, such as those set forth in Table 1 above, and retain protease activity.

Gene Expression Profile and Transcript Size of Endotheliase 2 in Normal and Tumor Tissues

In addition to expression in endothelial cells, endotheliase 2 is expressed in placenta, pancreas, thyroid gland, liver and lung tissues. It also is expressed at lower levels in mammary gland, salivary gland, kidney, trachea, esophagus, appendix, heart and fetal lung. Endotheliase 2 also is expressed in several tumor cell lines and, hence, in certain tumors, including lung and colon, including breast carcinoma, lung carcinomas, colon adenocarcinomas, pancreatic adenocarcinoma (GI-103), and ovarian carcinoma. It has also been detected in prostate and fibrosarcoma cell lines.

### C. Conjugates

Conjugates that are substrates for proteases on the surfaces of cells, particularly serine proteases, including type II membrane-bound serine proteases, and endotheliases are provided. Any cell surface protease, including cell-associated or localized proteases, is contemplated herein. Generally proteases expressed at high levels in active forms in essential tissues are not ideal target candidates. The proteases include those that are expressed on relatively limited numbers of cells or that are expressed at high levels in cells, such as tumor cells and endothelial cells and immune cells, that are involved in disease states or are present in diseases states in the locale of cells involved in the disease states. For example, endothelial cells by virtue of their role in angiogenesis are involved in numerous proliferative disorders; immune cells are involved in many disease processes including cancers and diseases and inflammatory disorders. Other cell surface proteases are expressed at higher levels in certain tumors than in normal cells. Whether or not such proteases have a role in the disorder their higher expression in cells involved in a disease state is sufficient for use for targeting therapeutic agents in the conjugates provided herein.

The conjugates, which contain a therapeutic agent, such as a cytotoxic agent, is activated upon cleavage by a cell surface protease, including cell-associated and cell-localized proteses. Exemplary of such proteases are the MTSPs, such as, but not limited to, MTSP1, MTSP3, MTsP4, MTSP6, MTSP7, MTSP9, MTSP10, MTSP12, MTSP20, MTSP22, MTSP25, urokinases and endotheliases. Hence, the conjugates targeted to such proteases are prodrugs in that the therapeutic agent is inactive as administered and is ultimately activated in the vicinity of the targeted cell or tissue. Although cell surface proteases, such as transmembrane proteases, are the intended targets, any released, shed or soluble forms of the proteases and others also can be targeted.

Thus, the conjugates, which contain a therapeutic agent, such as a cytotoxic agent, are substantially inactive prior to action by a cell surface protease, a peptidic moiety that is a substrate for a targeted cell surface protease (i.e., a peptidic substrate), and, optionally, a linker. The therapeutic agents in the conjugates are activated upon cleavage of the peptidic substrate of the conjugate by a cell surface protease. The therapeutic agents, such as cytotoxic agents, are released as the free yagent, or, alternatively, are released coupled to the portion of the peptidic substrate (P1-P2-P3-etc. (i.e., the N-terminus) or P1′-P2′-etc. (i.e., the C-terminus) that the agents were linked to in the conjugate, optionally via a linker. The cytotoxic agents, in these forms, are released in the vicinity of cells that express the proteases. Activation is effected, in certain embodiments, because the therapeutic agent, such as cytotoxic agent, following action of the cell surface protease, can cross the cell membrane or otherwise interact with the cell or tissue and exhibit therapeutic activity. In other embodiments, any remaining peptidic moieties or amino acids can be cleaved from therapeutic agent to render it active. The conjugates act as prodrugs because the therapeutic agents when conjugated are substantially inactive. Upon cleavage by the targeted protease, the therapeutic agent is released either in active form or in a form that is activated by the targeted cell, tissue or surrounding environment.

In one exemplary embodiment, the targeted agent is a cytotoxic agent and the conjugates for use in the methods and compositions provided herein have the formula:

(peptide^{l})ₛ-(linker)_{q}-(cytotoxic agent)ₜ

or a derivative thereof, where peptide^{l} is a peptidic substrate for a cell surface protease or a released, shed or otherwise unbound membrane protease, such as an MTSP; s is greater than or equal to 1, or is 1 to 6, or is 1 or 2, or is 1; linker is any linker; q is greater than or equal to 0, or is 0 to 4, or is 0 or 1; the cytotoxic agent is an anti-tumor, anti-cancer or anti mitotic agent, including anti-antiangiogenic agents; and t is 1 or more, or is 1 or 2. In these conjugates, the cytotoxic agent is covalently attached, optionally via a linker, to either the C-terminus or the N-terminus of the peptidic substrate. In embodiments where the therapeutic agent, such as a cytotoxic agent, is attached to the C-terminus of the peptidic substrate, the N-terminus optionally is capped. N-Terminal caps for use herein include, but are not limited to, acyl, sulfonyl and carbamoyl groups. In embodiments where the therapeutic agent is attached to the N-terminus of the peptidic substrate, the C-terminus is a carboxamide derivative.

In certain embodiments, peptide^{l} is a peptidic substrate for a cell surface protease or a soluble MTSP whereby, upon action of the protease, the conjugate, which is substantially inactive, is cleaved at the P1-P1′ bond to release a compound of the formula:

(peptide^{a})ₛ-(linker)_{q}-(therapeutic agent)ₜ

or a derivative thereof, that exhibits therapeutic activity, such as cytotoxic activity in vitro and in vivo. In these compounds, peptide^{a} is a truncated version of peptide^{l} resulting from cleavage at the P1-P1′ bond.

In another embodiment, the conjugates for use in the methods and compositions provided herein possess two therapeutic agents, such as cytotoxic agents, which are the same or different, linked to the C-terminus and the N-terminus, respectively, optionally via linkers linker¹ and linker², of a peptidic substrate for cell surface protease or a soluble MTSP. In this embodiment, the conjugates have the formula:

(therapeutic agent¹)ₓ-(linker¹)_{w}-(peptide^{l})ₛ-(linker²)_{q}-(therapeutic agent²)ₜ

or a derivative thereof, where peptide^{l} is a peptidic substrate for a cell surface protease, or a soluble MTSP; s is greater than or equal to 1, or is 1 to 6, or is 1 or 2, or is 1; linker¹ and linker² are each independently any linker and are the same or different; q and w are each independently greater than or equal to 0, or are 0 to 4, or are 0 or 1; the therapeutic agents, which are the same or different, are anti-tumor, anti-cancer or anti mitotic agents; and t and x are each independently 1 or more, or are 1 or 2.

In these embodiments, peptide^{l} is a peptidic substrate for a cell surface protease or a soluble MTSP whereby, upon action of the protease, the conjugate, which is substantially inactive, is cleaved at a point on the peptidic chain to release two compounds of the formulae:

(therapeutic agent¹)ₓ-(linker¹)_{w}-(peptide^{a1})ₛ; and

(peptide^{a2})ₛ-(linker²)_{q}-(therapeutic agent²)ₜ

or derivatives thereof. The released therapeutic agents are active or are further activated by the cell, tissue or surrounding environment. In these compounds, peptide^{a1} and peptide^{a2} are N-terminal and C-terminal truncated portions, respectively, of peptide^{l} resulting from cleavage at the P1-P1′ bond.

In one embodiment, the conjugates for use in the compositions and methods provided herein have formula I:

Xⁿ-(P6)ₘ-(P5)ₚ-(P4)ᵢ-(P3)ⱼ-(P2)ₗ-P1-(P1′)ᵤ-(P2′)ₖ-(P3′)ᵣ-(L)ₙ-Z

or a derivative thereof, where Z is a therapeutic agent; L is a linker; l, j, i, p and m are selected as follows:

l is 0 or 1; when l is 0, j, i, p and m are 0; when l is 1, j is 0 or 1; when j is 0, i, p and m are 0; when j is 1, i is 0 or 1; when i is 0, p and m are 0; when i is 1, p is 0 or 1; when p is 0, m is 0; when p is 1, m is 0 or 1;

u, k and r are selected as follows:

u is 0 or 1; when u is 0, k and r are 0; when u is 1, k is 0 or 1; when k is 0, r is 0; when k is 1, r is 0 or 1;

n is 0 or 1; Xⁿ is hydrogen, or an acyl, sulfonyl or carbamoyl cap; and P6 to P3′ are amino acid residues, as defined below. In this embodiment, the P6 to P3′ residues are linked by peptide bonds or peptide bond surrogates. Thus, the P6 to P3′ portion of the conjugate is a peptidic substrate, as defined herein.

In another embodiment, the conjugates for use in the compositions and methods provided herein have formula II:

Z-(L)ₙ-(P6)ₘ-(P5)ₚ-(P4)ᵢ-(P3)ⱼ-(P2)ₗ-P1-(P1′)ᵤ-(P2′)ₖ-(P3′)ᵣ-X^{c}

or a derivative thereof, where Z is a therapeutic agent; L is a linker; l, j, i, p and m are selected as follows:

l is 0 or 1; when l is 0, j, i, p and m are 0; when l is 1, j is 0 or 1; when j is 0, i, p and m are 0; when j is 1, i is 0 or 1; when i is 0, p and m are 0; when i is 1, p is 0 or 1; when p is 0, m is 0; when p is 1, m is 0 or 1;

u, k and r are selected as follows:

u is 0 or 1; when u is 0, k and r are 0; when u is 1, k is 0 or 1; when k is 0, r is 0; when k is 1, r is 0 or 1;

n is 0 or 1; X^{c}, together with the carbonyl group of the amino acid residue to which it is attached, forms a carboxylic acid or a carboxamide group; and P6 to P3′ are amino acid residues, as defined below. In this embodiment, the P6 to P3′ residues are linked by peptide bonds or peptide bond surrogates. Thus, the P6 to P3′ portion of the conjugate is a peptidic substrate, as defined herein.

In a further embodiment, the conjugates for use in the compositions and methods provided herein have formula III:

Z¹-(L¹)ₙ-(P6)ₘ-(P5)ₚ-(P4)ᵢ-(P3)ⱼ-(P2)ₗ-P1-(P1′)ᵤ-(P2′)ₖ-(P3′)ᵣ(L²)ᵥ-Z²

or a derivative thereof, where Z¹ and Z² are each therapeutic agents and are the same or different; L¹ and L² are each linkers and are the same or different; l, j, i, p and m are selected as follows:

l is 0 or 1; when l is 0, j, i, p and m are 0; when l is 1, j is 0 or 1; when j is 0, i, p and m are 0; when j is 1, i is 0 or 1; when i is 0, p and m are 0; when i is 1, p is 0 or 1; when p is 0, m is 0; when p is 1, m is 0 or 1;

u, k and r are selected as follows:

u is 0 or 1; when u is 0, k and r are 0; when u is 1, k is 0 or 1; when k is 0, r is 0; when k is 1, r is 0 or 1;

n and v are each independently 0 or 1; and P6 to P3′ are amino acid residues, as defined below. In this embodiment, the P6 to P3′ residues are linked by peptide bonds or peptide bond surrogates. Thus, the P6 to P3′ portion of the conjugate is a peptidic substrate, as defined herein.

In another embodiment, the conjugates for use in the compositions and methods provided herein have formula IV:

Xⁿ-(P6)ₘ-(P5)ₚ-(P4)ᵢ-(P3)ⱼ-(P2)ₗ-P1-(P1′)ᵤ-(P2′)ₖ-(P3′)ᵣ-(P4′)ₛ-(L)ₙ-Z

or a derivative thereof, where Z is a therapeutic agent; L is a linker; l, j, i, p and m are selected as follows:

l is 0 or 1; when l is 0, j, i, p and m are 0; when l is 1, j is 0 or 1; when j is 0, i, p and m are 0; when j is 1, i is 0 or 1; when i is 0, p and m are 0; when i is 1, p is 0 or 1; when p is 0, m is 0; when p is 1, m is 0 or 1;

u, k, r and s are selected as follows:

u is 0 or 1; when u is 0, k, r and s are 0; when u is 1, k is 0 or 1; when k is 0, r and s are 0; when k is 1, r is 0 or 1; when r is 0, s is 0; when r is 1, s is 0 or 1;

n is 0 or 1; Xⁿ is hydrogen, or an acyl, sulfonyl or carbamoyl cap; and P6 to P4′ are amino acid residues, as defined below. In this embodiment, the P6 to P4′ residues are linked by peptide bonds or peptide bond surrogates. Thus, the P6 to P4′ portion of the conjugate is a peptidic substrate, as defined herein. In another embodiment, the conjugates for use in the compositions and methods provided herein have formula V:

Z-(L)ₙ-(P6)ₘ-(P5)ₚ-(P4)ᵢ-(P3)ⱼ-(P2)ₗ-P1-(P1′)ᵤ-(P2′)ₖ-(P3′)ᵣ-(P4′)ₛ-X^{c}

or a derivative thereof, where Z is a therapeutic agent; L is a linker; i, j, i, p and m are selected as follows:

l is 0 or 1; when l is 0, j, i, p and m are 0; when l is 1, j is 0 or 1; when j is 0, i, p and m are 0; when j is 1, i is 0 or 1; when i is 0, p and m are 0; when i is 1, p is 0 or 1; when p is 0, m is 0; when p is 1, m is 0 or 1;

u, k, r and s are selected as follows:

u is 0 or 1; when u is 0, k, r and s are 0; when u is 1, k is 0 or 1; when k is 0, r and s are 0; when k is 1, r is 0 or 1; when r is 0, s is 0; when r is 1, s is 0 or 1;

n is 0 or 1; X^{c}, together with the carbonyl group of the amino acid residue to which it is attached, forms a carboxylic acid or a carboxamide group; and P6 to P4′ are amino acid residues, as defined below. In this embodiment, the P6 to P4′ residues are linked by peptide bonds or peptide bond surrogates. Thus, the P6 to P4′ portion of the conjugate is a peptidic substrate, as defined herein.

In a further embodiment, the conjugates for use in the compositions and methods provided herein have formula VI:

Z¹-(L¹)ₙ-(P6)ₘ-(P5)ₚ-(P4)ᵢ-(P3)ⱼ-(P2)ₗ-P1-(P1′)ᵤ-(P2′)ₖ-(P3′)ᵣ-(P4′)ₛ-(L²)ᵥ-Z²

or a derivative thereof, where Z¹ and Z² are each therapeutic agents and are the same or different; L¹ and L² are each linkers and are the same or different; l, j, i, p and m are selected as follows:

l is 0 or 1; when l is 0, j, i, p and m are 0; when l is 1, j is 0 or 1; when j is 0, i, p and m are 0; when j is 1, i is 0 or 1; when i is 0, p and m are 0; when i is 1, p is 0 or 1; when p is 0, m is 0; when p is 1, m is 0 or 1;

u, k, r and s are selected as follows:

u is 0 or 1; when u is 0, k, r and s are 0; when u is 1, k is 0 or 1; when k is 0, r and s are 0; when k is 1, r is 0 or 1; when r is 0, s is 0; when r is 1, s is 0 or 1;

n and v are each independently 0 or 1; and P6 to P4′ are amino acid residues, as defined below. In this embodiment, the P6 to P4′ residues are linked by peptide bonds or peptide bond surrogates. Thus, the P6 to P4′ portion of the conjugate is a peptidic substrate, as defined herein.

Exemplary peptidic substrates, therapeutic agents, linkers and exemplary conjugates of formulae I-VI are described in further detail below. It is intended herein that conjugates resulting from all combinations and/or permutations of the groups recited below for the variables of formulae I-VI are encompassed within the instant disclosure.

1. Peptidic Substrates

The peptidic substrates contemplated for use in the conjugates are substrates for the targeted cell surface protease or a soluble, shed or released form thereof, and contain a sufficient number of amino acid residues to render any therapeutic agent in the conjugate substantially inactive. In the exemplary embodiment where the therapeutic agent is, for example, doxorubicin, the conjugate is substantially inactive by virtue of the inability of the conjugated therapeutic agent to cross the cell membrane. In certain embodiments, the peptidic substrate contains at least 1, 2, 3, 4 or 5 amino acid residues, and can contain up to nine or ten residues. Longer peptidic substrates can be used in the conjugates as long as upon cleavage, the resulting therapeutic agent or therapeutic agent-amino acid or -peptidic moiety conjugate exhibits the desired therapeutic effect in vivo and in vitro.

Hence, exemplary peptidic substrates for use in the conjugates provided herein possess at least one amino acid (P1), two amino acids (P1-P1′), three amino acids (P2-P1-P1′) and typically contain four, five or six amino acid residues (P3-P2-P1-P1′, P4-P3-P2-P1-P1′ or P4-P3-P2-P1-P1′-P2′), where the P1-P1′ bond is the site of cleavage of cell surface protease, or a soluble, shed or released form thereof, including, but not limited to, a cell surface protease, such as a serine protease, including, for example, but not limited to, uPA bound to its receptor, MTSPs and endotheliases. The peptidic substrates optionally further possess a P5, P6 or P3′ amino acid residue, and, in certain embodiments, possess P7, P8, P9, P10, P4′, P5′, P6′ residues. Thus, the peptidic substrates for use in the conjugates provided herein are penta-, hexa-, hepta-, octa- and nona-peptidic substrates, and can contain 10, 11, 12, 13, 14, 15 or more residues as long as, upon cleavage of the conjugate by the protease, the resulting therapeutic agent or therapeutic agent-amino acid or -peptidic moiety conjugate exhibits the desired therapeutic effect in vivo and in vitro.

The peptidic substrates are conjugated to the therapeutic agent (or to a linker to which the therapeutic agent is linked) via the C-terminal residue (i.e., P1′, P2′ or P3′), or the N-terminal residue (i.e., P6, P5 or P4), or optionally an internal residue. The peptidic substrates, for example, can be straight chains, but can be cyclized or include cyclized portions.

In embodiments where the conjugation is via the C-terminus of the peptidic substrate, the peptidic substrate optionally possesses a cap, such as an acyl or carbamoyl cap at the N-terminus. In embodiments where conjugation is via the N-terminus of the peptidic substrate, the peptidic substrate further possess a terminal group, such as a carboxamide group, at the C-terminus.

The conjugates can contain a plurality of peptidic substrates and a plurality of therapeutic agents. For example, in conjugates that contain two therapeutic agents, which are the same or different, conjugation to the therapeutic agent(s) or linker linked thereto can be via the C-terminal and N-terminal residues of the peptidic substrate.

The methods described for selection of substrates above can be used to design suitable substrates. In addition, substrates can be designed based upon known specificities of other proteases. For example, the specificities of trypsin-like and trypsin family members can aid in design of possible substrates. The following summarizes substrate preferences for particular serine proteases (see, e.g., Harris et al. (2000) PNAS 97(14):7754-7759).

| | EXEMPLARY | EXEMPLARY | EXEMPLARY |
|---|---|---|---|
| PROTEASE | P1 RESIDUE(S) | P2 RESIDUE(S) | P3 RESIDUE(S) |
| Chymotrypsin | Tyr, Phe, Trp | — | — |
| Trypsin | Arg, Lys | — | — |
| Thrombin | Arg, Lys | Phe | Thr, Trp |
| Plasmin | Lys, Arg | Trp, Tyr, Met | Gln |
| Granzyme B | Asp | — | — |
| Human | Ala, Val, Ile | — | — |
| Neutrophil | | | |
| Elastase | | | |
| Tissue | Arg | Ser, Gly, Ala | Met, Tyr |
| Plasminogen | | | |
| Factor | | | |
| Urokinase | Arg | Ser, Ala | Thr, Ser |
| Factor Xa | Arg | Gly | — |

Typical protocols for preparation of the conjugates can include the steps of: 1) identification of a targeted protease; 2) expression and assay development; 3) substrate selection, such as, for example, by testing chromogenic or fluorogenic substrates to identify those cleaved by a selected target protease, by use of substrate phage display to identify peptidic substrates cleaved by a targeted protease, by use of a natural protein or peptide substrate or a natural inhibitor of the protease, and by use of combinatorial libraries to identify substrates cleaved by a targeted protease; 4) synthesis of conjugates containing the identified substrate; and 5) biological evaluation thereof, including, but not limited to, in vitro assays, cell culture assays, biological assays, and in vivo animal models (see, e.g., EXAMPLE 10).

A conjugate can be designed by any methods known to those of skill in the art. The following provides an exemplary protocol. First, a series of commercially available chromogenic and fluorogenic peptidic substrates can be tested for cleavage by the protease of interest (see Examples for lists of exemplary chromogenic and fluorogenic substrates and the table below). The peptidic portion of these substrates occupies the unprimed binding sites of the protease while the reporter group is located on the primed side of the scissle bond. Effective conjugates can then be designed based on the structure of the substrates that are efficiently cleaved by the protease.

The peptidic portion of these efficiently cleaved substrates can be used as the unprimed region of the conjugate, and Ser-therapeutic agent, such as a cytotoxic agent (e.g., doxorubicin), Ser-Leu-therapeutic agent or Ser-Ser-Leu-therapeutic agent can be used as the primed region of the conjugate. Cleavage of these conjugate prodrugs releases either Ser-therapeutic agent, Ser-Leu-therapeutic agent or Ser-Ser-Leu-therapeutic agent compounds. In another embodiment, the Ser in the released Ser-therapeutic agent may be replaced by other amino acid residues including, but not limited to, Ala, hSer, Abu, Thr, Met, nLeu and Val. In another embodiment, such as when the therapeutic agent is doxorubicin, the amino acid residue conjugated to the therapeutic agent possesses a hydrophobic side chain. Such amino acid residues include, but are not limited to, Leu, Abu, nLeu, nVal, CHA, hCHA, (hex)Gly, (allyl)Gly, (propargyl)Gly and (cyclopropyl)Ala. In another embodiment, such as when the therapeutic agent is taxol, the amino acid residue conjugated to the therapeutic agent possesses a side chain that is not sterically bulky. Such amino acid residues include, but are not limited to, Gly and Ala. The resulting P1′-therapeutic agent, P1′-P2′-therapeutic agent, or P1′-P2′-P3′-therapeutic agent compound can be further processed in vivo into active therapeutic agents.

Another approach to designing a conjugate prodrug for a protease substrate is to use substrate phage display to elucidate optimal subsite occupancy for the protease. The resulting information can then be used to design the peptidic, unprimed portion of the conjugate. As described above, the primed region of the conjugate can be fixed as Ser-therapeutic agent, Ser-Leu-therapeutic agent or Ser-Ser-Leu-therapeutic agent.

A third approach to design an effective prodrug conjugate involves the use of combinatorial fluorogenic substrate libraries to determine optimal residues for the unprimed region of a protease substrate. These selected sequences can then be used as the unprimed portion of the conjugate prodrug and, and Ser-therapeutic agent, (e.g., doxorubicin), Ser-Leu-therapeutic agent or Ser-Ser-Leu-therapeutic agent can be used as the primed region of the conjugate. These methods have been used in the design of the peptidic substrate portion of the conjugates provided herein. For example, sequences including GSGR (and related sequences such as TGR, SGR, extended variants and others herein) were based on or dervied from substrate phage display experiments using u-PA as the taret protease. Many matriptase conjugates, such as (R/K)-X-S-R and X-(R/K)-S-R, and related sequences as provided herein, were based on data from combinatorial libraries. In other embodiemnts, seqeuence sequences in natural substrates or natural inhibitors of a protease target, such as uPA, including VSAR, PGR (from P3-P1 of plasminogen) and related sequences, were used in design of u-PA-targetd conjugates. In other embodiments, sequences from chromgenic substrates, such as D-HHT-Gly-Arg, and related sequences, were used for design of ET-1-targeted conjugates.

Chromogenic/Fluorogenic Substrates

Chromogenic/fluorogenic substrates

| Enzyme | Substrate | Structure |
|---|---|---|
| MTSP1 | Spectrozyme t-PA | CH₃SO₂-D-HHT-Gly-Arg-pNA.AcOH |
| MTSP1 | S 2765 | N-α-Z-D-Arg-Gly-Arg-pNA.2HCl |
| MTSP3 | Spectrozyme fXIIa | H-D-CHT-Gly-Arg-pNA.2AcOH |
| MTSP4^{a} | Spec PL | H-D-Nle-HHT-Lys-pNA.2AcOH |
| MTSP5 | S 2765 | N-α-Z-D-Arg-Gly-Arg-pNA.2HCl |
| MTSP6 | spectrozyme t-PA | CH₃SO₂-D-HHT-Gly-Arg-pNA.AcOH |
| MTSP7 | S 2366 | pyroGlu-Pro-Arg-pNA.HCl |
| MTSP9 | Pefachrome fVIIa | CH₃SO₂-D-CHA-But-Arg-pNA |
| MTSP10 | spectrozyme t-PA | CH₃SO₂-D-HHT-Gly-Arg-pNA.AcOH |
| MTSP22 | S 2366 | pyroGlu-Pro-Arg-pNA.HCl |
| ET-1 | spectrozyme t-PA | CH₃SO₂-D-HHT-Gly-Arg-pNA.AcOH |
| ET-2 | S 2765 | N-α-Z-D-Arg-Gly-Arg-pNA.2HCl |
| u-PA | S-2444 | pyroGlu-Gly-Arg-pNA.HCl |

^{a}coupled assay, activation of plasminogen in the presence of Spec PL

Briefly, for a coupled assay, the ability of the protease to activate an enzyme, such as plasminogen or trypsinogen is tested. To perform these assays, a protease is incubated with a zymogen, such as plasminogen or trypsinogen, in the presence of a labelled known substrate, such as lys-plasminogen or Spec PL (for plasmin), for the zymogen. If protease activates the zymogen, the activated enzyme, such as plasmin and trypsin, will degrade the substrate, thereby changing the spectral properties of the substrate.

Exemplary Peptidic Substrates

The following description provides exemplary peptidic substrates for cleavage by proteases, such as MTSP1 (or matriptase), endotheliase 1 and urokinase, and a general discussion of properties of the residues. In a similar manner, peptidic substrates for cleavage by other cell surface proteases, or a soluble, shed or released form thereof, can be similarly designed by identifying peptidic substrates for the selected protease and then preparing conjugates that contain such peptidic substrates.

a. The P1 Residue

Amino acid residues for use at the P1 position of the peptidic substrates for use in the conjugates provided herein include Arg, Arg surrogates and Lys. Arg surrogates include unnatural amino acids that possess a group or moiety that functions in substantially the same way as the naturally occurring side chain of arginine to achieve substantially the same result (e.g., acting as the P1 residue in a substrate for a MTSP1, urokinase or endotheliase). Arg surrogates include, but are not limited to, α-amino acids that possess as the side chain any of the following: the side chain of homoarginine; guanidinoaminopropyl; guanidinoaminoethyl; (Me)₂arginine side chain; (Et)₂arginine side chain; (4-aminomethyl)phenylmethyl; 4-amidinophenylmethyl; 4-guanidinophenylmethyl; or the Arg surrogate is a conformationally constrained arginine analog such as:

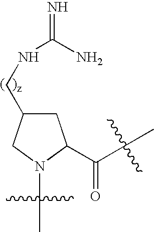

where z is 0 or 1 (see, e.g., Webb et al. (1991) J. Org. Chem. 56:3009); or the side chain is a conformationally constrained arginine side chain analog such as:

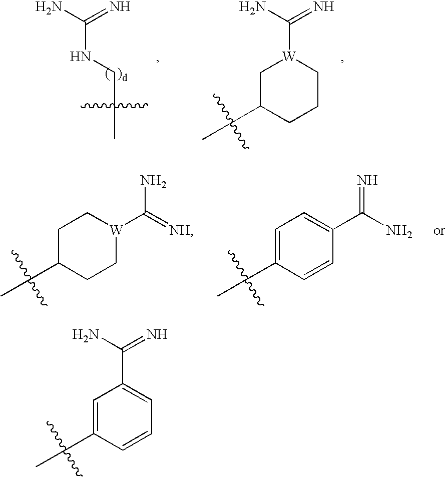

where d is an integer from 0 to 5, or 1 to 3; and W is N or CH; or a mono- or di-substituted N-alkyl derivative of the above groups, where alkyl is, in certain embodiments, lower alkyl, such as, for example, methyl.

In certain embodiments herein, the P1 residue is Arg.

b. The P2 Residue

In the conjugates provided herein, the P2 residue is selected from Phe, Ser, Gly, Ala, Ser(OMe), hSer, 1-methylHis, 3-methylHis, His, nVal, nLeu, Abu, (hS)Gly, Thr, Aib, CHA and Tyr. In another embodiment, the P2 residue is selected from Phe, Ser, Gly and Ala. In certain embodiments herein, the P2 residue is Ser or Ala. In another embodiment, the P2 residue is Gly or Ala.

c. The P3 Residue

Amino acid residues for use at the P3 position of the conjugates provided herein include Arg, Lys, Gln, Quat, Arg surrogates, Ser, Thr, hSer, dSer, Pro, (hS)Gly, Tyr, 4,4-dimethylThr, Asn, Met(O₂), Quat², Quat³, Quat⁴ and Quat⁵. In another embodiment, the P3 residue is selected from Arg, Lys, Gln, Quat and Arg surrogates. Arg surrogates include those described above for the P1 residue.

In certain embodiments, the P3 residue is Gin or Ser.

d. The P4 Residue

In the conjugates provided for use in the compositions and methods provided herein, the P4 residue is selected from Pro, Arg, Ser, Ala, Lys, Gly, nLeu, Leu, Tyr, Glu, Phe, Val, N,N-dimethylGly, β-Ala, Cys(Me), Gin, t-butylGly and nVal. In another embodiment, the P4 residue is selected from Pro, Arg, Ser, Ala, Lys, Gly, nLeu, Leu, Tyr, Glu, Phe and Val. In further embodiments, the P4 residue is selected from Pro, Arg, Ser, Ala, Lys, Gly, nLeu, Phe or Val. In certain embodiments herein, the P4 residue is Arg or Gly.

e. The P5 and P6 Residues

In certain embodiments herein, the peptidic substrates used in the conjugates contain a P5 and, optionally, a P6 residue. P5 residues include Ile, Arg and Arg surrogates. In another embodiment, P5 residues include Arg and Arg surrogates. Arg surrogates include those described above for the P1 residue. P6 residues include, for example, Leu, Val and Arg. In another embodiment, P6 residues include, for example, Leu.

f. The P1′ Residue

The P1′ residue of the conjugates provided herein is Gly, Ser, Ala, Leu, Ile, d-Ile, nLeu, Val, nVal, Aib, Abu, Met, 6-aminohexanoyl, Thr or hSer. In another embodiment, the P1′ residue of the conjugates provided herein is Gly, Ser, Ala, Leu, Ile, d-Ile, nLeu, Val, nVal, Aib, Abu, Met or 6-aminohexanoyl. In another embodiment, the P1′ residue is Ser, Ala, hSer, Abu, Thr, Met, nLeu or Val. In another embodiment, the P1′ residue is Gly or Ala. In another embodiment, the P1′ residue is Ser, Ala or Gly. In another embodiment, the P1′ residue is Leu, Abu, nLeu, nVal, CHA, hCHA, (hex)Gly, (allyl)Gly, (propargyl)Gly or (cyclopropyl)Ala. In certain embodiments herein, the P1′ residue is Ala, Ser, Gly, Ile or d-Ile.

g. The P2′ Residue

In certain embodiments herein, the conjugates provided herein possess a P2′ residue. P2′ residues for use herein include, but are not limited to, Gly, Ser, Ala, Leu, Ile, d-Ile, nLeu, Val, nVal, Aib, Abu, Met, 6-aminohexanoyl, hCHA, CHA, hexylGly, allylGly and Phe. In another embodiment, P2′ residues for use herein include, but are not limited to, Gly, Ser, Ala, Leu, Ile, d-Ile, nLeu, Val, nVal, Aib, Abu, Met and 6-aminohexanoyl. In another embodiment, the P2′ residue is Ser, hSer, Abu, nLeu, nVal, CHA, hCHA, (allyl)Gly or (hexyl)Gly. In another embodiment, the P2′ residue is Gly or Ala. In another embodiment, the P2′ residue is Leu, Abu, nLeu, nVal, CHA, hCHA, (hex)Gly, (allyl)Gly, (propargyl)Gly or (cyclopropyl)Ala. In further embodiments, the P2′ residues are Ala, Gly, Ile or d-Ile.

h. The P3′ Residue

In other embodiments herein, the peptidic substrates used in the conjugates provided herein include a P3′ residue. P3′ residues for use herein include, but are not limited to, Gly, Ser, Ala, Leu, Ile, nLeu, Val, nVal, Aib, Abu, Met, 6-aminohexanoyl, CHA and allylGly. In another embodiment, the P2′ residue is Ser, hSer, Abu, nLeu, nVal, CHA, hCHA, (allyl)Gly or (hexyl)Gly. In another embodiment, P3′ residues for use herein include, but are not limited to, Gly, Ser, Ala, Leu, Ile, nLeu, Val, nVal, Aib, Abu, Met and 6-aminohexanoyl. In another embodiment, the P3′ residue is Gly or Ala. In another embodiment, the P3′ residue is Leu, Abu, nLeu, nVal, CHA, hCHA, (hex)Gly, (allyl)Gly, (propargyl)Gly or (cyclopropyl)Ala.

i. The P4′ Residue

In other embodiments herein, the peptidic substrates used in the conjugates provided herein include a P4′ residue. P4′ residues for use herein include, but are not limited to, Gly, Ser, Ala, Leu, Ile, nLeu, Val, nVal, Aib, Abu, Met, 6-aminohexanoyl, CHA and allylGly. In another embodiment, P4′ residues for use herein include, but are not limited to, Gly, Ser, Ala, Leu, Ile, nLeu, Val, nVal, Aib, Abu, Met and 6-aminohexanoyl. In another embodiment, the P4′ residue is Gly or Ala. In another embodiment, the P4′ residue is Leu, Abu, nLeu, nVal, CHA, hCHA, (hex)Gly, (allyl)Gly, (propargyl)Gly or (cyclopropyl)Ala. In another embodiment, the P4′ residue is Leu.

j. Caps

1) Xⁿ (the N-terminal Cap)

In embodiments herein where the therapeutic agent is conjugated to the C-terminus of the peptidic substrate (i.e., where the conjugate has formula I), the N-terminus of the peptidic substrate optionally is capped with an acyl, sulfonyl or carbamoyl derivative. The cap is chosen, in certain embodiments, to increase the hydrophilicity of the conjugate. In embodiments where the peptidic substrate-therapeutic agent conjugate is sufficiently hydrophilic so as not to require further hydrophilicity, a non-hydrophilic N-terminal cap, such as an acetyl group, can be used. In embodiments where increased hydrophilicity is desired, the N-terminal amino acid is modified with a hydrophilic blocking group. Such blocking groups are chosen based upon the presence of hydrophilic functionality. Such blocking of the terminal amino group can also reduce or eliminate the enzymatic degradation of such peptidyl therapeutic agents by the action of exogenous amino peptidases which are present in the blood plasma of warm blooded animals.

N-Terminal blocking groups that increase the hydrophilicity of the conjugates and therefore increase the aqueous solubility of the conjugates include, but are not limited to, hydroxylated alkanoyl, polyhydroxylated alkanoyl, polyethylene glycol, glycosylates, sugars and crown ethers.

In certain embodiments herein, the N-terminal blocking group is one of the following:

a)

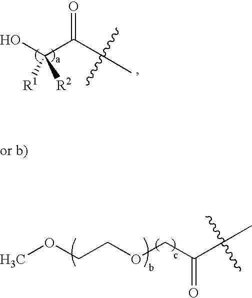

where R¹ and R² are selected from (i) or (ii) as follows:

(i) R¹ and R² are each independently:

a) hydrogen;

b) unsubstituted or substituted aryl, unsubstituted or substituted heterocyclyl, C₃-C₁₀ cycloalkyl, C₂-C₆ alkenyl, C₂-C₆ alkynyl, halogen, C₁-C₆ perfluoroalkyl, R⁴O—, R³C(O)NR³—, (R³)₂NC(O)—, (R³)₂N—C(NR³)—, R⁴S(O)ₑNH—, —CN, —NO₂, R³C(O)—, —N₃, —N(R³)₂, or R⁴OC(O)NR³—;

c) unsubstituted C₁-C₆ alkyl;

d) substituted C₁-C₆ alkyl wherein the substituent on the substituted C₁-C₆ alkyl is selected from unsubstituted or substituted aryl, unsubstituted or substituted heterocyclyl, C₃-C₁₀ cycloalkyl, C₂-C₆ alkenyl, C₂-C₆ alkynyl, R³O—, R⁴S(O)ₑNH—, R³C(O)NR³—, (R³)₂NC(O)—, (R³)₂N—C(NR³)—, —CN, R³C(O)—, —N₃, —N(R³)₂, and R⁴OC(O)—NR³—; or

(ii) R¹ and R² are combined to form —(CH₂)_{f}— where one of the carbon atoms optionally is replaced by a moiety selected from: —O—, —S(O)ₑ—, —NC(O)—, —NH— and —N(COR⁴)—;

R³ is selected from: hydrogen, unsubstituted or substituted aryl, unsubstituted or substituted heterocyclyl, C₁-C₆ alkyl and C₃-C₁₀ cycloalkyl;

R⁴ is selected from: unsubstituted or substituted aryl, unsubstituted or substituted heterocyclyl, C₁-C₆ alkyl and C₃-C₁₀ cycloalkyl;

e is 0, 1 or 2;

a is 1, 2, 3 or 4;

b is zero or an integer between 1 and 100; and

c is 0 to 10, provided that if b is zero, c is 1 to 10; and

f is 3, 4 or 5.

In certain embodiments, R¹ and R² are each independently hydrogen, OH, C₁-C₆ alkyl, C₁-C₆ alkoxy, C₁-C₆ aralkyl or aryl. In these embodiments, a is 1, 2, 3 or 4; b is 0 or an integer between 1 and 100; and c is 0 to 10, provided that if b is 0, c is 1 to 10.

In another embodiment, the N-terminal cap (Xⁿ) is hydrogen, or (i), (ii), (iii) or (iv) as follows:

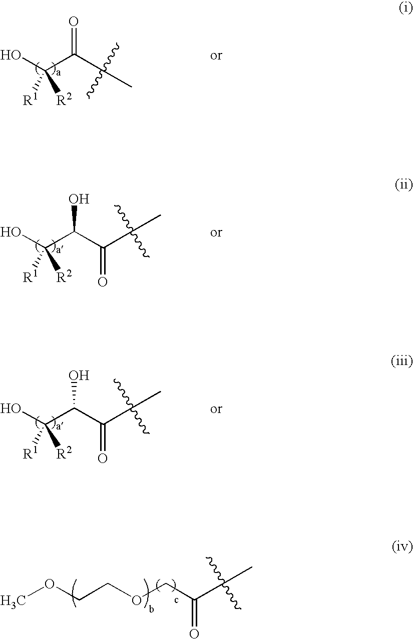

where R¹ and R² are each independently hydrogen, C₁-C₆ alkyl and aryl; a is 1, 2, 3 or 4; a′ is 0, 1, 2 or 3; b is 0 or an integer between 1 and 14; and c is 0 or 1, provided that if b is 0, c is 1.

In another embodimbent, Xⁿ is R³⁰O—C(O)—, R³¹R³²N—C(O)—, R³³(CH₂)ₖC(O)— or H—C(O)—; where k is an integer from 1 to 4, or is 1 or 2; R³⁰ is alkyl, aryl, heteroaryl, aralkyl or heteroaralkyl; R³¹ and R³² are each independently hydrogen, alkyl, aryl, heteroaryl, aralkyl, or heteroaralkyl; and R³³ is hydrogen, hydroxy, alkyl, alkenyl, alkynyl, alkoxy, aryl, aryloxy, heteroaryl, heteroaryloxy, aralkyl, aralkoxy, heteroaralkyl or heteroaralkoxy.

In certain embodiments herein, Xⁿ is hydrogen, acetyl, hydroxyacetyl, 2,3-dihydroxypropionyl, 2,3,4-trihydroxybutanoyl, PEG(1), PEG(2), PEG(4), PEG(6), PEG(14), PEG(15), PEG(16), PEG(17), PEG(18) or PEG(19). In other embodiments herein, Xⁿ is hydrogen, acetyl, hydroxyacetyl, succinyl, quinyl, gallyl, 4-imidazolylacetyl, cotininyl, 3-phosphonylpropionyl, gulonyl, 4-phosphonylbutyryl, glutaryl, ethoxysquaryl or PEG(2). In further embodiments, Xⁿ is hydrogen, acetyl, —C(O)NH₂, HOCH₂CH₂C(O)—, diaminopropanoyl, or NH₂—(CH₂)₅—C(O)—. In another embodiment, Xⁿ is hydrogen, acetyl, succinyl, glutaryl, PEG(2) or malonyl. In another embodiment, Xⁿ is hydrogen, acetyl, succinyl, glutaryl, PEG(2), malonyl, methoxycarbonyl, phenylsulfonyl, 3-methoxypropanoyl, ethoxycarbonyl, isobutoxycarbonyl, benzyloxycarbonyl, tert-butoxycarbonyl, 4-oxopentanoyl, 2-(2-methoxyethoxy)ethoxy)acetyl, 3,4-methylenedioxyphenylacetyl, 2-pyridylacetyl, phenoxyacetyl, phenylacetyl, methoxyacetyl, 2-methoxyethoxycarbonyl, 2-methoxyethoxyacetyl, 3-phenyl-2-hydroxypropanoyl, pent-4-ynoyl, 1-naphthylacetyl, hydroxyacetyl, 3-methoxycarbonylpropanoyl or formyl.

In certain embodiments herein, the N-terminal cap (Xⁿ) is acetyl, glutaryl, or related acyl, sulfonyl or carbamoyl derivatives. Capping groups include, but are not limited to, a simple N-acetyl residue through larger fragments that impact the overall physicochemical properties of the conjugate. Appropriate choice of the capping group allows delivery of either relatively hydrophilic or hydrophobic molecules to a target site. In one embodiment, Xⁿ is acetyl.

2) X^{c} (the C-terminal Cap)

In embodiments herein where the therapeutic agent is conjugated to the N-terminus of the peptidic substrate (i.e., where the conjugate has formula II), the C-terminus of the peptidic substrate is a carboxylic acid or a carboxamide derivative. Appropriate choice of the capping group allows delivery of either relatively hydrophilic or hydrophobic molecules to a target site.

In one embodiment, X^{c}, together with the carbonyl group to which it is attached, forms a carboxamide derivative of formula —C(O)NR^{d}R^{e}, where R^{d} and R^{e} are selected from (i) or (ii) as follows:

(i) R^{d} and R^{e} are each independently hydrogen, C₁-C₆-alkyl, —C₁-C₆-alkyl-OH, —C₁-C₆-alkyl-di-OH, —C₁-C₆-alkyl-tri-OH and

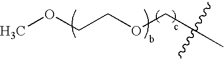

provided that at least one of R^{d} and R^{e} are not hydrogen or C₁-C₆-alkyl; or

(ii) R^{d} and R^{e} together form a —CH₂CH₂OCH₂CH₂— diradical;

b is zero or an integer between 1 and 100; and

c is 0 or 1, provided that if b is zero, c is 1.

In one embodiment, R^{d} is hydrogen and R^{e} is 2-hydroxyethyl.

2. Linkers

The conjugates optionally contain a linker (i.e., L, L¹ or L² of formulae I, II and III) that covalently binds the peptidic substrate to the therapeutic agent. The linkers are any that result in a conjugate in which the peptidic portion is a substrate for a cell surface protease and the therapeutic agent is substantially inactive when in the conjugate and is released in active form or in a form subsequently activated by the cell, tissue or environment of the targeted tissue.

For example, the linker can include of carbohydrate, peptide, diamine, arylamine, and/or hydrocarbon core structures. Linkers are desirably synthetically accessible, provide shelf-stable products, and do not possess any intrinsic biological activity that interferes with the conjugates activity. They can add desirable properties such as increasing solubility or serving to aid in trafficking the cleaved therapeutic agent in the cell. In certain embodiments, some linkers will be enzymatically cleaved in vitro and in vivo, and fragment to release active therapeutic agent or activatable therapeutic agent. In embodiments where the therapeutic agent is doxorubicin, the linker is, for example, a sugar and/or a peptide, such the aminosugar daunosamine.

In one embodiment, linkers for use herein include, but are not limited to, a biscarbonyl alkyl diradical whereby an amine moiety on the therapeutic agent is connected with the linker unit to form an amide bond and the amino terminus of the peptidic substrate is connected with the other end of the linker unit also forming an amide bond. Conversely, a diaminoalkyl diradical linker unit, whereby a carbonyl moiety on the cytotoxic agent is covalently attached to one of the amines of the linker unit while the other amine of the linker unit is covalently attached to the C-terminus of the peptidic substrate, also can be useful. Other such linker units which are stable to the physiological environment when not in the presence of a cell surface protease, but are cleavable upon the cleavage of the cell surface protease proteolytic cleavage site, are intended for use herein. Furthermore, linker units can be utilized that, upon cleavage of the cell surface protease proteolytic cleavage site, remain attached to the therapeutic agent but do not significantly decrease the therapeutic activity of such a post-cleavage therapeutic agent derivative when compared with an unmodified therapeutic agent.

In other embodiments, the linker is a diamine containing a cyclic alkyl moiety and, in certain embodiments, the diamine contains a bicycloalkylene moiety. Examples of such diamine linkers include, but are not limited to, 1,4-bis(aminomethyl)cyclohexane, 1,4-bis(aminomethyl)-cycloheptane, 1,3-bis(aminomethyl)cyclopentane, 1-amino-4-(aminomethyl)cyclohexane, 1,4-diaminocyclohexane and 1,4-bis(aminomethyl)-bicyclo[2.2.2]octane.

Other linkers include 1,ω-diaminoalkanes, including, but not limited to, 1,3-diaminopropane, and 1,ω-dicarbonylalkanes, including, but not limited to, oxalic, malonic, succinic, glutaric, adipic and pivalic acids.

Further linkers for use in the conjugates provided herein include self-eliminating linkers such as those of the following formulae:

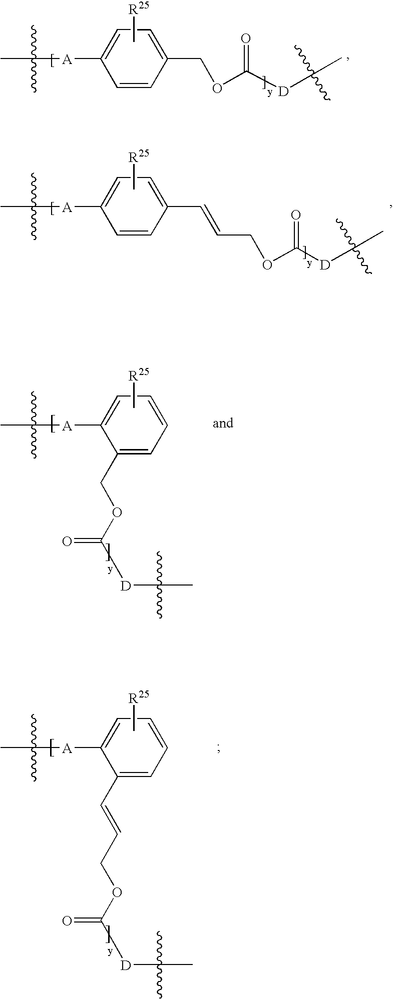

where A is NH or 0; D is N(H or alkyl) or 0; R²⁵ is H, alkyl, cycloalkyl, cycloalkylalkyl, aryl, heteroaryl optionally substituted with 1 or more, such as 1 to 3, substituents selected from halo, halo alkyl and alkyl, aralkyl, heteroaralkyl, alkenyl containing 1 to 2 double bonds, alkynyl containing 1 to 2 triple bonds, alk(en)(yn)yl groups, halo, pseudohalo, cyano, hydroxy, haloalkyl and polyhaloalkyl, such as, for example, halo lower alkyl, especially trifluoromethyl, formyl, alkylcarbonyl, arylcarbonyl that optionally is substituted with 1 or more, such as, for example, 1 to 3, substituents selected from, for example, halo, halo alkyl and alkyl, heteroarylcarbonyl, carboxy, alkoxycarbonyl, aryloxycarbonyl, aminoimino, alkoxycarbonylamino, aryloxycarbonylamino, aminocarbonyl, alkylaminocarbonyl, dialkylaminocarbonyl, arylaminocarbonyl, diarylaminocarbonyl, aralkylaminocarbonyl, alkoxy, aryloxy, perfluoroalkoxy, alkenyloxy, alkynyloxy, arylalkoxy, aminoalkyl, alkylaminoalkyl, dialkylaminoalkyl, arylaminoalkyl, amino, alkylamino, dialkylamino, arylamino, alkylarylamino, alkylcarbonylamino, arylcarbonylamino, azido, nitro, mercapto, alkylthio, arylthio, perfluoroalkylthio, thiocyano, isothiocyano, alkylsulfinyl, alkylsulfonyl, arylsulfinyl, arylsulfonyl, aminosulfonyl, alkylaminosulfonyl, dialkylaminosulfonyl and arylamino-sulfonyl.; and y is an integer from 1 to 3.

3. Therapeutic Agents

The conjugates are intended for modifying a variety of biological responses. Accordingly, the therapeutic agents are any agents, including proteins and polypeptides, small molecules and other molecules that possess or potentiate a desired biological activity. Such molecules include cytotoxic agents, such as, but are not limited to, a toxin such as abrin, ricin A, pseudomonas exotoxin, shiga toxin, diphtheria toxin and other such toxins and toxic portions and/or subunits or chains thereof; proteins such as, but not limited to, tumor necrosis factor, α-interferon, γ-interferon, nerve growth factor, platelet derived growth factor, tissue plasminogen activator; or, biological response modifiers such as, for example, lymphokines, interleukin-I (IL-1), interleukin-2 (IL-2), interleukin-6 (IL-6), granulocyte macrophage colony stimulating factor (GMCSF), granulocyte colony stimulating factor (G-CSF), erythropoietin (EPO), pro-coagulants such as tissue factor and tissue factor variants, pro-apoptotic agents such FAS-ligand, fibroblast growth factors (FGF), nerve growth factor and other growth factors. Each must be in a form that can enter a cell or otherwise exert a therapeutic effect when in the vicinity thereof.

Thus, therapeutic agents, include, but are not limited to, anti-tumor, anti-angiogenic, pro-apoptotic, anti-cancer and anti-mitotic agents. These are conjugated, optionally via a linker, to a substrate, such as peptidic substrate, which is a substrate for the protease.

Among the therapeutic agents are cytotoxic agents that include, in general, but are not limited to, alkylating agents, toxins, antiproliferative agents and tubulin binding agents. Classes of cytotoxic agents for use herein include, for example, the anthracycline family of drugs, the vinca drugs, the mitomycins, the bleomycins, the cytotoxic nucleosides, the pteridine family of drugs, diynenes, the maytansinoids, the epothilones, the taxanes and the podophyllotoxins.

Exemplary members of those classes include, for example, doxorubicin, carminomycin, daunorubicin, aminopterin, methotrexate, methopterin, dichloro-methotrexate, mitomycin C, porfiromycin, 5-fluorouracil, 6-mercaptopurine, cytosine arabinoside, podophyllotoxin, or podophyllotoxin derivatives such as etoposide or etoposide phosphate, melphalan, vinblastine, vincristine, leurosidine, vindesine, leurosine, maytansinol, epothilone A or B, taxotere, taxol and the like. Other such therapeutic agents include estramustine, cisplatin, combretastatin and analogs, and cyclophosphamide. One skilled in the art can make chemical modifications to the desired therapeutic agent in order to make reactions of that compound more convenient for purposes of preparing the conjugates.

Particular therapeutic agents include the following drugs. One skilled in the art understands that these structural formulae are exemplary only and that such compounds or derivatives or analogs thereof have acquired in the art different generic or trivial names.

a. The Methotrexate Group of Formula (1):

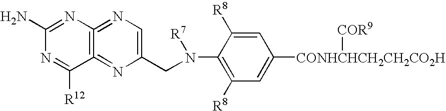

in which

R¹² is amino or hydroxy;

R⁷ is hydrogen or methyl;

R⁸ is hydrogen, fluoro, chloro, bromo or iodo;

R⁹ is hydroxy or a moiety which completes a salt of the carboxylic acid.

b. The Mitomycin Group of Formula (2):

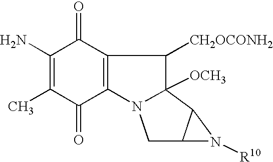

in which R¹⁰ is hydrogen or methyl.

c. The Bleomycin Group of Formula (3):

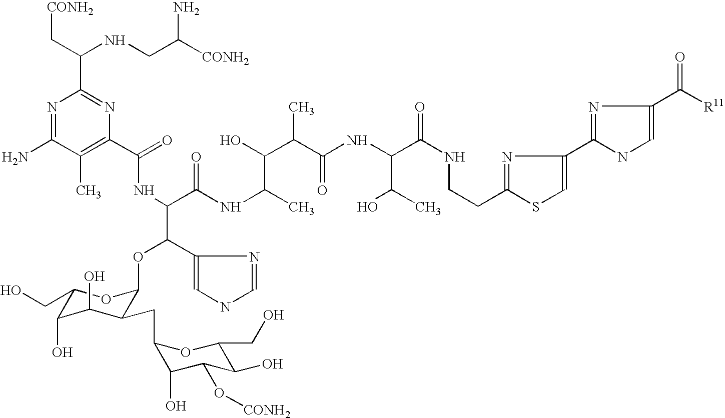

in which R¹¹ is hydroxy, amino, C₁-C₃ alkylamino, di(C₁-C₃ alkyl)amino, C₄-C₆ polymethylene amino, —NHCH₂CH₂CH₂CH₂NH—C(NH)NH₂ or —NHCH₂CH₂CH₂S⁺(CH₃)₂.

d. Melphalan of Formula (4):

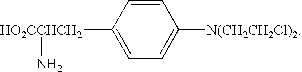

e. Mercaptopurine of Formula (5):

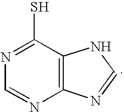

f. Cyotosine Arabinoside of Formula (6):

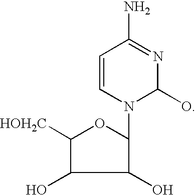

g. Podophyllotoxins of Formula (7):

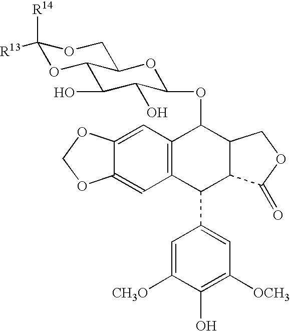

in which

R¹³ is hydrogen or methyl; and

R¹⁴ is methyl or thienyl or a phosphate salt thereof.

h. The Vinca Alkaloid Group of Drugs of Formula (8):

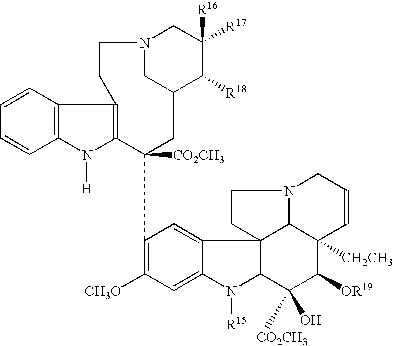

in which

when R¹⁷ and R¹⁸ are taken singly, R¹⁵ is H, CH₃ or CHO; and

R¹⁸ is H, and one of R¹⁶ and R¹⁷ is ethyl and the other is H or OH;

when R¹⁷ and R¹⁸ are taken together with the carbons to which they are attached, they form an oxirane ring in which case R¹⁶ is ethyl; and

R¹⁹ is hydrogen, (C₁-C₃ alkyl)-CO, or chlorosubstituted (C₁-C₃ alkyl)-CO.

The conjugates provided herein where the therapeutic agent is the vinca alkaloid vinblastine include those of formula:

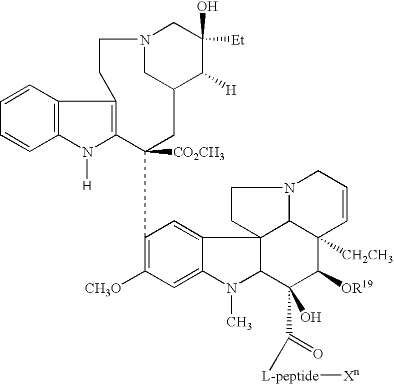

where the peptidic substrate is as described above for formulae I and II; L is a linker such as —NH—(CH₂)ᵤ—T—(CH₂)ᵤ—NH—; Xⁿ is

a) hydrogen,

b) —(C═O)R^{1a},

c)

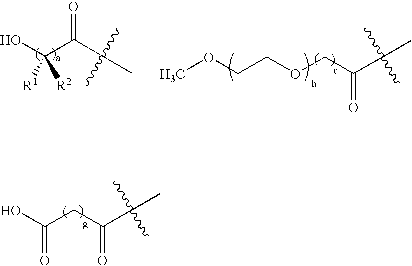

d)

e)

f) ethoxysquarate; and

g) cotininyl;

R¹ and R² are independently hydrogen, OH, C₁-C₆ alkyl, C₁-C₆ alkoxy, C₁-C₆ aralkyl and aryl;

R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl,

R¹⁹ is hydrogen, (C₁-C₃ alkyl)-CO, or chlorosubstituted (C₁-C₃ alkyl)-CO;

T is selected from cyclopentyl, cyclohexyl, cycloheptyl or bicyclo[2.2.2]octanyl;

a is 1, 2, 3 or 4;

b is zero or an integer between 1 and 100;

c is 0 or 1, provided that if b is zero, c is 1;

g is 1, 2 or 3;

u is 0, 1, 2 or 3;

or a pharmaceutically acceptable derivative thereof.

i. Difluoronucleosides of Formula (9):

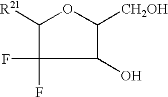

in which R²¹ is a base of one of the formulae:

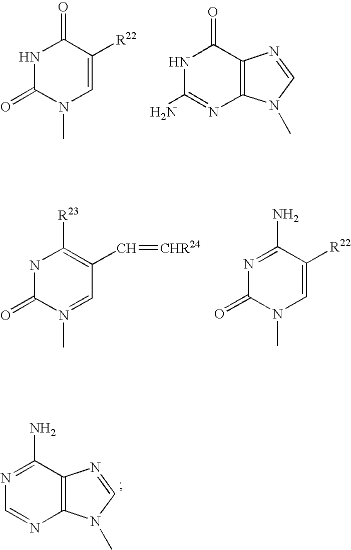

R²² is hydrogen, methyl, bromo, fluoro, chloro or iodo;

R²³ is —OH or —NH2;

R²⁴ is hydrogen, bromo, chloro or lodo.

j. Estramustine (10):

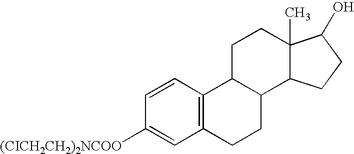

k. Cyclophosphamide (11):

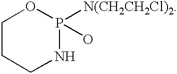

I. Anthracycline Antibiotics of Formula (12):

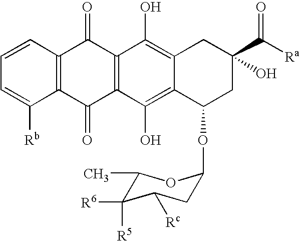

in which

R^{a} is —CH₃, —CH₂OH, —CH₂OCO(CH₂)₃CH₃, or —CH₂OCOCH(OC₂H₅)₂;

R^{b} is —OCH₃, —OH or —H;

R^{c} is —NH₂, —NHCOCF₃, 4-morpholinyl, 3-cyano-4-morpholinyl, 1-piperidinyl, 4-methoxy-1-piperidinyl, benzylamine, dibenzylamine, cyanomethylamine, or 1-cyano-2-methoxyethyl amine;

R⁵ is —OH —OTHP or —H; and

R⁶ is —OH or —H provided that R6 is not —OH when R⁵ is —OH or —OTHP.

Table 2, which follows, provides a number of anthracycline drugs and their generic or trivial names:

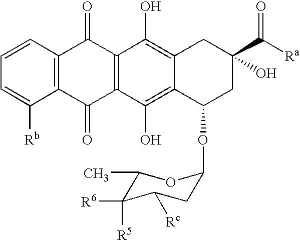

| Compound | R^{a} | R^{b} | R^{c} | R⁵ | R⁶ |
|---|---|---|---|---|---|
| daunorubicin^{a} | CH₃ | OCH₃ | NH₂ | OH | H |
| doxorubicin^{b} | CH₂OH | OCH₃ | NH₂ | OH | H |
| detorubicin | CH₂OCOCH(OC₂H₅)₂ | OCH₃ | NH₂ | OH | H |
| carminomycin | CH₃ | OH | NH₂ | OH | H |
| idarubicin | CH₃ | H | NH₂ | OH | H |
| epirubicin | CH₂OH | OCH₃ | NH₂ | OH | OH |
| esorubicin | CH₂OH | OCH₃ | NH₂ | H | H |
| THP | CH₂OH | OCH₃ | NH₂ | OTHP | H |
| AD-32 | CH₂OCO(CH₂)₃CH₃ | OCH₃ | NHCOCF₃ | OH | H |

^{a}daunorubicin is an alternative name for daunomycin
^{b}doxorubicin is an alternative name for adriamycin.

In one embodiment, when the therapeutic agent is doxorubicin, it is conjugated to the peptidic substrate via the amino group of the aminoglycoside moiety of doxorubicin.

m. Maytansinol

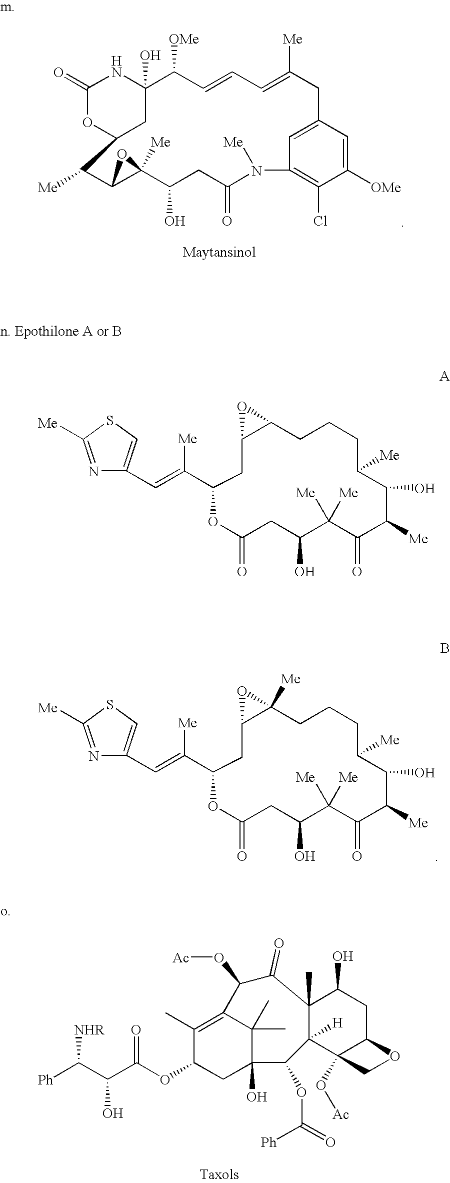

where R is PhC(O) or t-BuOC(O).

In one embodiment, when the therapeutic agent is taxol (R=C(O)Ph), the peptidic substrate is conjugated to the secondary hydroxyl group of the cyclohexane moiety of taxol.

p. Ribosome-Inactivating Proteins

Ribosome-inactivating proteins (RIPs), which include ricin, abrin and saporin, are plant proteins that catalytically inactivate eukaryotic ribosomes. RIPS inactivate ribosomes by interfering with the protein elongation step of protein synthesis. For example, the RIP saporin (hereinafter also referred to as SAP) has been shown to enzymatically inactivate 60S ribosomes by cleavage of the n-glycosidic bond of the adenine at position 4324 in the rat 28S ribosomal RNA (rRNA). Some RIPs, such as the toxins abrin and ricin, contain two constituent chains: a cell-binding chain that mediates binding to cell surface receptors and internalization of the molecule; and an enzymatically active chain responsible for protein synthesis inhibitory activity. Such RIPs are type II RIPs. Other RIPs, such as the saporins, are single chains and are designated type I RIPs. Because such RIPs lack a cell-binding chain, they are less toxic to whole cells than the RIPs that have two chains. Two chain RIPs are generally used for conjugation herein, unless a single chain is further conjugated to an agent, such as a growth factor that mediates binding and internalization.

Several structurally related RIP's have been isolated from seeds and leaves of the plant Saponaria officinalis (soapwort). Among these, SAP-6 is the most active and abundant, representing 7% of total seed proteins. Saporin is very stable, has a high isoelectric point, does not contain carbohydrates, and is resistant to denaturing agents, such as sodium dodecyl sulfate (SDS), and a variety of proteases. The amino acid sequences of several saporin-6 isoforms from seeds are known and there appear to be families of saporin RIPs differing in a few amino acid residues. Because saporin is a type I RIP, it does not possess a cell-binding chain. Consequently, its toxicity to whole cells is much lower than the other toxins, such as ricin and abrin. When internalized by eukaryotic cells, however, its cytotoxicity is 100- to 1000-fold more potent than ricin A chain.

4. Exemplary Conjugates

The conjugates provided herein, are prepared by identifying suitable peptidic substrates for the targeted cell surface protease, or a soluble, shed or released form thereof, and forming a conjugate of the peptidic substrate(s) with a therapeutic agent(s). Exemplary conjugates, containing peptidic substrates designed, for example, for cleavage by MTSP1, endotheliase 1 and urokinase, are described. It is understood that upon identification of a cell surface protease, including cell-associated and cell-localized proteases, or a soluble, shed or released form thereof, in or associated with a cell involved in a disease or other conditions of interest, or with a cell present in the vicinity of a cell or tissue involved in or associated with a disease or other condition of interest, suitable peptidic substrates therefor can be empirically designed and then conjugated to therapeutic agents as exemplified herein.

In certain embodiments, the conjugates for use in the compositions and methods provided herein include:

Ac-Leu-Arg-Ala-Quat-Gly-Arg-Ala-Ala-(therapeutic agent) (SEQ ID NO: 46);

Ac-Leu-Arg-Ala-Quat-Ala-Arg-Ala-Ala-(therapeutic agent) (SEQ ID NO: 47);

Ac-Leu-Arg-Ser-Quat-Gly-Arg-Ala-Ala-(therapeutic agent) (SEQ ID NO: 48);

Ac-Leu-Arg-Ser-Quat-Ala-Arg-Ala-Ala-(therapeutic agent) (SEQ ID NO: 49);

Ac-Leu-Arg-Pro-Arg-Phe-Lys-Ile-Ile-(therapeutic agent) (SEQ ID NO: 50);

Ac-Arg-Pro-Arg-Phe-Lys-Ile-Ile-(therapeutic agent) (SEQ ID NO: 51);

Ac-Pro-Arg-Phe-Lys-Ile-Ile-(therapeutic agent) (SEQ ID NO: 52);

Ac-Leu-Arg-Ser-Lys-Ser-Arg-Ala-Ala-(therapeutic agent) (SEQ ID NO: 53);

Ac-Arg-Ser-Lys-Ser-Arg-Ala-Ala-(therapeutic agent) (SEQ ID NO: 54);

Ac-Ser-Lys-Ser-Arg-Ala-Ala-(therapeutic agent) (SEQ ID NO: 55);

Ac-Leu-Arg-Pro-Arg-Phe-Arg-Ile-Ile-(therapeutic agent) (SEQ ID NO: 56);

Ac-Arg-Pro-Arg-Phe-Arg-Ile-Ile-(therapeutic agent) (SEQ ID NO: 57);

Ac-Pro-Arg-Phe-Arg-Ile-Ile-(therapeutic agent) (SEQ ID NO: 58);

Ac-Leu-Arg-Ser-Arg-Ser-Arg-Ala-Ala-(therapeutic agent) (SEQ ID NO: 59);

Ac-Arg-Ser-Arg-Ser-Arg-Ala-Ala-(therapeutic agent) (SEQ ID NO: 60); and

Ac-Ser-Arg-Ser-Arg-Ala-Ala-(therapeutic agent) (SEQ ID NO: 61).

In further embodiments herein, the conjugates are Ac-Leu-Arg-Ala-Quat-Gly-Arg-Ala-Ala-(therapeutic agent) (SEQ ID NO: 62); Ac-Leu-Arg-Ala-Quat-Ala-Arg-Ala-Ala-(therapeutic agent) (SEQ ID NO: 63); Ac-Leu-Arg-Ser-Quat-Gly-Arg-Ala-Ala-(therapeutic agent) (SEQ ID NO: 64); and Ac-Leu-Arg-Ser-Quat-Ala-Arg-Ala-Ala-(therapeutic agent) (SEQ ID NO: 65).

In other embodiments herein, the conjugates are

Ac-Leu-Arg-Pro-Arg-Phe-Lys-Ile-Ile-(therapeutic agent) (SEQ ID NO: 66);

Ac-Arg-Pro-Arg-Phe-Lys-Ile-Ile-(therapeutic agent) (SEQ ID NO: 67);

Ac-Pro-Arg-Phe-Lys-Ile-Ile-(therapeutic agent) (SEQ ID NO: 68);

Ac-Leu-Arg-Ser-Lys-Ser-Arg-Ala-Ala-(therapeutic agent) (SEQ ID NO: 69);

Ac-Arg-Ser-Lys-Ser-Arg-Ala-Ala-(therapeutic agent) (SEQ ID NO: 70);

Ac-Ser-Lys-Ser-Arg-Ala-Ala-(therapeutic agent) (SEQ ID NO: 71);

Ac-Leu-Arg-Pro-Arg-Phe-Arg-Ile-Ile-(therapeutic agent) (SEQ ID NO: 72);

Ac-Arg-Pro-Arg-Phe-Arg-Ile-Ile-(therapeutic agent) (SEQ ID NO: 73);

Ac-Pro-Arg-Phe-Arg-Ile-Ile-(therapeutic agent) (SEQ ID NO: 74);

Ac-Leu-Arg-Ser-Arg-Ser-Arg-Ala-Ala-(therapeutic agent) (SEQ ID NO: 75);

Ac-Arg-Ser-Arg-Ser-Arg-Ala-Ala-(therapeutic agent) (SEQ ID NO: 76); and

Ac-Ser-Arg-Ser-Arg-Ala-Ala-(therapeutic agent) (SEQ ID NO: 77).

In other embodiments, the conjugates for use herein include the following:

pyroGlu-Pro-Arg-Ala-Ala-(therapeutic agent) (SEQ ID NO: 78);

CH₃SO₂-D-HHT-Gly-Arg-Ala-Ala-(therapeutic agent) (SEQ ID NO: 79);

N-p-tosyl-Gly-Pro-Arg-Ala-Ala-(therapeutic agent) (SEQ ID NO: 80);

Benzoyl-Val-Gly-Arg-Ala-Ala-(therapeutic agent) (SEQ ID NO: 81);

CH₃SO₂-D-HHT-Gly-Arg-Ala-Ala-(therapeutic agent) (SEQ ID NO: 82);

N-α-Z-D-Arg-Gly-Arg-Ala-Ala-(therapeutic agent) (SEQ ID NO: 83) (Z=benzyloxycarbonyl);

pyroGlu-Gly-Arg-Ala-Ala-(therapeutic agent) (SEQ ID NO: 84);

H-D-Ile-Pro-Arg-Ala-Ala-(therapeutic agent) (SEQ ID NO: 85); Cbo-L-(γ)Glu(α-t-BuO)-Gly-Arg-Ala-Ala-(therapeutic agent) (SEQ ID NO: 86) (Cbo=carbobenzoxy);

H-D-Pro-Phe-Arg-Ala-Ala-(therapeutic agent) (SEQ ID NO: 87);

H-D-Val-Leu-Arg-Ala-Ala-(therapeutic agent) (SEQ ID NO: 88);

Bz-Ile-Glu(γ-OH)-Gly-Arg-Ala-Ala-(therapeutic agent) (SEQ ID NO: 89) (Bz=benzoyl);

Bz-Ile-Glu(γ-OMe)-Gly-Arg-Ala-Ala-(therapeutic agent) (SEQ ID NO: 90);

Benzoyl-Pro-Phe-Arg-Ala-Ala-(therapeutic agent) (SEQ ID NO: 91);

H-D-Phe-Pip-Arg-Ala-Ala-(therapeutic agent) (SEQ ID NO: 92);

H-D-Val-Leu-Lys-Ala-Ala-(therapeutic agent) (SEQ ID NO: 93);

H-D-Nle-HHT-Lys-Ala-Ala-(therapeutic agent) (SEQ ID NO: 94);

Pyr-Arg-Thr-Lys-Arg-Ala-Ala-(therapeutic agent) (SEQ ID NO: 95);

H-Arg-Gln-Arg-Arg-Ala-Ala-(therapeutic agent) (SEQ ID NO: 96);

Boc-Gln-Gly-Arg-Ala-Ala-(therapeutic agent) (SEQ ID NO: 97);

Z-Arg-Arg-Ala-Ala-(therapeutic agent) (SEQ ID NO: 98);

H-D-HHT-Ala-Arg-Ala-Ala-(therapeutic agent) (SEQ ID NO: 99);

H-D-CHT-Gly-Arg-Ala-Ala-(therapeutic agent) (SEQ ID NO: 100);

MeSO₂-dPhe-Pro-Arg-Ala-Ala-(therapeutic agent) (SEQ ID NO: 101);

δ-Z-D-Lys-Pro-Arg-Ala-Ala-(therapeutic agent) (SEQ ID NO: 102); and

CH₃SO₂-D-CHA-But-Arg-Ala-Ala-(therapeutic agent) (SEQ ID NO: 103).

In another embodiment, the conjugates for use in the compositions and methods provided herein include:

Ac-Arg-Gln-Ser-Arg-Ala-Ala-(therapeutic agent) (SEQ ID NO: 104);

Ac-Arg-Arg-Gln-Ser-Arg-Ala-Ala-(therapeutic agent) (SEQ ID NO: 105);

Ac-Leu-Arg-Arg-Gin-Ser-Arg-Ala-Ala-(therapeutic agent) (SEQ ID NO: 106);

Ac-Arg-Gln-Ser-Arg-Ala-(therapeutic agent) (SEQ ID NO: 107);

Ac-Arg-Arg-Gln-Ser-Arg-Ala-(therapeutic agent) (SEQ ID NO: 108);

Ac-Leu-Arg-Arg-Gln-Ser-Arg-Gly-Gly-(therapeutic agent) (SEQ ID NO: 109);

Ac-Leu-Arg-Arg-Gln-Ser-Arg-Ala-(therapeutic agent) (SEQ ID NO: 110);

Ac-Arg-Arg-Gln-Ser-Arg-Ile-(therapeutic agent) (SEQ ID NO: 111); and

Ac-Leu-Arg-Arg-Gln-Ser-Arg-Ala-Ile-(therapeutic agent) (SEQ ID NO: 112).

In certain embodiments, the conjugates for use in the compositions and methods provided herein include:

Ac-Leu-Arg-Ala-Quat-Gly-Arg-Ser-Leu-(therapeutic agent) (SEQ ID NO: 113);

Ac-Leu-Arg-Ala-Quat-Ala-Arg-Ser-Leu-(therapeutic,agent) (SEQ ID NO: 114);

Ac-Leu-Arg-Ser-Quat-Gly-Arg-Ser-Leu-(therapeutic agent) (SEQ ID NO: 115);

Ac-Leu-Arg-Ser-Quat-Ala-Arg-Ser-Leu-(therapeutic agent) (SEQ ID NO: 116);

Ac-Leu-Arg-Pro-Arg-Phe-Lys-Ser-Leu-(therapeutic agent) (SEQ ID NO: 117);

Ac-Arg-Pro-Arg-Phe-Lys-Ser-Leu-(therapeutic agent) (SEQ ID NO: 118);

Ac-Pro-Arg-Phe-Lys-Ser-Leu-(therapeutic agent) (SEQ ID NO: 119);

Ac-Leu-Arg-Ser-Lys-Ser-Arg-Ser-Leu-(therapeutic agent) (SEQ ID NO: 120);

Ac-Arg-Ser-Lys-Ser-Arg-Ser-Leu-(therapeutic agent) (SEQ ID NO: 121);

Ac-Ser-Lys-Ser-Arg-Ser-Leu-(therapeutic agent) (SEQ ID NO: 122);

Ac-Leu-Arg-Pro-Arg-Phe-Arg-Ser-Leu-(therapeutic agent) (SEQ ID NO: 123);

Ac-Arg-Pro-Arg-Phe-Arg-Ser-Leu-(therapeutic agent) (SEQ ID NO: 124);

Ac-Pro-Arg-Phe-Arg-Ser-Leu-(therapeutic agent) (SEQ ID NO: 125);

Ac-Leu-Arg-Ser-Arg-Ser-Arg-Ser-Leu-(therapeutic agent) (SEQ ID NO: 126);

Ac-Arg-Ser-Arg-Ser-Arg-Ser-Leu-(therapeutic agent) (SEQ ID NO: 127); and

Ac-Ser-Arg-Ser-Arg-Ser-Leu-(therapeutic agent) (SEQ ID NO: 128).

In further embodiments herein, the conjugates are Ac-Leu-Arg-Ala-Quat-Gly-Arg-Ser-Leu-(therapeutic agent) (SEQ ID NO: 129); Ac-Leu-Arg-Ala-Quat-Ala-Arg-Ser-Leu-(therapeutic agent) (SEQ ID NO: 130); Ac-Leu-Arg-Ser-Quat-Gly-Arg-Ser-Leu-(therapeutic agent) (SEQ ID NO: 131); and Ac-Leu-Arg-Ser-Quat-Ala-Arg-Ser-Leu-(therapeutic agent) (SEQ ID NO: 132).

In other embodiments herein, the conjugates are

Ac-Leu-Arg-Pro-Arg-Phe-Lys-Ser-Leu-(therapeutic agent) (SEQ ID NO: 133);

Ac-Arg-Pro-Arg-Phe-Lys-Ser-Leu-(therapeutic agent) (SEQ ID NO: 134);

Ac-Pro-Arg-Phe-Lys-Ser-Leu-(therapeutic agent) (SEQ ID NO: 135);

Ac-Leu-Arg-Ser-Lys-Ser-Arg-Ser-Leu-(therapeutic agent) (SEQ ID NO: 136);

Ac-Arg-Ser-Lys-Ser-Arg-Ser-Leu-(therapeutic agent) (SEQ ID NO: 137);

Ac-Ser-Lys-Ser-Arg-Ser-Leu-(therapeutic agent) (SEQ ID NO: 138);

Ac-Leu-Arg-Pro-Arg-Phe-Arg-Ser-Leu-(therapeutic agent) (SEQ ID NO: 139);

Ac-Arg-Pro-Arg-Phe-Arg-Ser-Leu-(therapeutic agent) (SEQ ID NO: 140);

Ac-Pro-Arg-Phe-Arg-Ser-Leu-(therapeutic agent) (SEQ ID NO: 141);

Ac-Leu-Arg-Ser-Arg-Ser-Arg-Ser-Leu-(therapeutic agent) (SEQ ID NO: 142);

Ac-Arg-Ser-Arg-Ser-Arg-Ser-Leu-(therapeutic agent) (SEQ ID NO: 143); and

Ac-Ser-Arg-Ser-Arg-Ser-Leu-(therapeutic agent) (SEQ ID NO: 144).

In other embodiments, the conjugates for use herein include the following:

pyroGlu-Pro-Arg-Ser-Leu-(therapeutic agent) (SEQ ID NO: 145); CH₃SO₂-D-HHT-Gly-Arg-Ser-Leu-(therapeutic agent) (SEQ ID NO: 146);

N-p-tosyl-Gly-Pro-Arg-Ser-Leu-(therapeutic agent) (SEQ ID NO: 147);

Benzoyl-Val-Gly-Arg-Ser-Leu-(therapeutic agent) (SEQ ID NO: 148);

CH₃SO₂-D-HHT-Gly-Arg-Ser-Leu-(therapeutic agent) (SEQ ID NO: 149);

N-α-Z-D-Arg-Gly-Arg-Ser-Leu-(therapeutic agent) (SEQ ID NO: 150) (Z=benzyloxycarbonyl);

pyroGlu-Gly-Arg-Ser-Leu-(therapeutic agent) (SEQ ID NO: 151);

H-D-Ile-Pro-Arg-Ser-Leu-(therapeutic agent) (SEQ ID NO: 152);

Cbo-L-(γ)Glu(α-t-BuO)-Gly-Arg-Ser-Leu-(therapeutic agent) (SEQ ID NO: 153) (Cbo=carbobenzoxy);

H-D-Pro-Phe-Arg-Ser-Leu-(therapeutic agent) (SEQ ID NO: 154);

H-D-Val-Leu-Arg-Ser-Leu-(therapeutic agent) (SEQ ID NO: 155);

Bz-Ile-Glu(γ-OH)-Gly-Arg-Ser-Leu-(therapeutic agent) (SEQ ID NO: 156) (Bz=benzoyl);

Bz-Ile-Glu(γ-OMe)-Gly-Arg-Ser-Leu-(therapeutic agent) (SEQ ID NO: 157);

Benzoyl-Pro-Phe-Arg-Ser-Leu-(therapeutic agent) (SEQ ID NO: 158);

H-D-Phe-Pip-Arg-Ser-Leu-(therapeutic agent) (SEQ ID NO: 159);

H-D-Val-Leu-Lys-Ser-Leu-(therapeutic agent) (SEQ ID NO: 160);

H-D-Nle-HHT-Lys-Ser-Leu-(therapeutic agent) (SEQ ID NO: 161);

Pyr-Arg-Thr-Lys-Arg-Ser-Leu-(therapeutic agent) (SEQ ID NO: 162);

H-Arg-Gln-Arg-Arg-Ser-Leu-(therapeutic agent) (SEQ ID NO: 163);

Boc-Gln-Gly-Arg-Ser-Leu-(therapeutic agent) (SEQ ID NO: 164);

Z-Arg-Arg-Ser-Leu-(therapeutic agent) (SEQ ID NO: 165);

H-D-HHT-Ala-Arg-Ser-Leu-(therapeutic agent) (SEQ ID NO: 166);

H-D-CHT-Gly-Arg-Ser-Leu-(therapeutic agent) (SEQ ID NO: 167);

MeSO₂-dPhe-Pro-Arg-Ser-Leu-(therapeutic agent) (SEQ ID NO: 168);

δ-Z-D-Lys-Pro-Arg-Ser-Leu-(therapeutic agent) (SEQ ID NO: 169); and

CH₃SO₂-D-CHA-But-Arg-Ser-Leu-(therapeutic agent) (SEQ ID NO: 170).

In another embodiment, the conjugates for use in the compositions and methods provided herein include:

Ac-Arg-Gln-Ser-Arg-Ser-Leu-(therapeutic agent) (SEQ ID NO: 171);

Ac-Arg-Arg-Gln-Ser-Arg-Ser-Leu-(therapeutic agent) (SEQ ID NO: 172);

Ac-Leu-Arg-Arg-Gln-Ser-Arg-Ser-Leu-(therapeutic agent) (SEQ ID NO: 173);

Ac-Arg-Gln-Ser-Arg-Leu-(therapeutic agent) (SEQ ID NO: 174);

Ac-Arg-Arg-Gln-Ser-Arg-Leu-(therapeutic agent) (SEQ ID NO: 175);

Ac-Leu-Arg-Arg-Gln-Ser-Arg-Ser-Leu-(therapeutic agent) (SEQ ID NO: 176);

Ac-Leu-Arg-Arg-Gln-Ser-Arg-Leu-(therapeutic agent) (SEQ ID NO: 177);

Ac-Arg-Arg-Gln-Ser-Arg-Leu-(therapeutic agent) (SEQ ID NO: 178); and

Ac-Leu-Arg-Arg-Gln-Ser-Arg-Ser-Leu-(therapeutic agent) (SEQ ID NO: 179).

In other embodiments, the conjugates provided herein include:

Ac-Arg-Gln-Gly-Arg-Ser-Leu-(therapeutic agent) (SEQ ID NO: 180);

Ac-Arg-Gln-Ala-Arg-Ser-Leu-(therapeutic agent) (SEQ ID NO: 181);

Ac-Arg-Gln-Phe-Arg-Ser-Leu-(therapeutic agent) (SEQ ID NO: 182);

Ac-Arg-Ser-Arg-Ser-Leu-(therapeutic agent) (SEQ ID NO: 183);

Ac-Arg-Gly-Arg-Ser-Leu-(therapeutic agent) (SEQ ID NO: 184);

Ac-Arg-Ala-Arg-Ser-Leu-(therapeutic agent) (SEQ ID NO: 185);

Ac-Arg-Phe-Arg-Ser-Leu-(therapeutic agent) (SEQ ID NO: 186);

Ac-Gln-Ser-Arg-Ser-Leu-(therapeutic agent) (SEQ ID NO: 187);

Ac-Gln-Gly-Arg-Ser-Leu-(therapeutic agent) (SEQ ID NO: 188);

Ac-Gln-Ala-Arg-Ser-Leu-(therapeutic agent) (SEQ ID NO: 189); and

Ac-Gln-Phe-Arg-Ser-Leu-(therapeutic agent) (SEQ ID NO: 190).

In further embodiments, the conjugates for use in the compositions and methods provided herein include:

Ac-Leu-Arg-Ala-Quat-Gly-Arg-Ser-Ser-Leu-(therapeutic agent) (SEQ ID NO: 191);

Ac-Leu-Arg-Ala-Quat-Ala-Arg-Ser-Ser-Leu-(therapeutic agent) (SEQ ID NO: 192);

Ac-Leu-Arg-Ser-Quat-Gly-Arg-Ser-Ser-Leu-(therapeutic agent) (SEQ ID NO: 193);

Ac-Leu-Arg-Ser-Quat-Ala-Arg-Ser-Ser-Leu-(therapeutic agent) (SEQ ID NO: 194);

Ac-Leu-Arg-Pro-Arg-Phe-Lys-Ser-Ser-Leu-(therapeutic agent) (SEQ ID NO: 195);

Ac-Arg-Pro-Arg-Phe-Lys-Ser-Ser-Leu-(therapeutic agent) (SEQ ID NO: 196);

Ac-Pro-Arg-Phe-Lys-Ser-Ser-Leu-(therapeutic agent) (SEQ ID NO: 197);

Ac-Leu-Arg-Ser-Lys-Ser-Arg-Ser-Ser-Leu-(therapeutic agent) (SEQ ID NO: 198);

Ac-Arg-Ser-Lys-Ser-Arg-Ser-Ser-Leu-(therapeutic agent) (SEQ ID NO: 199);

Ac-Ser-Lys-Ser-Arg-Ser-Ser-Leu-(therapeutic agent) (SEQ ID NO: 200);

Ac-Leu-Arg-Pro-Arg-Phe-Arg-Ser-Ser-Leu-(therapeutic agent) (SEQ ID NO: 201);

Ac-Arg-Pro-Arg-Phe-Arg-Ser-Ser-Leu-(therapeutic agent) (SEQ ID NO: 202);

Ac-Pro-Arg-Phe-Arg-Ser-Ser-Leu-(therapeutic agent) (SEQ ID NO: 203);

Ac-Leu-Arg-Ser-Arg-Ser-Arg-Ser-Ser-Leu-(therapeutic agent) (SEQ ID NO: 204);

Ac-Arg-Ser-Arg-Ser-Arg-Ser-Ser-Leu-(therapeutic agent) (SEQ ID NO: 205); and

Ac-Ser-Arg-Ser-Arg-Ser-Ser-Leu-(therapeutic agent) (SEQ ID NO: 206).

In further embodiments herein, the conjugates are Ac-Leu-Arg-Ala-Quat-Gly-Arg-Ser-Ser-Leu-(therapeutic agent) (SEQ ID NO: 207); Ac-Leu-Arg-Ala-Quat-Ala-Arg-Ser-Ser-Leu-(therapeutic agent) (SEQ ID NO: 208); Ac-Leu-Arg-Ser-Quat-Gly-Arg-Ser-Ser-Leu-(therapeutic agent) (SEQ ID NO: 209); and Ac-Leu-Arg-Ser-Quat-Ala-Arg-Ser-Ser-Leu-(therapeutic agent) (SEQ ID NO: 210).

In other embodiments herein, the conjugates are

Ac-Leu-Arg-Pro-Arg-Phe-Lys-Ser-Ser-Leu-(therapeutic agent) (SEQ ID NO: 211);

Ac-Arg-Pro-Arg-Phe-Lys-Ser-Ser-Leu-(therapeutic agent) (SEQ ID NO: 212);

Ac-Pro-Arg-Phe-Lys-Ser-Ser-Leu-(therapeutic agent) (SEQ ID NO: 213);

Ac-Leu-Arg-Ser-Lys-Ser-Arg-Ser-Ser-Leu-(therapeutic agent) (SEQ ID NO: 214);

Ac-Arg-Ser-Lys-Ser-Arg-Ser-Ser-Leu-(therapeutic agent) (SEQ ID NO: 215);

Ac-Ser-Lys-Ser-Arg-Ser-Ser-Leu-(therapeutic agent) (SEQ ID NO: 216);

Ac-Leu-Arg-Pro-Arg-Phe-Arg-Ser-Ser-Leu-(therapeutic agent) (SEQ ID NO: 217);

Ac-Arg-Pro-Arg-Phe-Arg-Ser-Ser-Leu-(therapeutic agent) (SEQ ID NO: 218);

Ac-Pro-Arg-Phe-Arg-Ser-Ser-Leu-(therapeutic agent) (SEQ ID NO: 219);

Ac-Leu-Arg-Ser-Arg-Ser-Arg-Ser-Ser-Leu-(therapeutic agent) (SEQ ID NO: 220);

Ac-Arg-Ser-Arg-Ser-Arg-Ser-Ser-Leu-(therapeutic agent) (SEQ ID NO: 221); and

Ac-Ser-Arg-Ser-Arg-Ser-Ser-Leu-(therapeutic agent) (SEQ ID NO: 222).

In other embodiments, the conjugates for use herein include the following:

pyroGlu-Pro-Arg-Ser-Ser-Leu-(therapeutic agent) (SEQ ID NO: 223);

CH₃SO₂-D-HHT-Gly-Arg-Ser-Ser-Leu-(therapeutic agent) (SEQ ID NO: 224);

N-p-tosyl-Gly-Pro-Arg-Ser-Ser-Leu-(therapeutic agent) (SEQ ID NO: 225); Benzoyl-Val-Gly-Arg-Ser-Ser-Leu-(therapeutic agent) (SEQ ID NO: 226); CH₃SO₂-D-HHT-Gly-Arg-Ser-Ser-Leu-(therapeutic agent) (SEQ ID NO: 227);

N-α-Z-D-Arg-Gly-Arg-Ser-Ser-Leu-(therapeutic agent) (SEQ ID NO: 228) (Z=benzyloxycarbonyl);

pyroGlu-Gly-Arg-Ser-Ser-Leu-(therapeutic agent) (SEQ ID NO: 229);

H-D-Ile-Pro-Arg-Ser-Ser-Leu-(therapeutic agent) (SEQ ID NO: 230);

Cbo-L-(γ)Glu(α-t-BuO)-Gly-Arg-Ser-Ser-Leu-(therapeutic agent) (SEQ ID NO: 231) (Cbo=carbobenzoxy);

H-D-Pro-Phe-Arg-Ser-Ser-Leu-(therapeutic agent) (SEQ ID NO: 232);

H-D-Val-Leu-Arg-Ser-Ser-Leu-(therapeutic agent) (SEQ ID NO: 233);

Bz-Ile-Glu(γ-OH)-Gly-Arg-Ser-Ser-Leu-(therapeutic agent) (SEQ ID NO: 234) (Bz=benzoyl);

Bz-Ile-Glu(γ-OMe)-Gly-Arg-Ser-Ser-Leu-(therapeutic agent) (SEQ ID NO: 235);

Benzoyl-Pro-Phe-Arg-Ser-Ser-Leu-(therapeutic agent) (SEQ ID NO: 236);

H-D-Phe-Pip-Arg-Ser-Ser-Leu-(therapeutic agent) (SEQ ID NO: 237);

H-D-Val-Leu-Lys-Ser-Ser-Leu-(therapeutic agent) (SEQ ID NO: 238);

H-D-Nle-HHT-Lys-Ser-Ser-Leu-(therapeutic agent) (SEQ ID NO: 239);

Pyr-Arg-Thr-Lys-Arg-Ser-Ser-Leu-(therapeutic agent) (SEQ ID NO: 240);

H-Arg-Gln-Arg-Arg-Ser-Ser-Leu-(therapeutic agent) (SEQ ID NO: 241);

Boc-Gln-Gly-Arg-Ser-Ser-Leu-(therapeutic agent) (SEQ ID NO: 242);

Z-Arg-Arg-Ser-Ser-Leu-(therapeutic agent) (SEQ ID NO: 243);

H-D-HHT-Ala-Arg-Ser-Ser-Leu-(therapeutic agent) (SEQ ID NO: 244);

H-D-CHT-Gly-Arg-Ser-Ser-Leu-(therapeutic agent) (SEQ ID NO: 245);

MeSO₂-dPhe-Pro-Arg-Ser-Ser-Leu-(therapeutic agent) (SEQ ID NO: 246);

δ-Z-D-Lys-Pro-Arg-Ser-Ser-Leu-(therapeutic agent) (SEQ ID NO: 247); and

CH₃SO₂-D-CHA-But-Arg-Ser-Ser-Leu-(therapeutic agent) (SEQ ID NO: 248).

In another embodiment, the conjugates for use in the compositions and methods provided herein include:

Ac-Arg-Gln-Ser-Arg-Ser-Ser-Leu-(therapeutic agent) (SEQ ID NO: 249);

Ac-Arg-Arg-Gln-Ser-Arg-Ser-Ser-Leu-(therapeutic agent) (SEQ ID NO: 250);

Ac-Leu-Arg-Arg-Gln-Ser-Arg-Ser-Ser-Leu-(therapeutic agent) (SEQ ID NO: 251);

Ac-Arg-Gln-Ser-Arg-Leu-(therapeutic agent) (SEQ ID NO: 252);

Ac-Arg-Arg-Gln-Ser-Arg-Leu-(therapeutic agent) (SEQ ID NO: 253);

Ac-Leu-Arg-Arg-Gln-Ser-Arg-Ser-Ser-Leu-(therapeutic agent) (SEQ ID NO: 254);

Ac-Leu-Arg-Arg-Gln-Ser-Arg-Ser-Leu-(therapeutic agent) (SEQ ID NO: 255);

Ac-Arg-Arg-Gln-Ser-Arg-Ser-Leu-(therapeutic agent) (SEQ ID NO: 256); and

Ac-Leu-Arg-Arg-Gln-Ser-Arg-Ser-Ser-Leu-(therapeutic agent) (SEQ ID NO: 257).

In other embodiments, the conjugates provided herein include:

Ac-Arg-Gln-Gly-Arg-Ser-Ser-Leu-(therapeutic agent) (SEQ ID NO: 258);

Ac-Arg-Gln-Ala-Arg-Ser-Ser-Leu-(therapeutic agent) (SEQ ID NO: 259);

Ac-Arg-Gln-Phe-Arg-Ser-Ser-Leu-(therapeutic agent) (SEQ ID NO: 260);

Ac-Arg-Ser-Arg-Ser-Ser-Leu-(therapeutic agent) (SEQ ID NO: 261);

Ac-Arg-Gly-Arg-Ser-Ser-Leu-(therapeutic agent) (SEQ ID NO: 262);

Ac-Arg-Ala-Arg-Ser-Ser-Leu-(therapeutic agent) (SEQ ID NO: 263);

Ac-Arg-Phe-Arg-Ser-Ser-Leu-(therapeutic agent) (SEQ ID NO: 264);

Ac-Gln-Ser-Arg-Ser-Ser-Leu-(therapeutic agent) (SEQ ID NO: 265);

Ac-Gln-Gly-Arg-Ser-Ser-Leu-(therapeutic agent) (SEQ ID NO: 266);

Ac-Gln-Ala-Arg-Ser-Ser-Leu-(therapeutic agent) (SEQ ID NO: 267); and

Ac-Gln-Phe-Arg-Ser-Ser-Leu-(therapeutic agent) (SEQ ID NO: 268).

In another embodiment, the conjugates provided herein include:

Ac-Gly-dSer-Ala-Arg-Ser-Ala-(therapeutic agent) (SEQ ID NO: 569);

Ac-Arg-Gly-dSer-Ala-Arg-Ser-Ala-(therapeutic agent) (SEQ ID NO: 570);

Ac-Gly-Ser-Gly-Arg-Ser-Ala-(therapetutic agent) (SEQ ID NO: 571);

Ac-Arg-Gly-Ser-Gly-Arg-Ser-Ala-(therapetutic agent) (SEQ ID NO: 572);

Ac-Leu-Arg-Gly-Ser-Gly-Arg-Ser-Ala-(therapetutic agent) (SEQ ID NO: 573);

Ac-Leu-Arg-Gly-dSer-Ala-Arg-Ser-Ala-(therapetutic agent) (SEQ ID NO: 574);

Ac-Cys(Me)-Pro-Gly-Arg-Val-Val-(therapeutic agent) (SEQ ID NO: 575);

Ac-Arg-Cys(Me)-Pro-Gly-Arg-Val-Val-(therapeutic agent) (SEQ ID NO: 577);

Ac-Arg-Arg-Cys(Me)-Pro-Gly-Arg-Val-Val-(therapeutic agent) (SEQ ID NO: 578);

Ac-Val-Ser-Ala-Arg-Met-Ala-(therapeutic agent) (SEQ ID NO: 579);

Ac-Ile-Val-Ser-Ala-Arg-Met-Ala-(therapeutic agent) (SEQ ID NO: 580);

Ac-Val-Ile-Val-Ser-Ala-Arg-Met-Ala-(therapeutic agent) (SEQ ID NO: 581);

Ac-Val-Ile-Val-Ser-Ala-Arg-nLeu-Ala-(therapeutic agent) (SEQ ID NO: 582);

Ac-Val-Ser-Ala-Arg-nLeu-Ala-(therapeutic agent) (SEQ ID NO: 583);

Ac-Ile-Val-Ser-Ala-Arg-nLeu-Ala-(therapeutic agent) (SEQ ID NO: 584);

Ac-Gly-Ser-Gly-Arg-Ser-Leu-(therapeutic agent) (SEQ ID NO: 585);

Ac-Gly-Ser-Gly-Arg-Ser-Ser-Leu-(therapeutic agent) (SEQ ID NO: 586);

Ac-Gly-Ser-Ala-Arg-Ser-Leu-(therapeutic agent) (SEQ ID NO: 587);

Ac-Ser-Gly-Arg-Ser-Leu-(therapeutic agent) (SEQ ID NO: 588);

Ac-Ser-Gly-Arg-Ser-Ser-Leu-(therapeutic agent) (SEQ ID NO: 589);

Ac-Ser-Ala-Arg-Ser-Leu-(therapeutic agent) (SEQ ID NO: 590);

Ac-Arg-Gly-Ser-Gly-Arg-Ser-Leu-(therapeutic agent) (SEQ ID NO: 591);

Ac-Arg-Gly-Ser-Gly-Arg-Ser-Ser-Leu-(therapeutic agent) (SEQ ID NO: 592);

Ac-Arg-Gly-Ser-Ala-Arg-Ser-Leu-(therapeutic agent) (SEQ ID NO: 593);

Ac-Leu-Arg-Gly-Ser-Gly-Arg-Ser-Leu-(therapeutic agent) (SEQ ID NO: 594);

Ac-Leu-Arg-Gly-Ser-Gly-Arg-Ser-Ser-Leu-(therapeutic agent) (SEQ ID NO: 595); and

Ac-Leu-Arg-Gly-Ser-Ala-Arg-Ser-Leu-(therapeutic agent) (SEQ ID NO: 596).

In another embodiment, the conjugates provided herein are selected from:

Ac-R-Q-G-R-S-L-(therapeutic agent) (SEQ ID NO: 491);

Ac-R-Q-G-R-S-S-L-(therapeutic agent) (SEQ ID NO: 492);

Ac-R-Q-G-R-S-nL-(therapeutic agent) (SEQ ID NO: 493);

Ac-R-Q-G-R-S-nV-(therapeutic agent) (SEQ ID NO: 494);

Ac-R-Q-G-R-S-F-(therapeutic agent) (SEQ ID NO: 495);

Ac-R-Q-G-R-A-L-(therapeutic agent) (SEQ ID NO: 496);

Ac-R-Q-G-R-A-L-(therapeutic agent) (SEQ ID NO: 497);

Ac-R-Q-G-R-A-nL-(therapeutic agent) (SEQ ID NO: 498);

Ac-R-Q-G-R-A-nL-(therapeutic agent) (SEQ ID NO: 499);

Ac-R-Q-G-R-A-nV-(therapeutic agent) (SEQ ID NO: 500);

Ac-R-Q-G-R-A-Cha-(therapeutic agent) (SEQ ID NO: 501);

Ac-R-Q-G-R-A-F-(therapeutic agent) (SEQ ID NO: 502);

Ac-R-N-G-R-S-L-(therapeutic agent) (SEQ ID NO: 503);

Ac-R-N-G-R-A-nL-(therapeutic agent) (SEQ ID NO: 504);

Ac-R-Q-A-R-S-L-(therapeutic agent) (SEQ ID NO: 505);

Ac-R-Q-A-R-S-nL-(therapeutic agent) (SEQ ID NO: 506);

Ac-R-Q-A-R-S-nV-(therapeutic agent) (SEQ ID NO: 507);

Ac-R-Q-A-A-S-Cha-(therapeutic agent) (SEQ ID NO: 508);

Ac-R-Q-A-R-S-S-Cha-(therapeutic agent) (SEQ ID NO: 509);

Ac-R-Q-A-R-T-nL-(therapeutic agent) (SEQ ID NO: 510);

Ac-R-Q-A-R-A-L-(therapeutic agent) (SEQ ID NO: 511);

Ac-R-Q-A-R-A-nL-(therapeutic agent) (SEQ ID NO: 512);

Ac-R-Q-A-R-A-nV-(therapeutic agent) (SEQ ID NO: 513);

Ac-R-Q-A-R-A-Cha-(therapeutic agent) (SEQ ID NO: 514);

Ac-R-Q-S-R-A-A-(therapeutic agent) (SEQ ID NO: 515);

Ac-R-Q-S-R-A-(therapeutic agent) (SEQ ID NO: 516);

Ac-R-Q-S-R-A-nL-(therapeutic agent) (SEQ ID NO: 517);

Ac-R-Q-S-R-A-L-(therapeutic agent) (SEQ ID NO: 518);

Ac-R-Q-S-R-A-nV-(therapeutic agent) (SEQ ID NO: 519);

Ac-R-Q-S-R-A-Cha-(therapeutic agent) (SEQ ID NO: 520);

Ac-R-Q-S-R-S-S-L-(therapeutic agent) (SEQ ID NO: 521);

Ac-R-Q-S-R-S-L-(therapeutic agent) (SEQ ID NO: 522);

Ac-R-Q-S-R-S-nL-(therapeutic agent) (SEQ ID NO: 523);

Ac-R-Q-S-R-S-nL-(therapeutic agent) (SEQ ID NO: 524);

Ac-R-Q-S-R-S-nV-(therapeutic agent) (SEQ ID NO: 525);

Ac-R-Q-S-R-S-allylG-(therapeutic agent) (SEQ ID NO: 526);

Ac-R-Q-S-R-S-Cha-(therapeutic agent) (SEQ ID NO: 527);

Ac-R-Q-S-R-T-nL-(therapeutic agent) (SEQ ID NO: 528);

Ac-R-Q-T-R-S-S-L-(therapeutic agent) (SEQ ID NO: 529);

Ac-R-Q-T-R-S-L-(therapeutic agent) (SEQ ID NO: 530);

Ac-R-N-S-R-S-nL-(therapeutic agent) (SEQ ID NO: 531);

Ac-R-Q-F-R-S-L-(therapeutic agent) (SEQ ID NO: 532);

Ac-R-Q-F-R-S-nL-(therapeutic agent) (SEQ ID NO: 534);

Ac-R-Q-F-R-S-nV-(therapeutic agent) (SEQ ID NO: 535);

Ac-R-Q-F-R-S-nL-(therapeutic agent) (SEQ ID NO: 536);

Ac-R-Q-F-R-S-Cha-(therapeutic agent) (SEQ ID NO: 537);

Ac-R-Q-F-R-A-L-(therapeutic agent) (SEQ ID NO: 538);

Ac-R-Q-F-R-A-nL-(therapeutic agent) (SEQ ID NO: 539);

Ac-R-Q-F-R-A-nV-(therapeutic agent) (SEQ ID NO: 540);

Ac-R-Q-F-R-A-Cha-(therapeutic agent) (SEQ ID NO: 541);

Ac-Q-S-R-S-S-nL-(therapeutic agent) (SEQ ID NO: 542);

MeOCO-Quat2-G-R-S-L-(therapeutic agent) (SEQ ID NO: 483);

MeOCO-Quat3-G-R-S-L-(therapeutic agent) (SEQ ID NO: 484);

MeOCO-Quat-G-R-S-L-(therapeutic agent) (SEQ ID NO: 485);

MeOCO-Quat4-G-R-S-L-(therapeutic agent) (SEQ ID NO: 486);

MeOCO-Quat5-G-R-S-L-(therapeutic agent) (SEQ ID NO: 487);

MeOCO-Quat2-G-R-S-S-L-(therapeutic agent) (SEQ ID NO: 488);

MeOCO-Quat4-G-R-S-L-(therapeutic agent) (SEQ ID NO: 489);

MeOCO-Quat2-G-R-S-L-(therapeutic agent) (SEQ ID NO: 490);

Ac-Q-G-R-S-L-(therapeutic agent) (SEQ ID NO: 445);

Ac-Q-G-R-S-S-L-(therapeutic agent) (SEQ ID NO: 446);

Ac-Q-G-R-A-S-L-(therapeutic agent) (SEQ ID NO: 447);

Ac-N-G-R-S-S-L-(therapeutic agent) (SEQ ID NO: 448);

Ac-Q-G-R-S-S-nL-(therapeutic agent) (SEQ ID NO: 449);

Ac-Q-G-R-S-S-nV-(therapeutic agent) (SEQ ID NO: 450);

Ac-Q-G-R-S-S-Cha-(therapeutic agent) (SEQ ID NO: 451);

Ac-Q-G-R-S-S-allylG-(therapeutic agent) (SEQ ID NO: 452);

Ac-Q-G-R-S-S-allylG-(therapeutic agent) (SEQ ID NO: 453);

Ac-Q-A-R-S-L-(therapeutic agent) (SEQ ID NO: 454);

Ac-Q-A-R-S-S-L-(therapeutic agent) (SEQ ID NO: 455);

Ac-Q-S-R-S-L-(therapeutic agent) (SEQ ID NO: 456);

Ac-Q-S-R-S-S-nV-(therapeutic agent) (SEQ ID NO: 457);

Ac-Q-S-R-S-S-Cha-(therapeutic agent) (SEQ ID NO: 458);

Ac-Q-S-R-S-S-L-(therapeutic agent) (SEQ ID NO: 459);

Ac-Q-T-R-S-S-L-(therapeutic agent) (SEQ ID NO: 460);

Ac-Q-Aib-R-S-S-Cha-(therapeutic agent) (SEQ ID NO: 461);

Ac-Q-Aib-R-S-S-L-(therapeutic agent) (SEQ ID NO: 462);

Ac-Q-Abu-R-S-S-Cha-(therapeutic agent) (SEQ ID NO: 463);

Ac-Q-Abu-R-S-S-L-(therapeutic agent) (SEQ ID NO: 464);

Ac-Q-Cha-R-S-S-Cha-(therapeutic agent) (SEQ ID NO: 465);

Ac-Q-F-R-S-L-(therapeutic agent) (SEQ ID NO: 466);

Ac-Q-F-R-S-S-L-(therapeutic agent) (SEQ ID NO: 467);

Ac-Q-Y-R-S-S-L-(therapeutic agent) (SEQ ID NO: 468);

Ac-R-G-R-S-L-(therapeutic agent) (SEQ ID NO: 469);

Ac-R-G-R-S-S-L-(therapeutic agent) (SEQ ID NO: 470);

Ac-R-G-R-S-S-Cha-(therapeutic agent) (SEQ ID NO: 471);

Ac-R-G-R-S-Cha-(therapeutic agent) (SEQ ID NO: 472);

Ac-R-A-R-S-L-(therapeutic agent) (SEQ ID NO: 473);

Ac-R-A-R-S-S-L-(therapeutic agent) (SEQ ID NO: 474);

Ac-R-S-R-S-L-(therapeutic agent) (SEQ ID NO: 475);

Ac-R-S-R-S-S-L-(therapeutic agent) (SEQ ID NO: 476);

Ac-R-S-R-S-Cha-(therapeutic agent) (SEQ ID NO: 477);

Ac-R-S-R-S-S-Cha-(therapeutic agent) (SEQ ID NO: 478);

Ac-R-F-R-S-L-(therapeutic agent) (SEQ ID NO: 479);

Ac-R-F-R-S-Cha-(therapeutic agent) (SEQ ID NO: 480);

Ac-Y-G-R-S-S-L-(therapeutic agent) (SEQ ID NO: 481);

Ac-M(O2)-S-R-S-L-(therapeutic agent) (SEQ ID NO: 482);

Ac-R-R-Q-S-R-A-A-(therapeutic agent) (SEQ ID NO: 105);

Ac-R-R-Q-S-R-I-(therapeutic agent) (SEQ ID NO: 610);

Ac-R-R-Q-S-R-S-S-L-(therapeutic agent) (SEQ ID NO: 543);

Ac-R-R-Q-S-R-S-L-(therapeutic agent) (SEQ ID NO: 544);

Ac-R-G-S-G-R-S-L-(therapeutic agent) (SEQ ID NO: 545);

Ac-R-G-S-G-R-S-nL-(therapeutic agent) (SEQ ID NO: 546);

Ac-R-G-S-G-R-A-nL-(therapeutic agent) (SEQ ID NO: 547);

Ac-R-G-S-G-R-S-S-L-(therapeutic agent) (SEQ ID NO: 548);

Ac-I-V-S-G-R-A-S-L-(therapeutic agent) (SEQ ID NO: 549);

Ac-R-R-Q-S-R-A-(therapeutic agent) (SEQ ID NO: 108);

Ac-R-R-Q-S-R-I-(therapeutic agent) (SEQ ID NO: 111);

Ac-L-R-R-Q-S-R-A-A-(therapeutic agent) (SEQ ID NO: 106);

Ac-L-R-R-Q-S-R-G-G-(therapeutic agent) (SEQ ID NO: 109);

Ac-L-R-R-Q-S-R-A-(therapeutic agent) (SEQ ID NO: 110);

Ac-L-R-R-Q-S-R-A-I-(therapeutic agent) (SEQ ID NO: 112);

Ac-L-R-R-Q-S-R-A-I-(therapeutic agent) (SEQ ID NO: 611);

Ac-L-R-R-Q-S-R-S-S-L-(therapeutic agent) (SEQ ID NO: 550); and

Ac-L-R-R-Q-S-R-S-L-(therapeutic agent) (SEQ ID NO: 551);

In another embodiment, the conjugates provided herein are selected from:

Ac-S-G-R-S-L-(therapeutic agent) (SEQ ID NO: 362);

Ac-S-G-R-S-S-L-(therapeutic agent) (SEQ ID NO: 363);

Ac-S-G-R-S-S-S-L-(therapeutic agent) (SEQ ID NO: 364);

Ac-S-G-R-S-nL-(therapeutic agent) (SEQ ID NO: 365);

Ac-S-G-R-S-nV-(therapeutic agent) (SEQ ID NO: 366); isomer 1

Ac-S-G-R-S-nV-(therapeutic agent) (SEQ ID NO: 367); isomer 2

Ac-S-G-R-S-G(hex)-(therapeutic agent) (SEQ ID NO: 368);

Ac-S-G-R-S-Cha-(therapeutic agent) (SEQ ID NO: 369);

Ac-S-G-R-S-hCha-(therapeutic agent) (SEQ ID NO: 370);

Ac-S-A-R-S-L-(therapeutic agent) (SEQ ID NO: 371);

Ac-S-A-R-S-S-L-(therapeutic agent) (SEQ ID NO: 372);

Ac-S-S-R-S-nL-(therapeutic agent) (SEQ ID NO: 373);

Ac-T-G-R-S-Abu-(therapeutic agent) (SEQ ID NO: 374);

Ac-T-G-R-S-L-(therapeutic agent) (SEQ ID NO: 375);

Ac-T-G-R-S-nV-(therapeutic agent) (SEQ ID NO: 376);

Ac-T-G-R-S-nL-(therapeutic agent) (SEQ ID NO: 377);

Ac-T-G-R-S-G(hex)-(therapeutic agent) (SEQ ID NO: 378);

Ac-T-G-R-S-Cha-(therapeutic agent) (SEQ ID NO: 379);

Ac-T-G-R-S-hCha-(therapeutic agent) (SEQ ID NO: 380);

Ac-T-G-R-T-Abu-(therapeutic agent) (SEQ ID NO: 381);

Ac-T-G-R-hS-nL-(therapeutic agent) (SEQ ID NO: 382);

Ac-T-G-R-Abu-nL-(therapeutic agent) (SEQ ID NO: 383);

Ac-T-G-R-Abu-nV-(therapeutic agent) (SEQ ID NO: 384);

Ac-T-G-F(Gn)-S-nL-(therapeutic agent) (SEQ ID NO: 385);

Ac-T-G-F(Gn)-S-Cha-(therapeutic agent) (SEQ ID NO: 386);

Ac-T-G-F(Gn)-Abu-nV-(therapeutic agent) (SEQ ID NO: 387);

Ac-T-G-K(alloc)-S-nL-(therapeutic agent) (SEQ ID NO: 388);

Ac-T-G-K-S-nL-(therapeutic agent) (SEQ ID NO: 389);

Ac-T-G-hR-S-nL-(therapeutic agent) (SEQ ID NO: 390);

Ac-(hS)G-G-R-S-nL-(therapeutic agent) (SEQ ID NO: 391);

MeOCO-T-G-R-S-nL-(therapeutic agent) (SEQ ID NO: 392);

PhSO2-T-G-R-S-nL-(therapeutic agent) (SEQ ID NO: 393);

MeOEtCO-T-G-R-S-nL-(therapeutic agent) (SEQ ID NO: 394);

MeO(EtO)2Ac-T-G-R-S-nL-(therapeutic agent) (SEQ ID NO: 395);

4-oxo-Pentanoyl-T-G-R-S-nL-(therapeutic agent) (SEQ ID NO: 396);

3,4-MethyidioxyPhAc-T-G-R-S-nL-(therapeutic agent) (SEQ ID NO: 397);

2-PyridylAc-T-G-R-S-nL-(therapeutic agent) (SEQ ID NO: 398);

PhOAc-T-G-R-S-nL-(therapeutic agent) (SEQ ID NO: 399);

L-3-PhLactyl-T-G-R-S-nL-(therapeutic agent) (SEQ ID NO: 400);

MeOAc-T-G-R-S-nL-(therapeutic agent) (SEQ ID NO: 401);

PhAc-T-G-R-S-nL-(therapeutic agent) (SEQ ID NO: 402);

MeOEtOCO-T-G-R-S-nL-(therapeutic agent) (SEQ ID NO: 403);

MeOEtOAc-T-G-R-S-nL-(therapeutic agent) (SEQ ID NO: 404);

HOOCButa-T-G-R-S-nL-(therapeutic agent) (SEQ ID NO: 405);

Z-T-G-R-S-nL-(therapeutic agent) (SEQ ID NO: 406);

EtOCO-T-G-R-S-nL-(therapeutic agent) (SEQ ID NO: 407);

βA-T-G-R-S-nL-(therapeutic agent) (SEQ ID NO: 408);

Pent-4-ynoyl-T-G-R-S-nL-(therapeutic agent) (SEQ ID NO: 409);

NapAc-T-G-R-S-nL-(therapeutic agent) (SEQ ID NO: 410);

iBoc-T-G-R-S-nL-(therapeutic agent) (SEQ ID NO: 411);

HOAc-T-G-R-S-nL-(therapeutic agent) (SEQ ID NO: 412);

MeSucc-T-G-R-S-nL-(therapeutic agent) (SEQ ID NO: 413);

N,N-diMeGly-T-G-R-S-nL-(therapeutic agent) (SEQ ID NO: 414);

Succ-T-G-R-S-nL-(therapeutic agent) (SEQ ID NO: 415);

HCO-T-G-R-S-nL-(therapeutic agent) (SEQ ID NO: 416);

Ac-T-A-R-S-nL-(therapeutic agent) (SEQ ID NO: 417);

Ac-T-A-F(Gn)-S-nL-(therapeutic agent) (SEQ ID NO: 418);

Ac-T-A-R-Abu-nV-(therapeutic agent) (SEQ ID NO: 419);

Ac-T-A-R-S-Abu-(therapeutic agent) (SEQ ID NO: 420);

Ac-T-A-R-T-Abu-(therapeutic agent) (SEQ ID NO: 421);

Ac-T-S(O-Me)-R-S-nL-(therapeutic agent) (SEQ ID NO: 422);

Ac-T-hS-R-S-nL-(therapeutic agent) (SEQ ID NO: 423);

Ac-T-(1-Me)H-R-S-nL-(therapeutic agent) (SEQ ID NO: 424);

Ac-T-(3-Me)H-R-S-nL-(therapeutic agent) (SEQ ID NO: 425);

Ac-T-H-R-S-nL-(therapeutic agent) (SEQ ID NO: 426);

Ac-T-Sar-R-S-nL-(therapeutic agent) (SEQ ID NO: 427);

Ac-T-nV-R-S-nL-(therapeutic agent) (SEQ ID NO: 428);

Ac-T-nL-R-S-nL-(therapeutic agent) (SEQ ID NO: 429);

Ac-T-A-R-S-Cha-(therapeutic agent) (SEQ ID NO: 430);

Ac-T-Abu-R-S-nL-(therapeutic agent) (SEQ ID NO: 431);

Ac-4,4diMeThr-G-R-S-nL-(therapeutic agent) (SEQ ID NO: 432);

Ac-hS-G-R-S-nL-(therapeutic agent) (SEQ ID NO: 433);

Ac-hS-G-R-hS-Cha-(therapeutic agent) (SEQ ID NO: 434);

Ac-hS-G-R-S-Cha-(therapeutic agent) (SEQ ID NO: 435);

Ac-hS-G-R-T-Cha-(therapeutic agent) (SEQ ID NO: 436);

Ac-hS-A-R-S-Cha-(therapeutic agent) (SEQ ID NO: 437);

Ac-N-G-R-S-nL-(therapeutic agent) (SEQ ID NO: 438);

Ac-Y-G-R-S-S-L-(therapeutic agent) (SEQ ID NO: 439);

Ac-Y-G-R-S-Cha-(therapeutic agent) (SEQ ID NO: 440);

Ac-Q-G-R-S-S-nL-(therapeutic agent) (SEQ ID NO: 441);

Ac-Q-G-R-S-S-nV-(therapeutic agent) (SEQ ID NO: 442);

Ac-L-R-G-S-G-R-S-A-(therapeutic agent) (SEQ ID NO: 573);

Ac-L-R-G-S-G-R-S-L-(therapeutic agent) (SEQ ID NO: 342);

Ac-L-R-G-S-G-R-S-L-(therapeutic agent) (SEQ ID NO: 343);

Ac-L-R-G-S-G-R-S-S-nL-(therapeutic agent) (SEQ ID NO: 344);

Ac-L-R-G-S-G-R-S-S-Cha-(therapeutic agent) (SEQ ID NO: 345);

Ac-L-R-G-dS-A-R-S-A-(therapeutic agent) (SEQ ID NO: 574);

Ac-L-R-G-S-A-R-S-S-L-(therapeutic agent) (SEQ ID NO: 346);

Ac-L-R-G-S-A-R-S-L-(therapeutic agent) (SEQ ID NO: 347);

Ac-L-R-G-S-A-R-S-S-Cha-(therapeutic agent) (SEQ ID NO: 348);

Ac-L-R-G-S-A-R-S-S-nV-(therapeutic agent) (SEQ ID NO: 349);

Ac-L-R-G-S-A-R-S-S-nL-(therapeutic agent) (SEQ ID NO: 350);

Ac-V-I-V-S-G-R-A-L-(therapeutic agent) (SEQ ID NO: 351);

Ac-V-I-V-S-A-R-S-L-(therapeutic agent) (SEQ ID NO: 352);

Ac-V-I-V-S-G-R-S-S-L-(therapeutic agent) (SEQ ID NO: 353);

Ac-V-I-V-S-A-R-M-A-(therapeutic agent) (SEQ ID NO: 354);

Ac-V-I-V-S-A-R-nL-A-(therapeutic agent) (SEQ ID NO: 355);

Ac-V-I-V-S-A-R-S-nL-(therapeutic agent) (SEQ ID NO: 356);

Ac-V-I-V-S-A-R-S-Cha-(therapeutic agent) (SEQ ID NO: 357);

Ac-V-I-V-S-A-R-S-Cha-(therapeutic agent) (SEQ ID NO: 358);

Ac-V-I-V-S-A-R-S-S-Cha-(therapeutic agent) (SEQ ID NO: 359);

Ac-R-R-(Me)C-P-G-R-V-V-(therapeutic agent) (SEQ ID NO: 360);

Ac-R-R-nV-P-A-R-S-L-(therapeutic agent) (SEQ ID NO: 361);

Ac-R-G-dS-A-R-S-A-(therapeutic agent) (SEQ ID NO: 309);

Ac-R-G-S-G-R-S-A-(therapeutic agent) (SEQ ID NO: 310);

Ac-R-G-S-G-R-A-L-(therapeutic agent) (SEQ ID NO: 311);

Ac-R-G-S-G-R-S-L-(therapeutic agent) (SEQ ID NO: 312);

Ac-R-G-S-G-R--S-nL-(therapeutic agent) (SEQ ID NO: 313);

Ac-R-G-S-G-R-A-nL-(therapeutic agent) (SEQ ID NO: 314);

Ac-R-G-S-G-R-S-S-L-(therapeutic agent) (SEQ ID NO: 315);

Ac-R-G-S-G-R-S-Cha-(therapeutic agent) (SEQ ID NO: 316);

Ac-R-G-S-G-R-S-S-Cha-(therapeutic agent) (SEQ ID NO: 317);

Ac-R-G-S-A-R-S-Cha-(therapeutic agent) (SEQ ID NO: 318);

Ac-R-G-S-A-R-S-S-(therapeutic agent) (SEQ ID NO: 319);

Ac-R-G-S-A-R-S-nV-(therapeutic agent) (SEQ ID NO: 320);

Ac-R-G-S-A-R-S-S-nV-(therapeutic agent) (SEQ ID NO: 321);

Ac-R-G-S-A-R-S-L-(therapeutic agent) (SEQ ID NO: 322);

Ac-R-(Me)C-P-G-R-V-V-(therapeutic agent) (SEQ ID NO: 323);

Ac-R-(Me)C-P-G-R-V-V-(therapeutic agent) (SEQ ID NO: 324);

Ac-R-C(Me)-P-G-R-S-L-(therapeutic agent) (SEQ ID NO: 325);

Ac-R-L-P-G-R-S-L-(therapeutic agent) (SEQ ID NO: 326);

Ac-R-V-P-G-R-S-L-(therapeutic agent) (SEQ ID NO: 327);

Ac-R-V-P-G-R-S-L-(therapeutic agent) (SEQ ID NO: 328);

Ac-R-nL-P-G-R-S-L-(therapeutic agent) (SEQ ID NO: 329);

Ac-R-G(tBu)-P-A-R-S-L-(therapeutic agent) (SEQ ID NO: 330);

Ac-R-L-P-A-R-S-L-(therapeutic agent) (SEQ ID NO: 331);

Ac-R-V-P-A-R-S-L-(therapeutic agent) (SEQ ID NO: 332);

Ac-R-nL-P-A-R-S-L-(therapeutic agent) (SEQ ID NO: 333);

Ac-I-V-S-G-R-A-L-(therapeutic agent) (SEQ ID NO: 334);

Ac-I-V-S-G-R-S-S-L-(therapeutic agent) (SEQ ID NO: 335);

Ac-I-V-S-G-R-A-S-L-(therapeutic agent) (SEQ ID NO: 336);

Ac-I-V-S-A-R-M-A-(therapeutic agent) (SEQ ID NO: 337);

Ac-I-V-S-A-R-nL-A-(therapeutic agent) (SEQ ID NO: 338);

Ac-I-V-S-A-R-S-L-(therapeutic agent) (SEQ ID NO: 339);

Ac-I-V-S-A-R-S-nL-(therapeutic agent) (SEQ ID NO: 340);

Ac-I-V-S-A-R-S-S-L-(therapeutic agent) (SEQ ID NO: 341);

Ac-G-S-G-R-S-A-(therapeutic agent) (SEQ ID NO: 585);

Ac-G-S-G-R-S-L-(therapeutic agent) (SEQ ID NO: 277);

Ac-G-S-G-R-A-L-(therapeutic agent) (SEQ ID NO: 278);

Ac-G-S-G-R-S-S-L-(therapeutic agent) (SEQ ID NO: 279);

Ac-G-S-G-R-L-(therapeutic agent) (SEQ ID NO: 280);

Ac-G-S-G-(4-guan)Phg-S-L-(therapeutic agent) (SEQ ID NO: 281);

Ac-G-S-G-R-S-S-Cha-(therapeutic agent) (SEQ ID NO: 282);

Ac-G-S-G-R-A-S-L-(therapeutic agent) (SEQ ID NO: 283);

Ac-G-S-G-R-S-nL-(therapeutic agent) (SEQ ID NO: 284);

Ac-G-T-G-R-S-nL-(therapeutic agent) (SEQ ID NO: 285);

Succ-bA-T-G-R-S-nL-(therapeutic agent) (SEQ ID NO: 286);

Ac-G-T-G-R-S-hCha-(therapeutic agent) (SEQ ID NO: 287);

Ac-G-hS-G-R-S-nL-(therapeutic agent) (SEQ ID NO: 288);

Ac-G-dS-A-R-S-A-(therapeutic agent) (SEQ ID NO: 289);

Ac-G-S-A-R-S-L-(therapeutic agent) (SEQ ID NO: 290);

Ac-G-S-A-R-S-S-Cha-(therapeutic agent) (SEQ ID NO: 291);

Ac-G-S-A-R-S-S-L-(therapeutic agent) (SEQ ID NO: 292);

Ac-G-S-A-R-A-S-L-(therapeutic agent) (SEQ ID NO: 293);

Ac-V-S-G-R-S-L-(therapeutic agent) (SEQ ID NO: 294);

Ac-V-S-G-R-A-L-(therapeutic agent) (SEQ ID NO: 295);

Ac-V-S-G-R-A-S-L-(therapeutic agent) (SEQ ID NO: 296);

Ac-V-S-G-R-S-S-L-(therapeutic agent) (SEQ ID NO: 297);

Ac-V-S-A-R-M-A-(therapeutic agent) (SEQ ID NO: 298);

Ac-V-S-A-R-nL-A-(therapeutic agent) (SEQ ID NO: 299);

Ac-V-S-A-R-S-nL-(therapeutic agent) (SEQ ID NO: 300);

Ac-V-S-A-R-S-L-(therapeutic agent) (SEQ ID NO: 301);

Ac-(Me)C-P-G-R-V-V-(therapeutic agent) (SEQ ID NO: 302);

Ac-(Me)C-P-G-R-V-V-(therapeutic agent) (SEQ ID NO: 303);

Ac-C(Me)-P-G-R-A-L-(therapeutic agent) (SEQ ID NO: 304);

Ac-C(Me)-P-G-R-S-L-(therapeutic agent) (SEQ ID NO: 305);

Ac-C(Me)-P-A-R-S-L-(therapeutic agent) (SEQ ID NO: 306);

Ac-C(Me)-P-A-R-A-S-L-(therapeutic agent) (SEQ ID NO: 307);and

Ac-G(tBu)-P-G-R-S-L-(therapeutic agent) (SEQ ID NO: 308);

In another embodiment, the conjugates provided herein are selected from:

Ac-Q-S-R-A-A-(therapeutic agent) (SEQ ID NO: 552);

Ac-Q-S-R-S-A-(therapeutic agent) (SEQ ID NO: 553);

Ac-Q-S-R-S-G-(therapeutic agent) (SEQ ID NO: 554);

Ac-R-S-R-A-A-(therapeutic agent) (SEQ ID NO: 555);

Ac-R-Q-S-R-A-A-(therapeutic agent) (SEQ ID NO: 556);

Ac-R-Q-S-R-S-A-(therapeutic agent) (SEQ ID NO: 557); and

Ac-R-Q-S-R-S-A-A-(therapeutic agent) (SEQ ID NO: 558);

In another embodiment, the conjugates provided herein are selected from:

Ac-R-G-S-G-R-S-A-(therapeutic agent) (SEQ ID NO: 559);

Ac-S-G-R-A-A-(therapeutic agent) (SEQ ID NO: 560);

Ac-S-G-R-S-A-(therapeutic agent) (SEQ ID NO: 561);

Ac-S-G-R-S-S-A-(therapeutic agent) (SEQ ID NO: 562);

Ac-S-G-R-A-S-A-(therapeutic agent) (SEQ ID NO: 563);

Ac-S-G-R-S-G-(therapeutic agent) (SEQ ID NO: 564);

Ac-S-G-R-S-S-G-(therapeutic agent) (SEQ ID NO: 565);

Ac-S-G-R-S-G-A-(therapeutic agent) (SEQ ID NO: 566);

Ac-S-G-R-S-G-G-(therapeutic agent) (SEQ ID NO: 567); and

Ac-G-T-G-R-S-G-G-(therapeutic agent) (SEQ ID NO: 568);

In another embodiment, the conjugates provided herein are selected from:

Ac-L-R-R-Q-S-R-A-A-(therapeutic agent) (SEQ ID NO: 597);

MeSO2-dA(Chx)-Abu-R-S-L-(therapeutic agent) (SEQ ID NO: 598);

Ac-R-A-R-S-L-(therapeutic agent) (SEQ ID NO: 599);

Ac-dA(Chx)-Abu-R-S-L-(therapeutic agent) (SEQ ID NO: 600);

Ac-dA(Chx)-Abu-R-S-S-L-(therapeutic agent) (SEQ ID NO: 601);

Ac-Q-G-R-S-S-L-(therapeutic agent) (SEQ ID NO: 602);

MeOCO-dhF-P(OH)-R-S-S-L-(therapeutic agent) (SEQ ID NO: 603);

MeOCO-Quat4-G-R-S-L-(therapeutic agent) (SEQ ID NO: 604);

Ac-dCha-P(OH)-R-S-S-L-(therapeutic agent) (SEQ ID NO: 605);

Ac-dCha-Abu-R-S-S-A-(therapeutic agent) (SEQ ID NO: 606);

MeOCO-Quat2-G-R-S-L-(therapeutic agent) (SEQ ID NO: 607);

MeOCO-Quat3-G-R-S-L-(therapeutic agent) (SEQ ID NO: 608); and

MeOCO-Quat-G-R-S-L-(therapeutic agent) (SEQ ID NO: 609).

It also understood that conjugates containing the above peptidic substrate portions can be prepared with other capping groups in place of Ac (see, e.g., the description herein of the capping groups Xⁿ). Therapeutic agents for use in the above conjugates include, for example, cytotoxic agents, such as, but not limited to, a toxin such as abrin, ricin A, pseudomonas exotoxin shiga toxin, diphtheria toxin and other such toxins and toxic portions thereof; proteins such as tumor necrosis factor, interferons, such as α-interferon and gamma-interferon, pro-coagulants such as tissue factor and tissue factor variants, pro-apoptotic agents such FAS-ligand, nerve growth factor, platelet derived growth factor, tissue plasminogen activator; biological response modifiers such as, for example, lymphokines, interleukin-I (IL-I), interleukin-2 (IL-2), interleukin-6 (IL-6), granulocyte macrophage colony stimulating factor (GMCSF), granulocyte colony stimulating factor (G-CSF), fibroblast growth factors (FGFs) and other growth factors, the methotrexate group of drugs, the anthracycline family of drugs, the vinca alkaloid drugs, the mitomycins, the bleomycins, the cytotoxic nucleosides including cytosine arabinosides and difluoronucleosides, the pteridine family of drugs, diynenes, the taxanes and the podophyllotoxins. All such conjugates are within the scope of the instant disclosure and can be prepared and used as disclosed herein.

Thus, the conjugates provided herein include, but are not limited to, those disclosed herein where the therapeutic agent is, e.g., doxorubicin, carminomycin, daunorubicin, detorubicin, idarubicin, epirubicin, esorubicin, THP, AD-32, aminopterin, methotrexate, methopterin, dichloromethotrexate, mitomycin C, porfiromycin, 5-fluorouracil, 6-mercaptopurine, cytosine arabinoside, podophyllotoxin, or podophyllotoxin derivatives such as etoposide or etoposide phosphate, melphalan, vinblastine, vincristine, leurosidine, vindesine, leurosine, taxol, estramustine, cisplatin, combretastatin and analogs, and cyclophosphamide. In one embodiment, the therapeutic agent is doxorubicin. In another embodiment, the therapeutic agent is taxol.

Any conjugates corresponding to the above conjugates or any conjugates disclosed herein where the P1′ and/or P2′ residues are Ile in place of Ala are within the scope of the instant disclosure and can be prepared and used as disclosed herein.

Any peptidic substrates formed by permutation and selection of amino acids from those set forth in the above definitions of P groups are contemplated.

### D. Preparation of the Conjugates

The peptidic substrates of the conjugates provided herein are synthesized from their constituent amino acids by conventional peptide synthesis techniques, such as by solid-phase technology. The peptidic substrates are then purified by reverse-phase high performance liquid chromatography (HPLC).

The peptide acids can be prepared from their constituent Fmoc-aminoacids. Standard methods of peptide synthesis are disclosed, for example, in the following works: Synthesis Notes Section, NovaBiochem Catalog 2002/3, Schroeder et al., “The Peptides”, Vol. 1, Academic Press 1965; Bodansky et al., “Peptide Synthesis”, Interscience Publishers, 1966; McOmie (ed.) “Protective Groups in Organic Chemistry”, Plenum Press, 1973, Barany et al., “The Peptides: Analysis, Synthesis, Biology” 2, Chapter 1, Academic Press, 1990, and Stewart et al., “Solid Phase Peptide Synthesis”, Second Edition, Pierce Chemical Company, 1994. The disclosures of these references are hereby incorporated by reference.

The pharmaceutically acceptable salts of the conjugates provided herein include the conventional non-toxic salts of the conjugates as formed, e.g., from non-toxic inorganic or organic acids. For example, such conventional non-toxic salts include those derived from inorganic acids such as hydrochloric, hydrobromic, sulfuric, sulfamic, phosphoric, nitric and the like: and the salts prepared from organic acids such as acetic, propionic, succinic, glycolic, stearic, lactic, malic, tartaric, citric, ascorbic, pamoic, maleic, hydroxymaleic, phenyl-acetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

The conjugates provided herein that contain the peptidic moieties containing the cell surface protease cleavage site and a therapeutic agent can similarly be synthesized by techniques known to those of skill in the art. For example, a free amine moiety on the therapeutic agent can be covalently attached to the peptidic substrate at the carboxyl terminus such that an amide bond is formed. Similarly, an amide bond can be formed by covalently coupling an amine moiety of the peptidic substrate and a carboxyl moiety of the therapeutic agent. For these purposes a reagent such as 2-(1H-benzotriazol-1-yl)-1,3,3-tetramethyl-uronium hexafluorophosphate (known as HBTU) and 1-hyroxybenzotriazole hydrate (known as HOBT), dicyclohexyl-carbodiimide (DCC), N-ethyl-N-(3-dimethylaminopropyl)-carbodiimide (EDC), diphenyl-phosphorylazide (DPPA), benzotriazol-1-yl-oxy-tris-(dimethylamino)-phosphonium hexafluorophosphate (BOP) and the like, used in combination or singularly, can be utilized.

Furthermore, the instant conjugates can be formed by a non-peptidyl bond between the cell surface protease cleavage site and a therapeutic agent. For example, the therapeutic agent can be covalently attached to the carboxyl terminus of the peptidic substrate via a hydroxyl moiety on the therapeutic agent, thereby forming an ester linkage. For this purpose a reagent such as a combination of HBTU and HOBT, a combination of BOP and imidazole, a combination of DCC and DMAP, and the like can be utilized. The carboxylic acid also can be activated by forming the nitro-phenyl ester or the like and reacted in the presence of DBU (1,8-diazabicyclo[5,4,O]undec-7-ene).

The instant conjugates also can be formed by attachment of the peptidic substrate to the therapeutic agent via a linker unit. Such linker units include, for example, a biscarbonyl alkyl diradical whereby an amine moiety on the therapeutic agent is connected with the linker unit to form an amide bond and the amino terminus of the peptidic substrate is connected with the other end of the linker unit also forming an amide bond. Conversely, a diaminoalkyl diradical linker unit, whereby a carbonyl moiety on the cytotoxic agent is covalently attached to one of the amines of the linker unit while the other amine of the linker unit is covalently attached to the C-terminus of the peptidic substrate, also can be useful. Other such linker units which are stable to the physiological environment when not in the presence of a cell surface protease, or a soluble, shed or released form thereof, but are cleavable upon the cleavage of the cell surface protease proteolytic cleavage site, or a soluble, shed or released form thereof, are also envisioned. Furthermore, linker units can be utilized that, upon cleavage of the cell surface protease proteolytic cleavage site, remain attached to the therapeutic agent but do not significantly decrease the therapeutic activity of such a post-cleavage therapeutic agent derivative when compared with an unmodified therapeutic agent.

One skilled in the art understands that in the synthesis of the conjugates provided herein, one can need to protect various reactive functionalities on the starting compounds and intermediates while a desired reaction is carried out on other portions of the molecule. After the desired reactions are complete, or at any desired time, normally such protecting groups will be removed by, for example, hydrolytic or hydrogenolytic means. Such protection and deprotection steps are conventional in organic chemistry. One skilled in the art is referred to Protective Groups in Organic Chemistry, McOmie, ed., Plenum Press, NY, N.Y. (1973); and, Protective Groups in Organic Synthesis, Greene, ed., John Wiley & Sons, NY, N.Y. (1991) for the teaching of protective groups which can be useful in the preparation of the conjugates provided herein.

By way of example only, useful amino-protecting groups can include, for example, C₁-C₁₀ alkanoyl groups such as formyl, acetyl, dichloroacetyl, propionyl, hexanoyl, 3,3-diethylhexanoyl, γ-chlorobutryl, and the like; C₁-C₁₀ alkoxycarbonyl and C₅-C₁₅ aryloxycarbonyl groups such as tert-butoxycarbonyl, benzyloxycarbonyl, allyloxycarbonyl, 4-nitrobenzyloxycarbonyl, fluorenylmethyloxycarbonyl and cinnamoyloxy-carbonyl; halo(C₁-C₁₀)-alkoxycarbonyl such as 2,2,2-trichloroethoxy-carbonyl; and C₁-C₁₅ arylalkyl and alkenyl group such as benzyl, phenethyl, allyl, trityl, and the like. Other commonly used amino-protecting groups are those in the form of enamines prepared with β-keto-esters such as methyl or ethyl acetoacetate.

Useful carboxy-protecting groups can include, for example, C₁-C₁₀ alkyl groups such as methyl, tert-butyl, decyl; halo C₁-C₁₀ alkyl such as 2,2,2-trichloroethyl, and 2-iodoethyl; C₅-C₁₅ arylalkyl such as benzyl, 4-methoxybenzy], 4-nitrobenzyl, triphenylmethyl, diphenyl-methyl; C₁-C₁₀ alkanoyloxymethyl such as acetoxy-methyl, propionoxymethyl and the like; and groups such as phenacyl, 4-halophenacyl, allyl, dimethylallyl, tri-(C₁-C₃ alkyl)silyl, such as trimethylsilyl, β-p-toluenesulfonylethyl, β-p-nitrophenyl-thioethyl, 2,4,6-trimethylbenzyl, β-methylthioethyl, phthalimidomethyl, 2,4-dinitro-phenylsulphenyl, 2-nitrobenzhydryl and related groups.

Similarly, useful hydroxy protecting groups can include, for example, the formyl group, the chloroacetyl group, the benzyl group, the benzhydryl group, the trityl group, the 4-nitrobenzyl group, the trimethylsilyl group, the phenacyl group, the tert-butyl group, the methoxymethyl group, the tetrahydropyranyl group, the tert-butyl-dimethylsilyl group and the like.

With respect to the embodiment of a peptidic substrate combined with the anthracycline antibiotic doxorubicin, the following Reaction Schemes illustrate the synthesis of the conjugates provided herein.

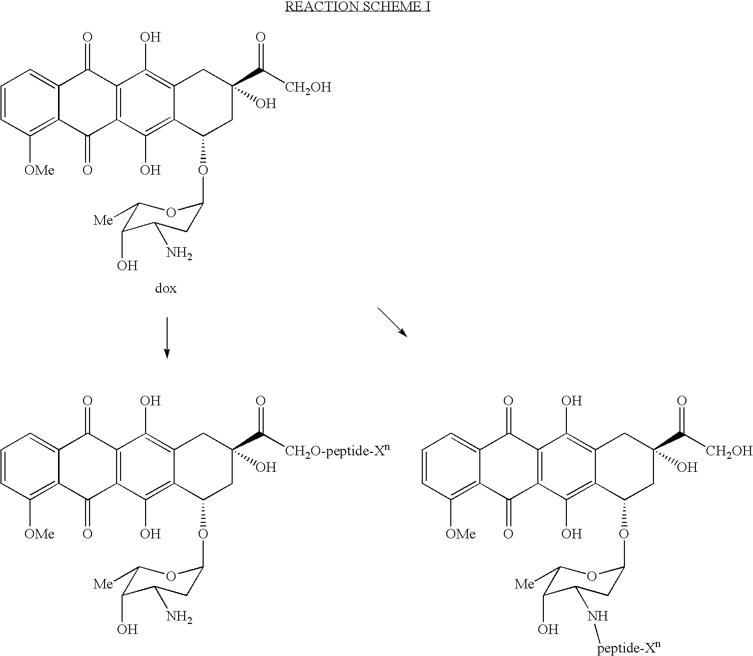

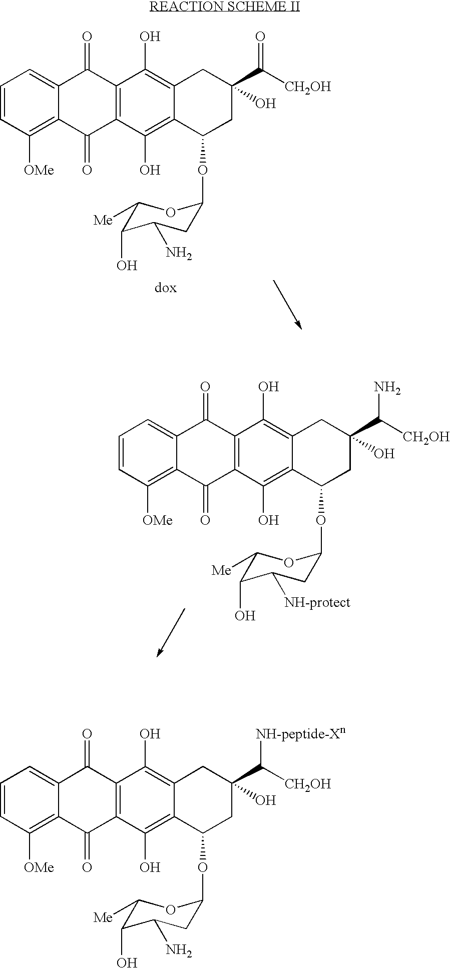

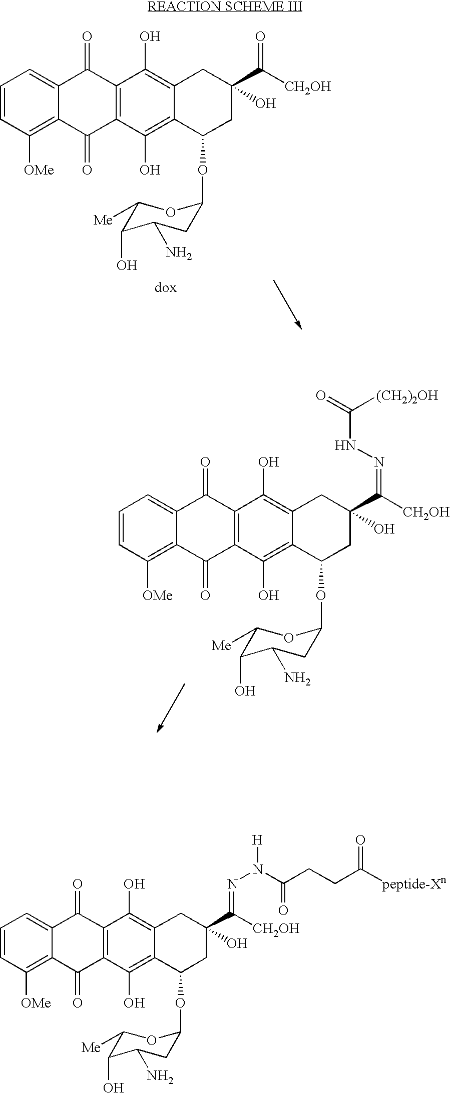

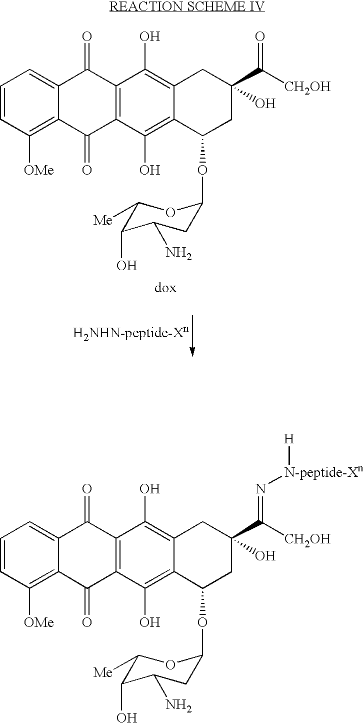

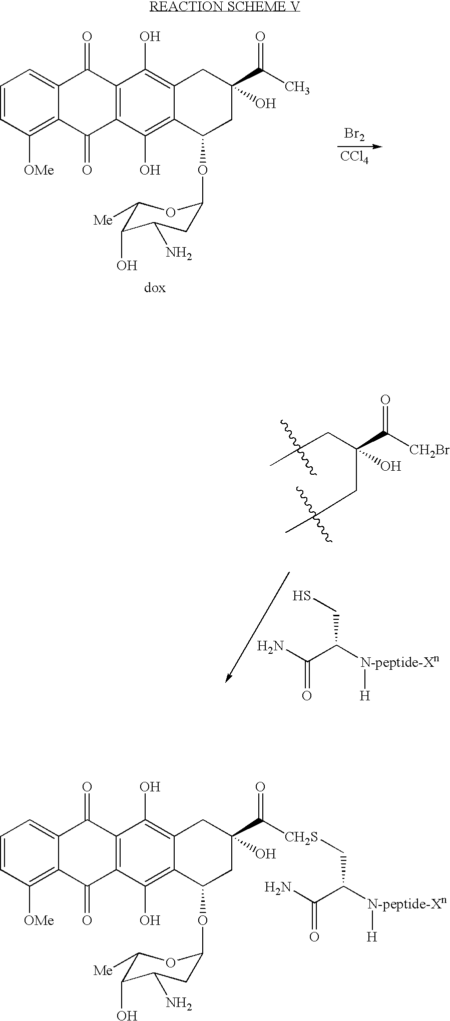

Reaction Scheme VI illustrates preparation of the conjugates provided herein of a peptidic substrate and the vinca alkaloid cytotoxic agent vinblastine wherein the attachment of vinblastine is at the C-terminus of the peptidic substrate. The use of the 1,3-diaminopropane linker is illustrative only; other linker units between the carbonyl of vinblastine and the C-terminus of the peptidic substrate are also envisioned (e.g., (CH₂)ᵤ—T—(CH₂)ᵤ). The acyl azide starting material is prepared from vinglasine by reaction with hydrazine (60-65° C., MeOH), followed by reaction with HCl/DMF/isoamyl nitrite. Furthermore, Reaction Scheme VI illustrates a synthesis of conjugates wherein the C4-hydroxy moiety is reacetylated following the addition of the linker unit. It is known that the desacetyl vinblastine conjugate also is efficacious and can be prepared by eliminating the steps shown in Reaction Scheme VI of protecting the primary amine of the linker and reacting the intermediate with acetic anhydride, followed by deprotection of the amine (see, e.g., International Patent Application Publication No. WO 98/10651). Conjugation of the peptidic substrate at other positions and functional groups of vinblastine can be readily accomplished by one of ordinary skill in the art and also is expected to provide conjugates that are substrates for cell surface proteases, or a soluble, shed or released form thereof.

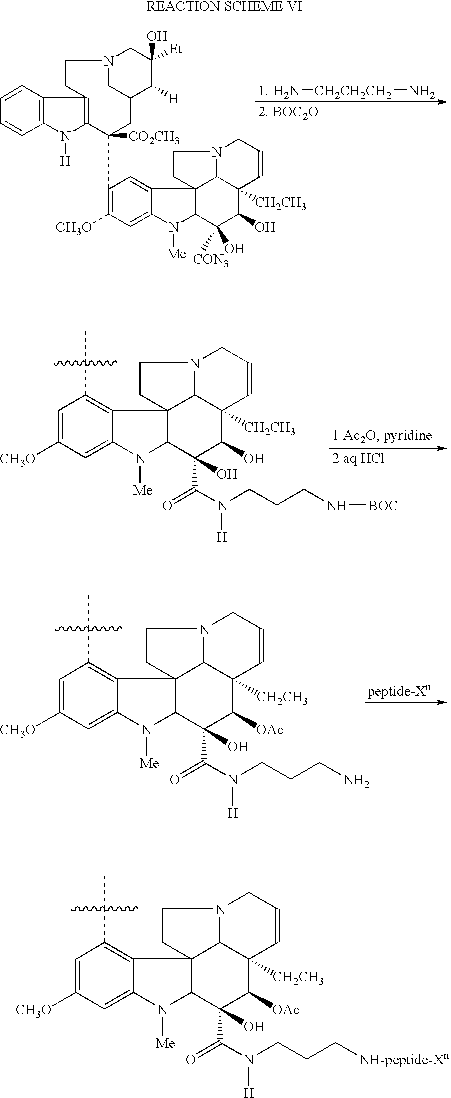

Reaction Scheme VII illustrates preparation of certain of the conjugates utilized in the compositions and methods provided herein wherein the peptidic substrates are combined with the vinca alkaloid cytotoxic agent vinblastine. Attachment of the N-terminus of the peptidic substrate to vinblastine is illustrated (S. P. Kandukuri et al. (1985) J. Med. Chem. 28:1079-1088).

It also is understood that conjugates can be prepared wherein the N-terminus of the peptidic substrate utilized in the compositions and methods provided herein is combined with one therapeutic agent, such as a cytotoxic agent, such as vinblastine, while the C-terminus is simultaneously attached to another cytotoxic agent, which is the same or different cytotoxic agent, such as doxorubicin. Reaction Scheme VIII illustrates the synthesis of such a polycytotoxic agent conjugate. Such a polycytotoxic conjugate can offer advantages over a conjugate containing only one cytotoxic agent.

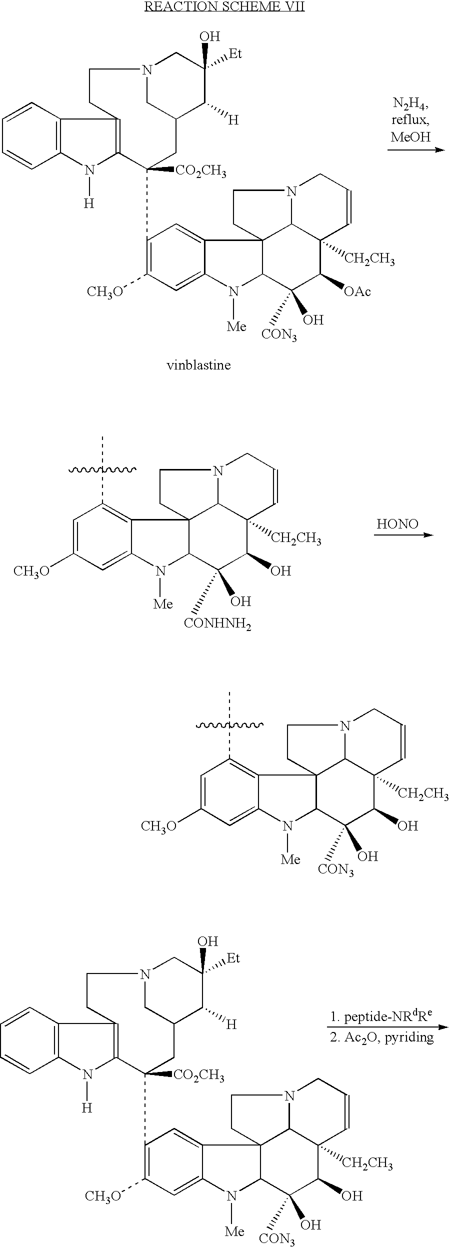

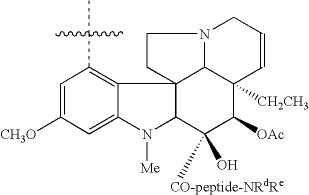

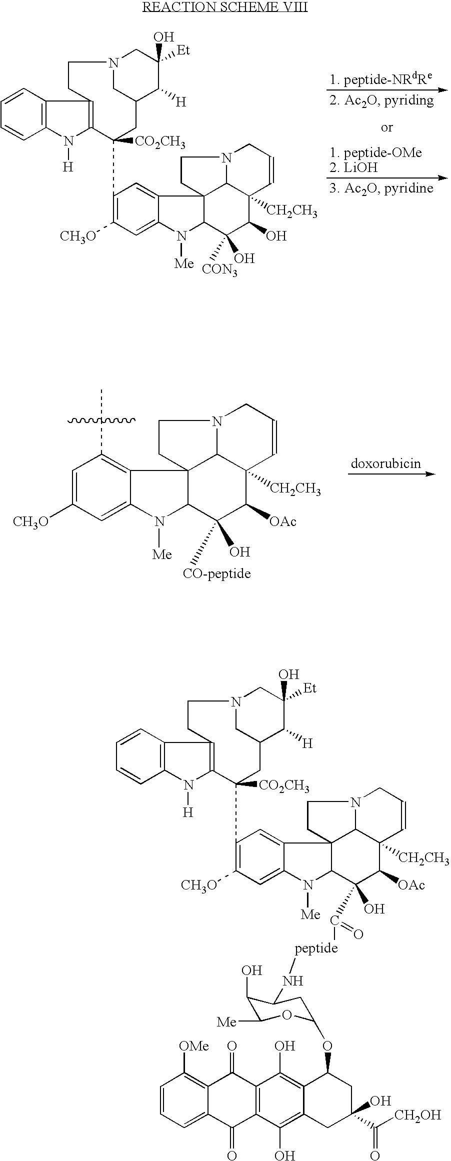

With respect to the embodiment of a peptidic substrate combined with desacetylvinblastine, the following Reaction Schemes IX and X illustrate the synthesis of the conjugates provided herein.

Reaction Scheme IX illustrates preparation of conjugates provided herein containing the peptidic substrates provided herein and the vinca alkaloid cytotoxic agent vinblastine wherein the attachment of the oxygen of the 4-desacetylvinblastine is at the C-terminus of the peptidic substrate. While other sequences of reactions can be useful in forming such conjugates, it is known that initial attachment of a single amino acid to the 4-oxygen and subsequent attachment of the remaining peptidic substrate sequence to that amino acid is an exemplarary method (see, International Patent Application Publication No. WO 99/28345). It also is known that 3,4-dihydro-3-hydroxy-4-oxo-1,2,3-benzotriazine (ODHBT) can be utilized in place of HOAt in the final coupling step.

Reaction Scheme X illustrates preparation of conjugates of the peptidic substrates provided herein wherein a hydroxy alkanoyl acid is used as a linker between the vinca drug and the peptidic substrate.

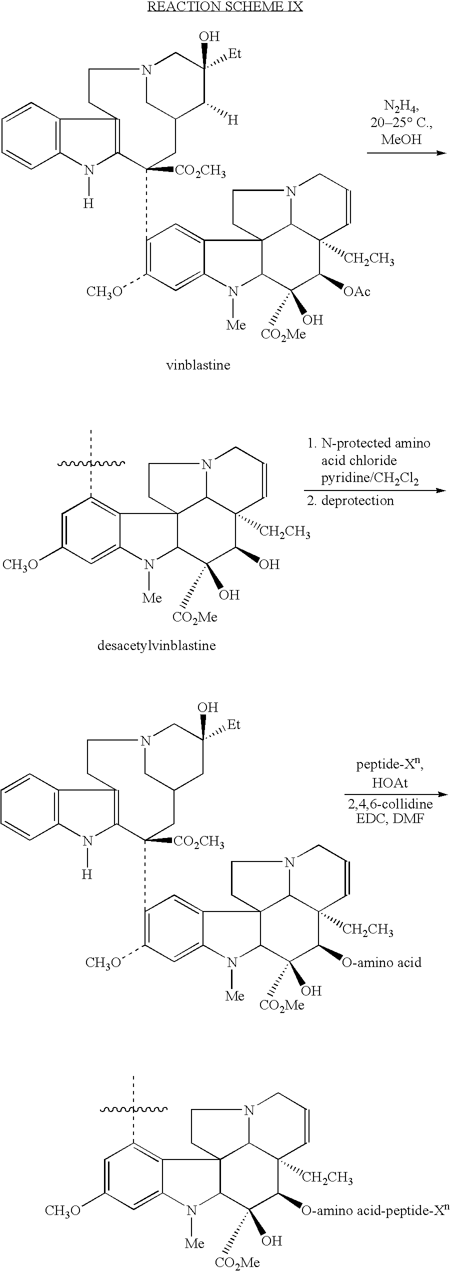

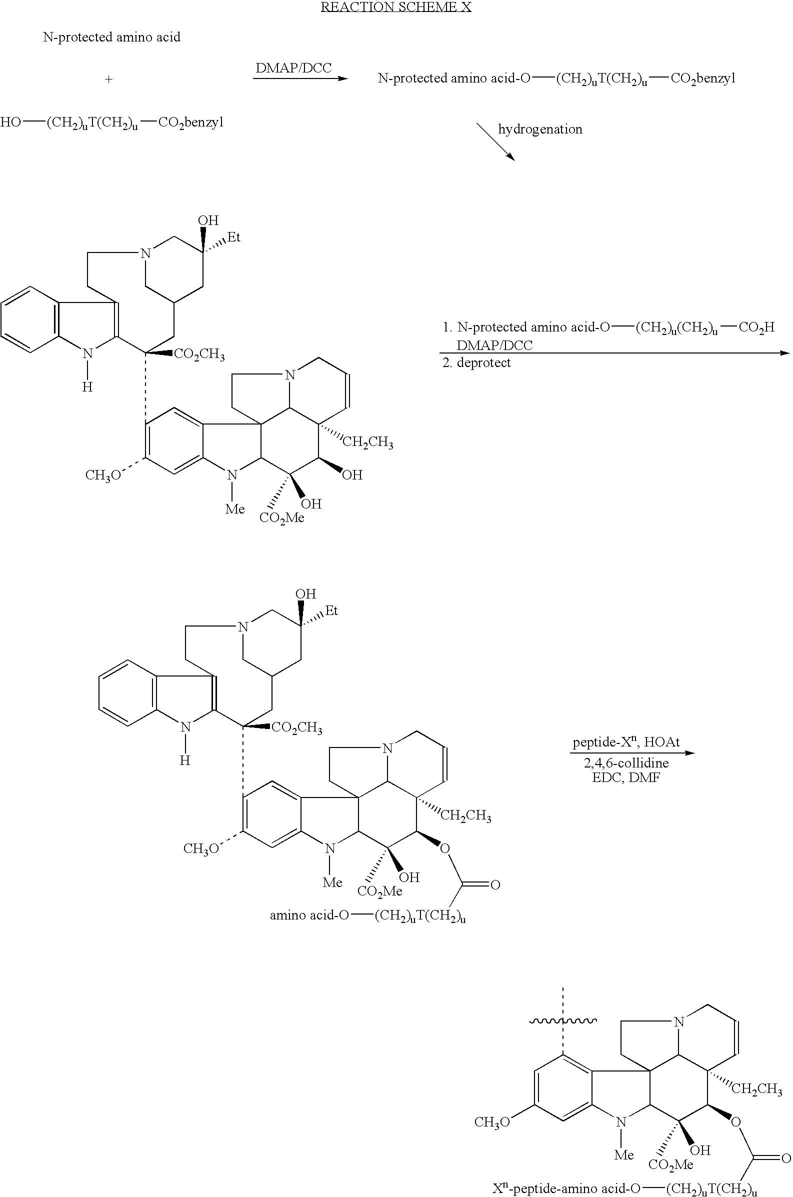

Taxol conjugates provided herein may be prepared by the general method provided below. The preparation of 7-Ala-Taxol and 7-Gly-Taxol is disclosed in Mathew et al (1992) J. Med. Chem. 35:145-151.

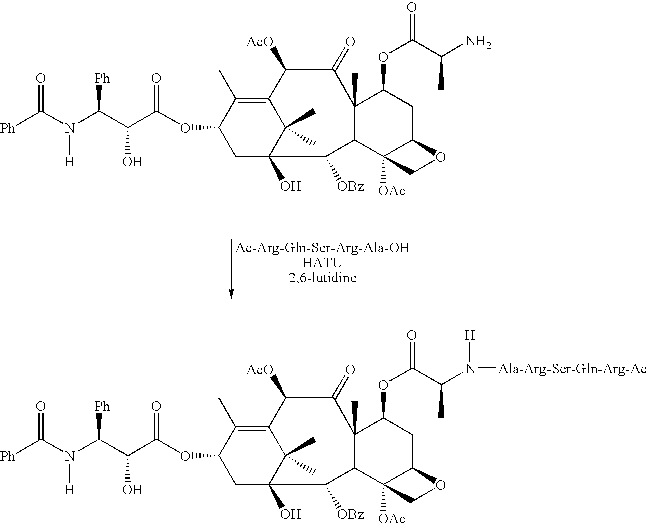

### E. Formulation and Administration of Pharmaceutical Compositions

The conjugates and compositions provided herein are used for treating, preventing, or ameliorating one or more symptoms of any disease or disorder that can be treated by targeting a cell or tissue that expresses a cell surface protease, particularly, a serine protease, on its surface at higher levels compared to other cells, or soluble, shed or released forms thereof. These include, but are not limited to, hyperproliferative diseases, such as cancer, any disease associated with aberrant or excessive angiogenesis, autoimmune disorders, inflammatory diseases and any other disease for which an appropriate cell surface protease, including cell-associated and cell-localized proteases, can be identified.

The pharmaceutical compositions provided herein contain therapeutically effective amounts of one or more of the conjugates provided herein that are useful in the prevention, treatment, or amelioration of one or more of the symptoms of diseases or disorders associated with undesired and/or uncontrolled angiogenesis or neovascularization. Such diseases or disorders include, but are not limited to, solid neoplasms, including lung, colon, esophageal, breast, ovarian and prostate cancers; vascular malformations and cardiovascular disorders, including, but not limited to, angiofibroma, angiolipoma, atherosclerosis, restenosis/reperfusion injury, arteriovenous malformations, hemangiomatosis and vascular adhesions, dyschondroplasia with vascular hematomas, hereditary hemorrhagic telangiectasia and Von Hipple Lindau syndrome; chronic inflammatory diseases and abherent wound repairs, including, but not limited to, diabetes mellitus, hemophiliac joints, inflammatory bowel disease, nonhealing fractures, rapidly progressing periodontitis, juvenile periodontitis, psoriasis, rheumatoid arthritis, venous stasis ulcers, granulations-burns, hypertrophic scars, liver cirrhosis, osteoradionecrosis, postoperative adhesions, pyogenic granuloma and systemic sclerosis; circulatory disorders, including, but not limited to, Raynaud's phenomenon; crest syndromes, including, but not limited to, calcinosis, esophageal, dyomotiloty, sclerodactyly and teangiectasis; dermatological disorders, including, but not limited to, systemic vasculitis, scleroderma, pyoderma gangrenosum, vasculopathy, venous, arterial ulcers, Sturge-Weber syndrome, Port-wine stains, blue rubber bleb nevus syndrome, Klippel-Trenaunay-Weber syndrome and Osler-Weber-Rendu syndrome; and ocular disorders, including, but not limited to, blindness caused by ocular neovascular disease, corneal graft neovascularization, macular degeneration in the eye, neovascular glaucoma, trachoma, diabetic retinopathy, myopic degeneration, retinopathy of prematurity, retrolental fibroplasia and corneal neovascularization.

The compositions contain one or more conjugates provided herein. The conjugates can be formulated into suitable pharmaceutical preparations such as, for example, solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Typically the cojugates described above are formulated into pharmaceutical compositions using techniques and procedures well known in the art (see, e.g., Ansel (1985) Introduction to Pharmaceutical Dosage Forms, Fourth Edition, p. 126)). Effective concentrations can be empirically determined using animal models, in vitro models or test subjects.

In the compositions, effective concentrations of one or more conjugates or pharmaceutically acceptable derivatives thereof is (are) mixed with a suitable pharmaceutical carrier or vehicle. The conjugates can be derivatized as the corresponding salts, esters, enol ethers or esters, acids, bases, solvates or hydrates prior to formulation, as described above. The concentrations of the conjugates in the compositions are effective for delivery of an amount, upon administration, that treats, prevents, or ameliorates one or more of the symptoms of diseases or disorders associated with undesired and/or uncontrolled angiogenesis or neovascularization. Such diseases or disorders include, but are not limited to, solid neoplasms; vascular malformations and cardiovascular disorders, including, but not limited to, angiofibroma, angiolipoma, atherosclerosis, restenosis/reperfusion injury, arteriovenous malformations, hemangiomatosis and vascular adhesions, dyschondroplasia with vascular hamartomas, hereditary hemorrhagic telangiectasia and Von Hipple Lindau syndrome; chronic inflammatory diseases and abherent wound repairs, including, but not limited to, diabetes mellitus, hemophiliac joints, inflammatory bowel disease, nonhealing fractures, rapidly progressing periodontitis, juvenile periodontitis, psoriasis, rheumatoid arthritis, venous stasis ulcers, granulations-burns, hypertrophic scars, liver cirrhosis, osteoradionecrosis, postoperative adhesions, pyogenic granuloma and systemic sclerosis; circulatory disorders, including, but not limited to, Raynaud's phenomenon; crest syndromes, including, but not limited to, calcinosis, esophageal, dyomotiloty, sclerodactyly and teangiectasis; dermatological disorders, including, but not limited to, systemic vasculitis, scleroderma, pyoderma gangrenosum, vasculopathy, venous, arterial ulcers, Sturge-Weber syndrome, Port-wine stains, blue rubber bleb nevus syndrome, Klippel-Trenaunay-Weber syndrome and Osler-Weber-Rendu syndrome; and ocular disorders, including, but not limited to, blindness caused by ocular neovascular disease, corneal graft neovascularization, macular degeneration in the eye, neovascular glaucoma, trachoma, diabetic retinopathy, myopic degeneration, retinopathy of prematurity, retrolental fibroplasia and corneal neovascularization.

The conjugates herein can be formulated into pharmaceutical compositions suitable for topical, local, intravenous and systemic application. Effective concentrations of one or more of the conjugates are mixed with a suitable pharmaceutical carrier or vehicle. The concentrations or amounts of the conjugates that are effective requires delivery of an amount, upon administration, that ameliorates the symptoms or treats the disease. Typically, the compositions are formulated for single dosage administration. Therapeutically effective concentrations and amounts can be determined empirically by testing the conjugates in known in vitro and in vivo systems, such as those described here; dosages for humans or other animals can then be extrapolated therefrom.

Upon mixing or addition of the conjugate(s) with the vehicle, the resulting mixture can be a solution, suspension, emulsion or other such composition. The form of the resulting mixture depends upon a number of factors, including the intended mode of administration and the solubility of the conjugate in the selected carrier or vehicle. The effective concentration is sufficient for ameliorating the symptoms of the disease, disorder or condition treated and can be empirically determined based upon in vitro and/or in vivo data, such as the data from the mouse xenograft model for tumors or rabbit ophthalmic model. If necessary, pharmaceutically acceptable salts or other derivatives of the conjugates can be prepared.

Pharmaceutical carriers or vehicles suitable for administration of the conjugates provided herein include any such carriers known to those skilled in the art to be suitable for the particular mode of administration. In addition, the conjugates can be formulated as the sole pharmaceutically active ingredient in the composition or can be combined with other active ingredients.

The conjugates can be administered by any appropriate route, for example, orally, parenterally, intravenously, intradermally, subcutaneously, or topically, in liquid, semi-liquid or solid form and are formulated in a manner suitable for each route of administration. Exemplary modes of administration depend upon the indication treated. Dermatological and ophthalmologic indications will typically be treated locally; whereas, tumors and vascular proliferative disorders, will typically be treated by systemic, intradermal or intramuscular, modes of administration.

The conjugate is included in the pharmaceutically acceptable carrier in an amount sufficient to exert a therapeutically useful effect in the absence of undesirable side effects on the patient treated. It is understood that number and degree of side effects depends upon the condition for which the conjugates are administered. For example, certain toxic and undesirable side effects are tolerated when treating life-threatening illnesses, such as tumors, that would not be tolerated when treating disorders of lesser consequence.

The concentration of conjugate in the composition will depend on absorption, inactivation and excretion rates thereof, the dosage schedule, and amount administered as well as other factors known to those of skill in the art.

Typically a therapeutically effective dosage should produce a serum concentration of active ingredient of from about 0.1 ng/ml to about 50-100 μg/ml. The pharmaceutical compositions typically should provide a dosage of from about 0.01 mg to about 100-2000 mg of conjugate, depending upon the conjugate selected as adjusted for body surface area and/or weight. Typically, for intravenous or systemic treatment a daily dosage of about between 0.05 and 0.5 mg/kg should be sufficient. Local application for ophthalmic disorders should provide about 1 ng up to 100 μg, generally about 1 μg to about 10 μg, per single dosage administration. It is understood that the amount to administer is a function of the conjugate selected, the indication treated, and possibly the side effects that will be tolerated. Dosages can be empirically determined using recognized models for each disorder.

Typically, the compositions are formulated for single dosage administration. To formulate a composition, the weight fraction of conjugate is dissolved, suspended, dispersed or otherwise mixed in a selected vehicle at an effective concentration such that the treated condition is relieved or ameliorated. Pharmaceutical carriers or vehicles suitable for administration of the conjugates provided herein include any such carriers known to those skilled in the art to be suitable for the particular mode of administration.

In addition, the conjugates can be formulated as the sole ingredient in the composition or can be combined with other active ingredients. Liposomal suspensions, including tissue-targeted liposomes, particularly tumor-targeted liposomes, also can be suitable as pharmaceutically acceptable carriers. These can be prepared according to methods known to those skilled in the art. For example, liposome formulations can be prepared as described in U.S. Pat. No. 4,522,811. Briefly, liposomes such as multilamellar vesicles (MLV's) can be formed by drying down egg phosphatidyl choline and brain phosphatidyl serine (7:3 molar ratio) on the inside of a flask. A solution of a conjugate provided herein in phosphate buffered saline lacking divalent cations (PBS) is added and the flask shaken until the lipid film is dispersed. The resulting vesicles are washed to remove unencapsulated conjugate, pelleted by centrifugation, and then resuspended in PBS.

The conjugate is included in the pharmaceutically acceptable carrier in an amount sufficient to exert a therapeutically useful effect in the absence of undesirable side effects on the patient treated. The therapeutically effective concentration can be determined empirically by testing the conjugates in in vitro and in vivo systems described herein (see, e.g., EXAMPLES 3 and 4) and then extrapolated therefrom for dosages for humans.

The concentration of conjugate in the pharmaceutical composition will depend on absorption, inactivation and excretion rates of the conjugate, the physicochemical characteristics of the conjugate, the dosage schedule, and amount administered as well as other factors known to those of skill in the art. For example, the amount that is delivered is sufficient to ameliorate one or more of the symptoms of diseases or disorders associated with undesired and/or uncontrolled angiogenesis or neovascularization, as described herein.

Typically a therapeutically effective dosage should produce a serum concentration of active ingredient of from about 0.1 ng/ml to about 50-100 μg/ml. The pharmaceutical compositions typically should provide a dosage of from about 0.001 mg to about 2000 mg of conjugate per kilo-gram of body weight per day. Pharmaceutical dosage unit forms are prepared to provide from about 1 mg to about 1000 mg and generally from about 10 to about 500 mg of the essential active ingredient or a combination of essential ingredients per dosage unit form.

The conjugate can be administered at once, or can be divided into a number of smaller doses to be administered at intervals of time. It is understood that the precise dosage and duration of treatment is a function of the disease being treated and can be determined empirically using known testing protocols or by extrapolation from in vivo or in vitro test data. It is to be noted that concentrations and dosage values can also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Exemplary pharmaceutically acceptable derivatives include acids, bases, enol ethers and esters, salts, esters, hydrates, solvates and conjugate forms. The derivative is selected such that its pharmacokinetic properties are superior to the corresponding neutral conjugate.

Thus, effective concentrations or amounts of one or more of the conjugates described herein or pharmaceutically acceptable derivatives thereof are mixed with a suitable pharmaceutical carrier or vehicle for systemic, topical or local administration to form pharmaceutical compositions. Conjugates are included in an amount effective for ameliorating one or more symptoms of, or for treating or preventing diseases or disorders associated with undesired and/or uncontrolled angiogenesis or neovascularization, as described herein. The concentration of conjugate in the composition will depend on absorption, inactivation, excretion rates of the conjugate, the dosage schedule, amount administered, particular formulation as well as other factors known to those of skill in the art.

The compositions are intended to be administered by a suitable route, including orally, parenterally, rectally, topically and locally. For oral administration, capsules and tablets are generally employed. The compositions are in liquid, semi-liquid or solid form and are formulated in a manner suitable for each route of administration. Exemplary modes of administration include parenteral and oral modes of administration.

Solutions or suspensions used for parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the conjugates exhibit insufficient solubility, methods for solubilizing conjugates can be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the conjugates also can be used in formulating effective pharmaceutical compositions.

Upon mixing or addition of the conjugate(s), the resulting mixture can be a solution, suspension, emulsion or the like. The form of the resulting mixture depends upon a number of factors, including the intended mode of administration and the solubility of the conjugate in the selected carrier or vehicle. The effective concentration is sufficient for ameliorating the symptoms of the disease, disorder or condition treated and can be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the conjugates or pharmaceutically acceptable derivatives thereof. The conjugates and derivatives thereof are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the conjugate sufficient to produce the desired therapeutic effect, in association with the required pharmaceutical carrier, vehicle or diluent. Examples of unit-dose forms include ampoules and syringes and individually packaged tablets or capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the conjugate: a diluent such as lactose, sucrose, dicalcium phosphate, or carboxymethyl-cellulose; a lubricant, such as magnesium stearate, calcium stearate and talc; and a binder such as starch, natural gums, such as gum acacia-gelatin, glucose, molasses, polyvinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing a conjugate as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered can also contain minor amounts of nontoxic auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the conjugate in an amount sufficient to alleviate the symptoms of the treated subject.

Dosage forms or compositions containing active ingredient in the range of 0.005% to 100% with the balance made up from non-toxic carrier can be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions can contain 0.001%-100% active ingredient, such as 0.1-85%, for example 75-95%.

The conjugates or pharmaceutically acceptable derivatives can be prepared with carriers that protect the conjugate against rapid elimination from the body, such as time release formulations or coatings. The compositions can include other conjugates to obtain desired combinations of properties. The conjugates provided herein, or pharmaceutically acceptable derivatives thereof as described herein, also can be advantageously administered for therapeutic or prophylactic purposes together with another pharmacological agent known in the general art to be of value in treating one or more of the diseases or medical conditions referred to hereinabove, such as diseases or disorders associated with undesired and/or uncontrolled angiogenesis or neovascularization. It is to be understood that such combination therapy constitutes a further aspect of the compositions and methods of treatment provided herein.

1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which can be enteric-coated, sugar-coated or film-coated. Capsules can be hard or soft gelatin capsules, while granules and powders can be provided in non-effervescent or effervescent form with the combination of other ingredients known to those skilled in the art.

In certain embodiments, the formulations are solid dosage forms, such as, for example, capsules or tablets. The tablets, pills, capsules, troches and other dosage forms can contain, for example, any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of conjugates which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the conjugate could be provided in a composition that protects it from the acidic environment of the stomach. For example, the composition can be formulated in an enteric coating that maintains its integrity in the stomach and releases the conjugate in the intestine. The composition also can be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The conjugates also can be administered as a component of an elixir, suspension, syrup, wafer, sprinkle, chewing gum or the like. A syrup can contain, in addition to the conjugates, sucrose as a sweetening agent and certain preservatives, dyes and colorings and flavors.

The conjugates also can be mixed with other active materials which do not impair the desired action, or with materials that supplement the desired action, such as antacids, H2 blockers, and diuretics. Higher concentrations, up to about 98% by weight of the conjugate can be included.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents also can be used in the above dosage forms. Flavoring and sweetening agents are used in compressed tablets, sugar-coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and can contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of conjugates which produce a pleasant taste sensation.

For a solid dosage form, the solution or suspension, in for example propylene carbonate, vegetable oils or triglycerides, for example the formulation can be encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, can be diluted with a sufficient quantity of a pharmaceutically acceptable liquid carrier, e.g., water, to be easily measured for administration.

Alternatively, liquid or semi-solid oral formulations can be prepared by dissolving or dispersing the conjugate or derivative thereof in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603. Briefly, such formulations include, but are not limited to, those containing a conjugate provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more anitoxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations can be coated as known by those of skill in the art in order to modify or sustain dissolution of the conjugate. Thus, for example, they can be coated with a conventional enterically digestible coating, such as phenylsalicylate, waxes and cellulose acetate phthalate.

2. Injectables, Solutions and Emulsions

Parenteral administration, generally characterized by injection, either subcutaneously, intramuscularly or intravenously also is contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) also is contemplated herein. Briefly, a conjugate provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The conjugate diffuses through the outer polymeric membrane in a release rate controlling step. The percentage of conjugate contained in such parenteral compositions is highly dependent on the specific nature thereof, as well as the activity of the conjugate and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the conjugate is adjusted so that an injection provides an effective amount to produce the desired pharmacological effect. The exact dose depends on the age, weight and condition of the patient or animal as is known in the art.

The unit-dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing a conjugate is an effective mode of administration. Another embodiment is a sterile aqueous or oily solution or suspension containing a conjugate injected as necessary to produce the desired pharmacological effect.

Injectables are designed for local and systemic administration. Typically a therapeutically effective dosage is formulated to contain a concentration of at least about 0.1% w/w up to about 90% w/w or more, genrally more than 1% w/w of the conjugate to the treated tissue(s). The conjugate can be administered at once, or can be divided into a number of smaller doses to be administered at intervals of time. It is understood that the precise dosage and duration of treatment is a function of the tissue being treated and can be determined empirically using known testing protocols or by extrapolation from in vivo or in vitro test data. It is to be noted that concentrations and dosage values can also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The conjugate can be suspended in micronized or other suitable form or can be derivatized to produce a more soluble product. The form of the resulting mixture depends upon a number of factors, including the intended mode of administration and the solubility of the conjugate in the selected carrier or vehicle. The effective concentration is sufficient for ameliorating the symptoms of the condition and can be empirically determined.

3. Lyophilized Powders

Of interest herein are also lyophilized powders, which can be reconstituted for administration as solutions, emulsions and other mixtures. They also can be reconstituted and formulated as solids or gels.

The sterile, lyophilized powder is prepared by dissolving a conjugate provided herein, or a pharmaceutically acceptable derivative thereof, in a suitable solvent. The solvent can contain an excipient which improves the stability or other pharmacological component of the powder or reconstituted solution, prepared from the powder. Excipients that can be used include, but are not limited to, dextrose, sorbital, fructose, corn syrup, xylitol, glycerin, glucose, sucrose or other suitable agent. The solvent can also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (such as 10-1000 mg, for example 100-500 mg) or multiple dosages of the conjugate. The lyophilized powder can be stored under appropriate conditions, such as at about 4° C. to room temperature.

Reconstitution of this lyophilized powder with water for injection provides a formulation for use in parenteral administration. For reconstitution, generally about 1-50 mg, such 5-35 mg or about 9-30 mg of lyophilized powder, is added per mL of sterile water or other suitable carrier. The precise amount depends upon the selected conjugate, intended subject, and other empircally determinable parameters. Hence the amount can be empirically determined.

4. Topical Administration

Topical mixtures are prepared as described for the local and systemic administration. The resulting mixture can be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The conjugates or pharmaceutically acceptable derivatives thereof can be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, generally less than 10 microns.

The conjugates can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the conjugate alone or in combination with other pharmaceutically acceptable excipients also can be administered.

These solutions, particularly those intended for ophthalmic use, can be formulated as 0.01%-10% isotonic solutions, pH about 5-7, with appropriate salts.

5. Compositions for other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more conjugates. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases can be used. Agents to raise the melting point of suppositories include spermaceti and wax. Rectal suppositories can be prepared either by the compressed method or by molding. The typical weight of a rectal suppository is about 2 to 3 gm.

Tablets and capsules for rectal administration are manufactured using the same pharmaceutically acceptable substance and by the same methods as for formulations for oral administration.

6. Articles of Manufacture

The conjugates or pharmaceutically acceptable derivatives can be packaged as articles of manufacture containing packaging material, a conjugate or pharmaceutically acceptable derivative thereof provided herein, which is used for treatment, prevention or amelioration of one or more symptoms associated with proliferative diseases or disorders, and a label that indicates that the conjugate or pharmaceutically acceptable derivative thereof is used for treatment, prevention or amelioration of one or more symptoms associated with proliferative diseases or disorders.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the conjugates and compositions provided herein are contemplated as are a variety of treatments for any disorder in which a cell surface protease, or a soluble, shed or secreted form thereof, is implicated.

### F. Evaluation of the Activity of the Conjugates

Standard physiological, pharmacological and biochemical procedures are available for testing the conjugates to identify those that possess therapeutic activity upon action of a cell surface protease or a soluble, shed, or released form thereof. In vitro and in vivo assays that can be used to evaluate therapeutic activity, such as cytotoxicity, of the conjugates will depend upon the therapeutic agent being tested.

Exemplary assays are discussed briefly below with reference to cytotoxic conjugates (see, also, Examples). It is understood that the particular activity assayed will depend upon the conjugated therapeutic agent.

1. In Vitro Assays

The therapeutic activity, such as cytotoxicity, of the conjugates provided herein can assessed by any assays normally used for assessing the therapeutic activity, such as cytotoxicity, of the unconjugated therapeutic agent. Numerous such assays are known, for example, assays can employ cells that express the targeted cell surface protease and the therapeutic activity of the therapeutic agent is assessed. For example, cytoxicity can be assessed by measuring cell viability or by measuring cell proliferation, such as by incorporation of a labeled nucleotide or other such label. Generally the activity is compared with cells that do not express the targeted protease.

For example, the cells will be any that express a targeted MTSP or endotheliase. Such cells can be obtained by choosing cells known to express the cell surface protease, such as by determining tissue expression profiles, as discussed above, or by screening a variety of cell lines with an antibody for a targeted protease, or for the protease activity in the presence of a labeled, such as a chromogenic, substrate for the protease in the presence and absence of a known inhibitor of the targeted protease.

Alternatively, nucleic acid encoding the protease can be introduced in a cell line that does not express the protease, and expressed therein to produce a cell line that expresses the protease of interest. The resulting recombinant cells can be used in cytotoxicity assays.

2. In Vivo Assays

Numerous animal models for assessing therapeutic activity are known. Any suitable in vivo model can be used. Exemplary are the mouse xenograft model and chicken embryo models.

Chicken Embryo Model

The CAM model (chick embryo chorioallantoic membrane model; Ossowski (1988) J. Cell Biol. 107:2437-2445), provides another method for evaluating the inhibitory activity of a test compound. In the CAM model, tumor cells invade through the chorioallantoic membrane containing CAM (with tumor cells in the presence of several serine protease inhibitors results in less or no invasion of the tumor cells through the membrane). Thus, the CAM assay is performed with CAM and tumor cells in the presence and absence of various concentrations of test compound. The invasiveness of tumor cells is measured under such conditions to provide an indication of the compound's inhibitory activity. A compound having inhibitory activity correlates with less tumor invasion.

Thus, the CAM assay is performed with CAM and tumor cells in the presence and absence of various concentrations of a test compound. A compound having activity correlates with a change in tumor invasion and/or tumor growth.

For example, the ability of a cell surface protease to liberate a therapeutic agent, such as a cytotoxic agent, or the activity of a conjugate agent can be assessed using this model. If the therapeutic agent is released from the compound and it is an inhibitory agent there will be less tumor invasion or a decrease in size of the tumor. If the therapeutic agent is inactive in the conjugate, there will be no effect on tumor invasion.

The CAM model also is used in a standard assay of angiogenesis (i.e., effect on formation of new blood vessels (Brooks et al. (1991) Methods in Molecular Biology 129:257-269). According to this model, a filter disc containing an angiogenesis inducer, such as basic fibroblast growth factor (bFGF) is placed onto the CAM. Diffusion of the cytokine into the CAM induces local angiogenesis, which can be measured in several ways such as by counting the number of blood vessel branch points within the CAM directly below the filter disc. The ability of identified compounds to inhibit cytokine-induced angiogenesis can be tested using this model. A test compound can either be added to the filter disc that contains the angiogenesis inducer, be placed directly on the membrane or be administered systemically. The extent of new blood vessel formation in the presence and/or absence of test compound can be compared using this model. The formation of fewer new blood vessels in the presence of a test compound would be indicative of anti-angiogenesis activity.

This can be adapted for use with the conjugates herein to 1) assess the activity of a therapeutic agent in the conjugate; and 2) to assess the ability of a particular cell surface protease to liberate a therapeutic agent from a conjugate.

Mouse Xenograft Model

In vivo activity can be a assessed using recognized animal models, such as the well-known mouse xenograft model for anti-tumor activity (see, e.g., Beitz et al. (1992) Cancer Research 52:227-230; Houghton et al. (1982) Cancer Res. 42:535-539; Bogden et al. (1981) Cancer (Philadelphia) 48:10-20; Hoogenhout et al. (1983) Int. J. Radiat. Oncol., Biol. Phys. 9:871-879; Stastny et al. (1993) Cancer Res. 53:5740-5744). The in vivo mouse solid tumor xenograft model is used in assays for that test an agent's ability to inhibit tumor cell proliferation and/or spontaneous metastasis. For example, a conjugate is evaluated for anti-tumor activity against any tumor subtype that expresses the targeted cell surface protease, e.g., an ovarian tumor, in a mouse tumor xenograft model. Nude mice are given one or more, such as four intravenous injections of the conjugate. Dosing material is prepared by mixing the test material with appropriate volumes of, for example, PBS/0.1% BSA to achieve the desired doses. Mice IV injections (250-300 ul) into the tail vein for the duration of the experiment, such as, for example, days 5, 12, 19 and 26, with day 1 designated as the day that the tumor cells are injected into the mice. Doses are either fixed or normalized for differences in body weight. Tumor volume is measured twice weekly for a selected period.

Female Balb/c nu/nu athymic mice (Roger Williams Hospital Animal Facility, Providence, R.I.), 8-12 weeks old are suitable mice. They should be maintained in an aseptic environment and selected such that body weights range from about 25-30 grams the day prior to dosing. Animals are maintained in a quarantined room and handled under aseptic conditions. Food and water are supplied ad libitum. Appropriate tumor cells can be obtained, for example, from the American Type Culture Collection (Rockville, Md.) and grown in modified Eagle's medium supplemented with 10% fetal calf serum. A selected number of days, such as five days prior to injection of the test material, mice receive a subcutaneous injection of tumor cells in the right rear flank.

Calipers are used to measure the dimensions of each tumor. Measurements (mm) of maximum and minimum width are performed prior to injection of the test material and at selected, such as bi-weekly, intervals for the duration of the experiment. Tumor volumes (mm³) can be computed, for example, using the formula:

Volume=[(width)²(length)]/2.

### G. Methods for Identifying Proteases to Target

Also provided are methods for identifying proteases to target conjugates for treatment of diseases. The methods involve identifying cell-surface protease-associated disease by identifying a cell involved in the disease process or a cell in the vicinity of the cell involved in the disease process. For example, if disease involves a particular tumor, a protease present on the particular tumor or on cells that a located in the vicinity thereof is identified. A cell surface protease on the cell for targeting and substrates therefor are then identified. Conjugates that target such proteases as provided herein can then be prepared.

The following examples are included for illustrative purposes only and are not intended to limit the scope of the invention.

### EXAMPLE 1

### General Procedures for Preparing Peptide-Doxorubicin Conjugates

Step A: Synthesis of Peptides on Wang Resin

Peptides were prepared automatically using an ABI 431A peptide synthesizer from Perseptive Biosystems on preloaded Wang resin (0.25 mmol). The ABI 431A uses HOBT, HBTU, DIEA activation. The synthesis of N-acetyl (or other amide) capped peptides involved the use of AcOH (or other respective carboxylic acid) during the final coupling step on the ABI 431A. Other N-terminal caps where attached manually by using the following reagents: For carbamates and sulfonamides the peptides were capped with ROCOCl or RSO₂Cl and DIEA (4 equivalents each, 1 hr) in DMF (3 mL).

Step B: Cleavage of Peptides from Wang Resin

The cleavage of peptides from Wang resin involved shaking the resin with 2 mL TFA/H₂O (95:5) for 45 min. The resin was removed by filtration and the filtrate was allowed to stand for an additional hour. The solution was concentrated to a residue. The crude peptide was analyzed by analytical HPLC (system A). Typical purity of the crude peptide ranged from 80% to 95%. The peptides were purified by preparative HPLC (system B) using an appropriate gradient (typically 10-30%). Pure fractions were then lyophilized to provide the desired peptide as a white solid. Typical yields were 20-50% and a purity of 96-99%.

Analytical HPLC Conditions (System A)

Column: Chromolith RP-18e 4.6 mm×100 mm from EM science

Gradient: 5-50% B in A over 6 min

Flow Rate: 4 mL/min

Solvent A: 0.1% TFA in water

Solvent B: 0.1% TFA in acetonitrile

Wavelength: 210 nm, 280 nm

Preparative HPLC Conditions (System B)

Column: Ultro 120 5 C18Q 150×20 mm from Peeke Scientific

Gradient: 0-20%, or 10-30% or 20-40% B in A over 40 min

Flow Rate: 18 mL/min

Solvent A: 0.1% TFA in water

Solvent B: acetonitrile

Wavelength: 214 nm

Step C: Coupling of Peptide acids to Doxorubicin

To a mixture of peptide acid (0.052 mmol, 1.2 equivalents), doxorubicin hydrochloride (0.043 mmol, 25 mg), and HATU (0.0604 mmol, 22.9 mg, 1.4 equivalents) was added DMF (1 mL) then 2,6-lutidine (0.17 mmol, 20 μL, 4 eqiuvalents). The mixture was mixed until a homogeneous solution was obtained. After 4 to 24 hours (monitor by HPLC system A) the reaction was diluted with water (9 mL) and directly purified by preparative HPLC (system D). Pure fractions were then lyophilized to provide the desired peptide doxorubicin conjugate as a fluffy red solid. The quality of the final conjugate was verified by analytical HPLC (system C) and mass spectroscopy. Typical yields were 10-30% with a purity of 95-99%. (Note: when the peptide acid contained a histidine residue DIEA was substituted as the base and the reaction time was shortened to 1 hour).

Deprotection of fluorenylmethylesters of peptide doxorubicin conjugates: In cases where free carboxylic acid is present in the conjugate a fluorenyl methyl ester was used to protect a carboxylic acid during coupling of the C-terminus of the peptide acid to doxorubicin, the flourenylmethyl group was subsequently removed with 10% morpholine in DMF for 1 hour.

Analytical HPLC Conditions (System C)

Column: Chromolith RP-18e 4.6 mm×100 mm from EM science

Gradient: 5-50% B in A over 6 min

Flow Rate: 4 mL/min

Solvent A: 0.1% TFA in water

Solvent B: 0.1% TFA in acetonitrile

Wavelength: 210 nm, 280 nm

Examples of retention times (min)

| | |
|---|---|
| Doxorubicin | 4.05 |
| Ac-Gly-Ser-Gly-Arg-Ser-nLeu-Dox | 4.34 |
| MeOCO-Thr-Gly-Arg-Ser-nLeu-Dox | 4.39 |
| PhSO2-Thr-Gly-Arg-Ser-nLeu-Dox | 4.83 |
| N,N-dimethylglycine-Thr-Gly-Arg-Ser-nLeu-Dox | 4.27 |
| Ac-Thr-Gly-Arg-Ser-nLeu-Dox | 4.32 |

Preparative HPLC Conditions (System D)

Column: Ultro 120 5 C18Q 150×20 mm from Peeke Scientific

Gradient: 10-30% B in A over 40 min

Flow Rate: 18 mL/min

Solvent A: 0.1% acetic acid in water

Solvent B: acetonitrile

Wavelength: 214 nm

### EXAMPLE 2

### Preparation of Ac-Gly-Ser-Gly-Arg-Ser-nLeu-Dox

Step A: Manual Synthesis of Ac-Gly-Ser(tBu)-Gly-Arg(Pbf)-Ser(tBu)-nLeu-Wang Resin

In a 250 mL fritted peptide synthesis vessel equipped with nitrogen agitation and vacumm assisted drainage, Fmoc-nL-Wang resin (nova-biochem, 3.3 grams, 0.9 mmol/g, 3 mmol) was pre-swelled for 30 min using DMF. The peptide was then elongated by repeating the 4 step procedure below a total of five times with the following Fmoc aminoacids: Fmoc-Ser(tBu)-OH, Fmoc-Arg(Pbf)-OH, Fmoc-Gly-OH, Fmoc-Ser(tBu)-OH, Fmoc-Gly-OH.

Iterative Coupling Procedure

1. the resin was mixed with 20% piperdine in DMF (100 mL) for 5 min then drained (repeat 3 times).

2. the resin was agitated with DMF (100 mL) for 30 sec then drained (repeat 3 times).

3. to a mixture of Fmoc-aminoacid (12 mmol), HOBT (12 mmol, 4 equivalents, 1.622 g), TBTU (11.7 mmol, 3.9 equivalents, 3.757 g), DMF (10 mL) and NMP (90 mL) was added DIEA (12 mmol, 4 equivalents, 2.10 mL). After stirring for 5 min to allow pre-activation, the solution was added to the synthesis vessel. The reaction was checked for completion by ninhydrin test and then drained. (If the ninhydrid test was blue, a double coupling (repeat step 3) was performed.

4. the resin was agitated with DMF (100 mL) for 30 sec then drained (repeat 3 times).

The elongated resin (Fmoc-Gly-Ser(tBu)-Gly-Arg(Pbf)-Ser(tBu)-nLeu-Wang resin) was treated to steps 1 and 2 above to remove the Fmoc group. A solution of acetic anhydride (15 mmol, 5 equivalents, 1.42 mL), DIEA (15 mmol, 5 equivalents, 2.62 mL), DMF (10 mL) and NMP (90 mL) was added to the reaction vessel. After 1 hour the resin was washed with DMF (100 mL, 3 times), CH₂Cl₂ (100 mL, 3 times) and MeOH (100 mL, 3 times). The resin was dried under vacuum for 15 hours.

Step B: Preparation of Ac-Gly-Ser-Gly-Arg-Ser-nLeu-OH

To the above synthesis vessel containing Ac-Gly-Ser(tBu)-Gly-Arg(Pbf)-Ser(tBu)-nLeu-Wang resin (3 mmol) was added TFA/H₂O (95:5, 50 mL). After gently agitation for 45 min the cleavage solution was collected and the filtrate was allowed to stand for an additional 90 min. The solution was concentrated to a residue. The crude peptide was analyzed by analytical HPLC (system A, RT=1.73, purity=90%). The residue was dissolved in water (50 mL) and hexanes (10 mL) and mixed. The hexanes layer was removed and the aqueous layer bubbled with nitrogen to evaporate any remaining hexanes. The crude peptide was purified by preparative HPLC (system E). Pure fractions were then lyophilized to provide Ac-Gly-Ser-Gly-Arg-Ser-nLeu-OH (1.04 g, 1.68 mmol, 56%) as a white solid. The purity was evaluated by analytical HPLC (system A, RT=1.73 min, 97% purity) and the constitution by mass spectrospcopy (ion observed at 617.9).

Preparative HPLC Conditions (System E)

Column: Waters Delta-Pak radial compression column, 15 um, 100A

Gradient: 5-15% B in A over 40 min

Flow Rate: 80 mL/min

Solvent A: 0.1% acetic acid in water

Solvent B: acetonitrile

Wavelength: 214 nm

Step C: Preparation of Ac-Gly-Ser-Gly-Arg-Ser-nLeu-Dox

To a mixture of Ac-Gly-Ser-Gly-Arg-Ser-nLeu-OH (1.68 mmol, 1.04 g, 1.1 equivalents), doxorubicin hydrochloride (1.53 mmol, 887.8 mg), and HATU (1.76 mmol, 669.6 mg, 1.15 equivalents) was added DMF (40 mL) then 2,6-lutidine (6.12 mmol, 709 μL, 4 eqiuvalents). The solution was stirred for 18 hours. The reaction was diluted with water (100 mL), acidified with acetic acid (400 μL) and purified immediately in three batches by preparative HPLC (system E). Each red colored fraction was analyzed by analytical HPLC (system F). Fractions of greater than 95% purity were then combined. The acetonitrile was removed under vacuum and the remaining solution was lyophilized to provide Ac-Gly-Ser-Gly-Arg-Ser-nLeu-Dox (0.682 mmol, 780 mg, 45%) as a fluffy red solid. The purity was evaluated by analytical HPLC (system F, RT=3.51 min, 95% purity) and the constitution by mass spectrospcopy (ion observed at 1143.5).

Analytical HPLC Conditions (System F)

Column: Chromolith RP-18e 4.6 mm×100 mm from EM science

Gradient: 20-40% B in A over 6 min

Flow Rate: 4 mL/min

Solvent A: 0.1% TFA in water

Solvent B: 0.1% TFA in acetonitrile

Wavelength: 210 nm, 280 nm

Preparative HPLC Conditions (System E)

Column: Waters Delta-Pak radial compression column, 15 um, 100A

Gradient: 15-25% B in A over 40 min

Flow Rate: 80 mL/min

Solvent A: 0.1% acetic acid in water

Solvent B: acetonitrile

Wavelength: 214 nm

### EXAMPLE 3

### General Procedures for Preparing Peptide-Taxol Conjugates

Step A: Synthesis of Peptides on Wang Resin

See Example 1, Step A.

Step B: Cleavage of Peptides from Wang Resin

See Example 1, Step B.

Step C: Coupling of Peptide Acids to 7-Gly-Taxol or 7-Ala-Taxol

To a mixture of peptide acid (0.0121 mmol, 1.1 equivalents), 7-Gly-Taxol of 7-Ala-Taxol (0.011 mmol), and HATU (0.0154 mmol, 5.9 mg, 1.4 equivalents) was added DMF (0.3 mL) then 2,6-lutidine (0.044 mmol, 5.1 μL, 4 eqiuvalents). The mixture was mixed until a homogeneous solution was obtained. After 4 to 24 hours (monitor by HPLC system H) the reaction was diluted with water (9 mL) and directly purified by preparative HPLC (system 1). Pure fractions were then lyophilized to provide the desired peptide taxol conjugate as a fluffy white solid. The quality of the final conjugate was verified by analytical HPLC (system H) and mass spectroscopy. Typical yields were 30-50% with a purity of 96-99%.

Analytical HPLC Conditions (System H)

Column: Chromolith RP-18e 4.6 mm×100 mm from EM science

Gradient: 5-90% B in A over 6 min

Flow Rate: 4 mL/min

Solvent A: 0.1% TFA in water

Solvent B: 0.1% TFA in acetonitrile

Wavelength: 210 nm, 280 nm

Examples of Retention Times (min)

| | |
|---|---|
| Ac-Gln-Ser-Arg-Ala-Ala-Taxol | 2.86 |
| Ac-Gln-Ser-Arg-Ser-Ala-Ala-Taxol | 2.79 |
| Ac-Ser-Gly-Arg-Ala-Ser-Ala-Taxol | 2.87 |
| Ac-Arg-Ser-Arg-Ala-Ala-Taxol | 2.80 |
| Ac-Ser-Gly-Arg-Ser-Ser-Ala-Taxol | 2.81 |

Preparative HPLC Conditions (System I)

Column: Ultro 120 5 C18Q 150×20 mm from Peeke Scientific

Gradient: 20-45% B in A over 40 min

Flow Rate: 18 mL/min

Solvent A: 0.1% TFA in water

Solvent B: acetonitrile

Wavelength: 214 nm

### EXAMPLE 4

### Preparation of N-Ac-Arg-Gln-Ser-Arg-Ala-Ala-DOX

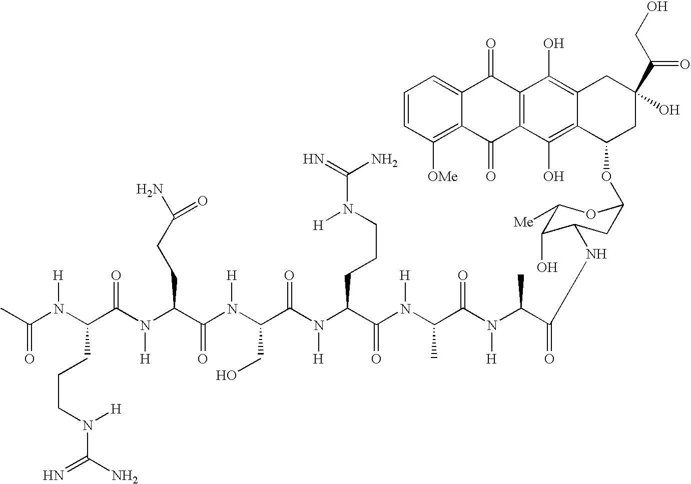

Step A: N-Ac-Arg-Gln-Ser-Arg-Ala-Ala-OH

Using the following general procedure, the N-acetyl peptidic substrate N-Ac-Arg-Gln-Ser-Arg-Ala-Ala-OH was synthesized in a peptide synthesis flask. Commencing with commercial Fmoc-Ala-Wang resin (0.35 g, 0.84 mmol, Nova), standard Fmoc-deprotection with 20% piperidine was followed by a sequential iterative coupling-Fmoc deprotection strategy. Each coupling employed a 3-fold excess (2.52 mmol) of Fmoc-Ala, Fmoc-Arg(Boc)₂, Fmoc-Ser(tBu), Fmoc-Gln(Trt) and Fmoc-Arg(Boc)₂, respectively. Couplings were achieved using PyBOP (2.52 mmol) and DIEA (2.52 mmol) in DMF solvent. During each coupling cycle, the Fmoc protecting group was removed using 20% piperidine in DMF. After removal of the N-terminal Fmoc group, capping with acetic anhydride (1.43 mmol, 1.7 equiv.), DMAP (0.25 mmol, 0.3 equiv.), and DIEA (1.26 mmole, 1.5 equiv.) afforded the resin-bound N-acetyl intermediate. The protected peptide resin was treated with 50% TFA in methylene chloride for 30 min to cleave the Wang resin and then the Boc, Trt and t-Bu protecting groups were removed with 70% TFA in methylene chloride. Solvent and other volatile byproducts were evaporated under reduced pressure and the crude product was dissolved in water and lyophilized to afford the title compound as a nearly colorless, amorphous solid. Mass spectral analysis confirmed the desired molecular weight. HPLC analysis indicated the product to be of approximately 95% purity. The peptide carboxylic acid intermediate can be further purified by trituration or by preparative HPLC, if desired.

Step B: N-Ac-Arg-Gln-Ser-Arg-Ala-Ala-DOX

The intermediate from Step A (20 mg, 0.027 mmol) was dissolved in dry DMF (0.8 mL) and was stirred at room temperature under a nitrogen atmosphere. To this solution was added doxorubicin hydrochloride (15.6 mg, 0.027 mmol), EDC (6.8 mg, 0.035 mmol), HOAt (4.8 mg, 0.035 mmol) and 2,6-lutidine (7.3 μL, 0.06 mmol). Stirring was continued until completion of the coupling, as monitored by analytical HPLC (system J, see below). The solution was filtered and the crude product was purified by C18 RP-HPLC (A=0.1% AcOH/H₂O; B=CH₃CN), gradient elution 100% to 60% A over 60 min). Homogeneous product fractions (evaluated by HPLC, system J) were pooled and lyophilized to afford the title compound as a light red solid.

HPLC conditions, System J:

Column: Phenomenex 15 cm #00F-3033-E0, C18

Eluant: Gradient 95:5 (A:B) to 25:75 (A:B) over 20 min.

A=0.1% TFA/H₂O, B=0.1% TFA/Acetonitrile

Flow: 1 mL/min.

Wavelength: 210 nm, 280 nm

Retention times: Doxorubicin=8.89 min.

N-Ac-Arg-Gln-Ser-Arg-Ala-Ala-Dox=8.4 min.

Physical Properties:

Molecular Formula: C₅₅H₇₈N₁₄O₂₀

Molecular Weight: 1255.3

Low Resolution Mass Spec: 628.2 (M+2/2)

Table 2 lists data for additional peptidic substrate-Doxorubicin conjugates. These conjugates were prepared from the appropriate amino acid precursors that were elaborated by the general procedures described in Example 4.

TABLE 2

| | | HPLC- |
|---|---|---|
| | Mass | Retention |
| Peptidic substrate-DOX Conjugate | Spectrum | Time (min.) |
| Acetyl-Arg-Arg-Gln-Ser-Arg-Ala-Ala-DOX | 471.2 | 8.23 |
| | (M + 3/3) | |
| Acetyl-Leu-Arg-Arg-Gln-Ser-Arg-Ala-Ala- | 509.2 | 8.60 |
| DOX | (M + 3/3) | |

### EXAMPLE 5

### Determination of Times to 50% Cleavage of Doxorubicin/Peptidic Substrate Conjugates by the Recombinant Protease Domain of MTSP1

One millimolar stock solutions were prepared for each peptidic substrate conjugate in double distilled water. Cleavage reactions were then performed in which 100 μM conjugate was mixed with 1 or 10 nM of the recombinantly-produced active single chain protease domain of MTSP1 (residue 615-855 in SEQ ID No. 2, encoded by nucleotides 1865-2582 in SEQ ID No. 1) in 29.2 mM Tris, pH 8.4, 29.2 mM Imidazole, 217 mM NaCl. Final reaction volume was 200 μL. These reactions were incubated in a water bath at 37° C. At times ranging from 2 to 128 minutes, 20 μL samples were removed, and enzymatic activity was stopped by the addition of trifluoroacetic acid to 0.33%. The amount of hydrolysis in each sample was measured by reverse phase HPLC. Percent hydrolysis was then calculated by dividing the area under the product peak by the sum of the areas under substrate and product peaks. Percent unhydrolyzed substrate was plotted against log of reaction times, and the plots were fit to sigmoidal curves using Prism software from Graphpad Inc. (San Diego, Calif.) to determine times at which 50% of each substrate was cleaved.

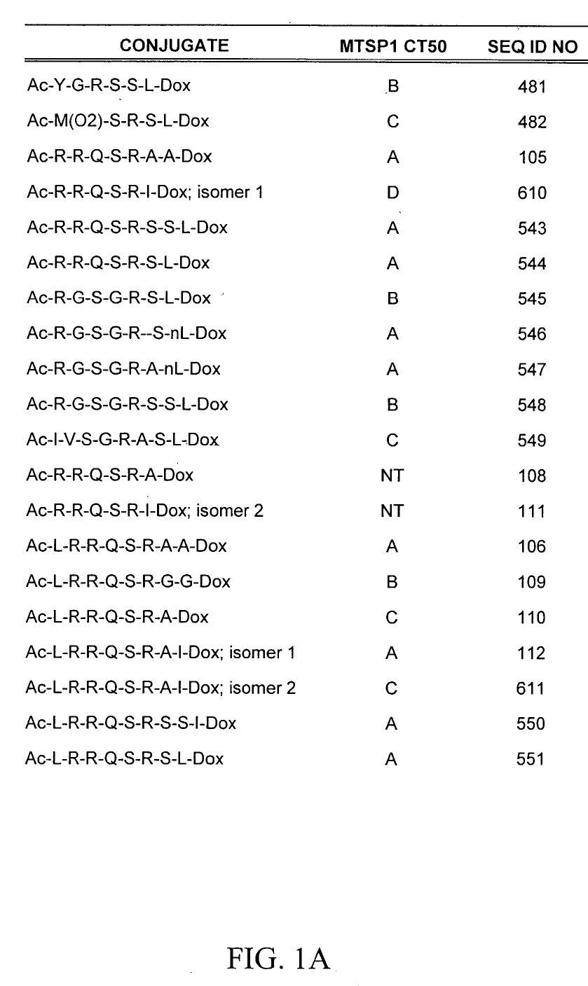

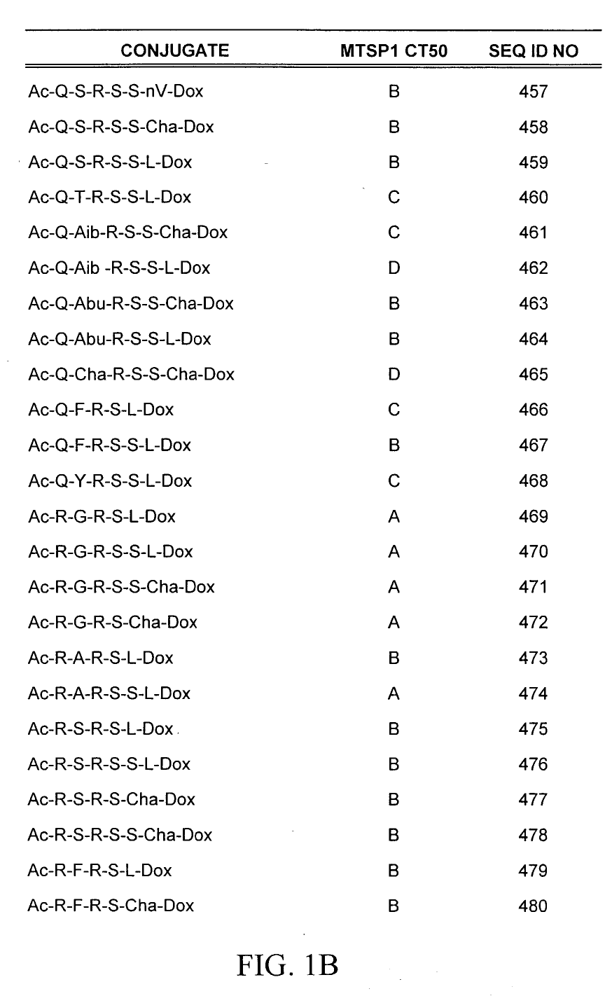

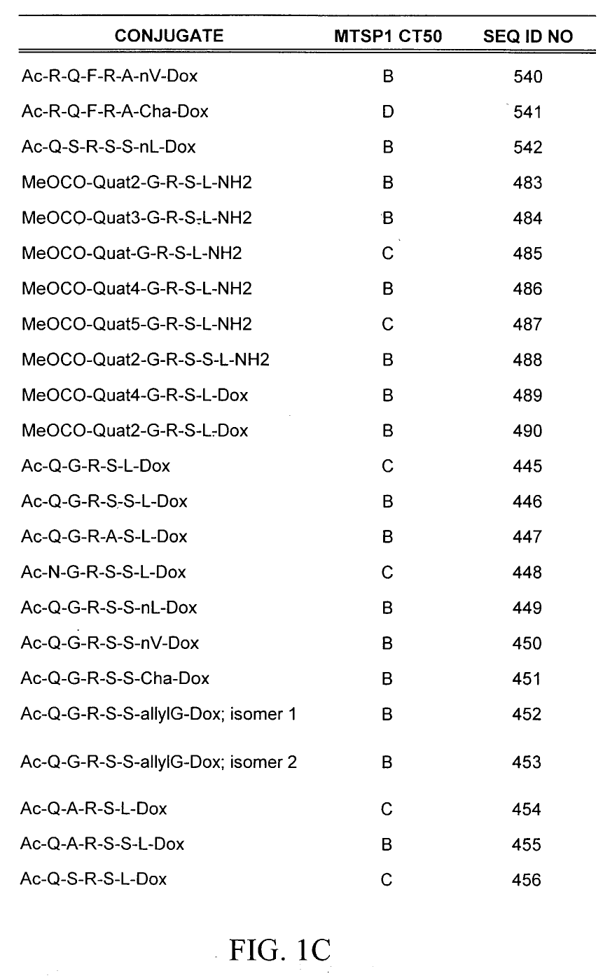

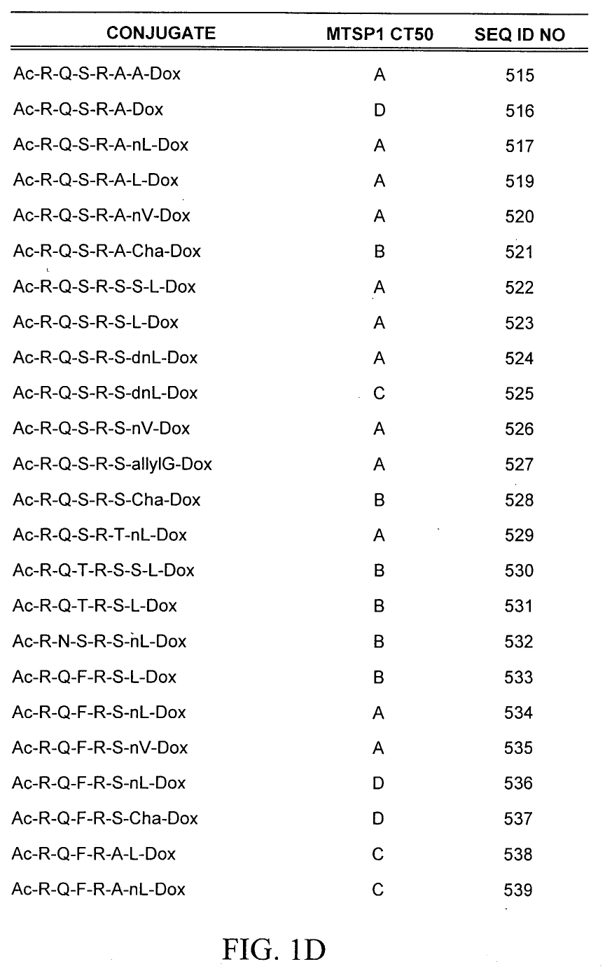

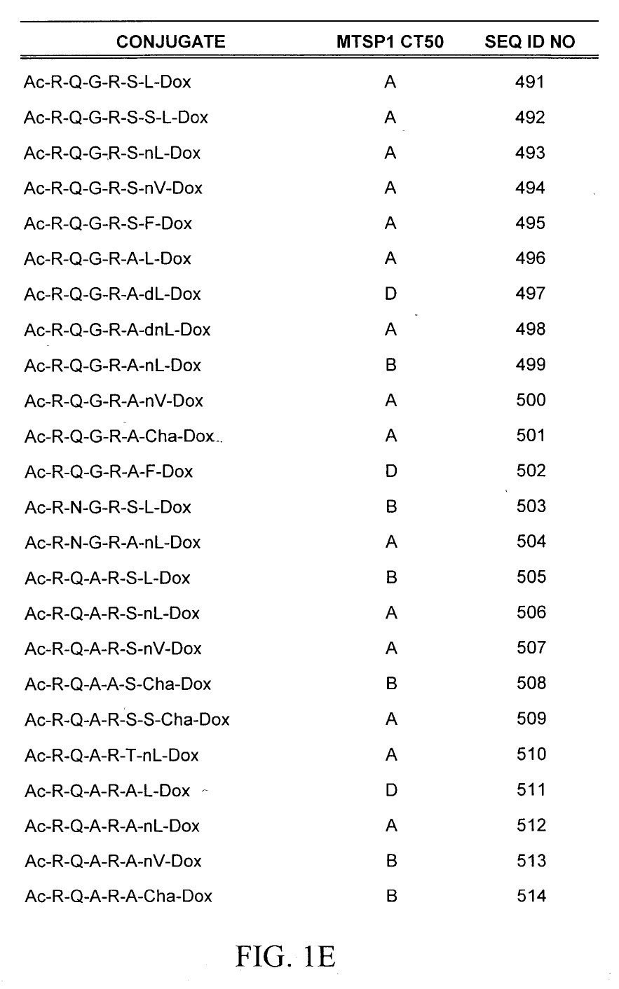

Results for certain of the conjugates provided herein are shown in FIG. 1 (conditions: 1 nM MTSP1 with 100 μM conjugate at 37° C. in 12 mM tris(hydroxymethyl)aminomethane, pH 8.0, 25 mM NaCl, 0.5 mM CaCl₂; reactions were quenched with 0.33% trifluoroacetic acid).

### EXAMPLE 6

### In Vitro Assay of Cytotoxicity of Conjugates

The cytotoxicity of the conjugates also can be tested to confirm that the conjugates act as prodrugs. The conjugates are tested against a line of cells, which is known to be killed by unmodified cytotoxic agent, using-an Alamar Blue assay. Cells, such as LNCaP cells (The American Type Culture Collection (Rockville, Md.)), that express a cell surface protease, such as MTSP1 or endotheliase, are seeded in 96 well plates at a density of 1×104 cells/well (0.1 mL/well). A plate containing medium alone is used as a control. The cells are incubated for 3 days at 37° C. and 20 μL of Alamar Blue is added to the assay well(s). After 7 h of incubation, cell killing is measured using an EL-310 plate reader at 570 and 600 nm. Values for cell killing are expressed as the percentage reduction in cell numbers relative to the media controls.

### EXAMPLE 7

### In Vivo Efficacy of Conjugates

Tumor cells are trypsinized, resuspended in the growth medium and centrifuged for 6 min at 200×g. The cells are resuspended in serum-free α-MEM and counted. The appropriate volume of this solution containing the desired number of cells is then transferred to a conical centrifuge tube, centrifuged as before and resuspended in the appropriate volume of a cold 1:1 mixture of α-MEM-Matrigel. The suspension is kept on ice until the animals are inoculated.

Male nude mice 10 weeks of age are used. Mice are individually weighed and assigned to groups (n=10 per group) with no more than a 2-gram difference in weight between individual mice within each group. On day 1, mice are inoculated subcutaneously with the tumor cell line. ach mouse is inoculated with, for example, 0.5 mL of 0.5×10⁶ to 10⁸ tumor cells/mL in a 60% solution of ice-cold Matrigel and α-MEM. Then, 24 h later, conjugate administration began. Vehicle-treated mice are injected with 5% dextrose in water. At the end of a predetermined time, such as 18 days to two months or more, the mice are sacrificed, and tumor size and mass or other parameters are measured. Tumor size and mass or the other parameters for conjugate-treated mice are compared to vehicle-treated mice to determine efficacy of the conjugate.

Following inoculation with the tumor cells the mice are treated under one of three protocols:

Protocol A

One day after cell inoculation the animals are dosed with 1 to 100, or 3 to 50, or 5 to 25, or 7 to 22 μmol/kg, including 7.2 or 17.9 μmol/kg, of test conjugate, unmodified cytotoxic agent or vehicle control (sterile water). Dosages of the conjugate and cytotoxic agent are initially the maximum non-lethal amount, but can be subsequently titrated lower. Identical doses are administered at 24 hour intervals for 5 days. At the end of 5.5 weeks or other suitable interval, the mice are sacrificed and weights of any tumors present are measured. The animals' weights are determined at the beginning and end of the assay.

Protocol B At 14-15 days after cell inoculation, the animals are dosed with 1 to 100, or 3 to 50, or 5 to 25, or 7 to 22 μmol/kg, including 7.2 or 17.9 μmol/kg, of test conjugate, unmodified cytotoxic agent, or vehicle control (sterile water). Dosages of the conjugate and cytotoxic agent are initially the maximum non-lethal amount, but can be subsequently titrated lower. Identical doses are administered at 24 hour intervals for 5 days. At the end of 5.5 weeks or other suitable interval, the mice are sacrificed and weights of any tumors present are measured. The animals' weights are determined at the beginning and end of the assay.

Protocol C

One day after cell inoculation, the animals are dosed by interperitoneal administration with 1 to 100, or 3 to 50, or 5 to 25, or 7 to 22 μmol/kg, including 7.2 or 17.9 μmol/kg, of test conjugate, unmodified cytotoxic agent, or vehicle control (sterile water). Dosages of the conjugate and cytotoxic agent are initially the maximum non-lethal amount, but can be subsequently titrated lower. Identical doses are administered at 7 day intervals for 5 weeks. At the end of 5.5 weeks or other suitable interval, the mice are sacrificed and weights of any tumors present are measured. The animals' weights are determined at the beginning and end of the assay.

### EXAMPLE 8

### Gene Expression Profiles of Exemplary MTSPs and Domain Organization

Gene Expression Profile of MTSP1 in Normal Tissues, Cancer Cells and Cancer Tissues

To obtain information regarding the tissue distribution and gene expression level of MTSP1, the DNA insert from a Pichia pastoris expression vector, pPIC9K-MTSP1, containing the encoding nucleic acid, was used to probe a blot containing RNA from 76 different human tissues (catalog number 7775-1; human multiple tissue expression (MTE) array; CLONTECH, Palo Alto, Calif.). Significant expression was observed in the colon (ascending, transverse and descending), rectum, trachea, esophagus and duodenum. Moderate expression levels were observed in the jejunum, ileum, ilocecum, stomach, prostate, pituitary gland, appendix, kidney, lung, placenta, pancreas, thyroid gland, salivary gland, mammary gland, fetal kidney, and fetal lung. Lower expression levels were seen in the spleen, thymus, peripheral blood leukocyte, lymph node, bone marrow, bladder, uterus, liver, adrenal gland, fetal heart, fetal liver, fetal spleen, and fetal thymus. A significant amount of the MTSP1 transcript was also detected in colorectal adenocarcinoma cell line (SW480), Burkitt's lymphoma cell line (Daudi), and leukemia cell line (HL-60). RT-PCR of the MTSP1 transcript in several human primary tumors xenografted in athymic nude mice was performed using gene-specific primers. A high level of MTSP1 transcript was detected in colon adenocarcinoma (CX-1) and pancreatic adenocarcinoma (GI-103). Moderate levels were observed in another colon adenocarcinoma (GI-112), ovarian carcinoma (GI-102), lung carcinoma (LX-1), and breast carcinoma (GI-101). Another lung carcinoma (GI-117) expressed a low level of the MTSP1 transcript. A similar RT-PCR was performed to detect the presence of the MTSP1 transcript in PC-3 and LNCaP cell lines. Both cell lines expressed significant amounts of MTSP1 transcript. MTSP1 also is a marker for ovarian cancer.

Gene Expression Profile of the Serine Protease MTSP3 in Normal and Tumor Tissues

To obtain information regarding the tissue distribution of the MTSP3 transcripts, the DNA insert encoding the MTSP3 protease domain was used to probe a RNA blot composed of 76 different human tissues (catalog number 7775-1; human multiple tissue expression (MTE) array; CLONTECH, Palo Alto, Calif.). The expression pattern observed in decreasing signal level was: trachea=colon (descending)=esophagus>colon (ascending)>colon (transverse)=rectum>ileum>duodenum>jejunum>bladder>ilocecum>stomach>kidney>appendix. It also is expressed less abundantly in fetal kidney, and in two tumor cell lines, HeLa S3 and leukemia, K-562. Northern analysis using RNA blots (catalog numbers 7780-1, 7765-1 & 7782-1; human 12-lane, human muscle and human digestive system multiple tissue northern (MTN) blots; CLONTECH) confirmed that the expression was detected most abundantly in the colon, moderately in the esophagus, small intestine, bladder and kidney, and less abundantly in stomach and rectum. A single transcript of ˜2.2 kb was detected.

Amplification of the MTSP3 transcript in several human primary tumors xenografted in mouse was performed using gene-specific primers. The MTSP3 transcript was detected in lung carcinoma (LX-1), colon adenocarcinoma (CX-1), colon adenocarcinoma (GI-112) and ovarian carcinoma (GI-102). No apparent signal was detected in another form of lung carcinoma (GI-117), breast carcinoma (GI-101), pancreatic adenocarcinoma (GI-103) and prostatic adenocarcinoma (PC3).

Gene Expression Profile of MTSP4 in Normal and Tumor Tissues

To obtain information regarding the gene expression profile of the MTSP4 transcript, a DNA fragment encoding part of the LDL receptor domain and the protease domain was used to probe an RNA blot composed of 76 different human tissues (catalog number 7775-1; human multiple tissue expression (MTE) array; CLONTECH). As in the northern analysis of gel blot, a very strong signal was observed in the liver. Signals in other tissues were observed in (decreasing signal level): fetal liver>heart=kidney=adrenal gland=testis fetal heart and kidney=skeletal muscle=bladder=placenta>brain spinal cord=colon=stomach=spleen=lymph node=bone marrow=trachea=uterus=pancreas=salivary gland=mammary gland=lung. MTSP4 also is expressed less abundantly in several tumor cell lines including HeLa S3=leukemia K-562=Burkitt's lymphomas (Raji and Daudi)=colorectal adenocarcinoma (SW480)>lung carcinoma (A549)=leukemia MOLT-4=leukemia HL-60. PCR of the MTSP4 transcript from cDNA libraries made from several human primary tumors xenografted in nude mice (human tumor multiple tissue cDNA panel, catalog number K1522-1, CLONTECH) was performed using MTSP4-specific primers. The MTSP4 transcript was detected in breast carcinoma (GI-101), lung carcinoma (LX-1), colon adenocarcinoma (GI-112) and pancreatic adenocarcinoma (GI-103). No apparent signal was detected in another form of lung carcinoma (GI-117), colon adenocarcinoma (CX-1), ovarian carcinoma (GI-102). and prostatic adenocarcinoma (PC3). The MTSP4 transcript was also detected in LNCaP and PC-3 prostate cancer cell lines as well as in HT-1080 human fibrosarcoma cell line.

Gene Expression Profile of MTSP6 in Normal and Tumor Tissues

To obtain information regarding the gene expression profile of the MTSP6 transcript, a 495 bp DNA fragment obtained from PCR reaction with primers Ch 17-NSP-3 and NSP-4AS was used to probe an RNA blot composed of 76 different human tissues (catalog number 7775-1; human multiple tissue expression (MTE) array; CLONTECH). The strongest signal was observed in duodenum. Signals in other tissues were observed in (decreased signal level): Stomach>trachea=mammary gland=thyroid gland=salivary gland=pituitary gland=pancreas>kidney>lung>jejunum=ileum=ilocecum=appendix=fetal kidney>fetal lung. Very weak signals also can be detected in several other tissues. MTSP6 also is expressed in several tumor cell lines including HeLa S3>colorectal adenocarcinoma (SW480)>leukemia MOLT-4>leukemia K-562. PCR analysis of the MTSP6 transcript from cDNA libraries made from several human primary tumors xenografted in nude mice (human tumor multiple tissue cDNA panel, catalog number K1522-1, CLONTECH) was performed using MTSP6-specific primers (Ch17-NSP-3 and Ch17-NSP2AS). The MTSP6 transcript was strongly detected in lung carcinoma (LX-1), moderately detected in pancreatic adenocarcinoma (GI-103), weakly detected in ovarian carcinoma (GI-102); and very weakly detected in colon adenocarcinoma (GI-112 and CX-1), breast carcinoma (GI-101), lung carcinoma (GI-117) and prostatic adenocarcinoma (PC3). The MTSP6 transcript was also detected in breast cancer cell line MDA-MB-231, prostate cancer cell line PC-3, but not in HT-1080 human fibrosarcoma cell line. MTSP6 also is expressed in mammary gland carcinoma cDNA (Clontech).

Gene Expression Profile of MTSP9 in Normal, Tumor Tissues and Cell Lines

To obtain a gene expression profile of the MTSP9 transcript, the MTSP9 cDNA fragment obtained from human pancreas was used to probe a dot blot composed of RNA extracted from 76 different human tissues (Human Multiple Tissue Expression (MTE) Array; Clontech, Palo Alto, Calif.; catalog no. 7775-1). The results of this analysis indicate that MTSP9 is highly expressed in the esophagus and expressed at a low level in many other tissues. The MTSP9 transcript is found in kidney (adult and fetal), spleen (adult and fetal), placenta, liver (adult and fetal), thymus, peripheral blood leukocyte, lung (adult and fetal), pancreas, lymph node, bone marrow, trachea, uterus, prostate, esophagus, testes, ovary and the gland organs (mammary, adrenal, thyroid, pituitary and salivary). MTSP9 also is expressed in tumor esophagus tissues, in a lung carcinoma (A549 cell line) and, at a low level, in a colorectal carcinoma (SW480), lymphoma (Raji and Daudi), a cervical carcinoma (HeLaS3) and leukemia (HL-60, K-562 and MOLT-4) cell lines.

Gene Expression Profile of MTSP10 in Normal and Tumor Tissues

To obtain information regarding the gene expression profile of the MTSP10 transcript, PCR analysis was carried out on cDNA panels made from several human adult tissues (Clontech, Cat. #K1420-1) cDNA panel using MTSP10-specific primers. MTSP10 transcript was detected in pancreas, lung and kidney. MTSP10 transcript was also detected in small intestine Marathon-Ready cDNA (Clontech). PCR of the MTSP10 transcript from cDNA libraries made from several human primary tumors xenografted in nude mice (human tumor multiple tissue cDNA panel, catalog number K1522-1, CLONTECH) was also performed. The MTSP10 transcript was detected in breast carcinoma (GI-101), lung carcinoma (LX-1 and GI-117), ovarian carcinoma (GI-102), and pancreatic adenocarcinoma (GI-103). The MTSP10 transcript can be weakly detected in prostatic adenocarcinoma (PC3). No apparent signal was detected in two forms of colon adenocarcinomas (GI-112 and CX-1). The MTSP10 transcript was also detected in CWR22R prostate tumor grown on nude mice.

Domain Organization and Gene Expression Profile of MTSP12 in Normal and Tumor Tissues

Domain Organization of MTSP12PD1, -PD2 and -PD3 and Homology to other Serine Proteases

Sequence and protein domain analyses of the translated MTSP12PD1, -PD2 and -PD3 nucleotide sequences indicate that these three serine proteases are contiguous. The sequence order is as follows: MTSP12-PD1 is found at the N terminus followed by MTSP12-PD2, and MTSP12-PD3 is at the C terminus. MTSP12-PD1 and -PD2 contain a trypsin-like serine protease domain (aa 236 to aa 465 and aa 537 to aa 765 for MTSP12-PD1 and -PD2, respectively) characterized by the presence of a protease activation cleavage site ( . . . R₂₃₆↓I₂₃₇VGGMEAS . . . , and . . . R₅₃₇↓V₅₃₈VGGFGAA . . . , for MTSP12-PD1 and -PD2, respectively, and where ¦ indicates a protease activation cleavage site) and the catalytic triad residues (His₂₇₇, Asp₃₂₆ and Ser₄₂₁ in MTSP12-PD1; His₅₇₈, Asp₆₂₆ and Ser₇₂₁ in MTSP12-PD2) in 3 highly-conserved regions of the catalytic domain. MTSP12-PD3 contains a serine protease domain (aa 861 to aa 1087); it has a protease activation cleavage site ( . . . R₈₆₀↓I₈₆₁VGGSAAG . . . ) and has the catalytic His₉₀₂ and Asp₉₄₉, but it has a Ala₁₀₄₃ instead of the conserved catalytic serine found in serine proteases. Several domains are found upstream of the MTSP12-PD1 serine protease domain and these include a transmembrane domain (aa 28 to aa 50), a SEA (sea urchin sperm protein-enterokinase-agrin) domain (aa 51 to aa 170) and an LDLa (low density lipoprotein receptor class a) domain (aa 187 to aa 225). There are 5 possible N-linked glycosylation sites (N₁₁₆SS, N₅₈₁HT, N₆₇₂AT, N₆₉₇FS and N₈₂₀ST). In the protease domain of MTSP12-PD1, there is an unpaired cysteine (C₃₄₆) in a single chain form of the protease domain and the following Cys pairings are noted: C₂₆₂-C₂₇₈; C₃₆₀-C₄₂₇; C₄₁₇-C₄₄₆; C₃₉₂-C₄₀₆. In the protease domain of MTSP12-PD2, there is an unpaired cysteine (C₆₄₆) in a single chain form of the protease domain, and the following Cys pairings are noted: C₅₆₃-C₅₇₉; C₆₆₀-C₇₂₇; C₆₉₂-C₇₀₆; C₇₁₇-C₇₄₆. In the protease domain of MTSP12-PD3, there is an unpaired cysteine (C₉₆₉) in a single chain form of the protease domain, and the following Cys pairings are noted: C₈₈₇-C₉₀₃; C₉₈₃-C₁₀₄₉; C₁₀₁₄-C₁₀₂₈; C₁₀₃₉-C₁₀₆₈.

Alignment (blastp; http://www.ncbi.nlm.nih.gov/BLAST) of the respective MTSP12-PD1, MTSP12-PD2 and MTSP12-PD3 protein sequences to known serine proteases deposited in the public database showed a 45%, 45% and 48% identity to matriptase, a 44%, 43% and 41% identity with DESC1/endotheliase 1, a 44%, 43% and 48% identity to prostamin (AB030036), a 43%, 39% and 39% identity to spinesin (TMPRSS5; NM_{—}030770), and a 40%, 38% and 38% identity to marapsin (NM_{—}031948). The clone has about 93% homology at the nucleotide and encoded protein levels to a clone and encoded provided described in International PCT application No. WO 02/00860 (see SEQ ID Nos. 38 and 97 therein). The encoded protein described in the PCT application, however, includes the Sequence set forth in SEQ ID No. 271 between amino acids Leu373 and Val374 of SEQ ID No. 20, as well as an additional extended sequence of amino acids beteween amino acids Ala48 and Phe49 of SEQ ID No. 20 and lacks amino acids 91-124 of SEQ ID No. 20. The protein provided in International PCT application No. WO02/00860 can be used in the methods provided herein.

Gene and Tissue Expression Profile of MTSP12

To obtain information regarding the tissue distribution profile of the MTSP12PD1, -PD2 and -PD3 transcripts, 3 cDNA probes were prepared. Data indicate that the MTSP12PD1, -PD2 and -PD3 transcript is expressed at a low level in most of the 76 tissues and cell lines, but at a higher level in the lymph node and testes.

To compare the expression profile of MTSP12PD1, -PD2 and -PD3 in a range of normal human and matched tumor tissues, a matched tumor/normal expression array (catalog number 7840-1; http://www.clontech.com) composed of 68 paired cDNA samples from individual patients was used. Results show that the MTSP12PD1, -PD2 and -PD3 transcript is expressed at a low level in a number of normal tissues including breast, uterus, colon, ovary, lung, kidney and rectum, but is not differentially expressed in any of the matched tumors. It also is expressed at a low level in several tumor cell lines, including HeLa (cervical carcinoma), Daudi (Burkitt's lymphoma), K562 (chronic myelogenous leukemia), HL-60 (premyelocytic leukemia), G361 (melanoma), A549 (lung carcinoma), MOLT-4 (lymphoblastic leukemia), SW480 (colorectal adenocarcinoma), and Raji (Burkitt's lymphoma).

Several SMART™ 5′-RACE cDNA libraries (catalog number K1811-1; http://www.clontech.com) prepared from normal breast, normal testes, normal prostate, prostate cancer cell lines and breast cancer cell lines were analyzed for the presence of MTSP12PD1, -PD2 and -PD3 transcript by RT-PCR using two sets of gene-specific primers. The MTSP12-PD2 and -PD3 transcript was detected in normal prostate, PC-3, LNCaP, normal breast, MDA-MB-231, MDA-MB-361, MDA-MB-453 and DU4475, but higher levels were observed in normal breast and MDA-MB-231. The MTSP12-PD1 transcript was detected in the same tissues and cell lines, except higher levels were observed in normal breast, MDA-MB-231 and DU4475.

Gene Expression Profile of MTSP20 in Normal, Tumor Tissues and Cell Lines

To obtain information regarding the gene expression profile of the MTSP20 transcript, the MTSP20 cDNA fragment obtained from human lung tissue was used to probe a dot blot composed of RNA extracted from 76 different human tissues (Human Multiple Tissue Expression (MTE) Array; Clontech, Palo Alto, Calif.; catalog no. 7775-1). The results indicate that RNA encoding MTSP20 is expressed in a variety of tissues. The MTSP20 transcript is found in liver, lymph node, cerebellum, pancreas, prostate, uterus, testis, glands (adrenal, thyroid and salivary), thymus, kidney and spleen. Lower transcript level can be found in lung, placenta, bladder, ovary, digestive system, circulatory system and other parts of the the brain. MTSP20 is also expressed in certain tumor cell lines including lung carcinoma (A519), colorectal carcinoma (SW480), lymphoma (Raji and Daudi), cervical carcinoma (HeLaS3) and leukemia (HL-60, K-562 and MOLT-4) cell lines.

Gene Expression Profile of MTSP22 in Normal, Tumor Tissues and Cell Lines

MTSP22 is expressed in the uterine tissue, thymus, adipose tissue, and lymph node. It may also be expressed in lung, stomach, uterine, breast, ovarian, prostate and in other tumors. To obtain information regarding the gene expression profile of the MTSP22 transcript, the cDNA fragment encoding the entire serine protease domain was used to probe a dot blot composed of RNA extracted from 72 different human tissues (Human Multiple Tissue Expression (MTE) Array; Clontech, Palo Alto, Calif.; catalog no. 7776-1) as well as a dot blot composed of normalized cDNA from 241 tumor and corresponding normal tissues from individual patients (Cancer Profiling Array, Clontech, catalog no. 7841-1). The results of MTE analysis indicated that MTSP22 transcript is expressed primarily in the esophagus. In the cancer profiling array analysis, MTSP22 is highly expressed in 3 of the 42 normal uterus tissue samples, but not in their matched tumor samples. In one of the 42 uterus samples, MTSP22 is expressed in tumor and its metastatic tissues, but not in the normal tissue counterpart. MTSP22 is also expressed in 2 of the 17 stomach tumors and 2 of the 21 lung tumors, but not in their normal tissue counterparts. MTSP22 is also expressed in the normal tissue of the only pancreas matched cDNA pair. PCR analysis was also performed using commercially available cDNA panel from several human adult tissues (Clontech, Cat. #K1420-1 and K1420-2) and primary tumors (Clontech Cat. #K1522-1) as well as several Marathon-Ready cDNAs (Clontech).

MTSP22 cDNA was detected in thymus, adipose tissue, and lymph node. Serine protease domain of MTSP22 and homology to other proteases.

Sequence analysis of the translated MTSP22 protease domain sequence revealed that MTSP22 contains a trypsin-like serine protease domain characterized by the presence of a protease activation cleavage site at the amino terminus of the domain and the catalytic triad residues (histidine, aspartate and serine) in three highly-conserved regions. Alignment of the protein sequence with that of endotheliase 1 (same as serine protease DESC1 protein; GenBank accession number AF064819) indicated that the two proteins share 50% sequence identity in their protease domains.

Gene Expression Profile of MTSP25 in Normal, Tumor Tissues and Cell Lines

MTSP25 is expressed in breast, colon, uterine, ovarian, kidney, prostate, testicular cancer tissue. It may also be expressed in lung, stomach, prostate and in other tumors. To obtain information regarding the gene expression profile of the MTSP25 transcript, a 369 bp DNA fragment containing MTSP25 protease domain sequence (obtained from a PCR reaction) was used to probe a dot blot composed of RNA extracted from 72 different human tissues (Human Multiple Tissue Expression (MTE) Array; Clontech, Palo Alto, Calif.; catalog no. 7776-1) as well as a dot blot composed of normalized cDNA from 241 tumor and corresponding normal tissues from individual patients (Cancer Profiling Array, Clontech, catalog no. 7841-1). The results of MTE analysis indicate that MTSP25 transcript is expressed weakly in the lymph node. In the cancer profiling array analysis, MTSP25 is highly expressed in all 4 prostate samples (in normal and cancer samples). In one of the 20 kidney cDNA pairs, MTSP25 is highly expressed in the tumor sample, but not in its normal tissue counterpart. MTSP25 is also expressed in 1 of the 50 breast cancer samples, but not in its normal tissue counterpart.

MTSP25 is also expressed in 3 of the 42 normal uterus samples, but not in their tumor counterparts. MTSP25 expression is also detected in 3 of the 14 ovarian cancer samples. Among these three samples, the expression of MTSP25 was also detected in one of the matched normal tissue counterparts. MTSP25 expression was also detected in 5 tumor samples in the 34 colon cDNA pairs.

PCR analysis was also performed using a commercially available cDNA panel from several human adult tissues (Clontech, Cat. #K1420-1 and K1420-2) as well as several Marathon-Ready cDNAs (Clontech). MTSP25 cDNA was strongly detected in testis and mammary gland adenocarcinoma, weakly detected in brain, placenta, lung, spleen, prostate, small intestine, colon, and leukocyte, and very weakly detected in heart, liver, and pancreas.

### EXAMPLE 9

Conjugates that have been prepared according to the procedures of Examples 1-4 by routine and minor modification of the procedures, such as using different Fmoc-amino acid building blocks, include:

Ac-R-Q-G-R-S-L-(Dox) (SEQ ID NO: 491);

Ac-R-Q-G-R-S-S-L-(Dox) (SEQ ID NO: 492);

Ac-R-Q-G-R-S-nL-(Dox) (SEQ ID NO: 493);

Ac-R-Q-G-R-S-nV-(Dox) (SEQ ID NO: 494);

Ac-R-Q-G-R-S-F-(Dox) (SEQ ID NO: 495);

Ac-R-Q-G-R-A-L-(Dox) (SEQ ID NO: 496);

Ac-R-Q-G-R-A-L-(Dox) (SEQ ID NO: 497);

Ac-R-Q-G-R-A-nL-(Dox) (SEQ ID NO: 498);

Ac-R-Q-G-R-A-nL-(Dox) (SEQ ID NO: 499);

Ac-R-Q-G-R-A-nV-(Dox) (SEQ ID NO: 500);

Ac-R-Q-G-R-A-Cha-(Dox) (SEQ ID NO: 501);

Ac-R-Q-G-R-A-F-(Dox) (SEQ ID NO: 502);

Ac-R-N-G-R-S-L-(Dox) (SEQ ID NO: 503);

Ac-R-N-G-R-A-nL-(Dox) (SEQ ID NO: 504);

Ac-R-Q-A-R-S-L-(Dox) (SEQ ID NO: 505);

Ac-R-Q-A-R-S-nL-(Dox) (SEQ ID NO: 506);

Ac-R-Q-A-R-S-nV-(Dox) (SEQ ID NO: 507);

Ac-R-Q-A-A-S-Cha-(Dox) (SEQ ID NO: 508);

Ac-R-Q-A-R-S-S-Cha-(Dox) (SEQ ID NO: 509);

Ac-R-Q-A-R-T-nL-(Dox) (SEQ ID NO: 510);

Ac-R-Q-A-R-A-L-(Dox) (SEQ ID NO: 511);

Ac-R-Q-A-R-A-nL-(Dox) (SEQ ID NO: 513);

Ac-R-Q-A-R-A-nV-(Dox) (SEQ ID NO: 514);

Ac-R-Q-A-R-A-Cha-(Dox) (SEQ ID NO: 515);

Ac-R-Q-S-R-A-A-(Dox) (SEQ ID NO: 516);

Ac-R-Q-S-R-A-(Dox) (SEQ ID NO: 517);

Ac-R-Q-S-R-A-nL-(Dox) (SEQ ID NO: 518);

Ac-R-Q-S-R-A-L-(Dox) (SEQ ID NO: 519);

Ac-R-Q-S-R-A-nV-(Dox) (SEQ ID NO: 520);

Ac-R-Q-S-R-A-Cha-(Dox) (SEQ ID NO: 521);

Ac-R-Q-S-R-S-S-L-(Dox) (SEQ ID NO: 522);

Ac-R-Q-S-R-S-L-(Dox) (SEQ ID NO: 523);

Ac-R-Q-S-R-S-dnL-(Dox) (SEQ ID NO: 524);

Ac-R-Q-S-R-S-nL-(Dox) (SEQ ID NO: 525);

Ac-R-Q-S-R-S-nV-(Dox) (SEQ ID NO: 526);

Ac-R-Q-S-R-S-allylG-(Dox) (SEQ ID NO: 527);

Ac-R-Q-S-R-S-Cha-(Dox) (SEQ ID NO: 528);

Ac-R-Q-S-R-T-nL-(Dox) (SEQ ID NO: 529);

Ac-R-Q-T-R-S-S-L-(Dox) (SEQ ID NO: 530);

Ac-R-Q-T-R-S-L-(Dox) (SEQ ID NO: 531);

Ac-R-N-S-R-S-nL-(Dox) (SEQ ID NO: 532);

Ac-R-Q-F-R-S-L-(Dox) (SEQ ID NO: 533);

Ac-R-Q-F-R-S-nL-(Dox) (SEQ ID NO: 534);

Ac-R-Q-F-R-S-nV-(Dox) (SEQ ID NO: 535);

Ac-R-Q-F-R-S-nL-(Dox) (SEQ ID NO: 536);

Ac-R-Q-F-R-S-Cha-(Dox) (SEQ ID NO: 537);

Ac-R-Q-F-R-A-L-(Dox) (SEQ ID NO: 538);

Ac-R-Q-F-R-A-nL-(Dox) (SEQ ID NO: 539);

Ac-R-Q-F-R-A-nV-(Dox) (SEQ ID NO: 540);

Ac-R-Q-F-R-A-Cha-(Dox) (SEQ ID NO: 541);

Ac-Q-S-R-S-S-nL-(Dox) (SEQ ID NO: 542);

MeOCO-Quat2-G-R-S-L-NH2 (SEQ ID NO: 483);

MeOCO-Quat3-G-R-S-L-NH2 (SEQ ID NO: 484);

MeOCO-Quat-G-R-S-L-NH2 (SEQ ID NO: 485);

MeOCO-Quat4-G-R-S-L-NH2 (SEQ ID NO: 486);

MeOCO-Quat5-G-R-S-L-NH2 (SEQ ID NO: 487);

MeOCO-Quat2-G-R-S-S-L-NH2 (SEQ ID NO: 488);

MeOCO-Quat4-G-R-S-L-(Dox) (SEQ ID NO: 489);

MeOCO-Quat2-G-R-S-L-(Dox) (SEQ ID NO: 490);

Ac-Q-G-R-S-L-(Dox) (SEQ ID NO: 445);

Ac-Q-G-R-S-S-L-(Dox) (SEQ ID NO: 446);

Ac-Q-G-R-A-S-L-(Dox) (SEQ ID NO: 447);

Ac-N-G-R-S-S-L-(Dox) (SEQ ID NO: 448);

Ac-Q-G-R-S-S-nL-(Dox) (SEQ ID NO: 449);

Ac-Q-G-R-S-S-nV-(Dox) (SEQ ID NO: 450);

Ac-Q-G-R-S-S-Cha-(Dox) (SEQ ID NO: 451);

Ac-Q-G-R-S-S-allylG-(Dox) (SEQ ID NO: 452);

Ac-Q-G-R-S-S-allylG-(Dox) (SEQ ID NO: 453);

Ac-Q-A-R-S-L-(Dox) (SEQ ID NO: 454);

Ac-Q-A-R-S-S-L-(Dox) (SEQ ID NO: 455);

Ac-Q-S-R-S-L-(Dox) (SEQ ID NO: 456);

Ac-Q-S-R-S-S-nV-(Dox) (SEQ ID NO: 457);

Ac-Q-S-R-S-S-Cha-(Dox) (SEQ ID NO: 458);

Ac-Q-S-R-S-S-L-(Dox) (SEQ ID NO: 459);

Ac-Q-T-R-S-S-L-(Dox) (SEQ ID NO: 460);

Ac-Q-Aib-R-S-S-Cha-(Dox) (SEQ ID NO: 461);

Ac-Q-Aib-R-S-S-L-(Dox) (SEQ ID NO: 462);

Ac-Q-Abu-R-S-S-Cha-(Dox) (SEQ ID NO: 463);

Ac-Q-Abu-R-S-S-L-(Dox) (SEQ ID NO: 464);

Ac-Q-Cha-R-S-S-Cha-(Dox) (SEQ ID NO: 465);

Ac-Q-F-R-S-L-(Dox) (SEQ ID NO: 466);

Ac-Q-F-R-S-S-L-(Dox) (SEQ ID NO: 467);

Ac-Q-Y-R-S-S-L-(Dox) (SEQ ID NO: 468);

Ac-R-G-R-S-L-(Dox) (SEQ ID NO: 469);

Ac-R-G-R-S-S-L-(Dox) (SEQ ID NO: 470);

Ac-R-G-R-S-S-Cha-(Dox) (SEQ ID NO: 471);

Ac-R-G-R-S-Cha-(Dox) (SEQ ID NO: 472);

Ac-R-A-R-S-L-(Dox) (SEQ ID NO: 473);

Ac-R-A-R-S-S-L-(Dox) (SEQ ID NO: 474);

Ac-R-S-R-S-L-(Dox) (SEQ ID NO: 475);

Ac-R-S-R-S-S-L-(Dox) (SEQ ID NO: 476);

Ac-R-S-R-S-Cha-(Dox) (SEQ ID NO: 477);

Ac-R-S-R-S-S-Cha-(Dox) (SEQ ID NO: 478);

Ac-R-F-R-S-L-(Dox) (SEQ ID NO: 479);

Ac-R-F-R-S-Cha-(Dox) (SEQ ID NO: 480);

Ac-Y-G-R-S-S-L-(Dox) (SEQ ID NO: 481);

Ac-M(O2)-S-R-S-L-(Dox) (SEQ ID NO: 482);

Ac-R-R-Q-S-R-A-A-(Dox) (SEQ ID NO: 105);

Ac-R-R-Q-S-R-I-(Dox) (SEQ ID NO: 610);

Ac-R-R-Q-S-R-S-S-L-(Dox) (SEQ ID NO: 543);

Ac-R-R-Q-S-R-S-L-(Dox) (SEQ ID NO: 544);

Ac-R-G-S-G-R-S-L-(Dox) (SEQ ID NO: 545);

Ac-R-G-S-G-R--S-nL-(Dox) (SEQ ID NO: 546);

Ac-R-G-S-G-R-A-nL-(Dox) (SEQ ID NO: 547);

Ac-R-G-S-G-R-S-S-L-(Dox) (SEQ ID NO: 548);

Ac-I-V-S-G-R-A-S-L-(Dox) (SEQ ID NO: 549);

Ac-R-R-Q-S-R-A-(Dox) (SEQ ID NO: 108);

Ac-R-R-Q-S-R-I-(Dox) (SEQ ID NO: 111);

Ac-L-R-R-Q-S-R-A-A-(Dox) (SEQ ID NO: 106);

Ac-L-R-R-Q-S-R-G-G-(Dox) (SEQ ID NO: 109);

Ac-L-R-R-Q-S-R-A-(Dox) (SEQ ID NO: 110);

Ac-L-R-R-Q-S-R-A-I-(Dox) (SEQ ID NO: 112);

Ac-L-R-R-Q-S-R-A-I-(Dox) (SEQ ID NO: 611);

Ac-L-R-R-Q-S-R-S-S-L-(Dox) (SEQ ID NO: 550);

Ac-L-R-R-Q-S-R-S-L-(Dox) (SEQ ID NO: 551);

Ac-S-G-R-S-L-(Dox) (SEQ ID NO: 362);

Ac-S-G-R-S-S-L-(Dox) (SEQ ID NO: 363);

Ac-S-G-R-S-S-S-L-(Dox) (SEQ ID NO: 364);

Ac-S-G-R-S-nL-(Dox) (SEQ ID NO: 365);

Ac-S-G-R-S-nV-(Dox) (SEQ ID NO: 366); isomer 1

Ac-S-G-R-S-nV-(Dox) (SEQ ID NO: 367); isomer 2

Ac-S-G-R-S-G(hex)-(Dox) (SEQ ID NO: 368);

Ac-S-G-R-S-Cha-(Dox) (SEQ ID NO: 369);

Ac-S-G-R-S-hCha-(Dox) (SEQ ID NO: 370);

Ac-S-A-R-S-L-(Dox) (SEQ ID NO: 371);

Ac-S-A-R-S-S-L-(Dox) (SEQ ID NO: 372);

Ac-S-S-R-S-nL-(Dox) (SEQ ID NO: 373);

Ac-T-G-R-S-Abu-(Dox) (SEQ ID NO: 374);

Ac-T-G-R-S-L-(Dox) (SEQ ID NO: 375);

Ac-T-G-R-S-nV-(Dox) (SEQ ID NO: 376);

Ac-T-G-R-S-nL-(Dox) (SEQ ID NO: 377);

Ac-T-G-R-S-G(hex)-(Dox) (SEQ ID NO: 378);

Ac-T-G-R-S-Cha-(Dox) (SEQ ID NO: 379);

Ac-T-G-R-S-hCha-(Dox) (SEQ ID NO: 380);

Ac-T-G-R-T-Abu-(Dox) (SEQ ID NO: 381);

Ac-T-G-R-hS-nL-(Dox) (SEQ ID NO: 382);

Ac-T-G-R-Abu-nL-(Dox) (SEQ ID NO: 383);

Ac-T-G-R-Abu-nV-(Dox) (SEQ ID NO: 384);

Ac-T-G-F(Gn)-S-nL-(Dox) (SEQ ID NO: 385);

Ac-T-G-F(Gn)-S-Cha-(Dox) (SEQ ID NO: 386);

Ac-T-G-F(Gn)-Abu-nV-(Dox) (SEQ ID NO: 387);

Ac-T-G-K(alloc)-S-nL-(Dox) (SEQ ID NO: 388);

Ac-T-G-K-S-nL-(Dox) (SEQ ID NO: 389);

Ac-T-G-hR-S-nL-(Dox) (SEQ ID NO: 390);

Ac-(hS)G-G-R-S-nL-(Dox) (SEQ ID NO: 391);

MeOCO-T-G-R-S-nL-(Dox) (SEQ ID NO: 392);

PhSO2-T-G-R-S-nL-(Dox) (SEQ ID NO: 393);

MeOEtCO-T-G-R-S-nL-(Dox) (SEQ ID NO: 394);

MeO(EtO)2Ac-T-G-R-S-nL-(Dox) (SEQ ID NO: 395);

4-oxo-Pentanoyl-T-G-R-S-nL-(Dox) (SEQ ID NO: 396);

3,4-MethyldioxyPhAc-T-G-R-S-nL-(Dox) (SEQ ID NO: 397);

2-PyridilAc-T-G-R-S-nL-(Dox) (SEQ ID NO: 398);

PhOAc-T-G-R-S-nL-(Dox) (SEQ ID NO: 399);

L-3-PhLactyl-T-G-R-S-nL-(Dox) (SEQ ID NO: 400);

MeOAc-T-G-R-S-nL-(Dox) (SEQ ID NO: 401);

PhAc-T-G-R-S-nL-(Dox) (SEQ ID NO: 402);

MeOEtOCO-T-G-R-S-nL-(Dox) (SEQ ID NO: 403);

MeOEtOAc-T-G-R-S-nL-(Dox) (SEQ ID NO: 404);

HOOCButa-T-G-R-S-nL-(Dox) (SEQ ID NO: 405);

Z-T-G-R-S-nL-(Dox) (SEQ ID NO: 406);

EtOCO-T-G-R-S-nL-(Dox) (SEQ ID NO: 407);

βA-T-G-R-S-nL-(Dox) (SEQ ID NO: 408);

Pent-4-ynoyl-T-G-R-S-nL-(Dox) (SEQ ID NO: 409);

NapAc-T-G-R-S-nL-(Dox) (SEQ ID NO: 410);

iBoc-T-G-R-S-nL-(Dox) (SEQ ID NO: 411);

HOAc-T-G-R-S-nL-(Dox) (SEQ ID NO: 412);

MeSucc-T-G-R-S-nL-(Dox) (SEQ ID NO: 413);

N,N-diMeGly-T-G-R-S-nL-(Dox) (SEQ ID NO: 414);

Succ-T-G-R-S-nL-(Dox) (SEQ ID NO: 415);

HCO-T-G-R-S-nL-(Dox) (SEQ ID NO: 416);

Ac-T-A-R-S-nL-(Dox) (SEQ ID NO: 417);

Ac-T-A-F(Gn)-S-nL-(Dox) (SEQ ID NO: 418);

Ac-T-A-R-Abu-nV-(Dox) (SEQ ID NO: 419);

Ac-T-A-R-S-Abu-(Dox) (SEQ ID NO: 420);

Ac-T-A-R-T-Abu-(Dox) (SEQ ID NO: 421);

Ac-T-S(O-Me)-R-S-nL-(Dox) (SEQ ID NO: 422);

Ac-T-hS-R-S-nL-(Dox) (SEQ ID NO: 423);

Ac-T-(1-Me)H-R-S-nL-(Dox) (SEQ ID NO: 424);

Ac-T-(3-Me)H-R-S-nL-(Dox) (SEQ ID NO: 425);

Ac-T-H-R-S-nL-(Dox) (SEQ ID NO: 426);

Ac-T-Sar-R-S-nL-(Dox) (SEQ ID NO: 427);

Ac-T-nV-R-S-nL-(Dox) (SEQ ID NO: 428);

Ac-T-nL-R-S-nL-(Dox) (SEQ ID NO: 429);

Ac-T-A-R-S-Cha-(Dox) (SEQ ID NO: 430);

Ac-T-Abu-R-S-nL-(Dox) (SEQ ID NO: 431);

Ac-4,4diMeThr-G-R-S-nL-(Dox) (SEQ ID NO: 432);

Ac-hS-G-R-S-nL-(Dox) (SEQ ID NO: 433);

Ac-hS-G-R-hS-Cha-(Dox) (SEQ ID NO: 434);

Ac-hS-G-R-S-Cha-(Dox) (SEQ ID NO: 435);

Ac-hS-G-R-T-Cha-(Dox) (SEQ ID NO: 436);

Ac-hS-A-R-S-Cha-(Dox) (SEQ ID NO: 437);

Ac-N-G-R-S-nL-(Dox) (SEQ ID NO: 438);

Ac-Y-G-R-S-S-L-(Dox) (SEQ ID NO: 439);

Ac-Y-G-R-S-Cha-(Dox) (SEQ ID NO: 440);

Ac-Q-G-R-S-S-nL-(Dox) (SEQ ID NO: 441);

Ac-Q-G-R-S-S-nV-(Dox) (SEQ ID NO: 442);

Ac-L-R-G-S-G-R-S-A-(Dox) (SEQ ID NO: 573);

Ac-L-R-G-S-G-R-S-L-(Dox) (SEQ ID NO: 342);

Ac-L-R-G-S-G-R-S-L-(Dox) (SEQ ID NO: 343);

Ac-L-R-G-S-G-R-S-S-nL-(Dox) (SEQ ID NO: 344);

Ac-L-R-G-S-G-R-S-S-Cha-(Dox) (SEQ ID NO: 345);

Ac-L-R-G-dS-A-R-S-A-(Dox) (SEQ ID NO: 574);

Ac-L-R-G-S-A-R-S-S-L-(Dox) (SEQ ID NO: 346);

Ac-L-R-G-S-A-R-S-L-(Dox) (SEQ ID NO: 347);

Ac-L-R-G-S-A-R-S-S-Cha-(Dox) (SEQ ID NO: 348);

Ac-L-R-G-S-A-R-S-S-nV-(Dox) (SEQ ID NO: 349);

Ac-L-R-G-S-A-R-S-S-nL-(Dox) (SEQ ID NO: 350);

Ac-V-I-V-S-G-R-A-L-(Dox) (SEQ ID NO: 351);

Ac-V-I-V-S-A-R-S-L-(Dox) (SEQ ID NO: 352);

Ac-V-I-V-S-G-R-S-S-L-(Dox) (SEQ ID NO: 353);

Ac-V-I-V-S-A-R-M-A-(Dox) (SEQ ID NO: 354);

Ac-V-I-V-S-A-R-nL-A-(Dox) (SEQ ID NO: 355);

Ac-V-I-V-S-A-R-S-nL-(Dox) (SEQ ID NO: 356);

Ac-V-I-V-S-A-R-S-Cha-(Dox) (SEQ ID NO: 357);

Ac-V-I-V-S-A-R-S-Cha-(Dox) (SEQ ID NO: 358);

Ac-V-I-V-S-A-R-S-S-Cha-(Dox) (SEQ ID NO: 359);

Ac-R-R-(Me)C-P-G-R-V-V-(Dox) (SEQ ID NO: 360);

Ac-R-R-nV-P-A-R-S-L-(Dox) (SEQ ID NO: 361);

Ac-R-G-dS-A-R-S-A-(Dox) (SEQ ID NO: 309);

Ac-R-G-S-G-R-S-A-(Dox) (SEQ ID NO: 310);

Ac-R-G-S-G-R-A-L-(Dox) (SEQ ID NO: 311);

Ac-R-G-S-G-R-S-L-(Dox) (SEQ ID NO: 312);

Ac-R-G-S-G-R--S-nL-(Dox) (SEQ ID NO: 313);

Ac-R-G-S-G-R-A-nL-(Dox) (SEQ ID NO: 314);

Ac-R-G-S-G-R-S-S-L-(Dox) (SEQ ID NO: 315);

Ac-R-G-S-G-R-S-Cha-(Dox) (SEQ ID NO: 316);

Ac-R-G-S-G-R-S-S-Cha-(Dox) (SEQ ID NO: 317);

Ac-R-G-S-A-R-S-Cha-(Dox) (SEQ ID NO: 318);

Ac-R-G-S-A-R-S-S-(Dox) (SEQ ID NO: 319);

Ac-R-G-S-A-R-S-nV-(Dox) (SEQ ID NO: 320);

Ac-R-G-S-A-R-S-S-nV-(Dox) (SEQ ID NO: 321);

Ac-R-G-S-A-R-S-L-(Dox) (SEQ ID NO: 322);

Ac-R-(Me)C-P-G-R-V-V-(Dox) (SEQ ID NO: 323);

Ac-R-(Me)C-P-G-R-V-V-(Dox) (SEQ ID NO: 324);

Ac-R-C(Me)-P-G-R-S-L-(Dox) (SEQ ID NO: 325);

Ac-R-L-P-G-R-S-L-(Dox) (SEQ ID NO: 326);

Ac-R-V-P-G-R-S-L-(Dox) (SEQ ID NO: 327);

Ac-R-V-P-G-R-S-L-(Dox) (SEQ ID NO: 328);

Ac-R-nL-P-G-R-S-L-(Dox) (SEQ ID NO: 329);

Ac-R-G(tBu)-P-A-R-S-L-(Dox) (SEQ ID NO: 330);

Ac-R-L-P-A-R-S-L-(Dox) (SEQ ID NO: 331);

Ac-R-V-P-A-R-S-L-(Dox) (SEQ ID NO: 332);

Ac-R-nL-P-A-R-S-L-(Dox) (SEQ ID NO: 333);

Ac-I-V-S-G-R-A-L-(Dox) (SEQ ID NO: 334);

Ac-I-V-S-G-R-S-S-L-(Dox) (SEQ ID NO: 335);

Ac-I-V-S-G-R-A-S-L-(Dox) (SEQ ID NO: 336);

Ac-I-V-S-A-R-M-A-(Dox) (SEQ ID NO: 337);

Ac-I-V-S-A-R-nL-A-(Dox) (SEQ ID NO: 338);

Ac-I-V-S-A-R-S-L-(Dox) (SEQ ID NO: 339);

Ac-I-V-S-A-R-S-nL-(Dox) (SEQ ID NO: 340);

Ac-I-V-S-A-R-S-S-L-(Dox) (SEQ ID NO: 341);

Ac-G-S-G-R-S-A-(Dox) (SEQ ID NO: 585);

Ac-G-S-G-R-S-L-(Dox) (SEQ ID NO: 277);

Ac-G-S-G-R-A-L-(Dox) (SEQ ID NO: 278);

Ac-G-S-G-R-S-S-L-(Dox) (SEQ ID NO: 279);

Ac-G-S-G-R-L-(Dox) (SEQ ID NO: 280);

Ac-G-S-G-(4-guan)Phg-S-L-NH2 (SEQ ID NO: 281);

Ac-G-S-G-R-S-S-Cha-(Dox) (SEQ ID NO: 282);

Ac-G-S-G-R-A-S-L-(Dox) (SEQ ID NO: 283);

Ac-G-S-G-R-S-nL-(Dox) (SEQ ID NO: 284);

Ac-G-T-G-R-S-nL-(Dox) (SEQ ID NO: 285);

Succ-bA-T-G-R-S-nL-(Dox) (SEQ ID NO: 286);

Ac-G-T-G-R-S-hCha-(Dox) (SEQ ID NO: 287);

Ac-G-hS-G-R-S-nL-(Dox) (SEQ ID NO: 288);

Ac-G-dS-A-R-S-A-(Dox) (SEQ ID NO: 289);

Ac-G-S-A-R-S-L-(Dox) (SEQ ID NO: 290);

Ac-G-S-A-R-S-S-Cha-(Dox) (SEQ ID NO: 291);

Ac-G-S-A-R-S-S-L-(Dox) (SEQ ID NO: 292);

Ac-G-S-A-R-A-S-L-(Dox) (SEQ ID NO: 293);

Ac-V-S-G-R-S-L-(Dox) (SEQ ID NO: 294);

Ac-V-S-G-R-A-L-(Dox) (SEQ ID NO: 295);

Ac-V-S-G-R-A-S-L-(Dox) (SEQ ID NO: 296);

Ac-V-S-G-R-S-S-L-(Dox) (SEQ ID NO: 297);

Ac-V-S-A-R-M-A-(Dox) (SEQ ID NO: 298);

Ac-V-S-A-R-nL-A-(Dox) (SEQ ID NO: 299);

Ac-V-S-A-R-S-nL-(Dox) (SEQ ID NO: 300);

Ac-V-S-A-R-S-L-(Dox) (SEQ ID NO: 301);

Ac-(Me)C-P-G-R-V-V-(Dox) (SEQ ID NO: 302);

Ac-(Me)C-P-G-R-V-V-(Dox) (SEQ ID NO: 303);

Ac-C(Me)-P-G-R-A-L-(Dox) (SEQ ID NO: 304);

Ac-C(Me)-P-G-R-S-L-(Dox) (SEQ ID NO: 305);

Ac-C(Me)-P-A-R-S-L-(Dox) (SEQ ID NO: 306);

Ac-C(Me)-P-A-R-A-S-L-(Dox) (SEQ ID NO: 307);

Ac-G(tBu)-P-G-R-S-L-(Dox) (SEQ ID NO: 308);

Ac-Q-S-R-A-A-(taxol) (SEQ ID NO: 552);

Ac-Q-S-R-S-A-(taxol) (SEQ ID NO: 553);

Ac-Q-S-R-S-G-(taxol) (SEQ ID NO: 554);

Ac-R-S-R-A-A-(taxol) (SEQ ID NO: 555);

Ac-R-Q-S-R-A-A-(taxol) (SEQ ID NO: 556);

Ac-R-Q-S-R-S-A-(taxol) (SEQ ID NO: 557);

Ac-R-Q-S-R-S-A-A-(taxol) (SEQ ID NO: 558);

Ac-R-G-S-G-R-S-A-(taxol) (SEQ ID NO: 559);

Ac-S-G-R-A-A-(taxol) (SEQ ID NO: 560);

Ac-S-G-R-S-A-(taxol) (SEQ ID NO: 561);

Ac-S-G-R-S-S-A-(taxol) (SEQ ID NO: 562);

Ac-S-G-R-A-S-A-(taxol) (SEQ ID NO: 563);

Ac-S-G-R-S-G-(taxol) (SEQ ID NO: 564);

Ac-S-G-R-S-S-G-(taxol) (SEQ ID NO: 565);

Ac-S-G-R-S-G-A-(taxol) (SEQ ID NO: 566);

Ac-S-G-R-S-G-G-(taxol) (SEQ ID NO:567);

Ac-G-T-G-R-S-G-G-(taxol) (SEQ ID NO: 568);

Ac-L-R-R-Q-S-R-A-A-(Dox) (SEQ ID NO: 597);

MeSO2-dA(Chx)-Abu-R-S-L-(Dox) (SEQ ID NO: 598);

Ac-R-A-R-S-L-(Dox) (SEQ ID NO: 599);

Ac-dA(Chx)-Abu-R-S-L-(Dox) (SEQ ID NO: 600);

Ac-dA(Chx)-Abu-R-S-S-L-(Dox) (SEQ ID NO: 601);

Ac-Q-G-R-S-S-L-(Dox) (SEQ ID NO: 602);

MeOCO-dhF-P(OH)-R-S-S-L-(Dox) (SEQ ID NO: 603);

MeOCO-Quat4-G-R-S-L-(Dox) (SEQ ID NO: 604);

As-dCha-P(OH)-R-S-S-L-(Dox) (SEQ ID NO: 605);

Ac-dCha-Abu-R-S-S-A-(taxol) (SEQ ID NO: 606);

MeOCO-Quat2-G-R-S-L-NH2 (SEQ ID NO: 607);

MeOCO-Quat3-G-R-S-L-NH2 (SEQ ID NO: 608); and

MeOCO-Quat-G-R-S-L-NH2 (SEQ ID NO: 609).

### EXAMPLE 10

### Pharmacokinetic Studies of Conjugates and Fraction of the Dose Metabolized to Doxorubicin and Leucine-Doxorubicin in Naïve and Tumor Bearing Mice.

Naïve or tumor bearing nude mice 8-12 weeks of age have been used for pharmacokinetic studies of the test conjugates. Tumor cells for implantation have been prepared following one of three protocols.

Protocol A Tumor Cells Collected from Tissue Culture

Tumor cells are trypsinized and resuspended in the growth medium and centrifuged for 6 min at 200×g. The cells are resuspended in serum-free medium and counted. The appropriate volume of the solution containing the desired number of cells is then transferred to a conical centrifuge tube, centrifuged as before and resuspended in the appropriate volume of a cold 1:1 mixture of cells in phenol free medium:matrigel. Each mouse is inoculated with 0.2-0.5 mL containing between 1×10⁶ and 1×10⁷ tumor cells subcutaneously or orthotopically.

Protocol B Tumor Cell Suspension Established tumors (200-1000 mm³) are dissected from mice, weighed and rinsed in tumor cell growth medium. The tumors are passed through a steel cell dissociation sieve. The cells are rinsed through the sieve with growth medium. The cells are centrifuged for 6 min at 200×g and resuspended in the appropriate volume of a cold 1:1 mixture of cells:matrigel. Each mouse is inoculated with 0.2-0.5 mL of tumor cells subcutaneously or orthotopically.

Protocol C Tumor Fragments

Alternatively a tumor measuring approximately 800 mm³ is dissected out of a mouse, rinsed in tumor cell growth medium and cut into 1-2 mm³ fragments. Each fragment is inoculated subcutaneously or orthotopically using a trocar needle.

Pharmacokinetic Study

Naïve or tumor bearing mice are individually weighed and assigned to groups. The mice are dosed with 1-100 umole/kg, including 30 umole/kg, 25 umole/kg, or 21.5 umole/kg of the test conjugate intraperitoneally or intravenously. At a given time point between 5 minutes and 24 hours after administration of the compound the mice are sacrificed. Blood is collected in a syringe containing protease inhibitors such as EDTA, AEBSF, Aprotinin, Leupeptin, Bestatin, Pepstanin A or E64 and transferred into a heparinized blood collection tube. The plasma is prepared by centrifugation. The tumors are collected and pulverized in liquid nitrogen. The resulting tumor powders are stored at −80° C. The tumor powders and plasma are extracted and analyzed for the parent test conjugate and its products including Leucine-doxorubicin (or norleucine-doxorubicin, etc.) and doxorubicin.

Looking at the delivery of the toxin to the tumor cells, and also looking at the parent conjugate and the levels of toxin (dox and nor-leu dox) in the plasma.

Results

For example, test conjugate (21.5 umole/kg of Ac-Gly-Ser-Gly-Arg-Ser-nLeu-Dox (see Example 2)) was administered to naive and tumor bearing (TB) mice intraperitoneally (IP) or intravenously (IV). One hour after administration plasma and tumor tissue was collected from the mice. Concentrations of the test conjugate and its products are compared. The results show that the conjugate does not get into the tumor, the toxins (norleu dox and dox—μM concentrations in the tumor at one hour following the single (both IP and IV) injection. There were lower levels of dox and nor-leu dox the plasma than in the tumor.

Extraction, Chromatography LC/MS Conditions

Plasma: Plasma samples are prepared using acetonitrile protein precipitation. A standard curve was constructed from addition of 5 to 20 μL volumes of a standard compound to 0.1 mL or 0.05 mL volumes of plasma on ice. The standard curve ranges from 10 ng/mL-1 ug/mL or from 100 ng/mL-4 ug/mL of the standard compound. Immediumtely after standard addition, acetonitrile is added to precipitate the proteins. The study plasma samples were prepared by thawing the frozen plasma samples on ice. The aliquots were added directly to the acetonitrile. After sample precipitation, the sample is mixed using vortex mixing. The precipitate was pelleted using centrifugation. The supernatent was dried using vacuum centrifugation. The sample was reconstituted with 0.15 mL of 30% acetonitrile—70% (0.01 M ammonium acetate with 0.1% formic acid). 0.01 mL of the sample was injected for LC-MS analysis. The HPLC conditions were a linear gradient of 20% acetonitrile—80% (10 mM ammonium acetate—0.1% formic acid) to 50% acetonitrile—50% (10 mM ammonium acetate—0.1% formic acid) in 1 minute at 0.3 mL/min in a 30×2.1 mm Zorbax SB C18 HPLC column. Detection was provided by a triple quad mass spectrometer with electrospray ionization. Doxorubicin was monitored using the m/z transition 544.1-396.8. Leucine-doxorubicin was monitored using 657.2-242.8. An exemplary parent conjugate was monitored using 1555.9-1555.9. Scanning LC-MS and fluorescence detection was used to identify cleavage products other than doxorubicin or leucine-doxorubicin (or norleucine-doxorubicin, etc.) in the plasma.

Tumor: Immediately after excision from the mouse, the tumor for analysis is weighed and placed into a mortar containing liquid nitrogen. With the mortar nested in a bed of dry ice, the tumor is ground into a fine powder while additional liquid nitrogen is added as needed to avoid thawing. When a homogeneous tumor powder is achieved, the remaining liquid nitrogen is allowed to boil off. The tumor powder is quantitatively transferred to a 15 ml conical tube that has been pre-chilled and is on dry ice. The sample is stored at −70° C. until analysis. The tumor powder is thawed on ice and vortex mixed with 0.01 M ammonium acetate in a 1 gram tumor/mL ammonium acetate solution concentration to form a slurry. An aliquot of 0.1 mL of the tumor slurry is precipitated with 0.5 mL acetonitrile. The supernatant is separated from the precipitated solids and then evaporated using vacuum centrifugation. Quantification of doxorubicin, leucine-doxorubicin (or norleucine-docorubicin, etc.), is achieved by reference to a standard curve constructed from spiking measured amounts of standard compounds (doxorubicin, leucine-doxorubicin, etc.) into control tumor slurry. A typical standard curve ranges from 1 ng to 200 ng of compound per aliquot of tumor slurry. After the unknown samples and standards are processed and dried, the residue is reconstituted in 0.15 mL of 30% acetonitrile—70% (0.01 M ammonium acetate+0.1% formic acid). 10 μL of solution is injected onto a liquid chromatography—mass spectrometry system. The HPLC conditions were a linear gradient of 20% acetonitrile—80% (10 mM ammonium acetate—0.1% formic acid) to 50% acetonitrile—50% (10 mM ammonium acetate—0.1% formic acid) in 1 minute at 0.3 mL/min in a 30×2.1 mm Zorbax SB C18 HPLC column. Detection was provided by a triple quad mass spectrometer with electrospray ionization. Doxorubicin was monitored using the m/z transition 544.1-396.8. Leucine-doxorubicin was monitored using 657.2-242.8.

Since modifications will be apparent to those of skill in this art, it is intended that this invention be limited only by the scope of the appended claims.

What is claimed is:

1. A conjugate, comprising a therapeutic agent and a peptidic substrate linked thereto optionally via a linker, wherein the peptidic substrate is proteolytically cleaved by a cell surface protease or a soluble, released or shed form thereof, to liberate the therapeutic agent, wherein the conjugate is not substantially cleaved by plasmin or prostate specific antigen (PSA).

2. The conjugate of claim 1, wherein the liberated therapeutic agent is active.

3. The conjugate of claim 1, wherein cleavage liberates the therapeutic agent in a form that requires further processing for activation.

4. The conjugate of claim 1 that comprises the components: (peptidic substrate)ₛ, (Linker)_{q}, and (therapeutic agent)ₜ;
wherein at least one peptidic substrate moiety is linked with or without a linker to at least one therapeutic agent, s is 1 to 6, q is 0 to t, and t is 1 to 6, wherein a cell surface protease that cleaves the peptidic substrate(s) results in delivery of the therapeutic agent to the cell.

5. The conjugate of claim 1, wherein the peptidic substrate comprises one amino acid or more, wherein, upon proteolytic cleavage of the conjugate, the resulting therapeutic agent is active or in a form that, upon further processing, is active.

6. The conjugate of claim 1, wherein the cell surface protease is a serine protease.

7. The conjugate of claim 1, wherein the cell surface protease is a type II transmembrane serine protease (MTSP) or an endotheliase.

8. The conjugate of claim 1, wherein the cell surface protease is selected from endotheliase 1, endotheliase 2, MTSP1, MTSP3, MTSP4, MTSP6, MTSP7, MTSP9, MTSP10, MTSP12, MTSP20, MTSP22, MTSP25, corin, enterokinase, human airway trypsin-like protease (HAT), TMPRSS2, hepsin, urokinase-type plasminogen activator (uPA), and TMPRSS4.

9. The conjugate of claim 1, wherein the cell surface protease comprises a polypeptide selected from the group consisting of
a polypeptide comprising the sequence of amino acids set forth in any of SEQ ID Nos. 2, 4, 6, 8, 10, 12, 14, 16, 18, 20, 22, 24, 26, 29, 31, 33, 35, 37, 39, 41, 43, 45, 270, 272, 274 and 276;
a polypeptide encoded by a sequence of nucleotides that hybridizes under conditions of high stringency to the sequence of nucleotides set forth in any of SEQ ID Nos 1, 3, 5, 7, 9, 11, 13, 15, 17, 19, 21, 23, 25, 28, 30, 32, 34, 36, 38, 40, 42, 44, 269, 273 and 275;
a polypeptide that comprises a sequence of amino acids having at least about 40% sequence identity with the sequence of amino acids set forth in SEQ ID Nos. 2, 4, 6, 8, 10, 12, 14, 16, 18, 20, 22, 24, 26, 29, 31, 33, 35, 37, 39, 41, 43, 45, 270, 272, 274 and 276; and
a polypeptide encoded by a splice variant of the sequence of nucleotides set forth in any of SEQ ID Nos. 2, 4, 6, 8, 10, 12, 14, 16, 18, 20, 22, 24, 26, 29, 31, 33, 35, 37, 39, 41, 43, 45, 270, 272, 274 and 276.

10. The conjugate of claim 1, wherein the therapeutic agent is a toxin, a small organic molecule, a nucleic acid, protein therapeutic agents, a cytokine or a growth factor.

11. The conjugate of claim 1, wherein the therapeutic agent is an anti-cancer agent.

12. The conjugate of claim 1, wherein the therapeutic agent is an anti-angiogenic agent.

13. The conjugate of claim 1, wherein the therapeutic agent is selected from abrin, ricin A, pseudomonas exotoxin shiga toxin, diphtheria toxin, a tumor necrosis factor, α-interferon, γ-interferon, nerve growth factor, tissue factor and tissue factor variants, FAS-ligand platelet derived growth factor, tissue plasminogen activator, interleukin-1 (IL-1), interleukin-2 (IL-2), interleukin-6 (IL-6), granulocyte macrophage colony stimulating factor (GMCSF), granulocyte colony stimulating factor (G-CSF), erythropoietin (EPO), nerve growth factor, fibroblast growth factors (FGFs), and epidermal growth factor.

14. The conjugate of claim 1, wherein the therapeutic agent is selected from alkylating agents, toxins, antiproliferative agents, pro-apoptotic agents, pro-coagulants, cytotoxic nucleosides and tubulin binding agents.

15. The conjugate of claim 1, wherein the therapeutic agent is selected from among the following classes of drugs:
a) anthracycline family of drugs,
b) vinca alkaloid drugs,
c) mitomycins,
d) bleomycins,
e) cytotoxic nucleosides,
f) pteridine family of drugs.
g) diynenes,
h) estramustine,
i) cyclophosphamide,
j) taxanes,
k) podophyllotoxins,
l) maytansanoids,
m) epothilones, and
n) combretastatin and analogs,
or pharmaceutically acceptable derivatives thereof.

16. The conjugate of claim 1, wherein the therapeutic agent is selected from among the following drugs:
a) doxorubicin,
b) carminomycin,
c) daunorubicin,
d) aminopterin,
e) methotrexate,
f) methopterin,
g) dichloromethotrexate,
h) mitomycin C,
i) porfiromycin,
j) 5-fluorouracil,
k) 6-mercaptopurine,
l) cytosine arabinoside,
m) podophyllotoxin,
n) etoposide,
o) etoposide phosphate,
p) melphalan,
q) vinblastine,
r) vincristine,
s) leurosidine,
t) vindesine,
u) estramustine,
v) cisplatin,
w) cyclophosphamide,
x) taxol,
y) leurositte,
z) 4-desacetylvinblastine,
aa) epothilone B,
bb) taxotere,
cc) maytansanol,
dd) epothilone A, and
ee) combretastatin and analogs;
or a pharmaceutically acceptable derivative thereof.

17. The conjugate of claim 1, further comprising a linker between the therapeutic agent and the peptidic substrate.

18. The conjugate of claim 17, wherein the linker comprises a carbohydrate, peptide, and/or hydrocarbon core.

19. The conjugate of claim 17, wherein the linker comprises:
a biscarbonyl alkyl diradical whereby an amine moiety on the therapeutic agent is connected with the linker unit to form an amide bond and the amino terminus of the peptidic substrate is connected with the other end of the linker unit also forming an amide bond; or
a diaminoalkyl diradical linker unit, whereby a carbonyl moiety on the therapeutic agent is covalently attached to one of the amines of the linker unit while the other amine of the linker unit is covalently attached to the C-terminus of the peptidic substrate; or
is a self-eliminating linker of the following formulae: 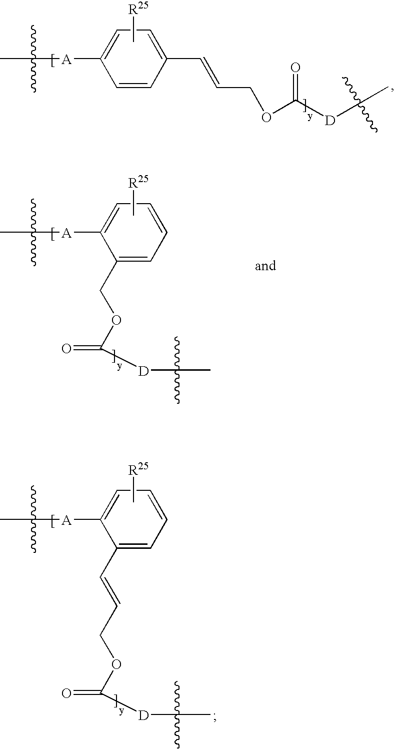
where A is NH or O; D is N(H or alkyl) or O; R²⁵ is H, alkyl, cycloalkyl, cycloalkylalkyl, aryl, heteroaryl optionally substituted with 1 or more, such as, for example, 1 to 3, substituents selected from halo, halo alkyl and alkyl, aralkyl, heteroaralkyl, alkenyl containing 1 to 2 double bonds, alkynyl containing 1 to 2 triple bonds, alk(en)(yn)yl groups, halo, pseudohalo, cyano, hydroxy, haloalkyl and polyhaloalkyl, such as, for example, halo lower alkyl, especially trifluoromethyl, formyl, alkylcarbonyl, arylcarbonyl that optionally is substituted with 1 or more, such as, for example, 1 to 3, substituents, for example, selected from halo, halo alkyl and alkyl, heteroarylcarbonyl, carboxy, alkoxycarbonyl, aryloxycarbonyl, aminoimino, alkoxycarbonylamino, aryloxycarbonylamino, aminocarbonyl, alkylaminocarbonyl, dialkylamino-carbonyl, arylaminocarbonyl, diarylaminocarbonyl, aralkylaminocarbonyl, alkoxy, aryloxy, perfluoroalkoxy, alkenyloxy, alkynyloxy, arylalkoxy, aminoalkyl, alkylaminoalkyl, dialkylaminoalkyl, arylaminoalkyl, amino, alkylamino, dialkylamino, arylamino, alkylarylamino, alkylcarbonylamino, arylcarbonylamino, azido, nitro, mercapto, alkylthio, arylthio, perfluoroalkylthio, thiocyano, isothiocyano, alkylsulfinyl, alkylsulfonyl, arylsulfinyl, arylsulfonyl, aminosulfonyl, alkylaminosulfonyl, dialkylaminosulfonyl and arylaminosulfonyl; and y is an integer from 1 to 3.

20. The conjugate of claim 17, wherein the linker is a diamine comprising a cyclic alkylene moiety.

21. The conjugate of claim 17, wherein the diamine contains a bicycloalkylene moiety.

22. The conjugate of claim 17, wherein the linker selected from 1,4-bis(aminomethyl)cyclohexane, 1,4-bis(aminomethyl)cycloheptane, 1,3-bis(aminomethyl)cyclopentane, 1-amino-4-(aminomethyl)cyclohexane, 1,4-diaminocyclohexane and 1,4-bis(aminomethyl)bicyclo[2.2.2]octane.

23. The conjugate of claim 17, wherein the linker is a 1,ω-diaminoalkane.

24. The conjugate of claim 17, wherein the linker is a 1,3-diaminopropane.

25. The conjugate of claim 17, wherein the linker is a 1,ω-dicarbonylalkane.

26. The conjugate of claim 25, wherein the linker selected from oxalic, malonic, succinic, glutaric, adipic and pivalic acids.

27. The conjugate of claim 1, wherein the peptidic substrate comprises P1 that is any amino acid.

28. The conjugate of claim 27, wherein P1 is a naturally-occurring amino acid.

29. The conjugate of claim 27, wherein P1 is an amino acid with an aromatic, branched, or branched aromatic side chain.

30. The conjugate of claim 1, wherein the peptidic substrate comprises P1, where P1 is selected from among Arg, Lys, Tyr, Phe, Trp, Ala, Val, Ile and Thr.

31. The conjugate of claim 1, wherein:
the peptidic substrate comprises a P1-P1′ bond;
the P1-P1′ bond is the site of cleavage by a cell surface protease;
P1 is selected from Arg, Lys, Tyr, Phe, Trp, Ala, Val, Ile and Thr; and
P1′ is Gly, Ser, Ala, Leu, Ile, d-Ile, nLeu, Val, nVal, Aib, Abu, Met or 6-aminohexanoyl.

32. The conjugate of claim 1, wherein the peptidic substrate comprises P1, wherein P1 is Arg, Lys or an Arg surrogate.

33. The conjugate of claim 1, further comprising a P2 residue selected from Phe, Ser, Gly and Ala.

34. The conjugate of claim 1, further comprising a P3 residue selected from Arg, Lys, Gln, Ser, Quat and Arg surrogates.

35. The conjugate of claim 1, further comprising a P4 residue selected from Pro, Arg, Ser, Ala, Lys, Gly, nLeu, Leu, Tyr, Glu, Phe and Val.

36. The conjugate of claim 1, further comprising a P5 residue selected from Arg and Arg surrogates.

37. The conjugate of claim 1, further comprising a P6 residue selected from Leu, Ile and Val.

38. The conjugate of claim 1, further comprising a P2′ residue selected from Gly, Ser, Ala, Leu, Ile, d-Ile, nLeu, Val, nVal, Aib, Abu, Met and 6-aminohexanoyl.

39. The conjugate of claim 1, further comprising a P3′ residue selected from Gly, Ser, Ala, Leu, Ile, nLeu, Val, nVal, Aib, Abu, Met and 6-aminohexanoyl.

40. The conjugate of claim 1, wherein:
the peptidic substrate comprises a 5-mer that has the formula:
P4-P3-P2-P1-P1′, wherein:
P1 is selected from among Arg, Lys, Tyr, Phe, Trp, Ala, Val, Ile and Thr
P2 is selected from Phe, Ser, Gly and Ala;
P3 is selected from Arg, Lys, Gln, Quat and Arg surrogates;
P4 is selected from Pro, Arg, Ser, Ala, Lys, Gly, nLeu, Leu, Tyr, Glu, Phe and Val; and
P1′ is Gly, Ser, Ala, Leu, Ile, d-Ile, nLeu, Val, nVal, Aib, Abu, Met or 6-aminohexanoyl.

41. The conjugate of claim 40, wherein:
the peptidic substrate optionally further comprises one or more of a P5 or P2′ amino acid residue, wherein:
P5 is Arg or an Arg surrogate; and
P2′ is selected from among Gly, Ser, Ala, Leu, Ile, d-Ile, nLeu, Val, nVal, Aib, Abu, Met and 6-aminohexanoyl.

42. The conjugate of claim 41, wherein:
if the peptidic substrate comprises a P5 amino acid residue, then the peptidic substrate optionally further comprises a P6 amino acid residue selected from Leu, Ile and Val; and
if the peptidic substrate comprises a P2′ amino acid residue, then the peptidic substrate optionally further comprises a P3′ amino acid residue selected from Gly, Ser, Ala, Leu, Ile, nLeu, Val, nVal, Aib, Abu, Met and 6-aminohexanoyl.

43. The conjugate of claim 1, wherein:
the therapeutic agent is conjugated directly or via a linker to the C terminus of the peptidic substrate.

44. The conjugate of claim 1, wherein:
the peptidic substrate comprises a cap at the N-terminus.

45. The conjugate of claim 1, wherein the cap is a hydrophilic blocking group.

46. The conjugate of claim 1, wherein the cap is an acyl, sulfonyl or carbamoyl derivative.

47. The conjugate of claim 45, wherein the blocking group is selected from among hydroxylated alkanoyls, polyhydroxylated alkanoyls, polyethylene glycols, glycosylates, sugars and crown ethers.

48. The conjugate of claim 43 that has formula I:
Xⁿ-(P6)ₘ-(P5)ₚ-(P4)ₗ-(P3)ⱼ-(P2)ᵢ-P1-(P1′)ᵤ-(P2′)ₖ-(P3′)ᵣ-(L)ₙ-Z
or a derivative thereof, wherein:
Z is a therapeutic agent;
L is a linker;
l, j, i, p and m are selected as follows:
l is 0 or 1; when l is 0, j, i, p and m are 0; when l is 1, j is 0 or 1; when j is 0, i, p and m are 0; when j is 1, i is 0 or 1; when i is 0, p and m are 0; when i is 1, p is 0 or 1; when p is 0, m is 0; when p is 1, m is 0 or 1;
u, k and r are selected as follows:
u is 0 or 1; when u is 0, k and r are 0; when u is 1, k is 0 or 1; when k is 0, r is 0; when k is 1, r is 0 or 1;
n is 0 or 1;
Xⁿ is hydrogen, or an acyl, sulfonyl or carbamoyl cap;
P1 is selected from among Arg, Lys, Tyr, Phe, Trp, Ala, Val, Ile and Thr;
P1′ is Gly, Ser, Ala, Leu, Ile, d-Ile, nLeu, Val, nVal, Aib, Abu, Met or 6-aminohexanoyl;
P2 is selected from Phe, Ser, Gly and Ala;
P3 is selected from Arg, Lys, Gln, Ser, Quat and Arg surrogates;
P4 is selected from Pro, Arg, Ser, Ala, Lys, Gly, nLeu, Leu, Tyr, Glu, Phe and Val;
P5 is selected from Arg and Arg surrogates;
P6 is selected from Leu, Ile and Val;
P2′ is selected from Gly, Ser, Ala, Leu, Ile, d-Ile, nLeu, Val, nVal, Aib, Abu, Met and 6-aminohexanoyl; and
P3′ is selected from Gly, Ser, Ala, Leu, Ile, nLeu, Val, nVal, Aib, Abu, Met and 6-aminohexanoyl.

49. The conjugate of claim 48, wherein P1 is Arg, Lys or an Arg surrogate.

50. The conjugate of claim 1, wherein:
the therapeutic agent is conjugated directly or via a linker to the N terminus of the peptidic substrate.

51. The conjugate of claim 50, wherein:
the C-terminus of the peptidic substrate is a carboxylic acid or a carboxamide derivative.

52. The conjugate of claim 50 that has formula II:
Z-(L)ₙ-(P6)ₘ-(P5)ₚ-(P4)ᵢ-(P3)ⱼ-(P2)ₗ-P1-(P1′)ᵤ-(P2′)ₖ-(P3′)ᵣ-X^{c}
or a derivative thereof, wherein:
Z is a therapeutic agent;
L is a linker;
l, j, i, p and m are selected as follows:
l is 0 or 1; when l is 0, j, i, p and m are 0; when l is 1, j is 0 or 1; when j is 0, i, p and m are 0; when j is 1, i is 0 or 1; when i is 0, p and m are 0; when i is 1, p is 0 or 1; when p is 0, m is 0; when p is 1, m is 0 or 1;
u, k and r are selected as follows:
u is 0 or 1; when u is 0, k and r are 0; when u is 1, k is 0 or 1; when k is 0, r is 0; when k is 1, r is 0 or 1;
n is 0 or 1;
X^{c}, together with the carbonyl group of the amino acid residue to which it is attached, forms a carboxylic acid or a carboxamide group;
P1 is selected from among Arg, Lys, Tyr, Phe, Trp, Ala, Val, Ile and Thr;
P1′ is Gly, Ser, Ala, Leu, Ile, d-Ile, nLeu, Val, nVal, Aib, Abu, Met or 6-aminohexanoyl;
P2 is selected from Phe, Ser, Gly and Ala;
P3 is selected from Arg, Lys, Gln, Ser, Quat and Arg surrogates;
P4 is selected from Pro, Arg, Ser, Ala, Lys, Gly, nLeu, Leu, Tyr, Glu, Phe and Val;
P5 is selected from Arg and Arg surrogates;
P6 is selected from Leu, Ile and Val;
P2′ is selected from Gly, Ser, Ala, Leu, Ile, d-Ile, nLeu, Val, nVal, Aib, Abu, Met and 6-aminohexanoyl; and
P3′ is selected from Gly, Ser, Ala, Leu, Ile, nLeu, Val, nVal, Aib, Abu, Met and 6-aminohexanoyl.

53. The conjugate of claim 52, wherein P1 is Arg, Lys or an Arg surrogate.

54. The conjugate of claim 1, wherein a first therapeutic agent is attached, optionally via a first linker, to the N-terminus of the peptidic substrate; and
a second therapeutic agent, which are the same or different from the first therapeutic agent, is attached, optionally via a second linker, which are the same or different from the first linker, to the C-terminus of the peptidic substrate.

55. The conjugate of claim 54 that has formula III:
Z¹-(L¹)ₙ-(P6)ₘ-(P5)ₚ-(P4)ᵢ-(P3)ⱼ-(P2)ₗ-P1-(P1′)ᵤ-(P2′)ₖ-(P3′)ᵣ-(L²)ᵥ-Z²
or a derivative thereof, wherein:
Z¹ and Z² are each therapeutic agents and are the same or different;
L¹ and L² are each linkers and are the same or different;
l, j, i, p and m are selected as follows:
l is 0 or 1; when l is 0, j, i, p and m are 0; when l is 1, j is 0 or 1; when j is 0, i, p and m are 0; when j is 1, i is 0 or 1; when i is 0, p and m are 0; when i is 1, p is 0 or 1; when p is 0, m is 0; when p is 1, m is 0 or 1;
u, k and r are selected as follows:
u is 0 or 1; when u is 0, k and r are 0; when u is 1, k is 0 or 1; when k is 0, r is 0; when k is 1, r is 0 or 1;
n and v are each independently 0 or 1;
P1 is selected from among Arg, Lys, Tyr, Phe, Trp, Ala, Val, Ile and Thr;
P1′ is Gly, Ser, Ala, Leu, Ile, d-Ile, nLeu, Val, nVal, Aib, Abu, Met or 6-aminohexanoyl;
P2 is selected from Phe, Ser, Gly and Ala;
P3 is selected from Arg, Lys, Gln, Ser, Quat and Arg surrogates;
P4 is selected from Pro, Arg, Ser, Ala, Lys, Gly, nLeu, Leu, Tyr, Glu, Phe and Val;
P5 is selected from Arg and Arg surrogates;
P6 is selected from Leu, Ile and Val;
P2′ is selected from Gly, Ser, Ala, Leu, Ile, d-Ile, nLeu, Val, nVal, Aib, Abu, Met and 6-aminohexanoyl; and
P3′ is selected from Gly, Ser, Ala, Leu, Ile, nLeu, Val, nVal, Aib, Abu, Met and 6-aminohexanoyl.

56. The conjugate of claim 55, wherein P1 is Arg, Lys or an Arg surrogate.

57. The conjugate of claim 1, selected from:
Ac-Leu-Arg-Ala-Quat-Gly-Arg-Ala-Ala-(therapeutic agent) (SEQ ID NO: 46);
Ac-Leu-Arg-Ala-Quat-Ala-Arg-Ala-Ala-(therapeutic agent) (SEQ ID NO: 47);
Ac-Leu-Arg-Ser-Quat-Gly-Arg-Ala-Ala-(therapeutic agent) (SEQ ID NO: 48);
Ac-Leu-Arg-Ser-Quat-Ala-Arg-Ala-Ala-(therapeutic agent) (SEQ ID NO: 49);
Ac-Leu-Arg-Pro-Arg-Phe-Lys-Ile-Ile-(therapeutic agent) (SEQ ID NO: 50);
Ac-Arg-Pro-Arg-Phe-Lys-Ile-Ile-(therapeutic agent) (SEQ ID NO: 51);
Ac-Pro-Arg-Phe-Lys-Ile-Ile-(therapeutic agent) (SEQ ID NO: 52);
Ac-Leu-Arg-Ser-Lys-Ser-Arg-Ala-Ala-(therapeutic agent) (SEQ ID NO: 53);
Ac-Arg-Ser-Lys-Ser-Arg-Ala-Ala-(therapeutic agent) (SEQ ID NO: 54);
Ac-Ser-Lys-Ser-Arg-Ala-Ala-(therapeutic agent) (SEQ ID NO: 55);
Ac-Leu-Arg-Pro-Arg-Phe-Arg-Ile-Ile-(therapeutic agent) (SEQ ID NO: 56);
Ac-Arg-Pro-Arg-Phe-Arg-Ile-Ile-(therapeutic agent) (SEQ ID NO:57);
Ac-Pro-Arg-Phe-Arg-Ile-Ile-(therapeutic agent) (SEQ ID NO: 58);
Ac-Leu-Arg-Ser-Arg-Ser-Arg-Ala-Ala-(therapeutic agent) (SEQ ID NO: 59);
Ac-Arg-Ser-Arg-Ser-Arg-Ala-Ala-(therapeutic agent) (SEQ ID NO: 60);
Ac-Ser-Arg-Ser-Arg-Ala-Ala-(therapeutic agent) (SEQ ID NO: 61);
Ac-Leu-Arg-Ala-Quat-Gly-Arg-Ala-Ala-(therapeutic agent) (SEQ ID NO: 62);
Ac-Leu-Arg-Ala-Quat-Ala-Arg-Ala-Ala-(therapeutic agent) (SEQ ID NO: 63);
Ac-Leu-Arg-Ser-Quat-Gly-Arg-Ala-Ala-(therapeutic agent) (SEQ ID NO: 64);
Ac-Leu-Arg-Ser-Quat-Ala-Arg-Ala-Ala-(therapeutic agent) (SEQ ID NO: 65);
Ac-Leu-Arg-Pro-Arg-Phe-Lys-Ile-Ile-(therapeutic agent) (SEQ ID NO: 66);
Ac-Arg-Pro-Arg-Phe-Lys-Ile-Ile-(therapeutic agent) (SEQ ID NO: 67);
Ac-Pro-Arg-Phe-Lys-Ile-Ile-(therapeutic agent) (SEQ ID NO: 68);
Ac-Leu-Arg-Ser-Lys-Ser-Arg-Ala-Ala-(therapeutic agent) (SEQ ID NO: 69);
Ac-Arg-Ser-Lys-Ser-Arg-Ala-Ala-(therapeutic agent) (SEQ ID NO: 70);
Ac-Ser-Lys-Ser-Arg-Ala-Ala-(therapeutic agent) (SEQ ID NO: 71);
Ac-Leu-Arg-Pro-Arg-Phe-Arg-Ile-Ile-(therapeutic agent) (SEQ ID NO: 72);
Ac-Arg-Pro-Arg-Phe-Arg-Ile-Ile-(therapeutic agent) (SEQ ID NO: 73);
Ac-Pro-Arg-Phe-Arg-Ile-Ile-(therapeutic agent) (SEQ ID NO: 74);
Ac-Leu-Arg-Ser-Arg-Ser-Arg-Ala-Ala-(therapeutic agent) (SEQ ID NO: 75);
Ac-Arg-Ser-Arg-Ser-Arg-Ala-Ala-(therapeutic agent) (SEQ ID NO: 76); and
Ac-Ser-Arg-Ser-Arg-Ala-Ala-(therapeutic agent) (SEQ ID NO: 77)
pyroGlu-Pro-Arg-Ala-Ala-(therapeutic agent) (SEQ ID NO: 78);
CH₃SO₂-D-HHT-Gly-Arg-Ala-Ala-(therapeutic agent) (SEQ ID NO: 79);
N-p-tosyl-GIy-Pro-Arg-Ala-Ala-(therapeutic agent) (SEQ ID NO: 80);
BenzoyI-Val-Gly-Arg-Ala-Ala-(therapeutic agent) (SEQ ID NO: 81);
CH₃SO₂-D-HHT-Gly-Arg-Ala-Ala-(therapeutic agent) (SEQ ID NO: 82);
N-α-Z-D-Arg-Gly-Arg-Ala-Ala-(therapeutic agent) in which Z is benzyloxycarbonyl (SEQ ID NO: 83);
pyroGlu-Gly-Arg-Ala-Ala-(therapeutic agent) (SEQ ID NO: 84);
H-D-Ile-Pro-Arg-Ala-Ala-(therapeutic agent) (SEQ ID NO: 85);
Cbo-L-(γ)Glu(α-t-BuO)-Gly-Arg-Ala-Ala-(therapeutic agent) in which Cbo is carbobenzoxy (SEQ ID NO: 86);
H-D-Pro-Phe-Arg-Ala-Ala-(therapeutic agent) (SEQ ID NO: 87);
H-D-Val-Leu-Arg-Ala-Ala-(therapeutic agent) (SEQ ID NO: 88);
Bz-Ile-Glu(γ-OH)-Gly-Arg-Ala-Ala-(therapeutic agent) in which Bz is benzoyl (SEQ ID NO: 89);
Bz-Ile-Glu(γ-OMe)-Gly-Arg-Ala-Ala-(therapeutic agent) (SEQ ID NO: 90);
Bz-Pro-Phe-Arg-Ala-Ala-(therapeutic agent) (SEQ ID NO: 91);
H-D-Phe-Pip-Arg-Ala-Ala-(therapeutic agent) (SEQ ID NO: 92);
H-D-Val-Leu-Lys-Ala-Ala-(therapeutic agent) (SEQ ID NO: 93);
H-D-Nle-HHT-Lys-Ala-Ala-(therapeutic agent) (SEQ ID NO: 94);
Pyr-Arg-Thr-Lys-Arg-Ala-Ala-(therapeutic agent) (SEQ ID NO: 95);
H-Arg-Gln-Arg-Arg-Ala-Ala-(therapeutic agent) (SEQ ID NO: 96);
Boc-Gln-Gly-Arg-Ala-Ala-(therapeutic agent) (SEQ ID NO: 97);
Z-Arg-Arg-Ala-Ala-(therapeutic agent) (SEQ ID NO: 98);
H-D-HHT-Ala-Arg-Ala-Ala-(therapeutic agent) (SEQ ID NO: 99);
H-D-CHT-Gly-Arg-Ala-Ala-(therapeutic agent) (SEQ ID NO: 100);
MeSO₂-dPhe-Pro-Arg-Ala-Ala-(therapeutic agent) (SEQ ID NO: 101);
δ-Z-D-Lys-Pro-Arg-Ala-Ala-(therapeutic agent) (SEQ ID NO: 102);
CH₃SO₂-D-CHA-But-Arg-Ala-Ala-(therapeutic agent) (SEQ ID NO: 103);
Ac-Arg-Gln-Ser-Arg-Ala-Ala-(therapeutic agent) (SEQ ID NO: 104);
Ac-Arg-Arg-Gln-Ser-Arg-Ala-Ala-(therapeutic agent) (SEQ ID NO: 105);
Ac-Leu-Arg-Arg-Gln-Ser-Arg-Ala-Ala-(therapeutic agent) (SEQ ID NO: 106);
Ac-Arg-Gln-Ser-Arg-Ala-(therapeutic agent) (SEQ ID NO: 107);
Ac-Arg-Arg-Gln-Ser-Arg-Ala-(therapeutic agent) (SEQ ID NO: 108);
Ac-Leu-Arg-Arg-Gln-Ser-Arg-Gly-Gly-(therapeutic agent) (SEQ ID NO: 109);
Ac-Leu-Arg-Arg-Gln-Ser-Arg-Ala-(therapeutic agent) (SEQ ID NO: 110);
Ac-Arg-Arg-Gln-Ser-Arg-Ile-(therapeutic agent) (SEQ ID NO: 111);
Ac-Leu-Arg-Arg-Gln-Ser-Arg-Ala-Ile-(therapeutic agent) (SEQ ID NO: 112);
Ac-Leu-Arg-Ala-Quat-Gly-Arg-Ser-Leu-(therapeutic agent) (SEQ ID NO: 113);
Ac-Leu-Arg-Ala-Quat-Ala-Arg-Ser-Leu-(therapeutic agent) (SEQ ID NO: 114);
Ac-Leu-Arg-Ser-Quat-Gly-Arg-Ser-Leu-(therapeutic agent) (SEQ ID NO: 115);
Ac-Leu-Arg-Ser-Quat-Ala-Arg-Ser-Leu-(therapeutic agent) (SEQ ID NO: 116);
Ac-Leu-Arg-Pro-Arg-Phe-Lys-Ser-Leu-(therapeutic agent) (SEQ ID NO: 117);
Ac-Arg-Pro-Arg-Phe-Lys-Ser-Leu-(therapeutic agent) (SEQ ID NO: 118);
Ac-Pro-Arg-Phe-Lys-Ser-Leu-(therapeutic agent) (SEQ ID NO: 11 9);
Ac-Leu-Arg-Ser-Lys-Ser-Arg-Ser-Leu-(therapeutic agent) (SEQ ID NO: 120);
Ac-Arg-Ser-Lys-Ser-Arg-Ser-Leu-(therapeutic agent) (SEQ ID NO: 121);
Ac-Ser-Lys-Ser-Arg-Ser-Leu-(therapeutic agent) (SEQ ID NO: 122);
Ac-Leu-Arg-Pro-Arg-Phe-Arg-Ser-Leu-(therapeutic agent) (SEQ ID NO: 123);
Ac-Arg-Pro-Arg-Phe-Arg-Ser-Leu-(therapeutic agent) (SEQ ID NO: 124);
Ac-Pro-Arg-Phe-Arg-Ser-Leu-(therapeutic agent) (SEQ ID NO: 125);
Ac-Leu-Arg-Ser-Arg-Ser-Arg-Ser-Leu-(therapeutic agent) (SEQ ID NO: 126);
Ac-Arg-Ser-Arg-Ser-Arg-Ser-Leu-(therapeutic agent) (SEQ ID NO: 127);
Ac-Ser-Arg-Ser-Arg-Ser-Leu-(therapeutic agent) (SEQ ID NO: 128);
Ac-Leu-Arg-Ala-Quat-Gly-Arg-Ser-Leu-(therapeutic agent) (SEQ ID NO: 129);
Ac-Leu-Arg-Ala-Quat-Ala-Arg-Ser-Leu-(therapeutic agent) (SEQ ID NO: 130);
Ac-Leu-Arg-Ser-Quat-Gly-Arg-Ser-Leu-(therapeutic agent) (SEQ ID NO: 131);
Ac-Leu-Arg-Ser-Quat-Ala-Arg-Ser-Leu-(therapeutic agent) (SEQ ID NO: 132);
Ac-Leu-Arg-Pro-Arg-Phe-Lys-Ser-Leu-(therapeutic agent) (SEQ ID NO: 133);
Ac-Arg-Pro-Arg-Phe-Lys-Ser-Leu-(therapeutic agent) (SEQ ID NO: 134);
Ac-Pro-Arg-Phe-Lys-Ser-Leu-(therapeutic agent) (SEQ ID NO: 135);
Ac-Leu-Arg-Ser-Lys-Ser-Arg-Ser-Leu-(therapeutic agent) (SEQ ID NO: 136);
Ac-Arg-Ser-Lys-Ser-Arg-Ser-Leu-(therapeutic agent) (SEQ ID NO: 137);
Ac-Ser-Lys-Ser-Arg-Ser-Leu-(therapeutic agent) (SEQ ID NO: 138);
Ac-Leu-Arg-Pro-Arg-Phe-Arg-Ser-Leu-(therapeutic agent) (SEQ ID NO: 139);
Ac-Arg-Pro-Arg-Phe-Arg-Ser-Leu-(therapeutic agent) (SEQ ID NO: 140);
Ac-Pro-Arg-Phe-Arg-Ser-Leu-(therapeutic agent) (SEQ ID NO: 141);
Ac-Leu-Arg-Ser-Arg-Ser-Arg-Ser-Leu-(therapeutic agent) (SEQ ID NO: 142);
Ac-Arg-Ser-Arg-Ser-Arg-Ser-Leu-(therapeutic agent) (SEQ ID NO: 143);
Ac-Ser-Arg-Ser-Arg-Ser-Leu-(therapeutic agent) (SEQ ID NO: 144);
pyroGlu-Pro-Arg-Ser-Leu-(therapeutic agent) (SEQ ID NO: 145);
CH₃SO₂-D-HHT-Gly-Arg-Ser-Leu-(therapeutic agent) (SEQ ID NO: 146);
N-p-tosyl-Gly-Pro-Arg-Ser-Leu-(therapeutic agent) (SEQ ID NO: 147);
Benzoyl-Val-Gly-Arg-Ser-Leu-(therapeutic agent) (SEQ ID NO: 148);
CH₃SO₂-D-HHT-Gly-Arg-Ser-Leu-(therapeutic agent) (SEQ ID NO: 149);
N-α-Z-D-Arg-Gly-Arg-Ser-Leu-(therapeutic agent) (SEQ ID NO: 150) (Z=benzyloxycarbonyl);
pyroGlu-Gly-Arg-Ser-Leu-(therapeutic agent) (SEQ ID NO: 151);
H-D-Ile-Pro-Arg-Ser-Leu-(therapeutic agent) (SEQ ID NO: 152);
Cbo-L-(γ)Glu(α-t-BuO)-Gly-Arg-Ser-Leu-(therapeutic agent) (SEQ ID NO: 153) (Cbo=carbobenzoxy);
H-D-Pro-Phe-Arg-Ser-Leu-(therapeutic agent) (SEQ ID NO: 154);
H-D-Val-Leu-Arg-Ser-Leu-(therapeutic agent) (SEQ ID NO: 155);
Bz-Ile-Glu(γ-OH)-Gly-Arg-Ser-Leu-(therapeutic agent) (SEQ ID NO: 156) (Bz=benzoyl);
Bz-Ile-Glu(γ-OMe)-Gly-Arg-Ser-Leu-(therapeutic agent) (SEQ ID NO: 157);
Benzoyl-Pro-Phe-Arg-Ser-Leu-(therapeutic agent) (SEQ ID NO: 158);
H-D-Phe-Pip-Arg-Ser-Leu-(therapeutic agent) (SEQ ID NO: 159);
H-D-Val-Leu-Lys-Ser-Leu-(therapeutic agent) (SEQ ID NO: 160);
H-D-Nle-HHT-Lys-Ser-Leu-(therapeutic agent) (SEQ ID NO: 161);
Pyr-Arg-Thr-Lys-Arg-Ser-Leu-(therapeutic agent) (SEQ ID NO: 162);
H-Arg-Gln-Arg-Arg-Ser-Leu-(therapeutic agent) (SEQ ID NO: 163);
Boc-Gln-Gly-Arg-Ser-Leu-(therapeutic agent) (SEQ ID NO: 164);
Z-Arg-Arg-Ser-Leu-(therapeutic agent) (SEQ ID NO: 165);
H-D-HHT-Ala-Arg-Ser-Leu-(therapeutic agent) (SEQ ID NO: 166);
H-D-CHT-Gly-Arg-Ser-Leu-(therapeutic agent) (SEQ ID NO: 167);
MeSO₂-dPhe-Pro-Arg-Ser-Leu-(therapeutic agent) (SEQ ID NO: 168);
δ-Z-D-Lys-Pro-Arg-Ser-Leu-(therapeutic agent) (SEQ ID NO: 169);
CH₃SO₂-D-CHA-But-Arg-Ser-Leu-(therapeutic agent) (SEQ ID NO: 170);
Ac-Arg-Gln-Ser-Arg-Ser-Leu-(therapeutic agent) (SEQ ID NO: 171);
Ac-Arg-Arg-Gln-Ser-Arg-Ser-Leu-(therapeutic agent) (SEQ ID NO: 172);
Ac-Leu-Arg-Arg-Gln-Ser-Arg-Ser-Leu-(therapeutic agent) (SEQ ID NO: 173);
Ac-Arg-Gln-Ser-Arg-Leu-(therapeutic agent) (SEQ ID NO: 174);
Ac-Arg-Arg-Gln-Ser-Arg-Leu-(therapeutic agent) (SEQ ID NO: 175);
Ac-Leu-Arg-Arg-Gln-Ser-Arg-Ser-Leu-(therapeutic agent) (SEQ ID NO: 176);
Ac-Leu-Arg-Arg-Gln-Ser-Arg-Leu-(therapeutic agent) (SEQ ID NO: 177);
Ac-Arg-Arg-Gln-Ser-Arg-Leu-(therapeutic agent) (SEQ ID NO: 178);
Ac-Leu-Arg-Arg-Gln-Ser-Arg-Ser-Leu-(therapeutic agent) (SEQ ID NO: 179);
Ac-Arg-Gln-Gly-Arg-Ser-Leu-(therapeutic agent) (SEQ ID NO: 180);
Ac-Arg-Gln-Ala-Arg-Ser-Leu-(therapeutic agent) (SEQ ID NO: 181);
Ac-Arg-Gln-Phe-Arg-Ser-Leu-(therapeutic agent) (SEQ ID NO: 182);
Ac-Arg-Ser-Arg-Ser-Leu-(therapeutic agent) (SEQ ID NO: 183);
Ac-Arg-Gly-Arg-Ser-Leu-(therapeutic agent) (SEQ ID NO: 184);
Ac-Arg-Ala-Arg-Ser-Leu-(therapeutic agent) (SEQ ID NO: 185);
Ac-Arg-Phe-Arg-Ser-Leu-(therapeutic agent) (SEQ ID NO: 186);
Ac-Gln-Ser-Arg-Ser-Leu-(therapeutic agent) (SEQ ID NO: 187);
Ac-Gln-Gly-Arg-Ser-Leu-(therapeutic agent) (SEQ ID NO: 188);
Ac-Gln-Ala-Arg-Ser-Leu-(therapeutic agent) (SEQ ID NO: 189);
Ac-Gln-Phe-Arg-Ser-Leu-(therapeutic agent) (SEQ ID NO: 190);
Ac-Leu-Arg-Ala-Quat-Gly-Arg-Ser-Ser-Leu-(therapeutic agent) (SEQ ID NO: 191);
Ac-Leu-Arg-Ala-Quat-Ala-Arg-Ser-Ser-Leu-(therapeutic agent) (SEQ ID NO: 192);
Ac-Leu-Arg-Ser-Quat-Gly-Arg-Ser-Ser-Leu-(therapeutic agent) (SEQ ID NO: 193);
Ac-Leu-Arg-Ser-Quat-Ala-Arg-Ser-Ser-Leu-(therapeutic agent) (SEQ ID NO: 194);
Ac-Leu-Arg-Pro-Arg-Phe-Lys-Ser-Ser-Leu-(therapeutic agent) (SEQ ID NO: 195);
Ac-Arg-Pro-Arg-Phe-Lys-Ser-Ser-Leu-(therapeutic agent) (SEQ ID NO: 196);
Ac-Pro-Arg-Phe-Lys-Ser-Ser-Leu-(therapeutic agent) (SEQ ID NO: 197);
Ac-Leu-Arg-Ser-Lys-Ser-Arg-Ser-Ser-Leu-(therapeutic agent) (SEQ ID NO: 198);
Ac-Arg-Ser-Lys-Ser-Arg-Ser-Ser-Leu-(therapeutic agent) (SEQ ID NO: 199);
Ac-Ser-Lys-Ser-Arg-Ser-Ser-Leu-(therapeutic agent) (SEQ ID NO: 200);
Ac-Leu-Arg-Pro-Arg-Phe-Arg-Ser-Ser-Leu-(therapeutic agent) (SEQ ID NO: 201);
Ac-Arg-Pro-Arg-Phe-Arg-Ser-Ser-Leu-(therapeutic agent) (SEQ ID NO: 202);
Ac-Pro-Arg-Phe-Arg-Ser-Ser-Leu-(therapeutic agent) (SEQ ID NO: 203);
Ac-Leu-Arg-Ser-Arg-Ser-Arg-Ser-Ser-Leu-(therapeutic agent) (SEQ ID NO: 204);
Ac-Arg-Ser-Arg-Ser-Arg-Ser-Ser-Leu-(therapeutic agent) (SEQ ID NO: 205);
Ac-Ser-Arg-Ser-Arg-Ser-Ser-Leu-(therapeutic agent) (SEQ ID NO: 206);
Ac-Leu-Arg-Ala-Quat-Gly-Arg-Ser-Ser-Leu-(therapeutic agent) (SEQ ID NO: 207);
Ac-Leu-Arg-Ala-Quat-Ala-Arg-Ser-Ser-Leu-(therapeutic agent) (SEQ ID NO: 208);
Ac-Leu-Arg-Ser-Quat-Gly-Arg-Ser-Ser-Leu-(therapeutic agent) (SEQ ID NO: 209);
Ac-Leu-Arg-Ser-Quat-Ala-Arg-Ser-Ser-Leu-(therapeutic agent) (SEQ ID NO: 210);
Ac-Leu-Arg-Pro-Arg-Phe-Lys-Ser-Ser-Leu-(therapeutic agent) (SEQ ID NO: 211);
Ac-Arg-Pro-Arg-Phe-Lys-Ser-Ser-Leu-(therapeutic agent) (SEQ ID NO: 212);
Ac-Pro-Arg-Phe-Lys-Ser-Ser-Leu-(therapeutic agent) (SEQ ID NO: 213);
Ac-Leu-Arg-Ser-Lys-Ser-Arg-Ser-Ser-Leu-(therapeutic agent) (SEQ ID NO: 214);
Ac-Arg-Ser-Lys-Ser-Arg-Ser-Ser-Leu-(therapeutic agent) (SEQ ID NO: 215);
Ac-Ser-Lys-Ser-Arg-Ser-Ser-Leu-(therapeutic agent) (SEQ ID NO: 216);
Ac-Leu-Arg-Pro-Arg-Phe-Arg-Ser-Ser-Leu-(therapeutic agent) (SEQ ID NO: 217);
Ac-Arg-Pro-Arg-Phe-Arg-Ser-Ser-Leu-(therapeutic agent) (SEQ ID NO: 218);
Ac-Pro-Arg-Phe-Arg-Ser-Ser-Leu-(therapeutic agent) (SEQ ID NO: 219);
Ac-Leu-Arg-Ser-Arg-Ser-Arg-Ser-Ser-Leu-(therapeutic agent) (SEQ ID NO: 220);
Ac-Arg-Ser-Arg-Ser-Arg-Ser-Ser-Leu-(therapeutic agent) (SEQ ID NO: 221);
Ac-Ser-Arg-Ser-Arg-Ser-Ser-Leu-(therapeutic agent) (SEQ ID NO: 222);
pyroGlu-Pro-Arg-Ser-Ser-Leu-(therapeutic agent) (SEQ ID NO: 223);
CH₃SO₂-D-HHT-Gly-Arg-Ser-Ser-Leu-(therapeutic agent) (SEQ ID NO: 224);
N-p-tosyl-Gly-Pro-Arg-Ser-Ser-Leu-(therapeutic agent) (SEQ ID NO: 225);
Benzoyl-Val-Gly-Arg-Ser-Ser-Leu-(therapeutic agent) (SEQ ID NO: 226);
CH₃SO₂-D-HHT-Gly-Arg-Ser-Ser-Leu-(therapeutic agent) (SEQ ID NO: 227);
N-α-Z-D-Arg-Gly-Arg-Ser-Ser-Leu-(therapeutic agent) (SEQ ID NO: 228) (Z=benzyloxycarbonyl);
pyroGlu-Gly-Arg-Ser-Ser-Leu-(therapeutic agent) (SEQ ID NO: 229);
H-D-Ile-Pro-Arg-Ser-Ser-Leu-(therapeutic agent) (SEQ ID NO: 230); Cbo-L-(γ)Glu(α-t-BuO)-Gly-Arg-Ser-Ser-Leu-(therapeutic agent) (SEQ ID NO: 231) (Cbo=carbobenzoxy);
H-D-Pro-Phe-Arg-Ser-Ser-Leu-(therapeutic agent) (SEQ ID NO: 232);
H-D-Val-Leu-Arg-Ser-Ser-Leu-(therapeutic agent) (SEQ ID NO: 233);
Bz-Ile-Glu(γ-OH)-Gly-Arg-Ser-Ser-Leu-(therapeutic agent) (SEQ ID NO: 234) (Bz=benzoyl);
Bz-Ile-Glu(γ-OMe)-Gly-Arg-Ser-Ser-Leu-(therapeutic agent) (SEQ ID NO: 235);
Benzoyl-Pro-Phe-Arg-Ser-Ser-Leu-(therapeutic agent) (SEQ ID NO: 236);
H-D-Phe-Pip-Arg-Ser-Ser-Leu-(therapeutic agent) (SEQ ID NO: 237);
H-D-Val-Leu-Lys-Ser-Ser-Leu-(therapeutic agent) (SEQ ID NO: 238);
H-D-Nle-HHT-Lys-Ser-Ser-Leu-(therapeutic agent) (SEQ ID NO: 239);
Pyr-Arg-Thr-Lys-Arg-Ser-Ser-Leu-(therapeutic agent) (SEQ ID NO: 240);
H-Arg-Gln-Arg-Arg-Ser-Ser-Leu-(therapeutic agent) (SEQ ID NO: 241);
Boc-Gln-Gly-Arg-Ser-Ser-Leu-(therapeutic agent) (SEQ ID NO: 242);
Z-Arg-Arg-Ser-Ser-Leu-(therapeutic agent) (SEQ ID NO: 243);
H-D-HHT-Ala-Arg-Ser-Ser-Leu-(therapeutic agent) (SEQ ID NO: 244);
H-D-CHT-Gly-Arg-Ser-Ser-Leu-(therapeutic agent) (SEQ ID NO: 245);
MeSO₂-dPhe-Pro-Arg-Ser-Ser-Leu-(therapeutic agent) (SEQ ID NO: 246);
δ-Z-D-Lys-Pro-Arg-Ser-Ser-Leu-(therapeutic agent) (SEQ ID NO: 247);
CH₃SO₂-D-CHA-But-Arg-Ser-Ser-Leu-(therapeutic agent) (SEQ ID NO: 248);
Ac-Arg-Gln-Ser-Arg-Ser-Ser-Leu-(therapeutic agent) (SEQ ID NO: 249);
Ac-Arg-Arg-Gln-Ser-Arg-Ser-Ser-Leu-(therapeutic agent) (SEQ ID NO: 250);
Ac-Leu-Arg-Arg-Gln-Ser-Arg-Ser-Ser-Leu-(therapeutic agent) (SEQ ID NO: 251);
Ac-Arg-Gln-Ser-Arg-Leu-(therapeutic agent) (SEQ ID NO: 252);
Ac-Arg-Arg-Gln-Ser-Arg-Leu-(therapeutic agent) (SEQ ID NO: 253);
Ac-Leu-Arg-Arg-Gln-Ser-Arg-Ser-Ser-Leu-(therapeutic agent) (SEQ ID NO: 254);
Ac-Leu-Arg-Arg-Gln-Ser-Arg-Ser-Leu-(therapeutic agent) (SEQ ID NO: 255);
Ac-Arg-Arg-Gln-Ser-Arg-Ser-Leu-(therapeutic agent) (SEQ ID NO: 256);
Ac-Leu-Arg-Arg-Gln-Ser-Arg-Ser-Ser-Leu-(therapeutic agent) (SEQ ID NO: 257);
Ac-Arg-Gln-Gly-Arg-Ser-Ser-Leu-(therapeutic agent) (SEQ ID NO: 258);
Ac-Arg-Gln-Ala-Arg-Ser-Ser-Leu-(therapeutic agent) (SEQ ID NO: 259);
Ac-Arg-Gln-Phe-Arg-Ser-Ser-Leu-(therapeutic agent) (SEQ ID NO: 260);
Ac-Arg-Ser-Arg-Ser-Ser-Leu-(therapeutic agent) (SEQ ID NO: 261);
Ac-Arg-Gly-Arg-Ser-Ser-Leu-(therapeutic agent) (SEQ ID NO: 262);
Ac-Arg-Ala-Arg-Ser-Ser-Leu-(therapeutic agent) (SEQ ID NO: 263);
Ac-Arg-Phe-Arg-Ser-Ser-Leu-(therapeutic agent) (SEQ ID NO: 264);
Ac-Gln-Ser-Arg-Ser-Ser-Leu-(therapeutic agent) (SEQ ID NO: 265);
Ac-Gln-Gly-Arg-Ser-Ser-Leu-(therapeutic agent) (SEQ ID NO: 266);
Ac-Gln-Ala-Arg-Ser-Ser-Leu-(therapeutic agent) (SEQ ID NO: 267); and
Ac-Gln-Phe-Arg-Ser-Ser-Leu-(therapeutic agent) (SEQ ID NO: 268).

58. The conjugate of claim 35, wherein P4 is selected from Pro, Arg, Ser, Ala, Lys, Gly, nLeu, Phe and Val.

59. The conjugate of claim 35, wherein:
P2, P3 and/or P4 is/are selected from Pro, Arg, Ser, Ala, Lys, Gly, nLeu, Leu, Tyr, GLu, Phe and Val.

60. The conjugate of claim 35, wherein:
P2, P3 and/or P4 is/are selected from Pro, Arg, Ser, Ala, Lys, Gly, nLeu, Tyr, Glu, Leu Phe and Val; and
P1 is any amino acid.

61. The conjugate of claim 60, wherein P1 is a naturally-occurring amino acid.

62. The conjugate of claim 60, wherein P1 is an amimo acid with an aromatic, branched, or branched aromatic side chain.

63. The conjugate of claim 60, wherein P1 is selected from among Arg, Lys, Tyr, Phe, Trp, Ala, Val, Ile and Thr.

64. The conjugate of claim 60, wherein P1 is Arg, Lys or an Arg surrogate.

65. The conjugate of claim 1, wherein the protease is located at the cell surface by virtue of a specific binding interaction with a receptor therefor.

66. The conjugate of claim 65, wherein the cell surface protease is urokinase plasminogen activator (u-PA) bound to urokinase plasminogen activator receptor (u-PAR).

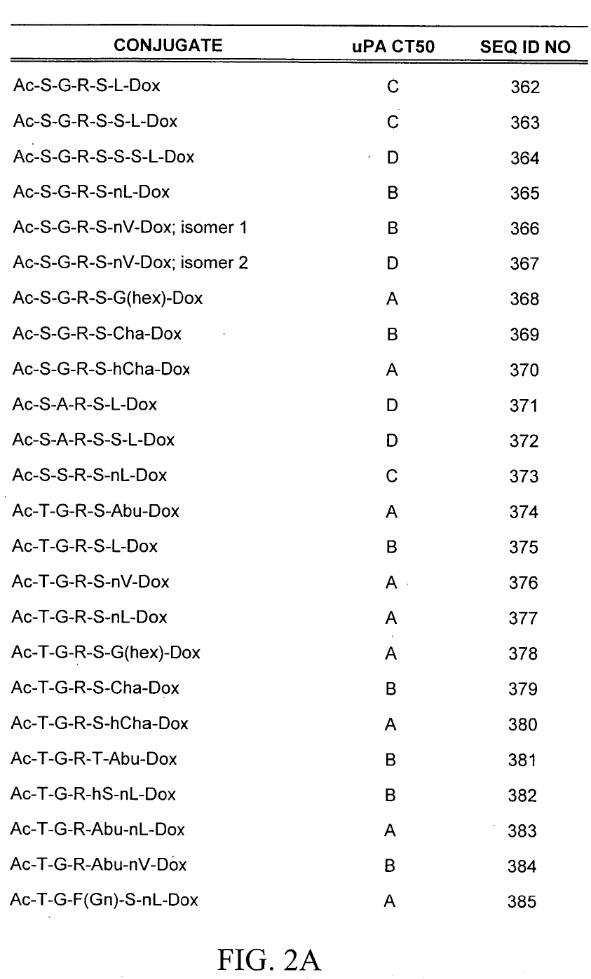

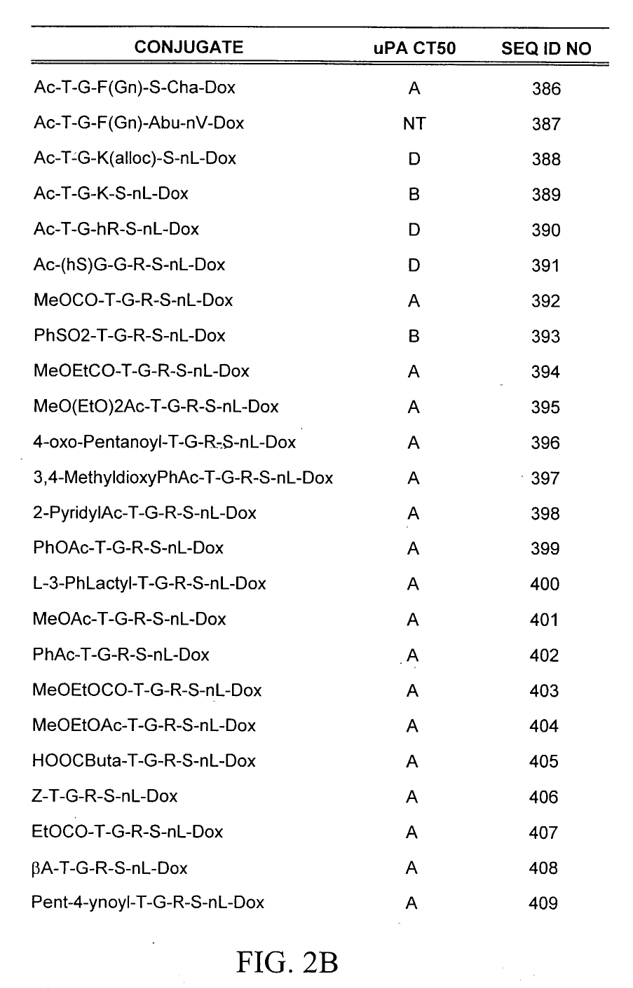

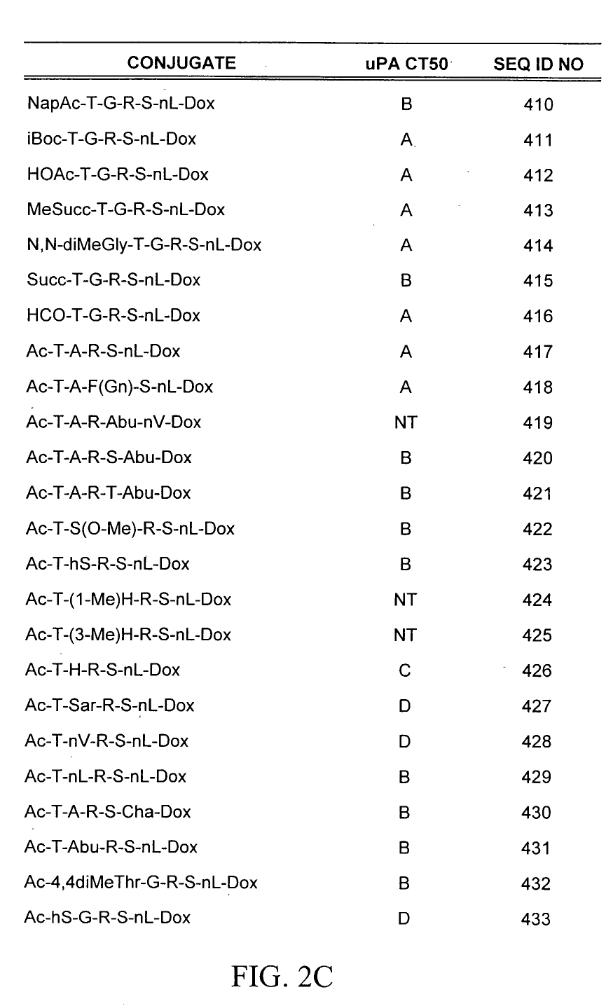

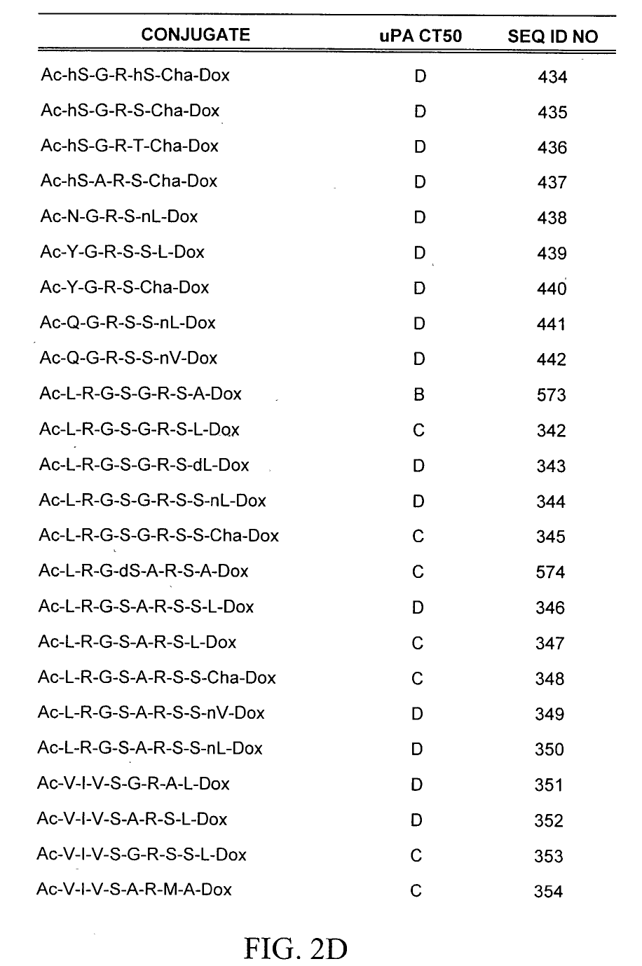

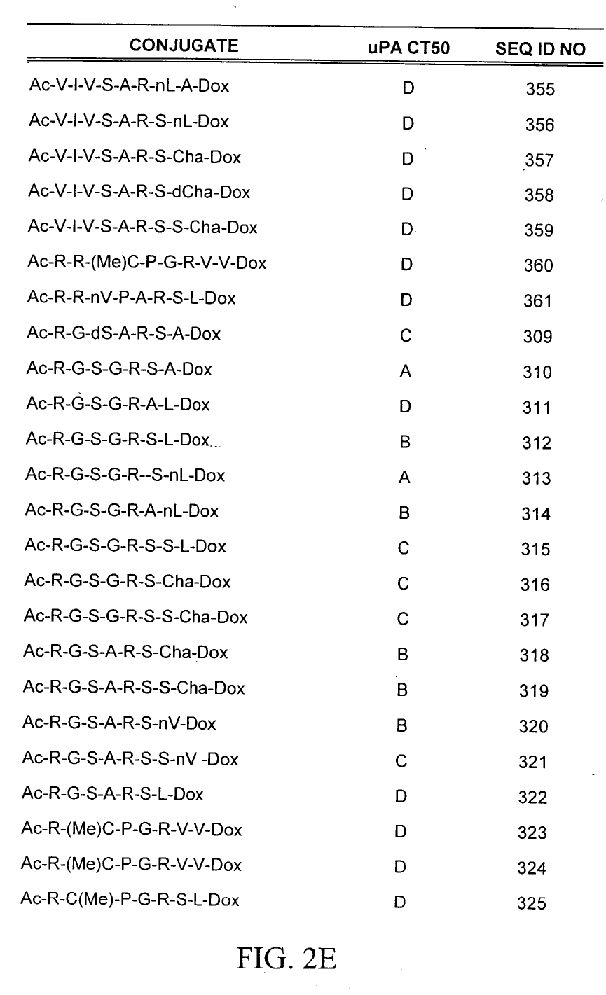

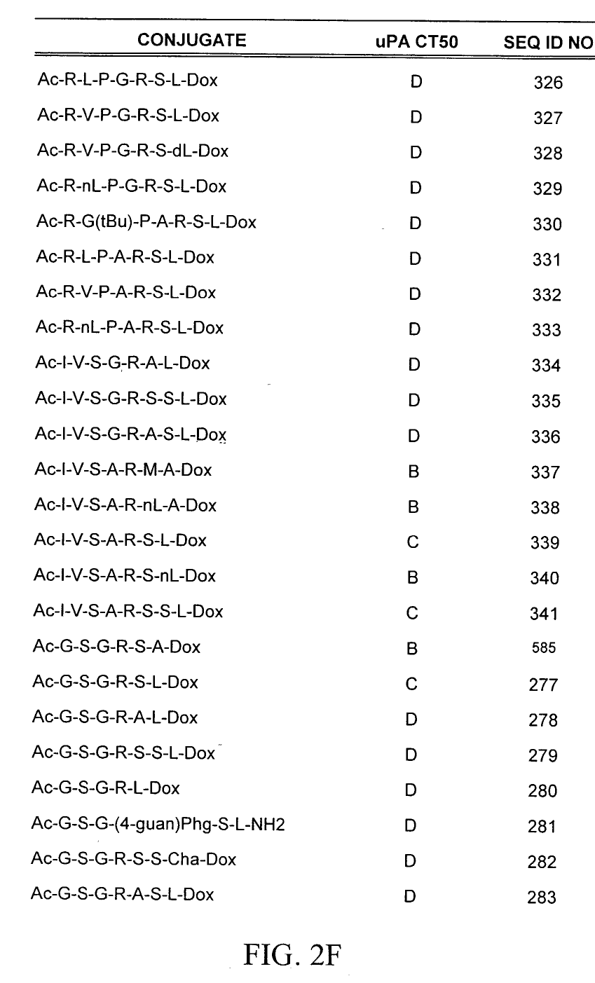

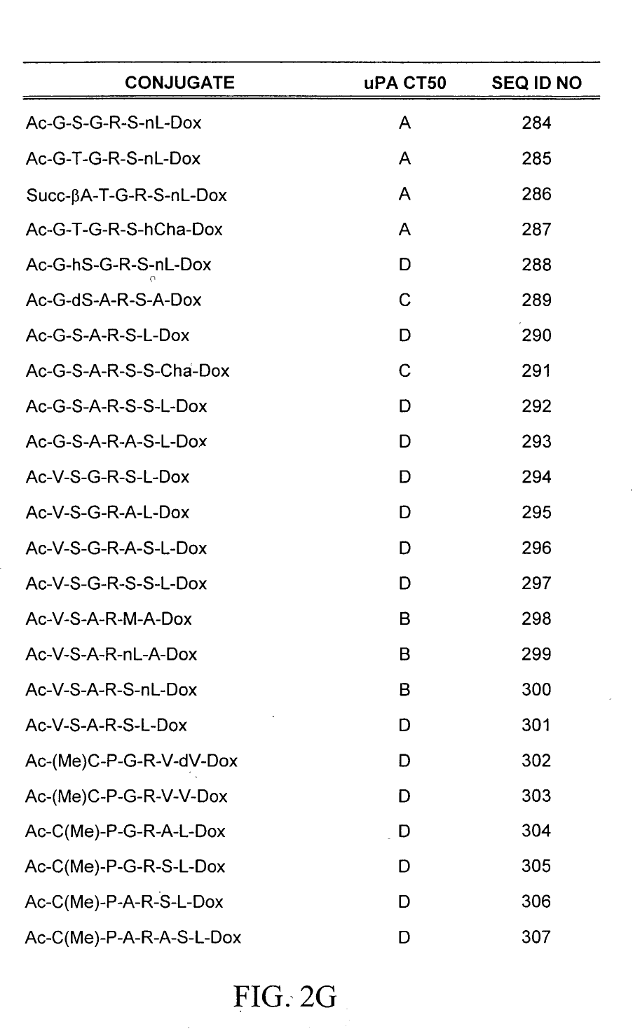

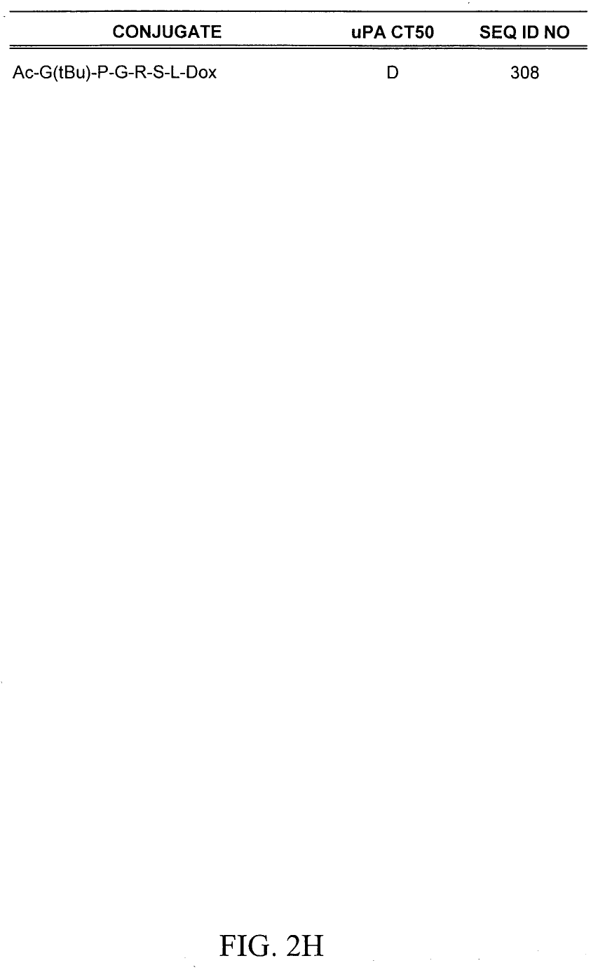

67. The conjugate of claim 1, that comprises a peptidic substrate of the formula P6-P5-P4-P3-P2-P1-P1′-P2′-P3′, wherein each of P1, P2, P3, P4, P5, P6, P1′ and P2′ are selected from residues set forth in FIGS. 1 and 2, and P6, P5, P4, P2′ and P3′ are optional.

68. The conjugate of claim 67, wherein:
P6 is optional and is selected from L, V, R;
P5 is optional and is selected from R, I, L;
P4 is optional and is selected from G, C, V;
P3 is selected from S, dS, P, A or G;
P2 is selected from A or G;
P1 is R;
P1′ is S, V, M or nL;
P2′ is optional and is selected S, L, A or V; and
P3′ is optional and is L.

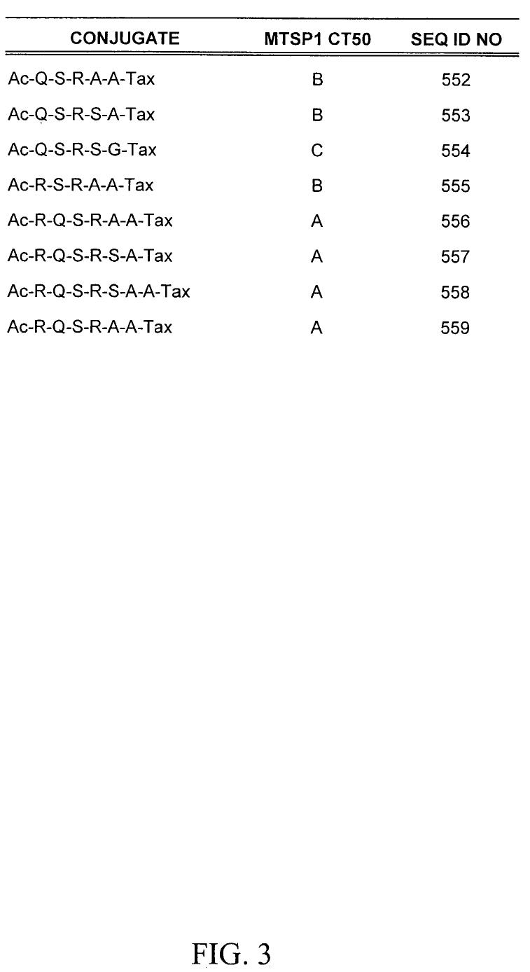

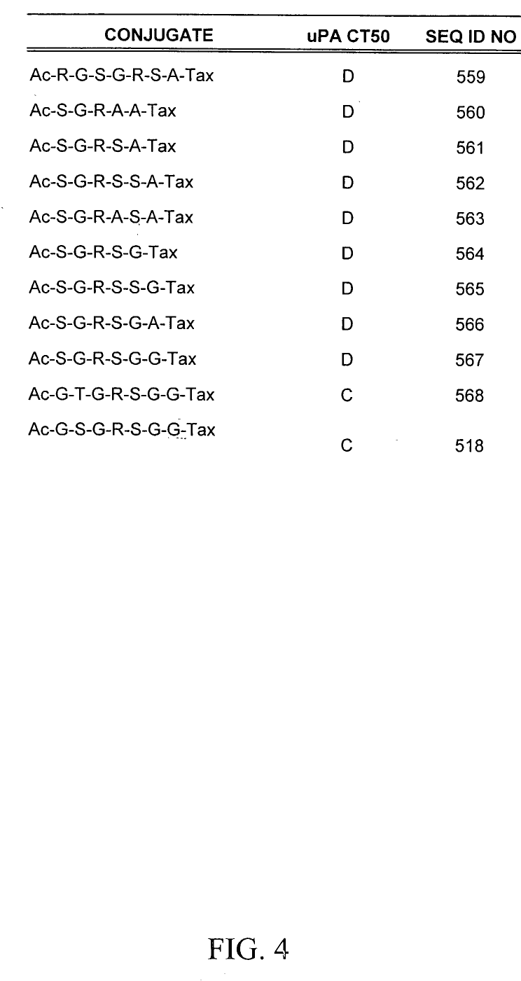

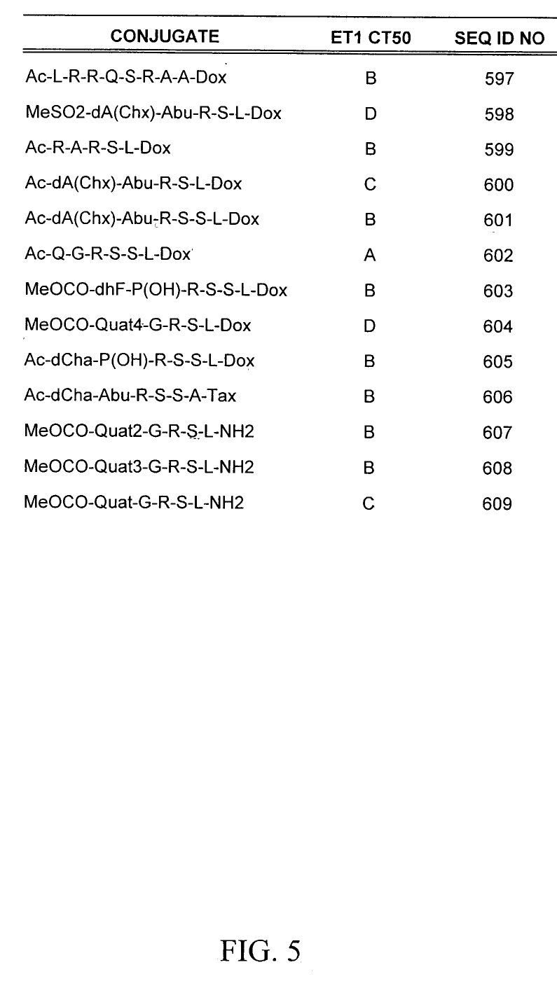

69. A conjugate selected from among those set forth in FIGS. 1-5, wherein the therapeutic agent doxorubicin (Dox) or taxol (Tax) optionally is replaced with any therapeutic agent.

70. The conjugate of claim 65, wherein the therapeutic agent is a toxin, a small organic molecule, a nucleic acid, protein therapeutic agents, a cytokine or a growth factor.

71. The conjugate of claim 65, wherein the therapeutic agent is an anti-cancer agent.

72. The conjugate of claim 65, wherein the therapeutic agent is an anti-angiogenic agent.

73. The conjugate of claim 65, wherein the therapeutic agent is selected from abrin, ricin A, pseudomonas exotoxin shiga toxin, diphtheria toxin, a tumor necrosis factor, α-interferon, γ-interferon, nerve growth factor, tissue factor and tissue factor variants, FAS-ligand platelet derived growth factor, tissue plasminogen activator, interleukin-1 (IL-1), interleukin-2 (IL-2), interleukin-6 (IL-6), granulocyte macrophage colony stimulating factor (GMCSF), granulocyte colony stimulating factor (G-CSF), erythropoietin (EPO), nerve growth factor, fibroblast growth factors (FGFs), and epidermal growth factor.

74. The conjugate of claim 65, wherein the therapeutic agent is selected from alkylating agents, toxins, antiproliferative agents, pro-apoptotic agents, pro-coagulants, cytotoxic nucleosides and tubulin binding agents.

75. The conjugate of claim 65, wherein the therapeutic agent is selected from among the following classes of drugs:
a) anthracycline family of drugs,
b) vinca alkaloid drugs,
c) mitomycins,
d) bleomycins,
e) cytotoxic nucleosides,
f) pteridine family of drugs.
g) diynenes,
h) estramustine,
i) cyclophosphamide,
j) taxanes,
k) podophyllotoxins,
l) maytansanoids,
m) epothilones, and
n) combretastatin and analogs,
or pharmaceutically acceptable derivatives thereof.

76. The conjugate of claim 65, wherein the therapeutic agent is selected from among the following drugs:
a) doxorubicin,
b) carminomycin,
c) daunorubicin,
d) aminopterin,
e) methotrexate,
f) methopterin,
g) dichloromethotrexate,
h) mitomycin C,
i) porfiromycin,
j) 5-fluorouracil,
k) 6-mercaptopurine,
l) cytosine arabinoside,
m) podophyllotoxin,
n) etoposide,
o) etoposide phosphate,
p) melphalan,
q) vinblastine,
r) vincristine,
s) leurosidine,
t) vindesine,
u) estramustine,
v) cisplatin,
w) cyclophosphamide,
x) taxol,
y) leurositte,
z) 4-desacetylvinblastine,
aa) epothilone B,
bb) taxotere,
cc) maytansanol,
dd) epothilone A, and
ee) combretastatin and analogs;
or a pharmaceutically acceptable derivative thereof.

77. The conjugate of claim 65, further comprising a linker between the therapeutic agent and the peptidic substrate.

78. The conjugate of claim 65, wherein the linker comprises a carbohydrate, peptide, and/or hydrocarbon core.

79. The conjugate of claim 77, wherein the linker comprises:
a biscarbonyl alkyl diradical whereby an amine moiety on the therapeutic agent is connected with the linker unit to form an amide bond and the amino terminus of the peptidic substrate is connected with the other end of the linker unit also forming an amide bond; or
a diaminoalkyl diradical linker unit, whereby a carbonyl moiety on the therapeutic agent is covalently attached to one of the amines of the linker unit while the other amine of the linker unit is covalently attached to the C-terminus of the peptidic substrate; or
is a self-eliminating linker of the following formulae: 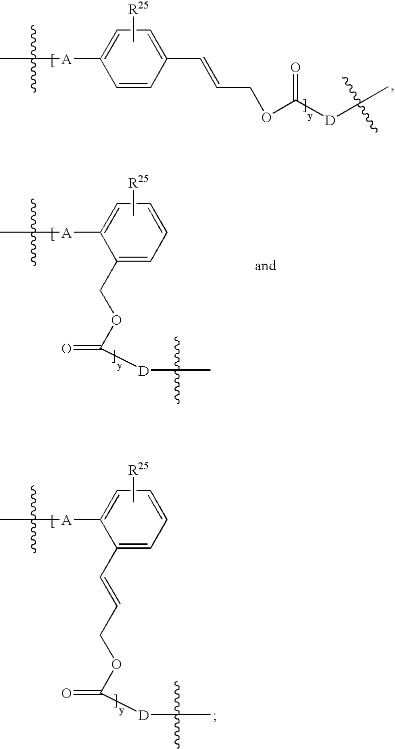
where A is NH or O; D is N(H or alkyl) or O; R²⁵ is H, alkyl, cycloalkyl, cycloalkylalkyl, aryl, heteroaryl optionally substituted with 1 or more, such as 1 to 3, substituents selected from, for example, halo, halo alkyl and alkyl, aralkyl, heteroaralkyl, alkenyl containing 1 to 2 double bonds, alkynyl containing 1 to 2 triple bonds, alk(en)(yn)yl groups, halo, pseudohalo, cyano, hydroxy, haloalkyl and polyhaloalkyl, such as, for example, halo lower alkyl, including trifluoromethyl, formyl, alkylcarbonyl, arylcarbonyl that optionally is substituted with 1 or more, such as, for example, 1 to 3, substituents selected from, for example, halo, halo alkyl and alkyl, heteroarylcarbonyl, carboxy, alkoxycarbonyl, aryloxycarbonyl, aminoimino, alkoxycarbonylamino, aryloxycarbonylamino, aminocarbonyl, alkylaminocarbonyl, dialkylaminocarbonyl, arylaminocarbonyl, diarylaminocarbonyl, aralkylaminocarbonyl, alkoxy, aryloxy, perfluoroalkoxy, alkenyloxy, alkynyloxy, arylalkoxy, aminoalkyl, alkyl-aminoalkyl, dialkylaminoalkyl, arylaminoalkyl, amino, alkylamino, dialkylamino, arylamino, alkylarylamino, alkylcarbonylamino, arylcarbonylamino, azido, nitro, mercapto, alkylthio, arylthio, perfluoroalkylthio, thiocyano, isothiocyano, alkylsulfinyl, alkylsulfonyl, arylsulfinyl, arylsulfonyl, aminosulfonyl, alkylaminosulfonyl, dialkylaminosulfonyl and arylaminosulfonyl; and y is an integer from 1 to 3.

80. The conjugate of claim 77, wherein the linker is a diamine comprising a cyclic alkylene moiety.

81. The conjugate of claim 77, wherein the diamine contains a bicycloalkylene moiety.

82. The conjugate of claim 77, wherein the linker selected from 1,4-bis(aminomethyl)cyclohexane, 1,4-bis(aminomethyl)cycloheptane, 1,3-bis(aminomethyl)cyclopentane, 1-amino-4-(aminomethyl)cyclohexane, 1,4-diaminocyclohexane and 1,4-bis(aminomethyl)bicyclo[2.2.2]octane.

83. The conjugate of claim 77, wherein the linker is a 1,ω-diaminoalkane.

84. The conjugate of claim 77, wherein the linker is a 1,3-diaminopropane.

85. The conjugate of claim 77, wherein the linker is a 1,ω-dicarbonylalkane.

86. The conjugate of claim 77, wherein the linker is selected from oxalic, malonic, succinic, glutaric, adipic and pivalic acids.

87. The conjugate of claim 1, wherein:
the peptidic substrate comprises a P1-P1′ bond;
the P1-P1′ bond is the site of cleavage by a cell surface protease;
P1 is selected from Arg, Lys, Tyr, Phe, Trp, Ala, Val, Ile and Thr; and
P1′ is Gly, Ser, hSer, Thr, Ala, Leu, Ile, d-Ile, nLeu, Val, nVal, Aib, Abu, Met or 6-aminohexanoyl.

88. The conjugate of claim 87, further comprising a P2 residue selected from Phe, Ser, Gly, Ala, Ser(OMe), hSer, 1-methylHis, 3-methylHis, His, nVal, nLeu, Abu, (hS)Gly, Thr, Aib, CHA and Tyr.

89. The conjugate of claim 88, further comprising a P3 residue selected from Arg, Lys, Gin, Quat, Arg surrogates, Ser, Thr, hSer, dSer, Pro, (hS)Gly, Tyr, 4,4-dimethylThr, Asn, Met(O₂), Quat², Quat³, Quat⁴ and Quat⁵.

90. The conjugate of claim 89, further comprising a P4 residue selected from Pro, Arg, Ser, Ala, Lys, Gly, nLeu, Leu, Tyr, Glu, Phe, Val, N,N-dimethylGly, β-Ala, Cys(Me), Gln, t-butylGly and nVal.

91. The conjugate of claim 90, further comprising a P5 residue selected from Ile, Arg and Arg surrogates.

92. The conjugate of claim 91, further comprising a P6 residue selected from Val, Leu, Ile and Val.

93. The conjugate of claim 87, further comprising a P2′ residue selected from Gly, Ser, Ala, Leu, Ile, d-Ile, nLeu, Val, nVal, Aib, Abu, Met, 6-aminohexanoyl, hCHA, CHA, hexylGly, allylGly and Phe.

94. The conjugate of claim 93, further comprising a P3′ residue selected from Gly, Ser, Ala, Leu, Ile, nLeu, Val, nVal, Aib, Abu, Met, 6-aminohexanoyl, CHA and allylGly.

95. The conjugate of claim 94, further comprising a P4′ residue selected from Gly, Ser, Ala, Leu, Ile, nLeu, Val, nVal, Aib, Abu, Met, 6-aminohexanoyl, CHA and allylGly.

96. The conjugate of claim 95, wherein P4′ is Gly, Ser, Ala, Leu, Ile, nLeu, Val, nVal, Aib, Abu, Met and 6-aminohexanoyl.

97. The conjugate of claim 96, wherein P4′ is Leu.

98. The conjugate of claim 1, wherein:
the peptidic substrate comprises a 5-mer that has the formula:
P4-P3-P2-P1-P1′, wherein:
P1 is selected from among Arg, Lys, Tyr, Phe, Trp, Ala, Val, Ile and Thr;
P2 is selected from Phe, Ser, Gly, Ala, Ser(OMe), hSer, 1-methylHis, 3-methylHis, His, nVal, nLeu, Abu, (hS)Gly, Thr, Aib, CHA and Tyr;
P3 is selected from Arg, Lys, Gln, Quat, Arg surrogates, Ser, Thr, hSer, dSer, Pro, (hS)Gly, Tyr, 4,4-dimethylThr, Asn, Met(O₂), Quat², Quat³, Quat⁴ and Quat⁵;
P4 is selected from Pro, Arg, Ser, Ala, Lys, Gly, nLeu, Leu, Tyr, Glu, Phe, Val, N,N-dimethylGly, β-Ala, Cys(Me), Gln, t-butylGly and nVal; and
P1′ is Gly, Ser, hSer, Thr, Ala, Leu, Ile, d-Ile, nLeu, Val, nVal, Aib, Abu, Met or 6-aminohexanoyl.

99. The conjugate of claim 40, wherein:
the peptidic substrate optionally further comprises one or more of a P5 or P2′ amino acid residue, wherein:
P5 is Ile, Arg or an Arg surrogate; and
P2′ is selected from among Gly, Ser, Ala, Leu, Ile, d-Ile, nLeu, Val, nVal, Aib, Abu, Met, 6-aminohexanoyl, hCHA, CHA, hexylGly, allylGly and Phe.

100. The conjugate of claim 41, wherein:
if the peptidic substrate comprises a P5 amino acid residue, then the peptidic substrate optionally further comprises a P6 amino acid residue selected from Arg, Leu, Ile and Val; and
if the peptidic substrate comprises a P2′ amino acid residue, then the peptidic substrate optionally further comprises a P3′ amino acid residue selected from Gly, Ser, Ala, Leu, Ile, nLeu, Val, nVal, Aib, Abu, Met, 6-aminohexanoyl, CHA and allylGly; and
if the peptidic substrate comprises a P3′ amino acid residue, then the peptidic substrate optionally further comprises a P4′ amino acid residue selected from Gly, Ser, Ala, Leu, Ile, nLeu, Val, nVal, Aib, Abu, Met, 6-aminohexanoyl, CHA and allylGly.

101. The conjugate of claim 43 that has formula IV:
Xⁿ-(P6)ₘ-(P5)ₚ-(P4)ᵢ-(P3)ⱼ-(P2)ₗ-P1-(P1′)ᵤ-(P2′)ₖ-(P3′)ᵣ-(P4′)ₛ-(L)ₙ-Z
or a derivative thereof, wherein:
Z is a therapeutic agent;
L is a linker;
l, j, i, p and m are selected as follows:
l is 0 or 1; when l is 0, j, i, p and m are 0; when l is 1, j is 0 or 1; when j is 0, i, p and m are 0; when j is 1, i is 0 or 1; when i is 0, p and m are 0; when i is 1, p is 0 or 1; when p is 0, m is 0; when p is 1, m is 0 or 1;
u, k, r and s are selected as follows:
u is 0 or 1; when u is 0, k, r and s are 0; when u is 1, k is 0 or 1; when k is 0, r and s are 0; when k is 1, r is 0 or 1; when r is 0, s is 0; when r is 1, s is 0 or 1;
n is 0 or 1;
Xⁿ is hydrogen, or an acyl, sulfonyl or carbamoyl cap;
P1 is selected from among Arg, Lys, Tyr, Phe, Trp, Ala, Val, Ile and Thr;
P1′ is Gly, Ser, Ala, Leu, Ile, d-Ile, nLeu, Val, nVal, Aib, Abu, Met or 6-aminohexanoyl;
P2 is selected from Phe, Ser, Gly and Ala;
P3 is selected from Arg, Lys, Gin, Ser, Quat and Arg surrogates;
P4 is selected from Pro, Arg, Ser, Ala, Lys, Gly, nLeu, Leu, Tyr, Glu, Phe and Val;
P5 is selected from Arg and Arg surrogates;
P6 is selected from Leu, Ile and Val;
P2′ is selected from Gly, Ser, Ala, Leu, Ile, d-Ile, nLeu, Val, nVal, Aib, Abu, Met and 6-aminohexanoyl;
P3′ is selected from Gly, Ser, Ala, Leu, Ile, nLeu, Val, nVal, Aib, Abu, Met and 6-aminohexanoyl; and
P4′ is selected from Gly, Ser, Ala, Leu, ile, nLeu, Val, nVal, Aib, Abu, Met and 6-aminohexanoyl.

102. The conjugate of claim 50 that has formula V:
Z-(L)ₙ-(P6)ₘ-(P5)ₚ-(P4)ᵢ-(P3)ⱼ-(P2)ₗ-P1-(P1′)ᵤ-(P2′)ₖ-(P3′)ᵣ-(P4′)ₛ-X^{c}
or a derivative thereof, wherein:
Z is a therapeutic agent;
L is a linker;
l, j, i, p and m are selected as follows:
l is 0 or 1; when l is 0, j, i, p and m are 0; when l is 1, j is 0 or 1; when j is 0, i, p and m are 0; when j is 1, i is 0 or 1; when i is 0, p and m are 0; when i is 1, p is 0 or 1; when p is 0, m is 0; when p is 1, m is 0 or 1;
u, k, r and s are selected as follows:
u is 0 or 1; when u is 0, k, r and s are 0; when u is 1, k is 0 or 1; when k is 0, r and s are 0; when k is 1, r is 0 or 1; when r is 0, s is 0; when r is 1, s is 0 or 1;
n is 0 or 1;
X^{c}, together with the carbonyl group of the amino acid residue to which it is attached, forms a carboxylic acid or a carboxamide group;
P1 is selected from among Arg, Lys, Tyr, Phe, Trp, Ala, Val, Ile and Thr;
P1′ is Gly, Ser, Ala, Leu, Ile, d-Ile, nLeu, Val, nVal, Aib, Abu, Met or 6-aminohexanoyl;
P2 is selected from Phe, Ser, Gly and Ala;
P3 is selected from Arg, Lys, Gln, Ser, Quat and Arg surrogates;
P4 is selected from Pro, Arg, Ser, Ala, Lys, Gly, nLeu, Leu, Tyr, Glu, Phe and Val;
P5 is selected from Arg and Arg surrogates;
P6 is selected from Leu, Ile and Val;
P2′ is selected from Gly, Ser, Ala, Leu, Ile, d-Ile, nLeu, Val, nVal, Aib, Abu, Met and 6-aminohexanoyl;
P3′ is selected from Gly, Ser, Ala, Leu, Ile, nLeu, Val, nVal, Aib, Abu, Met and 6-aminohexanoyl; and
P4′ is selected from Gly, Ser, Ala, Leu, Ile, nLeu, Val, nVal, Aib, Abu, Met and 6-aminohexanoyl.

103. The conjugate of claim 54 that has formula VI:
Z¹-(L¹)ₙ-(P6)ₘ-(P5)ₚ-(P4)ᵢ-(P3)ⱼ-(P2)ₗ-P1-(P1′)ᵤ-(P2′)ₖ-(P3′)ᵣ-(P4′)ₛ-(L²)ᵥ-Z²
or a derivative thereof, wherein:
Z¹ and Z² are each therapeutic agents and are the same or different;
L¹ and L² are each linkers and are the same or different;
l, j, i, p and m are selected as follows:
l is 0 or 1; when l is 0, j, i, p and m are 0; when l is 1, j is 0 or 1; when j is 0, i, p and m are 0; when j is 1, i is 0 or 1; when i is 0, p and m are 0; when i is 1, p is 0 or 1; when p is 0, m is 0; when p is 1, m is 0 or 1;
u, k, r and s are selected as follows:
u is 0 or 1; when u is 0, k, r and s are 0; when u is 1, k is 0 or 1; when k is 0, r and s are 0; when k is 1, r is 0 or 1; when r is 0, s is 0; when r is 1, s is 0 or 1;
n and v are each independently 0 or 1;
P1 is selected from among Arg, Lys, Tyr, Phe, Trp, Ala, Val, Ile and Thr;
P1′ is Gly, Ser, Ala, Leu, Ile, d-Ile, nLeu, Val, nVal, Aib, Abu, Met or 6-aminohexanoyl;
P2 is selected from Phe, Ser, Gly and Ala;
P3 is selected from Arg, Lys, Gin, Ser, Quat and Arg surrogates;
P4 is selected from Pro, Arg, Ser, Ala, Lys, Gly, nLeu, Leu, Tyr, Glu, Phe and Val;
P5 is selected from Arg and Arg surrogates;
P6 is selected from Leu, Ile and Val;
P2′ is selected from Gly, Ser, Ala, Leu, Ile, d-Ile, nLeu, Val, nVal, Aib, Abu, Met and 6-aminohexanoyl;
P3′ is selected from Gly, Ser, Ala, Leu, Ile, nLeu, Val, nVal, Aib, Abu, Met and 6-aminohexanoyl; and
P4′ is selected from Gly, Ser, Ala, Leu, Ile, nLeu, Val, nVal, Aib, Abu, Met and 6-aminohexanoyl.

104. The conjugate of claim 1, selected from:
Ac-R-Q-G-R-S-L-(therapeutic agent) (SEQ ID NO: 491);
Ac-R-Q-G-R-S-S-L-(therapeutic agent) (SEQ ID NO: 492);
Ac-R-Q-G-R-S-nL-(therapeutic agent) (SEQ ID NO: 493);
Ac-R-Q-G-R-S-nV-(therapeutic agent) (SEQ ID NO: 494);
Ac-R-Q-G-R-S-F-(therapeutic agent) (SEQ ID NO: 495);
Ac-R-Q-G-R-A-L-(therapeutic agent) (SEQ ID NO: 496);
Ac-R-Q-G-R-A-L-(therapeutic agent) (SEQ ID NO: 497);
Ac-R-Q-G-R-A-nL-(therapeutic agent) (SEQ ID NO: 498);
Ac-R-Q-G-R-A-nL-(therapeutic agent) (SEQ ID NO: 499);
Ac-R-Q-G-R-A-nV-(therapeutic agent) (SEQ ID NO: 500);
Ac-R-Q-G-R-A-Cha-(therapeutic agent) (SEQ ID NO: 501);
Ac-R-Q-G-R-A-F-(therapeutic agent) (SEQ ID NO: 502);
Ac-R-N-G-R-S-L-(therapeutic agent) (SEQ ID NO: 503);
Ac-R-N-G-R-A-nL-(therapeutic agent) (SEQ ID NO: 504);
Ac-R-Q-A-R-S-L-(therapeutic agent) (SEQ ID NO: 505);
Ac-R-Q-A-R-S-nL-(therapeutic agent) (SEQ ID NO: 506);
Ac-R-Q-A-R-S-nV-(therapeutic agent) (SEQ ID NO: 507);
Ac-R-Q-A-A-S-Cha-(therapeutic agent) (SEQ ID NO: 508);
Ac-R-Q-A-R-S-S-Cha-(therapeutic agent) (SEQ ID NO: 509);
Ac-R-Q-A-R-T-nL-(therapeutic agent) (SEQ ID NO: 510);
Ac-R-Q-A-R-A-L-(therapeutic agent) (SEQ ID NO: 511);
Ac-R-Q-A-R-A-nL-(therapeutic agent) (SEQ ID NO: 512);
Ac-R-Q-A-R-A-nV-(therapeutic agent) (SEQ ID NO: 513);
Ac-R-Q-A-R-A-Cha-(therapeutic agent) (SEQ ID NO: 514);
Ac-R-Q-S-R-A-A-(therapeutic agent) (SEQ ID NO: 515);
Ac-R-Q-S-R-A-(therapeutic agent) (SEQ ID NO: 516);
Ac-R-Q-S-R-A-nL-(therapeutic agent) (SEQ ID NO: 517);
Ac-R-Q-S-R-A-L-(therapeutic agent) (SEQ ID NO: 518);
Ac-R-Q-S-R-A-nV-(therapeutic agent) (SEQ ID NO: 519);
Ac-R-Q-S-R-A-Cha-(therapeutic agent) (SEQ ID NO: 520);
Ac-R-Q-S-R-S-S-L-(therapeutic agent) (SEQ ID NO: 521);
Ac-R-Q-S-R-S-L-(therapeutic agent) (SEQ ID NO: 522);
Ac-R-Q-S-R-S-nL-(therapeutic agent) (SEQ ID NO: 523);
Ac-R-Q-S-R-S-nL-(therapeutic agent) (SEQ ID NO: 524);
Ac-R-Q-S-R-S-nV-(therapeutic agent) (SEQ ID NO: 525);
Ac-R-Q-S-R-S-allylG-(therapeutic agent) (SEQ ID NO: 526);
Ac-R-Q-S-R-S-Cha-(therapeutic agent) (SEQ ID NO: 527);
Ac-R-Q-S-R-T-nL-(therapeutic agent) (SEQ ID NO: 528);
Ac-R-Q-T-R-S-S-L-(therapeutic agent) (SEQ ID NO: 529);
Ac-R-Q-T-R-S-L-(therapeutic agent) (SEQ ID NO: 530);
Ac-R-N-S-R-S-nL-(therapeutic agent) (SEQ ID NO: 531);
Ac-R-Q-F-R-S-L-(therapeutic agent) (SEQ ID NO: 532);
Ac-R-Q-F-R-S-nL-(therapeutic agent) (SEQ ID NO: 534);
Ac-R-Q-F-R-S-nV-(therapeutic agent) (SEQ ID NO: 535);
Ac-R-Q-F-R-S-nL-(therapeutic agent) (SEQ ID NO: 536);
Ac-R-Q-F-R-S-Cha-(therapeutic agent) (SEQ ID NO: 537);
Ac-R-Q-F-R-A-L-(therapeutic agent) (SEQ ID NO: 538);
Ac-R-Q-F-R-A-nL-(therapeutic agent) (SEQ ID NO: 539);
Ac-R-Q-F-R-A-nV-(therapeutic agent) (SEQ ID NO: 540);
Ac-R-Q-F-R-A-Cha-(therapeutic agent) (SEQ ID NO: 541);
Ac-Q-S-R-S-S-nL-(therapeutic agent) (SEQ ID NO: 542);
MeOCO-Quat2-G-R-S-L-(therapeutic agent) (SEQ ID NO: 483);
MeOCO-Quat3-G-R-S-L-(therapeutic agent) (SEQ ID NO: 484);
MeOCO-Quat-G-R-S-L-(therapeutic agent) (SEQ ID NO: 485);
MeOCO-Quat4-G-R-S-L-(therapeutic agent) (SEQ ID NO: 486);
MeOCO-Quat5-G-R-S-L-(therapeutic agent) (SEQ ID NO: 487);
MeOCO-Quat2-G-R-S-S-L-(therapeutic agent) (SEQ ID NO: 488);
MeOCO-Quat4-G-R-S-L-(therapeutic agent) (SEQ ID NO: 489);
MeOCO-Quat2-G-R-S-L-(therapeutic agent) (SEQ ID NO: 490);
Ac-Q-G-R-S-L-(therapeutic agent) (SEQ ID NO: 445);
Ac-Q-G-R-S-S-L-(therapeutic agent) (SEQ ID NO: 446);
Ac-Q-G-R-A-S-L-(therapeutic agent) (SEQ ID NO: 447);
Ac-N-G-R-S-S-L-(therapeutic agent) (SEQ ID NO: 448);
Ac-Q-G-R-S-S-nL-(therapeutic agent) (SEQ ID NO: 449);
Ac-Q-G-R-S-S-nV-(therapeutic agent) (SEQ ID NO: 450);
Ac-Q-G-R-S-S-Cha-(therapeutic agent) (SEQ ID NO: 451);
Ac-Q-G-R-S-S-allylG-(therapeutic agent) (SEQ ID NO: 452);
Ac-Q-G-R-S-S-allylG-(therapeutic agent) (SEQ ID NO: 453);
Ac-Q-A-R-S-L-(therapeutic agent) (SEQ ID NO: 454);
Ac-Q-A-R-S-S-L-(therapeutic agent) (SEQ ID NO: 455);
Ac-Q-S-R-S-L-(therapeutic agent) (SEQ ID NO: 456);
Ac-Q-S-R-S-S-nV-(therapeutic agent) (SEQ ID NO: 457);
Ac-Q-S-R-S-S-Cha-(therapeutic agent) (SEQ ID NO: 458);
Ac-Q-S-R-S-S-L-(therapeutic agent) (SEQ ID NO: 459);
Ac-Q-T-R-S-S-L-(therapeutic agent) (SEQ ID NO: 460);
Ac-Q-Aib-R-S-S-Cha-(therapeutic agent) (SEQ ID NO: 461);
Ac-Q-Aib-R-S-S-L-(therapeutic agent) (SEQ ID NO: 462);
Ac-Q-Abu-R-S-S-Cha-(therapeutic agent) (SEQ ID NO: 463);
Ac-Q-Abu-R-S-S-L-(therapeutic agent) (SEQ ID NO: 464);
Ac-Q-Cha-R-S-S-Cha-(therapeutic agent) (SEQ ID NO: 465);
Ac-Q-F-R-S-L-(therapeutic agent) (SEQ ID NO: 466);
Ac-Q-F-R-S-S-L-(therapeutic agent) (SEQ ID NO: 467);
Ac-Q-Y-R-S-S-L-(therapeutic agent) (SEQ ID NO: 468);
Ac-R-G-R-S-L-(therapeutic agent) (SEQ ID NO: 469);
Ac-R-G-R-S-S-L-(therapeutic agent) (SEQ ID NO: 470);
Ac-R-G-R-S-S-Cha-(therapeutic agent) (SEQ ID NO: 471);
Ac-R-G-R-S-Cha-(therapeutic agent) (SEQ ID NO: 472);
Ac-R-A-R-S-L-(therapeutic agent) (SEQ ID NO: 473);
Ac-R-A-R-S-S-L-(therapeutic agent) (SEQ ID NO: 474);
Ac-R-S-R-S-L-(therapeutic agent) (SEQ ID NO: 475);
Ac-R-S-R-S-S-L-(therapeutic agent) (SEQ ID NO: 476);
Ac-R-S-RS-Cha-(therapeutic agent) (SEQ ID NO: 477);
Ac-R-S-R-S-S-Cha-(therapeutic agent) (SEQ ID NO: 478);
Ac-R-F-R-S-L-(therapeutic agent) (SEQ ID NO: 479);
Ac-R-F-R-S-Cha-(therapeutic agent) (SEQ ID NO: 480);
Ac-Y-G-R-S-S-L-(therapeutic agent) (SEQ ID NO: 481);
Ac-M(O2)-S-R-S-L-(therapeutic agent) (SEQ ID NO: 482);
Ac-R-R-Q-S-R-A-A-(therapeutic agent) (SEQ ID NO: 105);
Ac-R-R-Q-S-R-I-(therapeutic agent) (SEQ ID NO: 610);
Ac-R-R-Q-S-R-S-S-L-(therapeutic agent) (SEQ ID NO: 543);
Ac-R-R-Q-S-R-S-L-(therapeutic agent) (SEQ ID NO: 544);
Ac-R-G-S-G-R-S-L-(therapeutic agent) (SEQ ID NO: 545);
Ac-R-G-S-G-R-S-nL-(therapeutic agent) (SEQ ID NO: 546);
Ac-R-G-S-G-R-A-nL-(therapeutic agent) (SEQ ID NO: 547);
Ac-R-G-S-G-R-S-S-L-(therapeutic agent) (SEQ ID NO: 548);
Ac-I-V-S-G-R-A-S-L-(therapeutic agent) (SEQ ID NO: 549);
Ac-R-R-Q-S-R-A-(therapeutic agent) (SEQ ID NO: 108);
Ac-R-R-Q-S-R-I-(therapeutic agent) (SEQ ID NO: 111);
Ac-L-R-R-Q-S-R-A-A-(therapeutic agent) (SEQ ID NO: 106);
Ac-L-R-R-Q-S-R-G-G-(therapeutic agent) (SEQ ID NO: 109);
Ac-L-R-R-Q-S-R-A-(therapeutic agent) (SEQ ID NO: 110);
Ac-L-R-R-Q-S-R-A-I-(therapeutic agent) (SEQ ID NO: 112);
Ac-L-R-R-Q-S-R-A-I-(therapeutic agent) (SEQ ID NO: 611);
Ac-L-R-R-Q-S-R-S-S-L-(therapeutic agent) (SEQ ID NO: 550);
Ac-L-R-R-Q-S-R-S-L-(therapeutic agent) (SEQ ID NO: 551);
Ac-S-G-R-S-L-(therapeutic agent) (SEQ ID NO: 362);
Ac-S-G-R-S-S-L-(therapeutic agent) (SEQ ID NO: 363);
Ac-S-G-R-S-S-S-L-(therapeutic agent) (SEQ ID NO: 364);
Ac-S-G-R-S-nL-(therapeutic agent) (SEQ ID NO: 365);
Ac-S-G-R-S-nV-(therapeutic agent) (SEQ ID NO: 366); isomer 1
Ac-S-G-R-S-nV-(therapeutic agent) (SEQ ID NO: 367); isomer 2
Ac-S-G-R-S-G(hex)-(therapeutic agent) (SEQ ID NO: 368);
Ac-S-G-R-S-Cha-(therapeutic agent) (SEQ ID NO: 369);
Ac-S-G-R-S-hCha-(therapeutic agent) (SEQ ID NO: 370);
Ac-S-A-R-S-L-(therapeutic agent) (SEQ ID NO: 371);
Ac-S-A-R-S-S-L-(therapeutic agent) (SEQ ID NO: 372);
Ac-S-S-R-S-nL-(therapeutic agent) (SEQ ID NO: 373);
Ac-T-G-R-S-Abu-(therapeutic agent) (SEQ ID NO: 374);
Ac-T-G-R-S-L-(therapeutic agent) (SEQ ID NO: 375);
Ac-T-G-R-S-nV-(therapeutic agent) (SEQ ID NO: 376);
Ac-T-G-R-S-nL-(therapeutic agent) (SEQ ID NO: 377);
Ac-T-G-R-S-G(hex)-(therapeutic agent) (SEQ ID NO: 378);
Ac-T-G-R-S-Cha-(therapeutic agent) (SEQ ID NO: 379);
Ac-T-G-R-S-hCha-(therapeutic agent) (SEQ ID NO: 380);
Ac-T-G-R-T-Abu-(therapeutic agent) (SEQ ID NO: 381);
Ac-T-G-R-hS-nL-(therapeutic agent) (SEQ ID NO: 382);
Ac-T-G-R-Abu-nL-(therapeutic agent) (SEQ ID NO: 383);
Ac-T-G-R-Abu-nV-(therapeutic agent) (SEQ ID NO: 384);
Ac-T-G-F(Gn)-S-nL-(therapeutic agent) (SEQ ID NO: 385);
Ac-T-G-F(Gn)-S-Cha-(therapeutic agent) (SEQ ID NO: 386);
Ac-T-G-F(Gn)-Abu-nV-(therapeutic agent) (SEQ ID NO: 387);
Ac-T-G-K(alloc)-S-nL-(therapeutic agent) (SEQ ID NO: 388);
Ac-T-G-K-S-nL-(therapeutic agent) (SEQ ID NO: 389);
Ac-T-G-hR-S-nL-(therapeutic agent) (SEQ ID NO: 390);
Ac-(hS)G-G-R-S-nL-(therapeutic agent) (SEQ ID NO: 391);
MeOCO-T-G-R-S-nL-(therapeutic agent) (SEQ ID NO: 392);
PhSO2-T-G-R-S-nL-(therapeutic agent) (SEQ ID NO: 393);
MeOEtCO-T-G-R-S-nL-(therapeutic agent) (SEQ ID NO: 394);
Ac-T-G-R-S-nL-(therapeutic agent) (SEQ ID NO: 395);
4-oxo-Pentanoyl-T-G-R-S-nL-(therapeutic agent) (SEQ ID NO: 396);
3,4-MethyldioxyPhAc-T-G-R-S-nL-(therapeutic agent) (SEQ ID NO: 397);
2-PyridylAc-T-G-R-S-nL-(therapeutic agent) (SEQ ID NO: 398);
PhOAc-Ac-T-G-R-S-nL-(therapeutic agent) (SEQ ID NO: 399);
L-3-PhLactyl-T-G-R-S-nL-(therapeutic agent) (SEQ ID NO: 400);
MeOAc-T-G-R-S-nL-(therapeutic agent) (SEQ ID NO: 401);
PhAc-T-G-R-S-nL-(therapeutic agent) (SEQ ID NO: 402);
MeOEtOCO-T-G-R-S-nL-(therapeutic agent) (SEQ ID NO: 403);
MeOEtOAc-T-G-R-S-nL-(therapeutic agent) (SEQ ID NO: 404);
HOOCButa-T-G-R-S-nL-(therapeutic agent) (SEQ ID NO: 405);
Z-T-G-R-S-nL-(therapeutic agent) (SEQ ID NO: 406);
EtOCO-T-G-R-S-nL-(therapeutic agent) (SEQ ID NO: 407);
βA-T-G-R-S-nL-(therapeutic agent) (SEQ ID NO: 408);
Pent-4-ynoyl-T-G-R-S-nL-(therapeutic agent) (SEQ ID NO: 409);
NapAc-T-G-R-S-nL-(therapeutic agent) (SEQ ID NO: 410);
iBoc-T-G-R-S-nL-(therapeutic agent) (SEQ ID NO: 411);
HOAc-T-G-R-S-nL-(therapeutic agent) (SEQ ID NO: 412);
MeSucc-T-G-R-S-nL-(therapeutic agent) (SEQ ID NO: 413);
N,N-diMeGly-T-G-R-S-nL-(therapeutic agent) (SEQ ID NO: 414);
Succ-T-G-R-S-nL-(therapeutic agent) (SEQ ID NO: 415);
HCO-T-G-R-S-nL-(therapeutic agent) (SEQ ID NO: 416);
Ac-T-A-R-S-nL-(therapeutic agent) (SEQ ID NO: 417);
Ac-T-A-F(Gn)-S-nL-(therapeutic agent) (SEQ ID NO: 418);
Ac-T-A-R-Abu-nV-(therapeutic agent) (SEQ ID NO: 419);
Ac-T-A-R-S-Abu-(therapeutic agent) (SEQ ID NO: 420);
Ac-T-A-R-T-Abu-(therapeutic agent) (SEQ ID NO: 421);
Ac-T-S(O-Me)-R-S-nL-(therapeutic agent) (SEQ ID NO: 422);
Ac-T-hS-R-S-nL-(therapeutic agent) (SEQ ID NO: 423);
Ac-T-(1-Me)H-R-S-nL-(therapeutic agent) (SEQ ID NO: 424);
Ac-T-(3-Me)H-R-S-nL-(therapeutic agent) (SEQ ID NO: 425);
Ac-T-H-R-S-nL-(therapeutic agent) (SEQ ID NO: 426);
Ac-T-Sar-R-S-nL-(therapeutic agent) (SEQ ID NO: 427);
Ac-T-nV-R-S-nL-(therapeutic agent) (SEQ ID NO: 428);
Ac-T-nL-R-S-nL-(therapeutic agent) (SEQ ID NO: 429);
Ac-T-A-R-S-Cha-(therapeutic agent) (SEQ ID NO: 430);
Ac-T-Abu-R-S-nL-(therapeutic agent) (SEQ ID NO: 431);
Ac-4,4diMeThr-G-R-S-nL-(therapeutic agent) (SEQ ID NO: 432);
Ac-hS-G-R-S-nL-(therapeutic agent) (SEQ ID NO: 433);
Ac-hS-G-R-hS-Cha-(therapeutic agent) (SEQ ID NO: 434);
Ac-hS-G-R-S-Cha-(therapeutic agent) (SEQ ID NO: 435);
Ac-hS-G-R-T-Cha-(therapeutic agent) (SEQ ID NO: 436);
Ac-hS-A-R-S-Cha-(therapeutic agent) (SEQ ID NO: 437);
Ac-N-G-R-S-nL-(therapeutic agent) (SEQ ID NO: 438);
Ac-Y-G-R-S-S-L-(therapeutic agent) (SEQ ID NO: 439);
Ac-Y-G-R-S-Cha-(therapeutic agent) (SEQ ID NO: 440);
Ac-Q-G-R-S-S-nL-(therapeutic agent) (SEQ ID NO: 441);
Ac-Q-G-R-S-S-nV-(therapeutic agent) (SEQ ID NO: 442);
Ac-L-R-G-S-G-R-S-A-(therapeutic agent) (SEQ ID NO: 573);
Ac-L-R-G-S-G-R-S-L-(therapeutic agent) (SEQ ID NO: 342);
Ac-L-R-G-S-G-R-S-L-(therapeutic agent) (SEQ ID NO: 343);
Ac-L-R-G-S-G-R-S-S-nL-(therapeutic agent) (SEQ ID NO: 344);
Ac-L-R-G-S-G-R-S-S-Cha-(therapeutic agent) (SEQ ID NO: 345);
Ac-L-R-G-dS-A-R-S-A-(therapeutic agent) (SEQ ID NO: 574);
Ac-L-R-G-S-A-R-S-S-L-(therapeutic agent) (SEQ ID NO: 346);
Ac-L-R-G-S-A-R-S-L-(therapeutic agent) (SEQ ID NO: 347);
Ac-L-R-G-S-A-R-S-S-Cha-(therapeutic agent) (SEQ ID NO: 348);
Ac-L-R-G-S-A-R-S-S-nV-(therapeutic agent) (SEQ ID NO: 349);
Ac-L-R-G-S-A-R-S-S-nL-(therapeutic agent) (SEQ ID NO: 350);
Ac-V-I-V-S-G-R-A-L-(therapeutic agent) (SEQ ID NO: 351);
Ac-V-I-V-S-A-R-S-L-(therapeutic agent) (SEQ ID NO: 352);
Ac-V-I-V-S-G-R-S-S-L-(therapeutic agent) (SEQ ID NO: 353);
Ac-V-I-V-S-A-R-M-A-(therapeutic agent) (SEQ ID NO: 354);
Ac-V-I-V-S-A-R-nL-A-(therapeutic agent) (SEQ ID NO: 355);
Ac-V-I-V-S-A-R-S-nL-(therapeutic agent) (SEQ ID NO: 356);
Ac-V-I-V-S-A-R-S-Cha-(therapeutic agent) (SEQ ID NO: 357);
Ac-V-I-V-S-A-R-S-Cha-(therapeutic agent) (SEQ ID NO: 358);
Ac-V-I-V-S-A-R-S-S-Cha-(therapeutic agent) (SEQ ID NO: 359);
Ac-R-R-(Me)C-P-G-R-V-V-(therapeutic agent) (SEQ ID NO: 360);
Ac-R-R-nV-P-A-R-S-L-(therapeutic agent) (SEQ ID NO: 361);
Ac-R-G-dS-A-R-S-A-(therapeutic agent) (SEQ ID NO: 309);
Ac-R-G-S-G-R-S-A-(therapeutic agent) (SEQ ID NO: 310);
Ac-R-G-S-G-R-A-L-(therapeutic agent) (SEQ ID NO: 311);
Ac-R-G-S-G-R-S-L-(therapeutic agent) (SEQ ID NO: 312);
Ac-R-G-S-G-R--S-nL-(therapeutic agent) (SEQ ID NO: 313);
Ac-R-G-S-G-R-A-nL-(therapeutic agent) (SEQ ID NO: 314);
Ac-R-G-S-G-R-S-S-L-(therapeutic agent) (SEQ ID NO: 315);
Ac-R-G-S-G-R-S-Cha-(therapeutic agent) (SEQ ID NO: 316);
Ac-R-G-S-G-R-S-S-Cha-(therapeutic agent) (SEQ ID NO: 317);
Ac-R-G-S-A-R-S-Cha-(therapeutic agent) (SEQ ID NO: 318);
Ac-R-G-S-A-R-S-S-(therapeutic agent) (SEQ ID NO: 319);
Ac-R-G-S-A-R-S-nV-(therapeutic agent) (SEQ ID NO: 320);
Ac-R-G-S-A-R-S-S-nV-(therapeutic agent) (SEQ ID NO: 321);
Ac-R-G-S-A-R-S-L-(therapeutic agent) (SEQ ID NO: 322);
Ac-R-(Me)C-P-G-R-V-V-(therapeutic agent) (SEQ ID NO: 323);
Ac-R-(Me)C-P-G-R-V-V-(therapeutic agent) (SEQ ID NO: 324);
Ac-R-C(Me)-P-G-R-S-L-(therapeutic agent) (SEQ ID NO: 325);
Ac-R-L-P-G-R-S-L-(therapeutic agent) (SEQ ID NO: 326);
Ac-R-V-P-G-R-S-L-(therapeutic agent) (SEQ ID NO: 327);
Ac-R-V-P-G-R-S-L-(therapeutic agent) (SEQ ID NO: 328);
Ac-R-nL-P-G-R-S-L-(therapeutic agent) (SEQ ID NO: 329);
Ac-R-G(tBu)-P-A-R-S-L-(therapeutic agent) (SEQ ID NO: 330);
Ac-R-L-P-A-R-S-L-(therapeutic agent) (SEQ ID NO: 331);
Ac-R-V-P-A-R-S-L-(therapeutic agent) (SEQ ID NO: 332);
Ac-R-nL-P-A-R-S-L-(therapeutic agent) (SEQ ID NO: 333);
Ac-I-V-S-G-R-A-L-(therapeutic agent) (SEQ ID NO: 334);
Ac-I-V-S-G-R-S-S-L-(therapeutic agent) (SEQ ID NO: 335);
Ac-I-V-S-G-R-A-S-L-(therapeutic agent) (SEQ ID NO: 336);
Ac-I-V-S-A-R-M-A-(therapeutic agent) (SEQ ID NO: 337);
Ac-I-V-S-A-R-nL-A-(therapeutic agent) (SEQ ID NO: 338);
Ac-I-V-S-A-R-S-L-(therapeutic agent) (SEQ ID NO: 339);
Ac-I-V-S-A-R-S-nL-(therapeutic agent) (SEQ ID NO: 340);
Ac-I-V-S-A-R-S-S-L-(therapeutic agent) (SEQ ID NO: 341);
Ac-G-S-G-R-S-A-(therapeutic agent) (SEQ ID NO: 585);
Ac-G-S-G-R-S-L-(therapeutic agent) (SEQ ID NO: 277);
Ac-G-S-G-R-A-L-(therapeutic agent) (SEQ ID NO: 278);
Ac-G-S-G-R-S-S-L-(therapeutic agent) (SEQ ID NO: 279);
Ac-G-S-G-R-L-(therapeutic agent) (SEQ ID NO: 280);
Ac-G-S-G-(4-guan)Phg-S-L-(therapeutic agent) (SEQ ID NO: 281);
Ac-G-S-G-R-S-S-Cha-(therapeutic agent) (SEQ ID NO: 282);
Ac-G-S-G-R-A-S-L-(therapeutic agent) (SEQ ID NO: 283);
Ac-G-S-G-R-S-nL-(therapeutic agent) (SEQ ID NO: 284);
Ac-G-T-G-R-S-nL-(therapeutic agent) (SEQ ID NO: 285);
Succ-bA-T-G-R-S-nL-(therapeutic agent) (SEQ ID NO: 286);
Ac-G-T-G-R-S-hCha-(therapeutic agent) (SEQ ID NO: 287);
Ac-G-hS-G-R-S-nL-(therapeutic agent) (SEQ ID NO: 288);
Ac-G-dS-A-R-S-A-(therapeutic agent) (SEQ ID NO: 289);
Ac-G-S-A-R-S-L-(therapeutic agent) (SEQ ID NO: 290);
Ac-G-S-A-R-S-S-Cha-(therapeutic agent) (SEQ ID NO: 291);
Ac-G-S-A-R-S-S-L-(therapeutic agent) (SEQ ID NO: 292);
Ac-G-S-A-R-A-S-L-(therapeutic agent) (SEQ ID NO: 293);
Ac-V-S-G-R-S-L-(therapeutic agent) (SEQ ID NO: 294);
Ac-V-S-G-R-A-L-(therapeutic agent) (SEQ ID NO: 295);
Ac-V-S-G-R-A-S-L-(therapeutic agent) (SEQ ID NO: 296);
Ac-V-S-G-R-S-S-L-(therapeutic agent) (SEQ ID NO: 297);
Ac-V-S-A-R-M-A-(therapeutic agent) (SEQ ID NO: 298);
Ac-V-S-A-R-nL-A-(therapeutic agent) (SEQ ID NO: 299);
Ac-V-S-A-R-S-nL-(therapeutic agent) (SEQ ID NO: 300);
Ac-V-S-A-R-S-L-(therapeutic agent) (SEQ ID NO: 301);
Ac-(Me)C-P-G-R-V-V-(therapeutic agent) (SEQ ID NO: 302);
Ac-(Me)C-P-G-R-V-V-(therapeutic agent) (SEQ ID NO: 303);
Ac-C(Me)-P-G-R-A-L-(therapeutic agent) (SEQ ID NO: 304);
Ac-C(Me)-P-G-R-S-L-(therapeutic agent) (SEQ ID NO: 305);
Ac-C(Me)-P-A-R-S-L-(therapeutic agent) (SEQ ID NO: 306);
Ac-C(Me)-P-A-R-A-S-L-(therapeutic agent) (SEQ ID NO: 307);
Ac-G(tBu)-P-G-R-S-L-(therapeutic agent) (SEQ ID NO: 308);
Ac-Q-S-R-A-A-(therapeutic agent) (SEQ ID NO: 552);
Ac-Q-S-R-S-A-(therapeutic agent) (SEQ ID NO: 553);
Ac-Q-S-R-S-G-(therapeutic agent) (SEQ ID NO: 554);
Ac-R-S-R-A-A-(therapeutic agent) (SEQ ID NO: 555);
Ac-R-Q-S-R-A-A-(therapeutic agent) (SEQ ID NO: 556);
Ac-R-Q-S-R-S-A-(therapeutic agent) (SEQ ID NO: 557);
Ac-R-Q-S-R-S-A-A-(therapeutic agent) (SEQ ID NO: 558);
Ac-R-G-S-G-R-S-A-(therapeutic agent) (SEQ ID NO: 559);
Ac-S-G-R-A-A-(therapeutic agent) (SEQ ID NO: 560);
Ac-S-G-R-S-A-(therapeutic agent) (SEQ ID NO: 561);
Ac-S-G-R-S-S-A-(therapeutic agent) (SEQ ID NO: 562);
Ac-S-G-R-A-S-A-(therapeutic agent) (SEQ ID NO: 563);
Ac-S-G-R-S-G-(therapeutic agent) (SEQ ID NO: 564);
Ac-S-G-R-S-S-G-(therapeutic agent) (SEQ ID NO: 565);
Ac-S-G-R-S-G-A-(therapeutic agent) (SEQ ID NO: 566);
Ac-S-G-R-S-G-G-(therapeutic agent) (SEQ ID NO: 567);
Ac-G-T-G-R-S-G-G-(therapeutic agent) (SEQ ID NO: 568);
Ac-G-S-G-R-S-G-G-(therapeutic agent) (SEQ ID NO: 243)
Ac-L-R-R-Q-S-R-A-A-(therapeutic agent) (SEQ ID NO: 597);
MeSO2-dA(Chx)-Abu-R-S-L-(therapeutic agent) (SEQ ID NO: 598);
Ac-R-A-R-S-L-(therapeutic agent) (SEQ ID NO: 599);
Ac-dA(Chx)-Abu-R-S-L-(therapeutic agent) (SEQ ID NO: 600);
Ac-dA(Chx)-Abu-R-S-S-L-(therapeutic agent) (SEQ ID NO: 601);
Ac-Q-G-R-S-S-L-(therapeutic agent) (SEQ ID NO: 602);
MeOCO-dhF-P(OH)-R-S-S-L-(therapeutic agent) (SEQ ID NO: 603);
MeOCO-Quat4-G-R-S-L-(therapeutic agent) (SEQ ID NO: 604);
Ac-dCha-P(OH)-R-S-S-L-(therapeutic agent) (SEQ ID NO: 605);
Ac-dCha-Abu-R-S-S-A-(therapeutic agent) (SEQ ID NO: 606);
MeOCO-Quat2-G-R-S-L-(therapeutic agent) (SEQ ID NO: 607);
MeOCO-Quat3-G-R-S-L-(therapeutic agent) (SEQ ID NO: 608); and
MeOCO-Quat-G-R-S-L-(therapeutic agent) (SEQ ID NO: 609).

105. The conjugate of claim 35, wherein P4 is selected from Pro, Arg, Ser, Ala, Lys, Gly, nLeu, Leu, Tyr, Glu, Phe, Val, N,N-dimethylGly, β-Ala, Cys(Me), Gln, t-butylGly and nVal.

106. The conjugate of claim 1, that comprises a peptidic substrate of the formula P6-P5-P4-P3-P2-P1-P1′-P2′-P3′-P4′, wherein each of P1, P2, P3, P4, P5, P6, P1′ and P2′ are selected from residues set forth in FIGS. 1 and 2, and P6, P5, P4, P2′, P3′ and P4′ are optional.

107. The conjugate of claim 67, wherein:
P6 is optional and is selected from L, V, R;
P5 is optional and is selected from R, I, L;
P4 is optional and is selected from G, C, V;
P3 is selected from S, dS, P, A or G;
P2 is selected from A or G;
P1 is R;
P1′ is S, V, M or nL;
P2′ is optional and is selected S, L, A or V;
P3′ is optional and is L; and
P4′ is optional and is L.

108. A conjugate, comprising a therapeutic agent and a nucleic acid substrate linked thereto via a peptidic linker, wherein the peptidic linker is proteolytically cleaved by a cell surface protease or a soluble, released or shed form thereof, to liberate the therapeutic agent, wherein the conjugate is not substantially cleaved by plasmin or prostate specific antigen (PSA).

109. The conjugate of claim 108, wherein the nucleic acid is DNA.

110. The conjugate of claim 108, wherein the nucleic acid is RNA.

111. The conjugate of claim 108, wherein the nucleic acid is double-stranded RNA.

112. The conjugate of claim 67, wherein:
P6 is optional and is selected from L, V, R;
P5 is optional and is selected from R, I, L;
P4 is optional and is selected from G, C, V;
P3 is selected from S, dS, P, A or G;
P2 is selected from A or G;
P1 is R;
P1′ is T, Abu, hS, nV or A;
P2′ is optional and is selected S, L, A or V;
P3′ is optional and is L, nL, nV, G(hex), G(allyl), CHA, hCHA, or Abu; and
P4′ is optional and is L, nL, nV, G(hex), G(allyl), CHA, hCHA, or Abu.

113. The conjugate of claim 67, wherein:
P6 is optional and is selected from L, V, R;
P5 is optional and is selected from R, I, L;
P4 is optional and is selected from G, C, V;
P3 is selected from S, dS, P, A or G;
P2 is selected from A or G;
P1 is R;
P1′ is S, G or A;
P2′ is optional and is selected G or A;
P3′ is optional and is L, nL, nV, G(hex), G(allyl), CHA, hCHA, or Abu; and
P4′ is optional and is L, nL, nV, G(hex), G(allyl), CHA, hCHA, or Abu.

114. The conjugate of claim 1, wherein the therapeutic agent is taxol.

115. The conjugate of claim 1, wherein the therapeutic agent is doxorubicin.

116. A method of treatment of a disease, comprising administering a conjugate of claim 1 to a subject, wherein the disease is a cell-surface protease-associated disease.

117. The method of claim 116, wherein the disease is selected from the group consisting of autoimmune disesases, inflammatory diseases, infectious diseases and endocrine diseases.

118. The method of claim 116, wherein the disease is a proliferative disease.

119. A method of treatment of a cell-surface protease-associated disease, comprising administering a conjugate, comprising a therapeutic agent and a peptidic substrate linked thereto optionally via a linker, wherein the peptidic substrate is proteolytically cleaved by a cell surface protease or a soluble, released or shed form thereof to liberate the therapeutic agent, to a subject exhibiting symptoms of a cell-surface protease-associated disorder.

120. The method of claim 119, wherein the disease is selected from the group consisting of autoimmune disesases, inflammatory diseases, infectious diseases and endocrine diseases.

121. The method of claim 119, wherein the disease is a proliferative disease.

122. The method of claim 114, wherein the subject is a mammal.

123. The method of claim 120, wherein the mammal is a human.

124. The method of claim 118, wherein the disease is cancer.

125. The method of claim 118, wherein the disease is selected from ocular disorders, cardiovascular disorders, chronic inflammatory diseases, wounds, circulatory disorders, dermatological disorders and cancer.

126. The method of claim 118, wherein the disease is selected from rheumatoid arthritis, psoriasis, diabetic retinopathies, recurrence of pterygii, scarring from excimer laser surgery, scarring from glaucoma filtering surgery, macular degeneration anterior eye, crest syndromes, solid neoplasms and vascular tumors.

127. The method of claim 118, wherein the disease is selected from lung cancer, colon cancer, pancreatic cancer, esophageal cancer, breast cancer, ovarian cancer, prostate cancer, melanoma and Kaposi's sarcoma.

128. The method of claim 116, wherein the therapeutic agent is taxol.

129. The method of claim 116, wherein the therapeutic agent is doxorubicin.

130. A pharmaceutical composition, comprising the conjugate of claim 1 or a pharmaceutically acceptable derivative thereof, in a pharmaceutically acceptable carrier.

131. The pharmaceutical composition of claim 130 that is formulated for single dosage administration.

132. An article of manufacture, comprising packaging material, the conjugate of claim 1, or a pharmaceutically acceptable derivative thereof, contained within packaging material, which is used for treatment, prevention or amelioration of one or more symptoms associated with cell-surface protease-associated diseases or disorders, and a label that indicates that the conjugate or pharmaceutically acceptable derivative thereof is used for treatment, prevention or amelioration of one or more symptoms associated with cell-surface protease-associated diseases or disorders.

133. A method of preparing a conjugate of claim 1, comprising:
a) synthesizing the peptidic substrate;
b) optionally capping the peptidic substrate on either the N-terminus or the C-terminus;
c) optionally linking the non-capped terminus of the peptidic substrate to a linker;
d) coupling the peptidic substrate to a therapeutic agent, optionally via the linker, to form a conjugate; and
e) optionally, deprotecting the conjugate, if protected.

134. The method of claim 133, wherein, prior to step a), the method comprises a step of identifying a peptidic substrate for the protease.

135. A method, comprising:
a) selecting a disease;
b) identifying a cell involved in the disease process or a cell in the vicinity of the cell involved in the disease process; and
c) identifying a cell surface protease on the cell, thereby identifying proteases to target conjugates for treatment of diseases.

136. The method of claim 135, further comprising preparing a conjugate that targets the protease.
